(19) United States
(12) Patent Application Publication
(10) Pub. No.: US 2025/0032540 A1
(43) Pub. Date: Jan. 30, 2025

(54) COMPOSITIONS AND METHODS FOR CELLULAR IMMUNOTHERAPY

(71) Applicant: Fred Hutchinson Cancer Center, Seattle, WA (US)

(72) Inventors: Thomas M. SCHMITT, Seattle, WA (US); Megan S. MCAFEE, Wayland, MA (US); Aude G. CHAPUIS, Seattle, WA (US)

(21) Appl. No.: 18/257,277

(22) PCT Filed: Dec. 14, 2021
(86) PCT No.: PCT/US2021/063409

§ 371 Date: Jun. 13, 2023

Related U.S. Application Data
(60) Provisional application No. 63/125,347, filed on Dec. 14, 2020.

(51) Int. Cl. A61K 35/17; A61K 39/00; A61P 35/00; C07K 14/705; C07K 14/725; C12N 5/0783

(52) U.S. Cl. CPC A61K 35/17; A61K 39/4611; A61K 39/4632; A61K 39/464486; A61P 35/00; C07K 14/7051; C07K 14/70517; C07K 14/70521; C12N 5/0636; C07K 2319/03

(57) ABSTRACT

The present application concerns chimeric co-receptor constructs, especially CD8-alpha (CD8α), CD8-beta (CD8β) or CD3-zeta (CD3ζ) chains comprising an intracellular co-stimulatory domain. In particular, the application discloses fusion proteins comprising the extracellular domain of CD8-alpha (CD8α), CD8-beta (CD8β) or CD3-zeta (CD3ζ) chains, a transmembrane domain, and an intracellular co-stimulatory domain, especially that of CD28, 4-1BB (CD137), and others. These engineered polypeptides and expression constructs are useful to confer to, or improve, a desired activity or function of a host cell, such as an immune cell that targets a diseased or pathogenic cell (e.g. a cancer cell). These polypeptides may improve cellular function, such as in the context of adoptive cell therapy, for example, comprising CD4+ T cells expressing an antigen-specific receptor.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is 360056_46401WO_SEQUENCE_LISTING.txt. The text file is 285 KB, was created on Dec. 10, 2021, and is being submitted electronically via EFS-Web.

### BACKGROUND

Adoptive transfer of tumor-specific T-cells is an appealing strategy to eliminate existing tumors and requires the establishment of a robust population of antigen-specific T cells in vivo to eliminate existing tumor and prevent recurrences (Stromnes et al., Immunol. Rev. 257:145, 2014). Various adoptive T cell therapies have been developed using TCRs that specifically recognize cancer antigens (see, e.g., PCT Publication Nos. WO 2016/022400; WO 2018/170338; WO 2018/090057; WO 2017/112944; WO 2017/193104; WO 2018/058002; and WO 2013/071154). Some cell therapy compositions that target a single antigen comprise both CD8⁺ T cells (which naturally express MHC-I-restricted TCR) and CD4⁺ T cells (which naturally express MHC-II-restricted TCR) (see, e.g., Sommermeyer et al., Leukemia 30 (2): 1888 (2016)). Compositions and methods are needed for new or improved adoptive cell therapies.

### BRIEF DESCRIPTION OF THE DRAWINGS

FIG. 1 shows schematic diagrams of certain co-receptor fusion protein constructs according to the present disclosure. In Constructs 1-5, a portion of a human CD28 intracellular domain was fused to a truncated human CD8 co-receptor β chain (Constructs 1 (corresponding to Construct G in Table 1), 2 (corresponding to Construct A in Table 1), 3 (corresponding to Construct B in Table 1)), to a truncated human CD8 co-receptor α chain (Construct 5, corresponding to Construct D in Table 1), or to each of a truncated human CD8 co-receptor α chain and a truncated human CD8 co-receptor β chain (Construct 4, corresponding to Construct C in Table 1). In Constructs 6-8, a signaling portion of: a human 4-1BB intracellular domain (Construct 6, corresponding to Construct H in Table 1); a human ICOS intracellular domain (Construct 7, corresponding to Construct I in Table 1); or a human OX40 intracellular domain (Construct 8, corresponding to Construct J in Table 1) was fused to a truncated human CD8 co-receptor β chain.

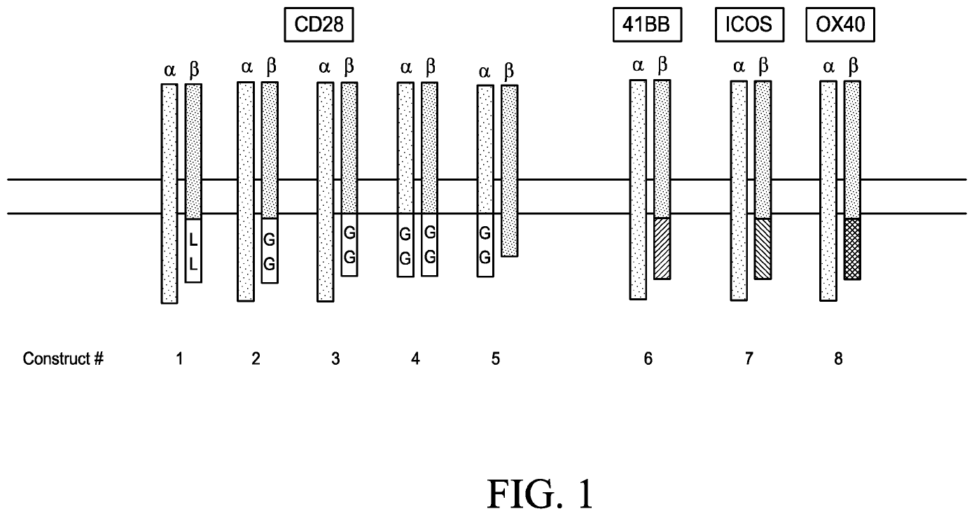

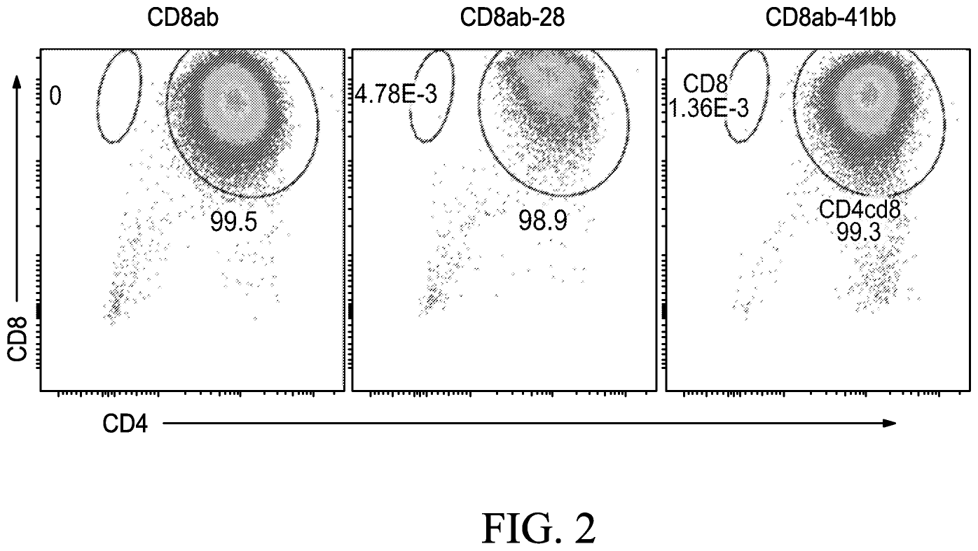

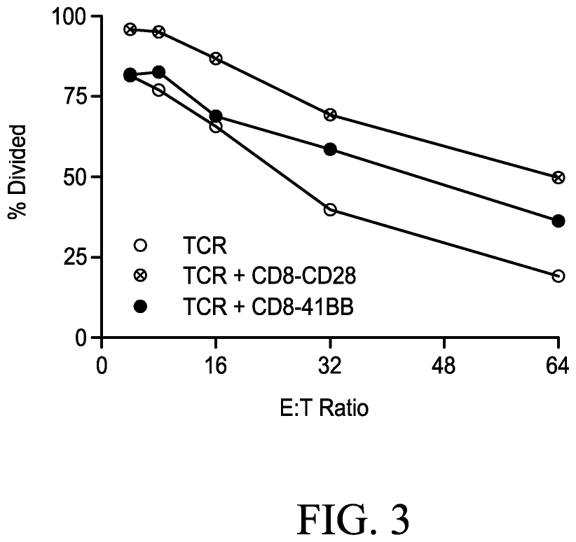

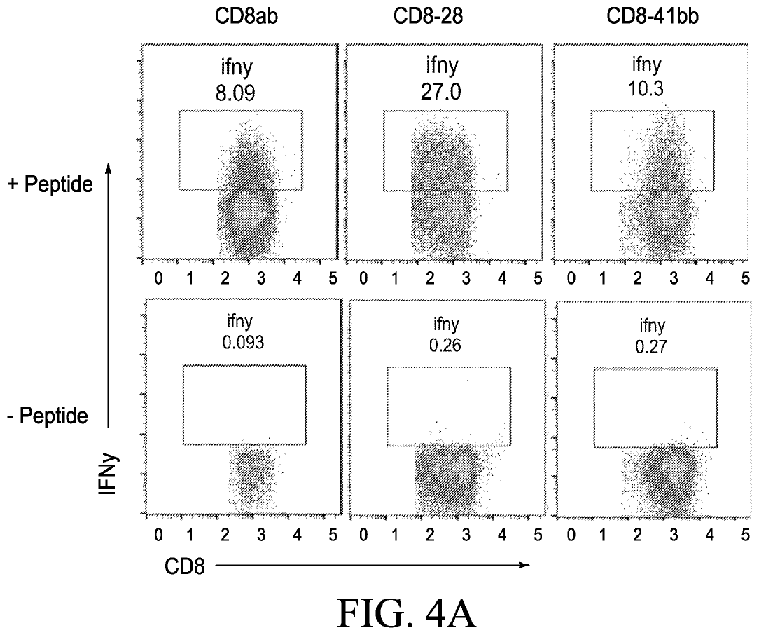

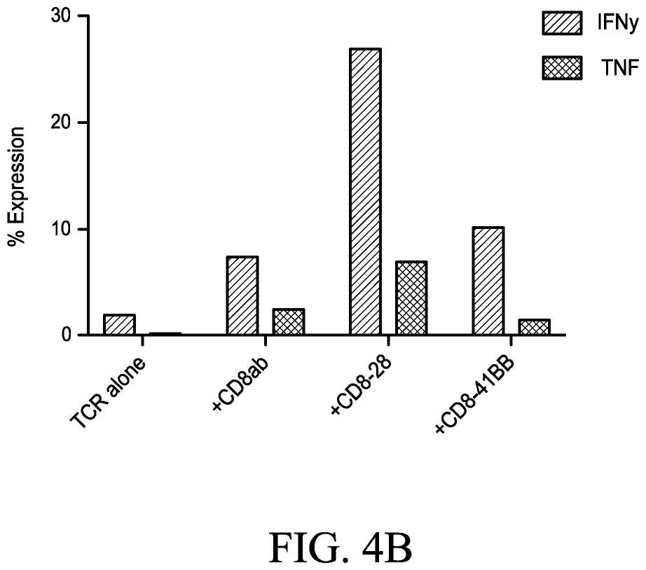

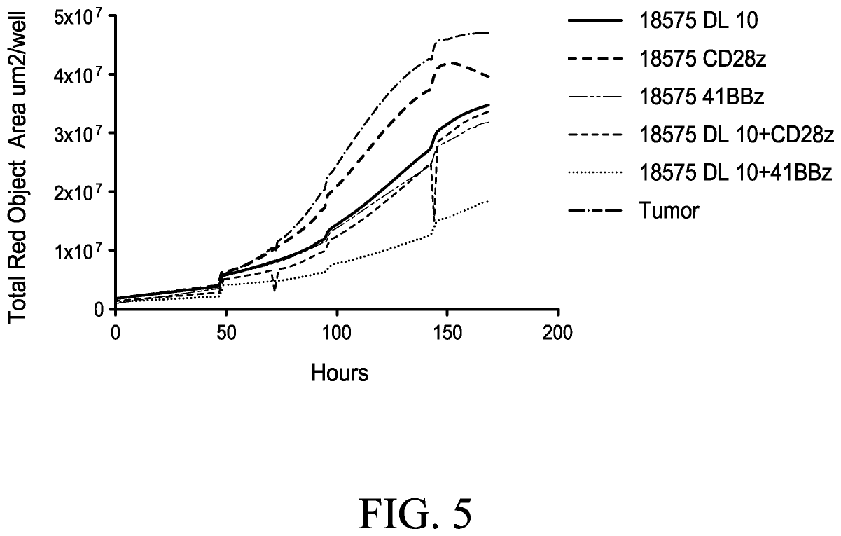

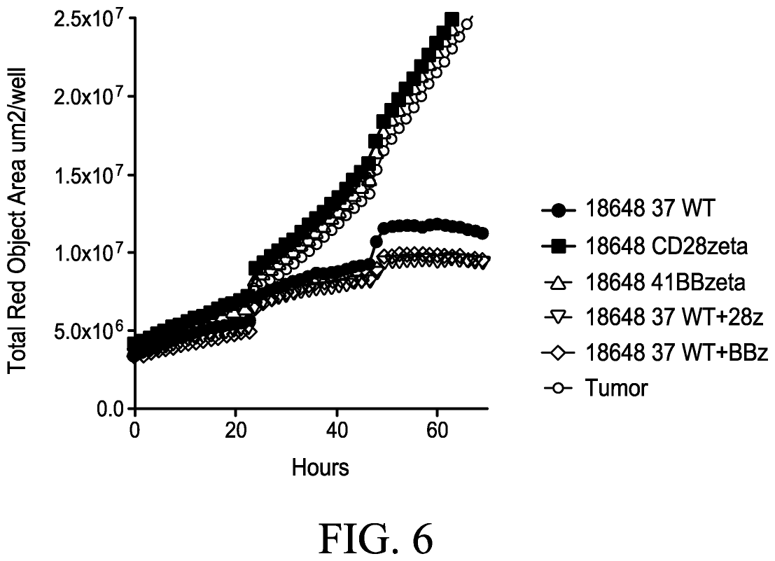

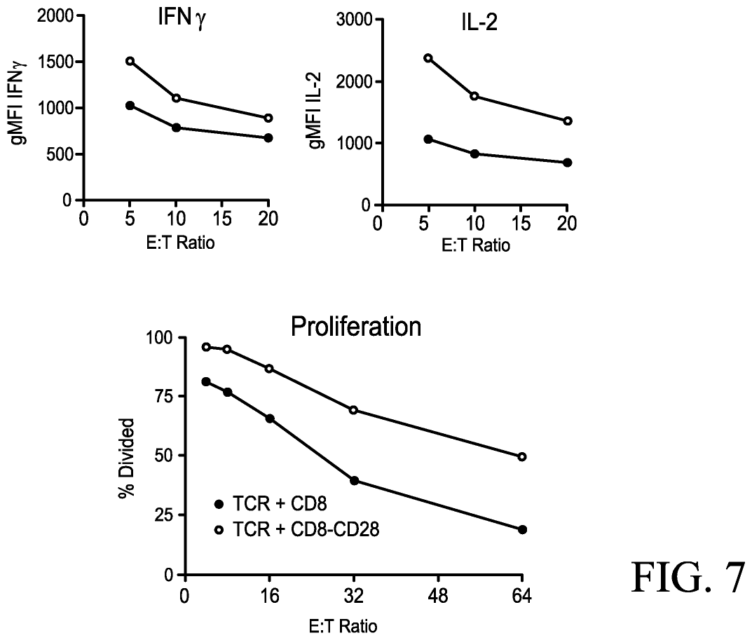

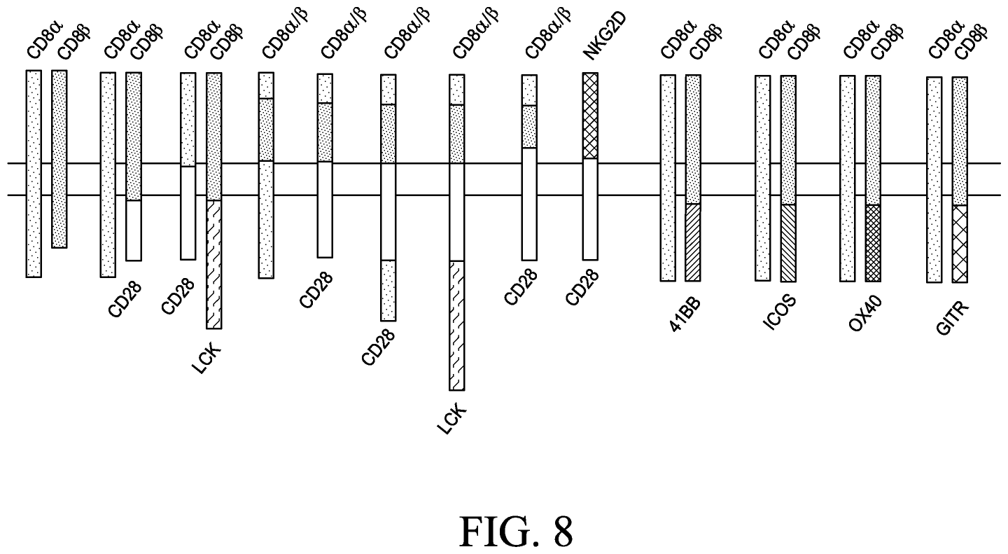

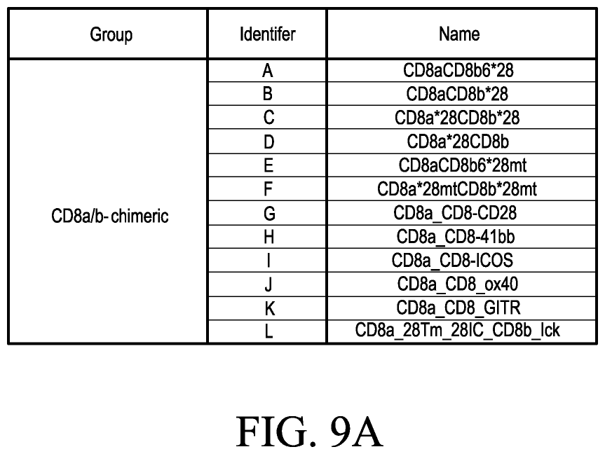

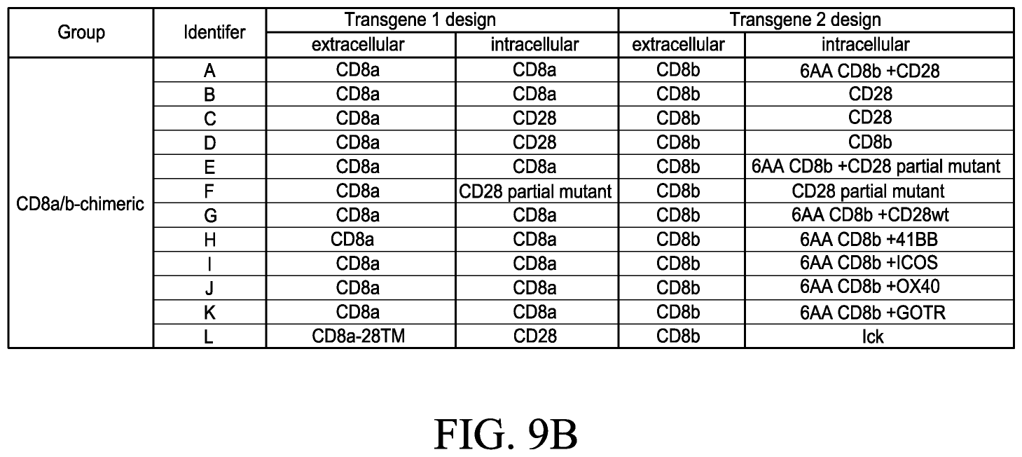

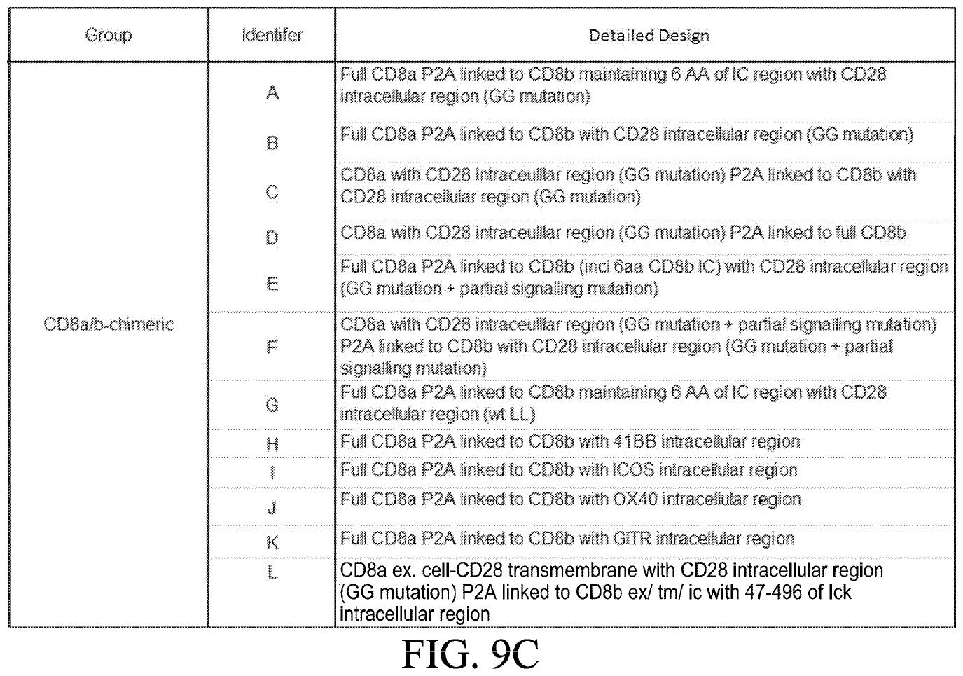

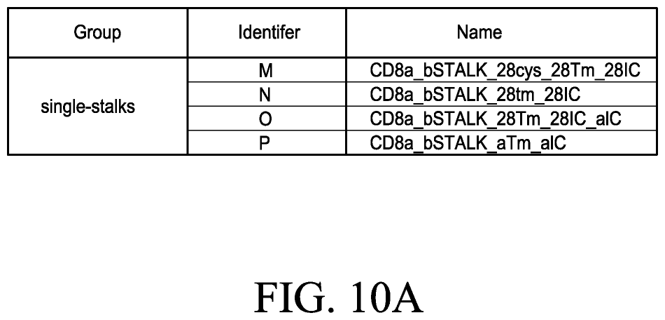

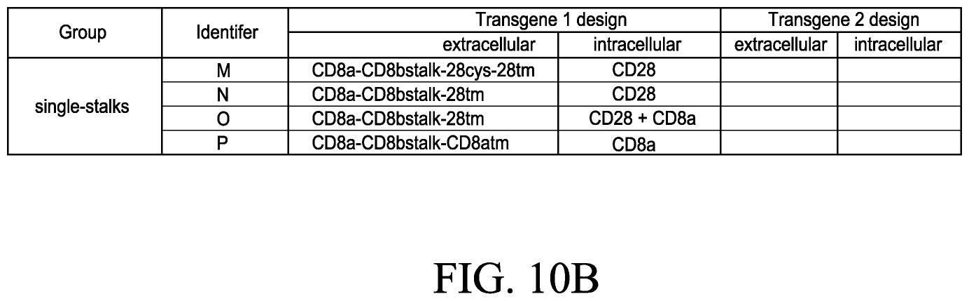

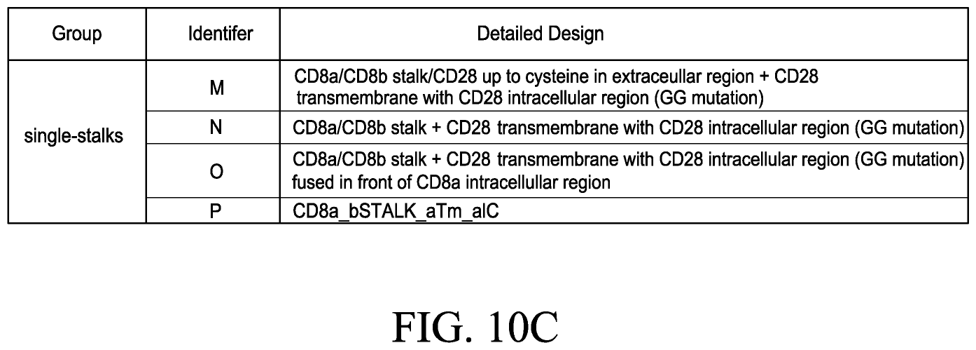

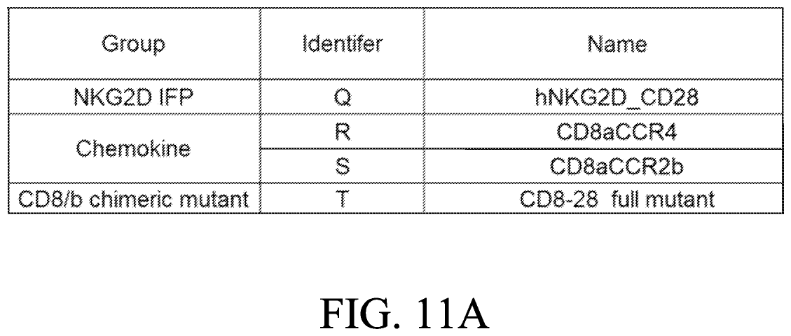

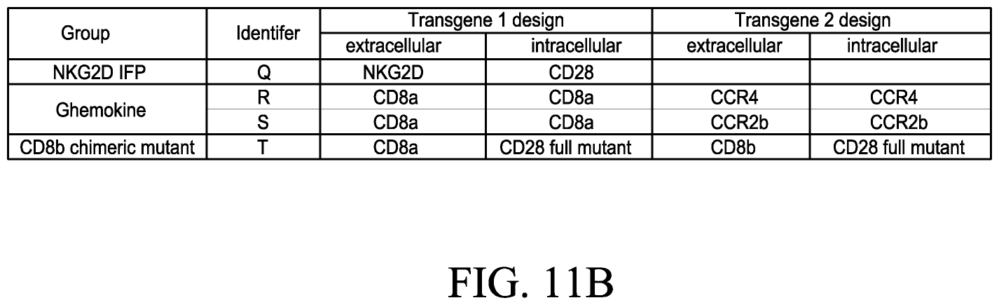

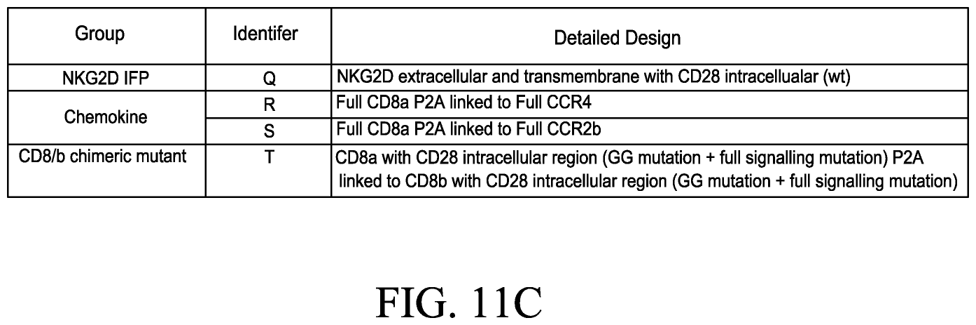

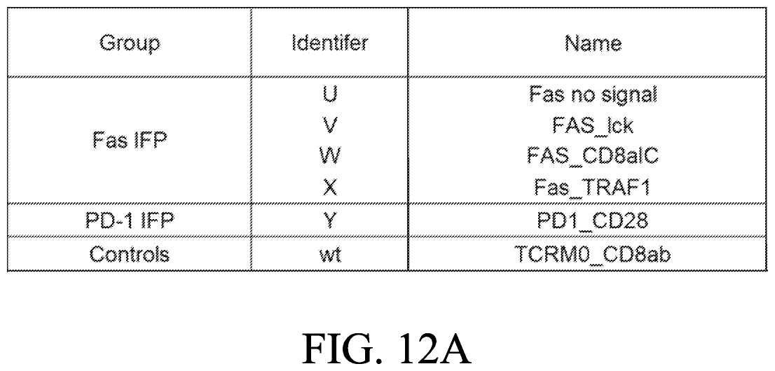

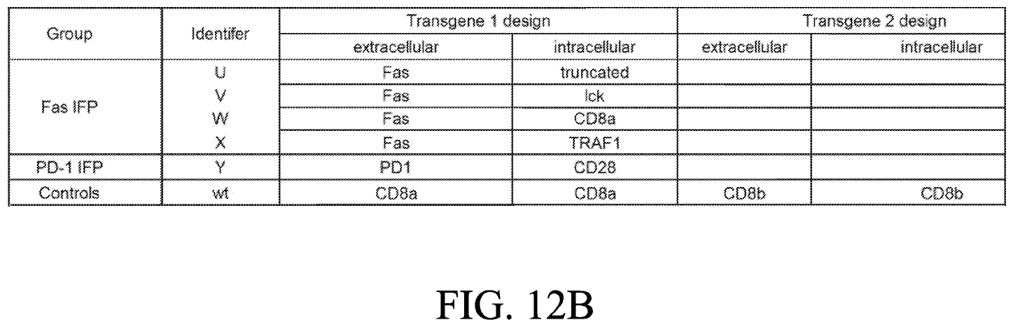

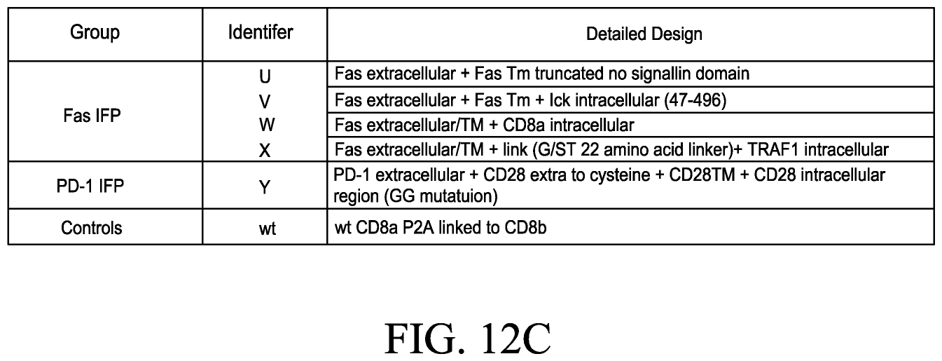

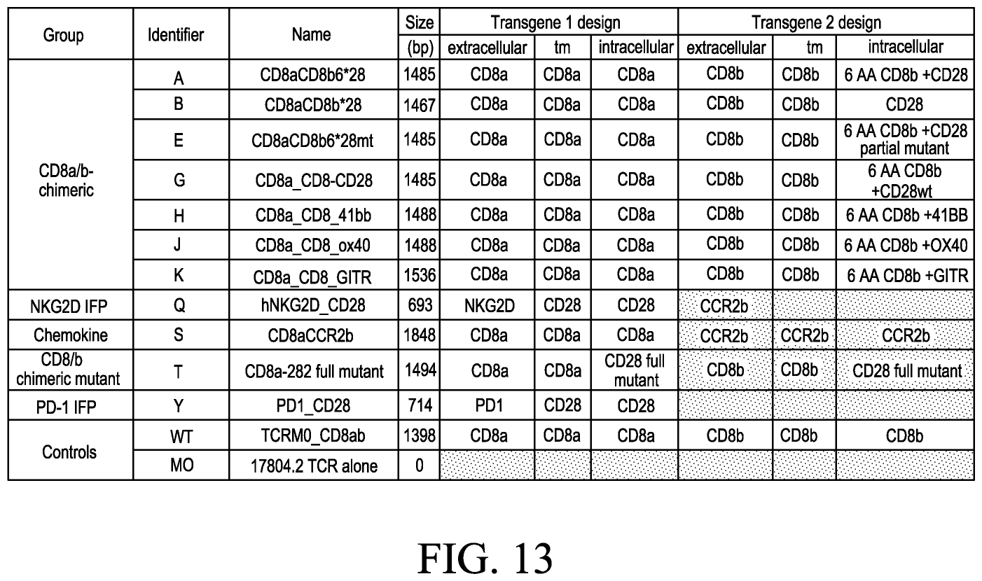

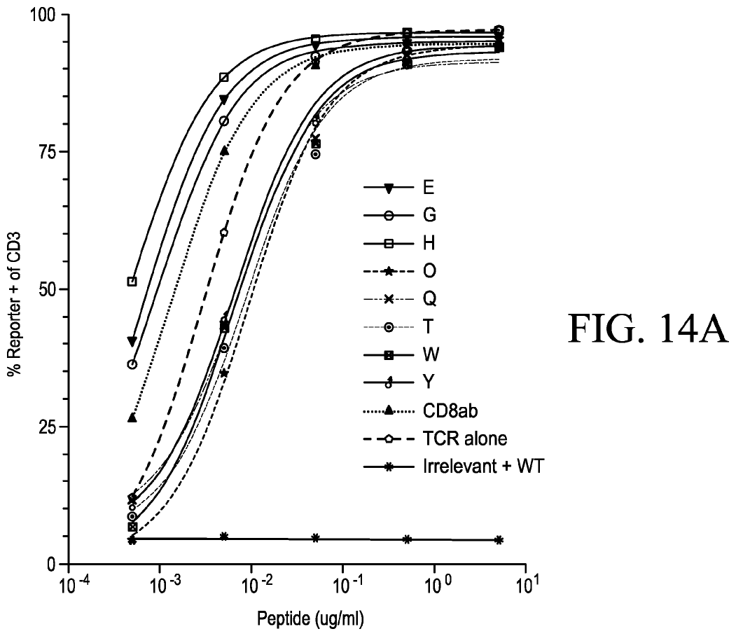

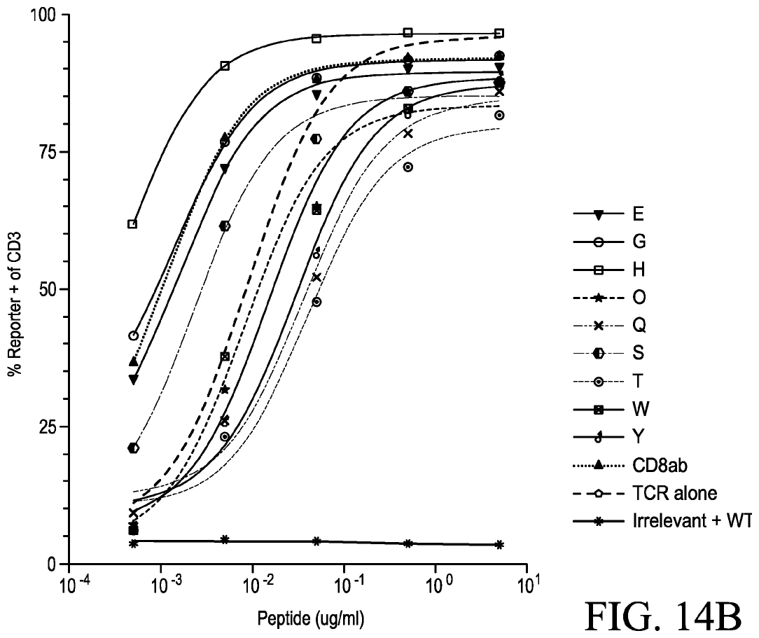

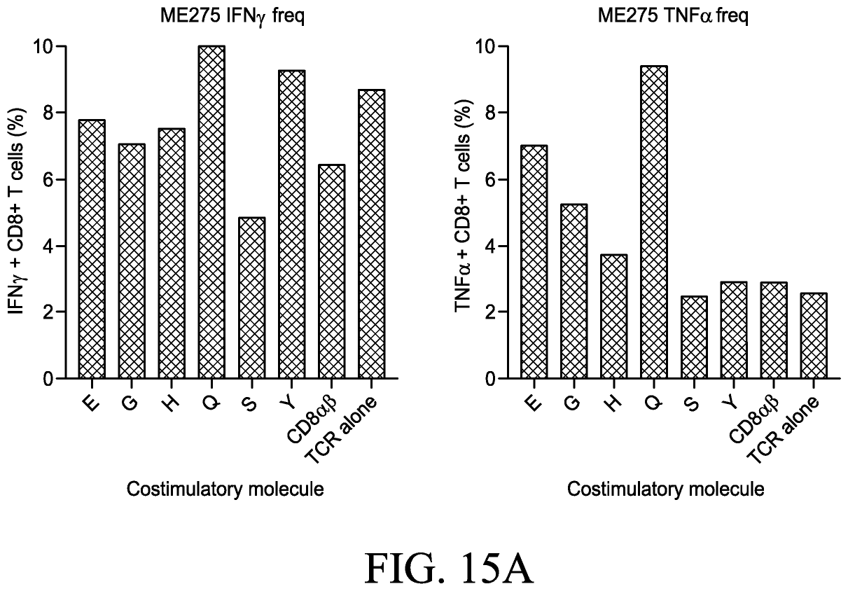

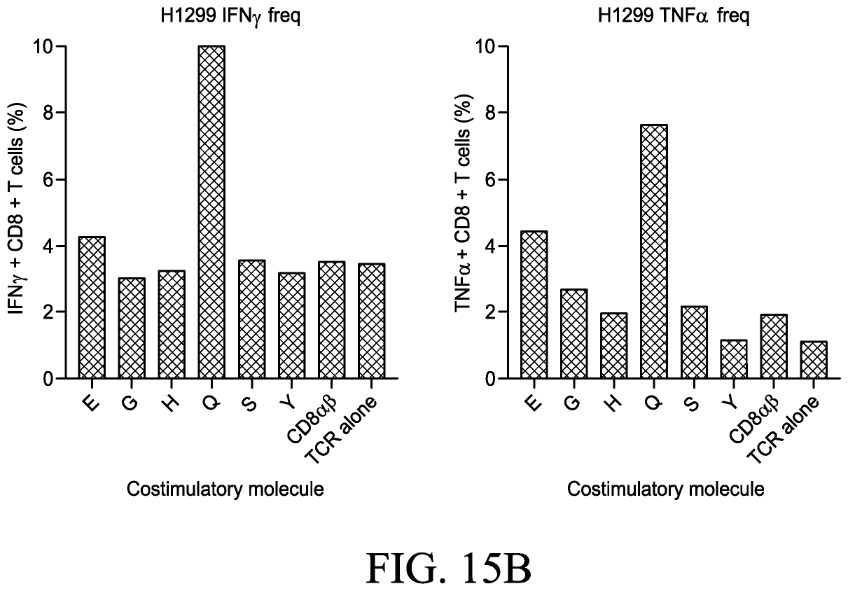

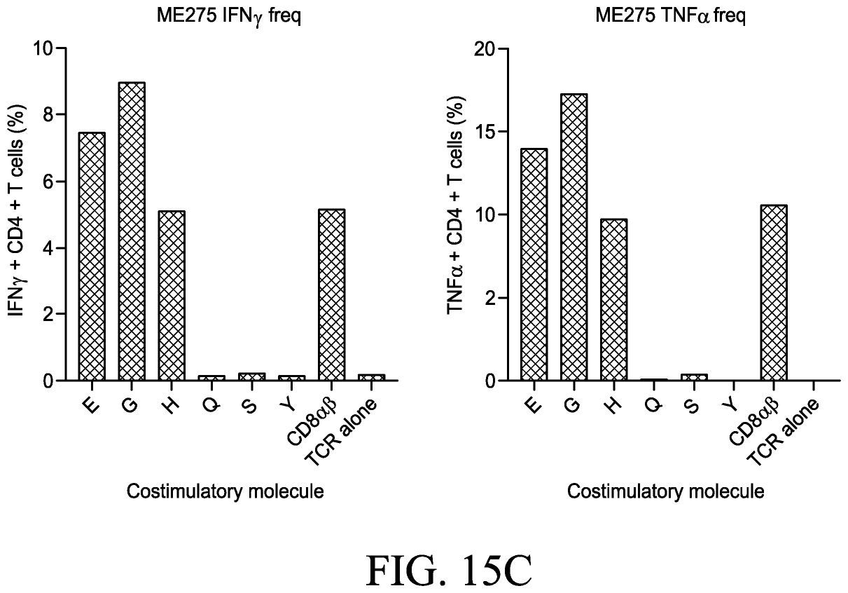

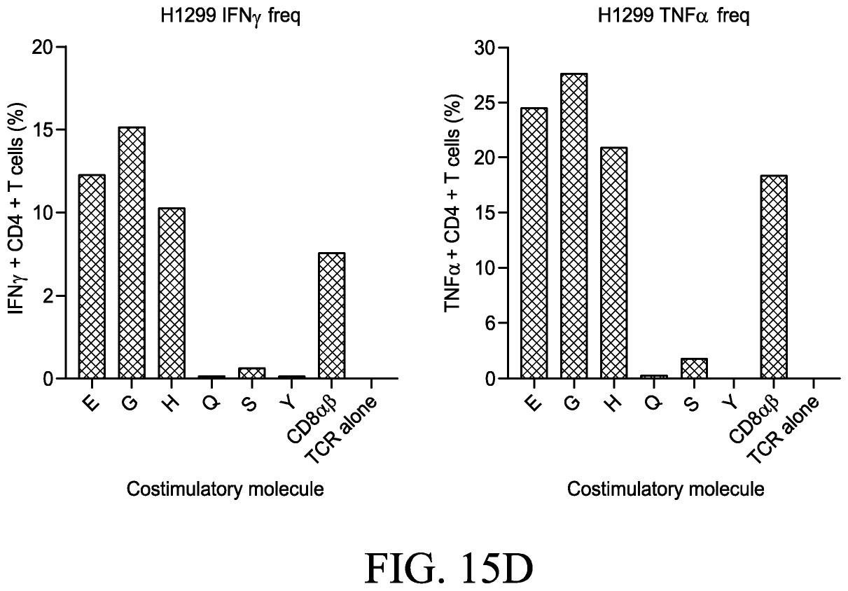

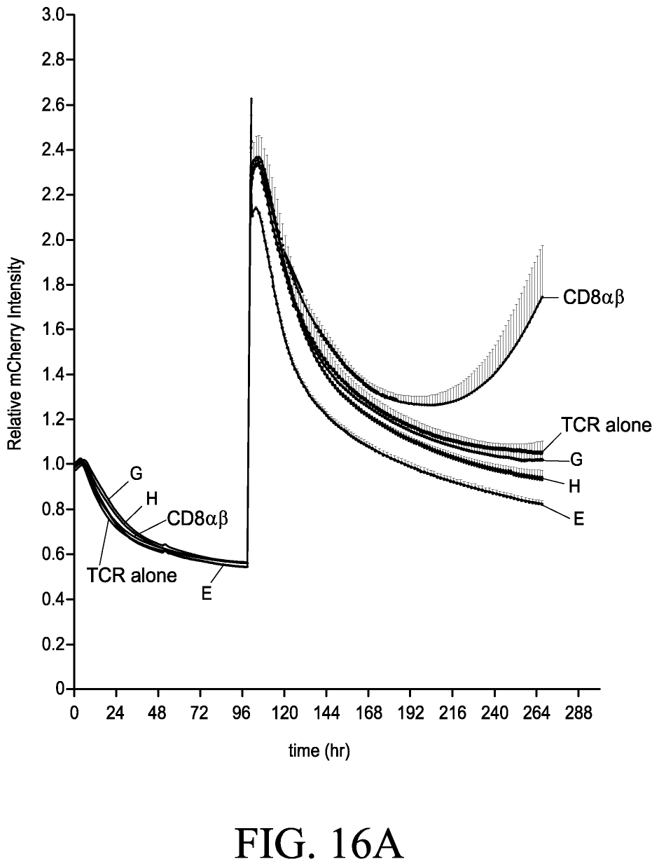

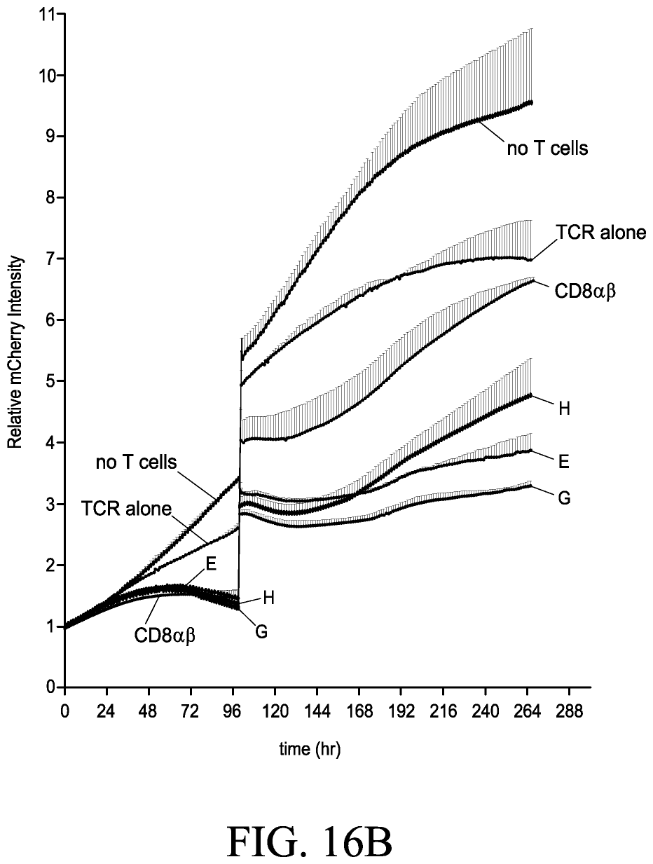

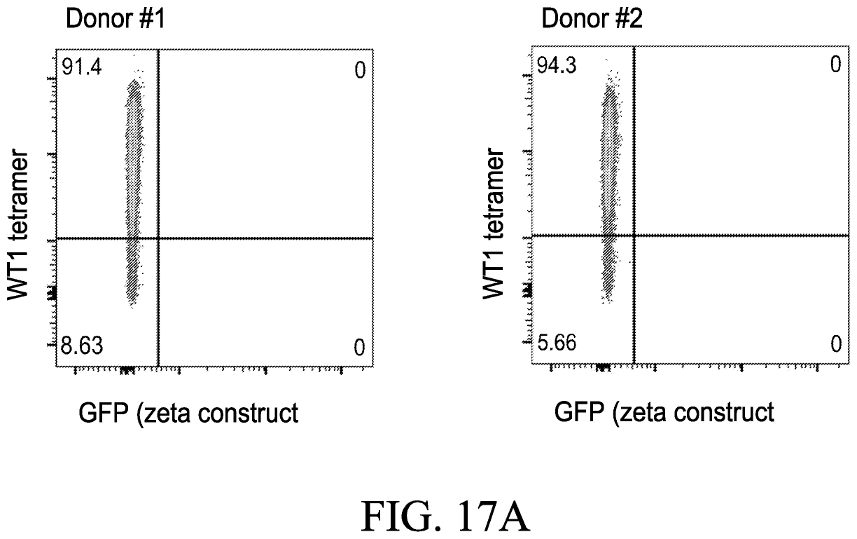

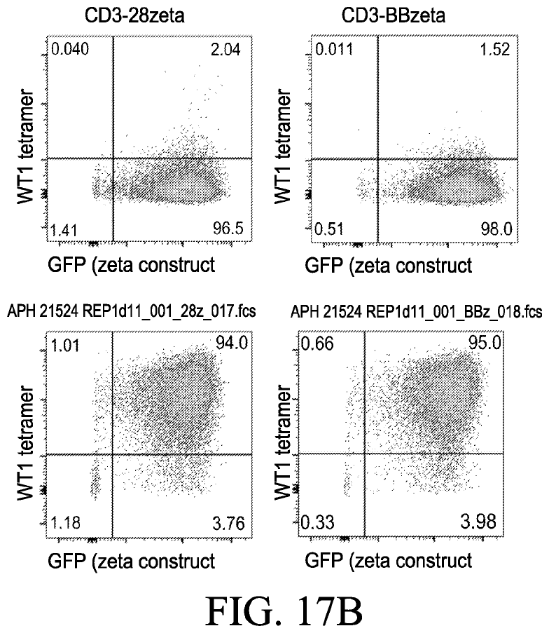

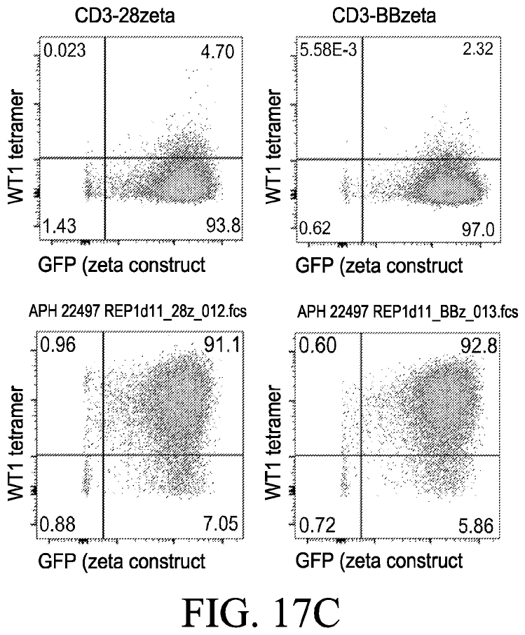

Each construct shown in FIG. 1 includes the depicted co-receptor fusion construct(s) with a cognate CD8α chain and/or CD8β chain. Each co-receptor fusion construct includes the extracellular and transmembrane domains of the CD8β or CD8α chain, respectively. Constructs 1, 2, 6, 7, and 8 further comprise a 6-amino acid sequence (HLCCRR; SEQ ID NO.:10) from the CD8β intracellular domain, located between the CD8β transmembrane domain and the CD28 intracellular domain. Construct 1 includes a dileucine motif (“LL”) that is native to a human CD28 intracellular domain. In Constructs 2-5, each leucine of the dileucine motif was replaced by a glycine amino acid. Other constructs (not shown) included constructs in which a native human CD28 diproline motif was altered and each proline in the motif was replaced with an alanine.

FIG. 2 shows CD8 expression by human primary CD4+ T cells transduced with a MHC-I-restricted TCR and one of the indicated CD8 co-receptor constructs. Cells were sorted on Day 7 following transduction with construct-containing lentivirus and expanded. Left panel: expression of wild-type CD8αβ in TCR-transduced cells. Middle panel: expression of CD8α/CD8β-CD28 fusion construct (“Construct 1” in FIG. 1). Right panel: expression of CD8α/CD8β-4-1BB fusion construct (“Construct 6” in FIG. 1).

FIG. 3 shows proliferation of human primary CD4+ T cells that were transduced with MHC-I-restricted TCR alone; TCR+Construct 1; or TCR+Construct 6, sorting on Day 7, and stimulation with antigen-expressing MEL275 cells at the indicated effector:target (E:T) ratios on Day 9 following a Rapid Expansion Protocol (REP).

FIGS. 4A and 4B show production of cytokines by CD4+ T cells transduced to express a MHC-I-restricted (MAGE-A1-278-specific) TCR, either alone (B) or with wild-type or chimeric CD8 co-receptor molecules (A, B), as indicated. (A) Data from flow cytometry experiments measuring interferon-gamma (shown as “ifnγ”) production by transduced T cells in the absence (bottom row) or presence (top row) of peptide antigen. (B) Expression of IFN-γ and TNFα by cells transduced with (from left to right) TCR alone; TCR+wild-type CD8 co-receptor; TCR+Construct 1 from FIG. 1; or TCR+Construct 6 from FIG. 1.

FIG. 5 shows specific killing of tumor cells by T cells, including T cells that express (i) a TCR that specifically binds to an antigen: MHC complex on the tumor cells and (ii) a fusion protein of the present disclosure comprising (a) an extracellular component comprising an extracellular domain (also known as an ectodomain) from CD3ζ, (b) a transmembrane component comprising a transmembrane domain from CD3ζ, and (c) intracellular component comprising (c)(i) a costimulatory domain from CD28 or 4-1BB, and, carboxy-terminal to (c)(i), (c)(ii) an intracellular signaling domain from CD3ζ. An IncuCyte® assay was used to quantitate killing; the Red Object Area on the y-axis represents the presence of tumor cells.

FIG. 6 shows specific killing of tumor cells (PANC-1) by T cells (2:1 E:T ratio), including T cells that express (i) a TCR that specifically binds to an antigen: MHC complex on the tumor cells and (ii) a fusion protein comprising (a) an extracellular region from CD3ζ, (b) a transmembrane region from CD3ζ, (c) a costimulatory domain from CD28 or 4-1BB, and (d) an intracellular signaling domain from CD3ζ. An IncuCyte® assay was used to quantitate killing; the Red Object Area on the y-axis represents the presence of tumor cells.

FIG. 7 shows (top left) IFN-γ production, (top right) IL-2 production, and (bottom) proliferation of T cells expressing either (i) a TCR and a wild-type CD8 co-receptor or (ii) a TCR and a CD8-CD28 fusion polypeptide of the present disclosure.

FIG. 8 shows schematic diagrams of certain fusion protein constructs according to the present disclosure. The parallel horizontal lines across the image represent a cell membrane, with the extracellular portion of the protein shown above the upper horizontal line, and the intracellular portion of the protein shown below the lower horizontal line. Source proteins for the fusion protein components are indicated. The proteins with an extracellular portion marked “CD8α/β” and the construct with an extracellular portion marked “NKG2D” were investigated for function without a cognate co-receptor.

FIGS. 9A-12C provide details of certain fusion protein constructs according to the present disclosure. In each of FIGS. 9A, 10A, 11A, and 12A, a group of constructs sharing a structural theme (e.g., groups are CD8α/β chimeras, single-stalk fusions, NKG2D immunomodulatory fusion protein, expression constructs encoding a CD8α and a chemokine receptor, CD8/B chimeric mutant, Fas immunomodulatory fusion proteins, PD-1_CD28 immunomodulatory fusion protein (used as a positive control), and wild-type CD8αβ control) is described, including the general design of the respective transgene or transgenes encoding the fusion or fusions. For expression constructs indicating two transgenes, the two encoded proteins can function as a pair. For constructs indicating one transgene, the encoded protein can function as a single protein, without associating with a cognate co-protein. In FIGS. 9B-9C, 10B-10C, 11B-11C, and 12B-12C, further details of the construct designs are provided.

FIGS. 13-16B relate to experiments in which cells were transduced to express a fusion protein-encoding construct along with a TCR. In various experiments, controls included cells transduced with wild-type CD8αβ, cells transduced with TCR alone, and cells transduced with an irrelevant TCR (i.e. not specific for the peptide antigen used in the experiment) with wild-type CD8αβ. The tested fusion protein constructs (either two-polypeptide or one-polypeptide; for two-polypeptide constructs, the two polypeptides were separated by a P2A self-cleaving peptide sequence (“/P2A/” below) included (see also FIG. 13): “E” (full-length CD8α chain/P2A/truncated CD8β chain (including six CD8β intracellular amino acids H-L-C-C-R-R (SEQ ID NO.:10)) fused to a CD28 intracellular region comprising a LL-to-GG mutation and partial signaling mutation, discussed herein); “G” (full-length CD8α chain/P2A/truncated CD8β chain (including six CD8β intracellular amino acids H-L-C-C-R-R (SEQ ID NO.:10)) fused to a wild-type CD28 intracellular domain); “H” (full-length CD8α chain/P2A/truncated CD8β chain (including six CD8β intracellular amino acids H-L-C-C-R-R SEQ ID NO.:10)) fused to a wild-type 4-1BB intracellular region); “O” ((N-terminal-to-C-terminal direction) an extracellular component comprising a CD8α Ig V-like domain and a CD8β stalk portion; a CD28 transmembrane region; a CD28 intracellular region comprising a LL-to-GG mutation; and a CD8α intracellular region); “Q” (single-chain fusion comprising a NKG2D extracellular region, a NKG2D transmembrane region, and a wild-type CD28 intracellular region), “S” (full-length CD8α chain/P2A/full-length CCR2b); “T” (truncated CD8α chain fused to CD28 intracellular region with full signaling mutations/P2A/truncated CD8β chain fused to CD28 intracellular domain with LL-to-GG and full signaling mutations); “W” (single-chain fusion comprising Fas extracellular region, Fas transmembrane region, and CD8α intracellular region); and “Y” (single-chain fusion comprising truncated PD-1 extracellular region, truncated CD28 extracellular region comprising, CD28 transmembrane region, and CD28 intracellular region).

FIGS. 14A and 14B show TCR signal activation in Jurkat reporter cells transduced with (i) a TCR and a fusion construct or (ii) a control construct (TCR alone, irrelevant TCR with wild-type CD8αβ, or wild-type CD8αβ). Jurkat cells were engineered to knock-out endogenous MHC class I and endogenous TCRα and TCRβ chains, with a neogreen reporter knocked-in downstream of Nur77. Nur77 gene expression is rapidly upregulated by TCR signaling. The percent reporter-positive of CD3-positive cells (% reporter+of CD3) on the y-axis represents the percentage of transduced Jurkat cells that were reporter-positive after co-culture with antigen peptide-loaded T2 cells (5:1 E:T ratio). FIG. 14A: transduced CD8+ Jurkat reporter cells. FIG. 14B: transduced CD4+ Jurkat reporter cells.

FIGS. 15A-15D show production of cytokines by CD8+ or CD4+ T cells transduced to express a MHC-I-restricted TCR alone, wild-type CD8αβ alone, or the TCR and a fusion protein construct of the present disclosure. Transduced T cells were stimulated with the tumor cell lines ME275 and H1299. After stimulation, the T cells were fixed, permeabilized, and stained for intracellular IFNγ and TNFα. FIG. 15A: transduced CD8+ T cells following stimulation with ME275 cells. FIG. 15B: transduced CD8+ T cells following stimulation with H1299 cells. FIG. 15C: transduced CD4+ T cells following stimulation with ME275 cells. FIG. 15D: transduced CD4+ T cells following stimulation with H1299 cells. The y-axis in each graph represents the percentage of T cells positive for intracellular IFN-γ or TNFα, as indicated.

FIGS. 16A and 16B show specific killing of tumor cells by CD8+ or CD4+ T cells transduced to express MHC class I-restricted TCR alone, wild-type CD8αβ alone, or the TCR and the indicated fusion construct. An IncuCyte® assay was used to quantitate killing; the Red Object Area on the y-axis represents the presence of tumor cells (mCherry-positive ME275 tumor cells). FIG. 16A: presence of tumor cells following co-culture with transduced CD8+ T cells. FIG. 16B: presence of tumor cells following co-culture with transduced CD4+ T cells.

FIGS. 17A-17C show transduction of donor T cells with a WT1-specific TCR, a CD3ζ fusion protein construct, or both. WT1-specific TCR transduction was evaluated using WT1 tetramers, as shown on the y-axis of each panel. The fusions were designed to co-express GFP. Co-receptor fusion transduction was monitored by GFP, as shown on the x-axis of each panel. The fusion constructs comprise (a) an extracellular component comprising (a) an extracellular component comprising an extracellular domain from CD3ζ, (b) a transmembrane domain from CD3ζ, and (c) intracellular component comprising (i) an intracellular domain from CD28 (28) or 4-1BB (BB), and (ii) an intracellular signaling domain from CD3ζ. FIG. 17A: T cells from donors 1 and 2, as indicated, were transduced with WT1-specific TCR only. FIG. 17B: T cells from donor 1 were (top row) transduced with the indicated fusion construct only, or (bottom row) co-transduced with fusion construct and WT1-specific TCR. FIG. 17C: T cells from donor 2 were transduced with co-receptor fusion constructs only, as indicated, (top row) or co-transduced with co-receptor fusion constructs and WT1-specific TCR (bottom row).

### DETAILED DESCRIPTION

The present relates, in part, to polypeptides, such as fusion proteins and other engineered proteins, that are useful to confer to, or improve, a desired activity of a host immune cell, such as an immune cell that targets a diseased or pathogenic cell (e.g. a cancer cell). Certain embodiments of the polypeptides can, for example, translate an external stimulus (e.g., binding to a ligand) to a preferred (e.g. stimulatory) signal in a host cell, or to prevent, reduce, attenuate, modulate, or abrogate an undesired signal in the host cell. Also provided are polynucleotides that encode any one or more of the polypeptides, and vectors that comprise a polynucleotide. In some contexts, a polynucleotide encodes one or more (e.g. two) polypeptides that can function provide a stimulatory signal to a host cell that expresses the same, and/or that can function to prevent, modulate, attenuate, or abrogate a suppressive signal. In some embodiments, two or more polypeptides can associate to form a multimer (e.g. a dimer) and confer advantageous functions when expressed at the surface of a host cell. In certain other embodiments, a polypeptide monomer can function at the surface of a host cell.

In certain embodiments, disclosed polypeptides may be advantageously employed to improve one or more cellular function, such as in the context of adoptive cell therapy. By way of example, in some embodiments, a stimulatory signal is conferred or improved, and/or a suppressive signal is reduced, prevented, or abrogated, in a host T cell for adoptive therapy against a disease or disorder such as a cancer. In particular embodiments, a CD4+ T cell can have improved function (e.g. cyotoxic and/or helper function and/or viability) against diseased cells. A CD4+ T cell may further express, or be engineered to further express, an antigen-specific binding protein such as a T cell receptor, which can, in some embodiments, comprise an MHC I-restricted T cell receptor. Such CD4+ T cells may be advantageously utilized in cell therapy, e.g. with or apart from CD8+ effector T cells that also target the diseased cells.

Some embodiments include a polynucleotide encoding (a) a first polypeptide, wherein the first polypeptide comprises (a)(i) an extracellular component comprising an extracellular domain from a CD8 β-chain (CD8β), or a functional portion or variant thereof that is capable of binding to a MHC Class I molecule, (a)(ii) a transmembrane domain from a CD8β, and (a)(iii) an intracellular component comprising (a)(iii)(1) a CD8β intracellular region amino acid sequence that comprises or consists of the amino acid sequence set forth in SEQ ID NO.:10 or SEQ ID NO.:9, and (a)(iii)(2) a costimulatory domain or a functional portion or variant thereof, wherein, optionally:
(1) the costimulatory domain or a functional portion or variant thereof is from one or more of CD28 (optionally comprising a LL→GG mutation, a partial signaling mutation, and/or a full signaling mutation), 4-1BB (CD137), OX40 (CD134), ICOS (CD278), GITR, CD27, CD2, CD5, ICAM-1 (CD54), LFA-1 (CD11a/CD18), GITR, CD30, CD40, BAFF-R, HVEM, LIGHT, NKG2C, SLAMF7, NKp80, CD160, B7-H3, a ligand that specifically binds with CD83, CD94, or DAP12; and/or
(2) the polynucleotide further encodes (b) a second polypeptide comprising CD8α polypeptide, wherein, optionally, the polynucleotide further comprises, disposed between the nucleotide sequence encoding (a) and the nucleotide sequence encoding (b), (c) a nucleotide sequence encoding any one or more of: a self-cleaving peptide; a furin cleavage sequence; and an internal ribosomal skip element (IRES), wherein, optionally, the nucleotide sequence of (c) separates the nucleotide sequence of (a) from the nucleotide sequence of (b); and/or
(3) the polynucleotide further encodes (d) a T cell receptor (TCR), wherein the TCR is optionally MHC-I-restricted; and/or
(4) the polynucleotide is comprised in a host cell, wherein the host cell comprises an immune system cell, wherein the immune system cell comprises a CD4⁺ T cell, a CD8⁺ T cell, a CD4 CD8-double negative T cell, a γδ T cell, a natural killer cell, a natural killer T cell, a dendritic cell, a naïve T cell, a central memory T cell, a stem cell memory T cell, an effector memory T cell, or any combination thereof.

In certain aspects, the present disclosure provides fusion proteins that comprise:
(i) an extracellular component comprising an extracellular domain (or an ectodomain) or functional portion thereof: (i)(1) from a CD8 co-receptor β-chain or a functional portion or variant thereof; (i)(2) from a CD8 co receptor α chain or a functional portion or variant thereof; (i)(3) that comprises an amino acid sequence from a CD8 co-receptor β-chain extracellular domain or a functional portion or variant thereof and an amino acid sequence from a CD8 co-receptor α chain, wherein the extracellular component of (i)(1)-(i)(3) is capable of binding to a MHC class I molecule; (i)(4) from a NKG2D extracellular domain (or ectodomain) or a functional portion or variant thereof; (i)(5) from a Fas extracellular domain (or ectodomain) or a functional portion or variant thereof; or (i)(6) from a PD-1 extracellular domain (or ectodomain) or a functional portion or variant thereof;
(ii) a transmembrane domain, optionally provided that the transmembrane domain is not a transmembrane domain from a CD8 co-receptor α-chain when the extracellular component comprises a full length extracellular domain from the CD8 co-receptor α chain; and
(iii) an intracellular component comprising a co-stimulatory domain or a functional portion or variant thereof.

In further embodiments, the extracellular component comprises a CD8 co-receptor β-chain, or a functional portion or variant thereof.

In some embodiments, the co-stimulatory domain comprises a co-stimulatory domain from one or more of CD28, 4-1BB (CD137), OX40 (CD134), ICOS (CD278), CD27, CD2, CD5, ICAM-1 (CD54), LFA-1 (CD11a/CD18), GITR, CD30, CD40, BAFF-R, HVEM, LIGHT, NKG2C, SLAMF7, NKp80, CD160, B7-H3, a ligand that specifically binds with CD83, CD94, DAP12, TRAF1, and LCK, and/or comprises a functional variant of a co-stimulatory domain thereof.

Also provided herein are fusion proteins that comprise:
(i) an extracellular component comprising an extracellular domain (or an ectodomain) from a CD8 co-receptor β-chain or a functional portion or variant thereof, or from a CD8 co receptor α-chain or a functional portion or variant thereof, that is capable of binding to a MHC class I molecule;
(ii) a transmembrane domain; and
(iii) an intracellular component comprising a co stimulatory domain from one, two, or three of:
(a) a variant sequence of CD28 comprising or consisting of an amino acid sequence having at least 80% identity to the amino acid sequence shown in SEQ ID NO: 19 or 20, provided that: (1) no Tyr residue corresponding to position 12, 27, 30, or 39 of SEQ ID NO:19 is substituted with Phe when the extracellular component comprises a full length extracellular domain from a CD8 co receptor α chain and the transmembrane domain comprises a transmembrane domain from the CD8 co receptor α chain; and/or (2) one or both of the leucine residues corresponding to positions 7 and 8 of SEQ ID NO: 19 is substituted for a different amino acid, wherein the different amino acid optionally comprises glycine;
(b) CD27, or a functional portion or variant thereof;
(c) 4-1BB, or a functional portion or variant thereof;
(d) ICOS, or a functional portion or variant thereof;
(e) OX40, or a functional portion or variant thereof;
(f) CD30, or a functional portion or variant thereof;
(g) LFA-1, or a functional portion or variant thereof;
(h) CD2, or a functional portion or variant thereof;
(i) CD7, or a functional portion or variant thereof;
(j) LIGHT, or a functional portion or variant thereof;
(k) NKG2C, or a functional portion or variant thereof;
(l) B7-H3, or a functional portion or variant thereof;
(j) GITR, or a functional portion or variant thereof;
(k) BAFF-R, or a functional portion or variant thereof;
(l) CD5, or a functional portion or variant thereof;
(m) HVEM, or a functional portion or variant thereof;
(n) CD160, or a functional portion or variant thereof;
(o) LFA-1, or a functional portion or variant thereof;
(p) SLAMF7, or a functional portion or variant thereof;
(q) NKp80, or a functional portion or variant thereof;
(r) ICAM-1, or a functional portion or variant thereof;
(s) CD94, or a functional portion or variant thereof;
(t) DAP12, or a functional portion or variant thereof;
(u) a ligand that specifically binds with CD83;
(v) Lck, or a functional portion or variant thereof, or TRAF1, or a functional portion or variant thereof.

Also provided herein are fusion proteins that comprise (i) an extracellular component comprising an extracellular domain (or an ectodomain) from a CD3 protein (e.g., CD3 ζ, CD3ε, CD3γ, or CD3δ), or a functional portion or variant thereof, (b) a transmembrane component comprising a transmembrane domain from a CD3 protein (e.g., CD3ζ, CD3ε, CD3γ, CD3δ) or a functional portion or variant thereof, and (c) an intracellular component comprising an intracellular domain from CD28 or 4-1BB, or a functional portion or variant thereof, and/or an intracellular signaling component from CD3 ζ or a functional portion or variant thereof. In certain embodiments, the intracellular component (c) of the fusion protein comprises an intracellular domain from CD28 or 4-1BB, or a functional portion or variant thereof, and an intracellular signaling component from CD3ζ, or a functional portion or variant thereof.

Also provided herein are fusion proteins that comprise:
(i) an extracellular component comprising an amino acid sequence from a CD8α extracellular domain, an amino acid sequence from a CD8β extracellular domain, and an optional amino acid sequence from a CD28 extracellular domain, a transmembrane domain from CD28 or from a CD8 co-receptor, and a CD28 intracellular domain (optionally comprising a LL→GG mutation);
(ii) an extracellular component comprising an amino acid sequence from a CD8α extracellular domain and an amino acid sequence from a CD8β extracellular domain, a CD28 transmembrane domain, and a CD28 intracellular domain (optionally comprising a LL→GG mutation);
(iii) an extracellular component comprising an amino acid sequence from a CD8α extracellular domain and an amino acid sequence from a CD8β extracellular domain, a CD28 transmembrane domain, a CD28 intracellular domain (optionally comprising a LL→GG mutation), and a CD8α intracellular domain;
(iv) an extracellular component comprising an amino acid sequence from a CD8α extracellular domain and an amino acid sequence from a CD8β extracellular domain, a CD8α transmembrane domain, and a CD8α intracellular domain;
(v) a NKG2D extracellular domain, a NKG2D or CD28 transmembrane domain, and a CD28 intracellular domain;
(vi) a Fas extracellular domain, a Fas transmembrane domain, and an intracellular amino acid sequence from Lck, which optionally comprises or consists of the amino acid sequence

PLQDNLVIALHSYEPSHDGDLGFEKGEQLRILEQSGEWWKAQSLTTGQEG
FIPFNFVAKANSLEPEPWFFKNLSRKDAERQLLAPGNTHGSFLIRESEST
AGSFSLSVRDFDQNQGEVVKHYKIRNLDNGGFYISPRITFPGLHELVRHY
TNASDGLCTRLSRPCQTQKPQKPWWEDEWEVPRETLKLVERLGAGQFGEV
WMGYYNGHTKVAVKSLKQGSMSPDAFLAEANLMKQLQHQRLVRLYAVVTQ
EPIYIITEYMENGSLVDFLKTPSGIKLTINKLLDMAAQIAEGMAFIEERN
YIHRDLRAANILVSDTLSCKIADFGLARLIEDNEYTAREGAKFPIKWTAP
EAINYGTFTIKSDVWSFGILLTEIVTHGRIPYPGMTNPEVIQNLERGYRM
VRPDNCPEELYQLMRLCWKERPEDRPTFDYLRSVLEDFFTATEGQYQPQ
P;

(vii) a Fas extracellular domain, a Fas transmembrane domain, and a CD8α intracellular domain;
(viii) a Fas extracellular domain, a Fas transmembrane domain, an optional linker, and a TRAF1 intracellular domain; or
(ix) a PD-1 extracellular domain, an amino acid sequence from a CD28 extracellular domain, a CD28 transmembrane domain, and a CD28 intracellular domain (optionally including a LL→GG mutation).

Also provided is a truncated or variant Fas polypeptide that comprises a Fas extracellular domain and a Fas transmembrane domain, and does not comprise a functional Fas intracellular signaling domain, and optionally does not comprise an intracellular domain.

Also provided are polynucleotides that encode, and/or host cells that express, a first polypeptide and a second polypeptide, wherein:
(i) the first polypeptide comprises a CD8α extracellular domain, a transmembrane domain, and a CD8α intracellular domain, and the second polypeptide comprises a CD8β extracellular domain, a transmembrane domain, and an intracellular domain comprising (1) the amino acid sequence HLCCRR and (2) a CD28 intracellular domain comprising a LL→GG mutation;
(ii) the first polypeptide comprises a CD8α extracellular domain, a transmembrane domain, and a CD8α intracellular domain, and the second polypeptide comprises a CD8β extracellular domain, a transmembrane domain, and a CD28 intracellular domain (optionally comprising a LL→GG mutation);
(iii) the first polypeptide comprises a CD8α extracellular domain, a transmembrane domain, and a CD28 intracellular domain (optionally comprising a LL→GG mutation), and the second polypeptide comprises a CD8β extracellular domain, a transmembrane domain, and a CD28 intracellular domain (optionally comprising a LL→GG mutation);
(iv) the first polypeptide comprises a CD8α extracellular domain, a transmembrane domain, and a CD28 intracellular domain, and the second polypeptide comprises a CD8β extracellular domain, a transmembrane domain, and a CD8β intracellular domain;
(v) the first polypeptide comprises a CD8α extracellular domain, a transmembrane domain, and a CD8α intracellular domain, and the second polypeptide comprises a CD8β extracellular domain, a transmembrane domain, and a CD28 intracellular domain (optionally comprising a LL→GG mutation and/or a mutation that reduces CD28 immune signaling as compared to a wild-type CD28, CD28 intracellular domain, wherein, further optionally, the CD28 intracellular domain comprises the amino acid sequence

RSKRSRGGHSDAMNMTARRAGPTRKHYQAYAAPRDFAAYRS);

(vi) the first polypeptide comprises a CD8α extracellular domain, a transmembrane domain, and a CD28 intracellular domain (optionally comprising a LL→GG mutation and/or a mutation that reduces CD28 immune signaling as compared to a wild-type CD28, CD28 intracellular domain, wherein, further optionally, the CD28 intracellular domain comprises the amino acid sequence RSKRSRGGHSDAMNMTARRAGPTRKHYQAYAAPRDFAAYRS), and the second polypeptide comprises a CD8β extracellular domain, a transmembrane domain, and a CD28 intracellular domain (optionally comprising a LL→GG mutation and/or a mutation that reduces CD28 immune signaling as compared to a wild-type CD28, CD28 intracellular domain, wherein, further optionally, the CD28 intracellular domain comprises the amino acid sequence

RSKRSRGGHSDAMNMTARRAGPTRKHYQAYAAPRDFAAYRS);

(vii) the first polypeptide comprises a CD8α extracellular domain, a transmembrane domain, and a CD8α intracellular domain, and the second polypeptide comprises a CD8β extracellular domain, a transmembrane domain, and an intracellular domain comprising (1) the amino acid sequence HLCCRR and (2) a CD28 intracellular domain;
(viii) the first polypeptide comprises a CD8α extracellular domain, a transmembrane domain, and a CD8α intracellular domain, and the second polypeptide comprises a CD8β extracellular domain, a transmembrane domain, and a 4-1BB intracellular domain;
(ix) the first polypeptide comprises a CD8α extracellular domain, a transmembrane domain, and a CD8α intracellular domain, and the second polypeptide comprises a CD8β extracellular domain, a transmembrane domain, and a ICOS intracellular domain;
(x) the first polypeptide comprises a CD8α extracellular domain, a transmembrane domain, and a CD8α intracellular domain, and the second polypeptide comprises a CD8β extracellular domain, a transmembrane domain, and a OX40 intracellular domain;
(xi) the first polypeptide comprises a CD8α extracellular domain, a transmembrane domain, and a CD8α intracellular domain, and the second polypeptide comprises a CD8β extracellular domain, a transmembrane domain, and a GITR intracellular domain;
(xii) the first polypeptide comprises a CD8α extracellular domain, a CD28 transmembrane domain, and a CD28 intracellular domain (optionally comprising a LL→GG mutation), and the second polypeptide comprises a CD8β extracellular domain, a CD8β transmembrane domain, and an intracellular domain comprising (1) a CD8β intracellular domain and (2) an intracellular amino acid sequence from Lck, optionally the amino acid sequence

PLQDNLVIALHSYEPSHDGDLGFEKGEQLRILEQSGEWWKAQSLTTGQEG
FIPFNFVAKANSLEPEPWFFKNLSRKDAERQLLAPGNTHGSFLIRESEST
AGSFSLSVRDFDQNQGEVVKHYKIRNLDNGGFYISPRITFPGLHELVRHY
TNASDGLCTRLSRPCQTQKPQKPWWEDEWEVPRETLKLVERLGAGQFGEV
WMGYYNGHTKVAVKSLKQGSMSPDAFLAEANLMKQLQHQRLVRLYAVVTQ
EPIYIITEYMENGSLVDFLKTPSGIKLTINKLLDMAAQIAEGMAFIEERN
YIHRDLRAANILVSDTLSCKIADFGLARLIEDNEYTAREGAKFPIKWTAP
EAINYGTFTIKSDVWSFGILLTEIVTHGRIPYPGMTNPEVIQNLERGYRM
VRPDNCPEELYQLMRLCWKERPEDRPTFDYLRSVLEDFFTATEGQYQP
QP;

(xiii) the first polypeptide comprises a CD8α and the second polypeptide comprises a CCR4;
(xiv) the first polypeptide comprises a CD8α and the second polypeptide comprises a CCR2b; or
(xv) the first polypeptide comprises a CD8α extracellular domain, a transmembrane domain, and a CD28 intracellular domain (optionally comprising a LL→GG mutation and/or a mutation that reduces or abrogates CD28 immune signaling as compared to a wild-type CD28 intracellular domain, wherein, further optionally, the CD28 intracellular domain comprises the amino acid sequence RSKRSRGGHSDAMNMTARRAGPTRKHFQAFAAPRDFAAFRS, and the second polypeptide comprises a CD8β extracellular domain, a transmembrane domain, and a CD28 intracellular domain (optionally comprising a LL→GG mutation and/or a mutation that reduces or abrogates CD28 immune signaling as compared to a wild-type CD28 intracellular domain, wherein, further optionally, the CD28 intracellular domain comprises the amino acid sequence

RSKRSRGGHSDAMNMTARRAGPTRKHFQAFAAPRDFAAFRS)

In certain embodiments, a polynucleotide encoding the first polypeptide is separated from a polynucleotide encoding the second polypeptide by a polypeptide encoding a self-cleaving peptide.

Features of certain embodiments of polypeptides of the present disclosure are provided herein, and include those fusion proteins and constructs shown and/or described in FIGS. 9A-13, Tables 1-5, and the Table of Sequences. Accordingly, in any of the presently disclosed embodiments, a polypeptide can comprise an amino acid sequence (e.g., extracellular domain, transmembrane domain, intracellular domain, or any combination thereof) as provided in Tables 1-5 and the Table of Sequences herein and/or shown and/or described in any one or more of FIGS. 9A-13.

Presently disclosed polypeptides and expression constructs can be useful for improving and/or modulating activation and/or one or more therapeutically relevant function of an immune cell (e.g. a T cell) expressing an antigen-specific binding protein, such as, for example, a T cell receptor (TCR). In certain embodiments, a polypeptide, expression construct, or first and second polypeptide, is expressed by a modified host cell (e.g., immune cell, such as, for example, a T cell (e.g. a CD4+ T cell, a CD8+ T cell, or both), NK cell, or NK-T cell) that expresses a binding protein (e.g., a TCR) specific for a target such as an antigen (e.g., tumor associated antigen or an antigen from a pathogen), wherein the polypeptide improves activation or stimulation of the host cell following binding to target, as compared to a reference or unmodified host cell (not expressing the polypeptide, but expressing the binding protein). In certain embodiments, a host cell expressing an antigen-specific binding protein and a polypeptide (or first and second polypeptides) as disclosed herein kills an antigen-expressing target cell more effectively than does a reference host cell expressing the antigen-specific binding protein and not expressing the polypeptide(s).

In certain embodiments, a modified CD4⁺ T cell comprises a polypeptide of the instant disclosure (e.g., comprising at least a portion of a CD8 co-receptor ectodomain or extracellular domain and optionally the CD8 transmembrane domain) and/or a first and second polypeptide as provided herein, and optionally a MHC-I-restricted binding protein (e.g., a TCR).

Also provided are host cells that comprise (i) a heterologous polynucleotide that encodes a polypeptide and/or first and second polypeptide. In some embodiments, the encoded fusion protein) comprises: (a) an extracellular component comprising an extracellular domain from a CD8 co-receptor α-chain; (b) a transmembrane domain from a CD8 co-receptor α-chain; and (c) an intracellular component comprising a co stimulatory domain from CD28, or a functional portion or variant thereof; and (ii) a heterologous polynucleotide encoding a binding protein that specifically binds to an antigen or an antigen: MHC complex. In further embodiments, the host cell comprises a human immune system cell; e.g., a CD4⁺ T cell.

Also provided herein are polynucleotides that encode the disclosed polypeptides and/or first and second polypeptides, as well as expression vectors that comprise the polynucleotides, and compositions that comprise the polypeptides, polynucleotides, vectors, and/or host cells.

In any of the presently disclosed embodiments, a polypeptide, expression construct, and/or first and second polypeptide can be expressed by a host cell, such as an immune cell.

In certain embodiments, methods are provided for treating a disease or condition using a polypeptide, polynucleotide, vector, modified host cell, or cell composition of the present disclosure. In certain embodiments, the presently disclosed polypeptides and host cells are useful in treating cancer.

These and other non-limiting embodiments are discussed further herein.

Prior to setting forth this disclosure in more detail, it may be helpful to an understanding thereof to provide definitions of certain terms to be used herein. Additional definitions are set forth throughout this disclosure.

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, is to be understood to include any integer within the recited range, unless otherwise indicated. As used herein, the term “about” means±20% of the indicated range, value, or structure, unless otherwise indicated. It should be understood that the terms “a” and “an” as used herein refer to “one or more” of the enumerated components. The use of the alternative (e.g., “or”) should be understood to mean either one, both, or any combination of the alternatives. As used herein, the terms “include,” “have,” and “comprise” are used synonymously, which terms and variants thereof are intended to be construed as non-limiting.

“Optional” or “optionally” means that the subsequently described element, component, event, or circumstance may or may not occur, and that the description includes instances in which the element, component, event, or circumstance occurs and instances in which they do not.

In addition, it should be understood that the individual constructs, or groups of constructs, derived from the various combinations of the structures and subunits described herein, are disclosed by the present application to the same extent as if each construct or group of constructs was set forth individually. Thus, selection of particular structures or particular subunits is within the scope of the present disclosure.

The term “consisting essentially of” is not equivalent to “comprising” and refers to the specified materials or steps of a claim, or to those that do not materially affect the basic characteristics of a claimed subject matter. For example, a protein domain, region, or module (e.g., a binding domain, hinge region, or linker) or a protein (which may have one or more domains, regions, or modules) “consists essentially of” a particular amino acid sequence when the amino acid sequence of a domain, region, module, or protein includes extensions, deletions, mutations, or a combination thereof (e.g., amino acids at the amino- or carboxy-terminus or between domains) that, in combination, contribute to at most 20% (e.g., at most 15%, 10%, 8%, 6%, 5%, 4%, 3%, 2% or 1%) of the length of a domain, region, module, or protein and do not substantially affect (i.e., do not reduce the activity by more than 50%, such as no more than 40%, 30%, 25%, 20%, 15%, 10%, 5%, or 1%) the activity of the domain(s), region(s), module(s), or protein (e.g., the target binding affinity of a binding protein).

In certain embodiments, any of the presently disclosed polypeptides can comprise, consist essentially of, or consist of the recited feature(s) (e.g. components, domains, and/or amino acid sequences)

As used herein, “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refer to compounds that have the same basic chemical structure as a naturally occurring amino acid, i.e., an α-carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refer to chemical compounds that have a structure that is different from the general chemical structure of an amino acid, but that function in a manner similar to a naturally occurring amino acid.

As used herein, “protein” or “polypeptide” refers to a polymer of amino acid residues. Proteins apply to naturally occurring amino acid polymers, as well as to amino acid polymers in which one or more amino acid residue is an artificial chemical mimetic of a corresponding naturally occurring amino acid and non-naturally occurring amino acid polymers. Variants of proteins, peptides, and polypeptides of this disclosure are also contemplated. In certain embodiments, variant proteins, peptides, and polypeptides comprise or consist of an amino acid sequence that is at least 70%, 75%, 80%, 85%, 90%, 91%, 92%, 93%, 94%, 95%, 96%, 97%, 98%, 99%, or 99.9% identical to an amino acid sequence of a defined or reference amino acid sequence as described herein. In certain embodiments, variation of a defined or reference amino acid sequence comprises or consists of one or more conservative amino acid substitutions. It will be understood that the terms “protein” and “polypeptide” are interchangeable herein, unless the context clearly provides otherwise.

As used herein, “mutation” refers to a change in the sequence of a nucleic acid molecule or polypeptide molecule as compared to a reference or wild-type nucleic acid molecule or polypeptide molecule, respectively. A mutation can result in several different types of change in sequence, including substitution, insertion or deletion of nucleotide(s) or amino acid(s).

A “conservative substitution” refers to amino acid substitutions that do not significantly affect or alter binding characteristics of a particular protein. Generally, conservative substitutions are ones in which a substituted amino acid residue is replaced with an amino acid residue having a similar side chain. Conservative substitutions include a substitution found in one of the following groups: Group 1: Alanine (Ala or A), Glycine (Gly or G), Serine (Ser or S), Threonine (Thr or T); Group 2: Aspartic acid (Asp or D), Glutamic acid (Glu or Z); Group 3: Asparagine (Asn or N), Glutamine (Gln or Q); Group 4: Arginine (Arg or R), Lysine (Lys or K), Histidine (His or H); Group 5: Isoleucine (Ile or I), Leucine (Leu or L), Methionine (Met or M), Valine (Val or V); and Group 6: Phenylalanine (Phe or F), Tyrosine (Tyr or Y), Tryptophan (Trp or W). Additionally or alternatively, amino acids can be grouped into conservative substitution groups by similar function, chemical structure, or composition (e.g., acidic, basic, aliphatic, aromatic, or sulfur-containing). For example, an aliphatic grouping may include, for purposes of substitution, Gly, Ala, Val, Leu, and Ile. Other conservative substitutions groups include: sulfur-containing: Met and Cysteine (Cys or C); acidic: Asp, Glu, Asn, and Gln; small aliphatic, nonpolar or slightly polar residues: Ala, Ser, Thr, Pro, and Gly; polar, negatively charged residues and their amides: Asp, Asn, Glu, and Gln; polar, positively charged residues: His, Arg, and Lys; large aliphatic, nonpolar residues: Met, Leu, Ile, Val, and Cys; and large aromatic residues: Phe, Tyr, and Trp. Additional information can be found in Creighton (1984) Proteins, W.H. Freeman and Company.

Disclosed polypeptides (e.g., engineered proteins, fusion proteins) may or may not comprise a signal peptide (also known as a leader sequence, leader peptide, or transit peptide). Signal peptides can target newly synthesized polypeptides to their appropriate location inside or outside (including membrane-spanning) the cell. A signal peptide may be removed from the polypeptide during or once localization or secretion is completed. Polypeptides that have a signal peptide can be referred to as a “pre-protein” or “precursor protein” and polypeptides having some or all of their signal peptide removed can be referred to as “mature” proteins or polypeptides. It will be understood that in some cases, removal of a signal peptide from a protein may leave behind one or more signal peptide amino acids on the protein, discussed further herein.

Table 3 shows amino acid sequences of certain proteins of the present disclosure with and without signal peptides. An example of a signal peptide native to CD8α isoform 1 comprises amino acids 1-21 of SEQ ID NO.:1. An example of a signal peptide native to CD8β isoform 1 comprises amino acids 1-21 of SEQ ID NO.:6. Certain signal peptide amino acid sequences are also provided in SEQ ID NOs.: 168-172 and 179. Signal peptides are annotated for various precursor protein sequences; e.g., the UniProt database. It will be appreciated that any suitable naturally occurring or engineered signal peptide can be employed. Certain signal peptides and characteristics of these are described in Owji et al., European Journal of Cell Biology 97 (6): 422-441 (2018); the signal peptides of which are incorporated herein by reference. Certain disclosed amino acid sequences comprise a signal peptide; such a signal peptide will be recognized by those of ordinary skill in the art, and the amino acid sequence resulting from removal of the signal peptide will also be recognized.

As used herein, “fusion protein” refers to a protein that, in a single chain, has at least two distinct domains, wherein the domains are not naturally found together in a protein. A polynucleotide encoding a fusion protein may be constructed using PCR, recombinantly engineered, or the like, or such fusion proteins can be synthesized. A fusion protein may further contain other components, such as a tag, a linker, or a transduction marker. In certain embodiments, a fusion protein expressed or produced by a host cell (e.g., a T cell) locates to the cell surface, where the fusion protein is anchored to the cell membrane (e.g., via a transmembrane component or domain) and comprises an extracellular component (e.g., capable of associating with a MHC molecule) and an intracellular component (e.g., containing a signaling domain, effector domain, co-stimulatory domain or portions or combinations thereof).

“Nucleic acid molecule” or “polynucleotide” refers to a polymeric compound including covalently linked nucleotides, which can be made up of natural subunits (e.g., purine or pyrimidine bases) or non-natural subunits (e.g., morpholine ring). Purine bases include adenine, guanine, hypoxanthine, and xanthine, and pyrimidine bases include uracil, thymine, and cytosine. Nucleic acid molecules include polyribonucleic acid (RNA), polydeoxyribonucleic acid (DNA), which includes cDNA, genomic DNA, and synthetic DNA, either of which may be single or double-stranded. If single-stranded, the nucleic acid molecule may be the coding strand or non-coding (anti-sense strand). A nucleic acid molecule encoding an amino acid sequence includes all nucleotide sequences that encode the same amino acid sequence. Some versions of the nucleotide sequences may also include intron(s) to the extent that the intron(s) would be removed through co- or post-transcriptional mechanisms. In other words, different nucleotide sequences may encode the same amino acid sequence as the result of the redundancy or degeneracy of the genetic code, or by splicing.

In some embodiments, the polynucleotide (e.g. mRNA) comprises a modified nucleoside, a cap-1 structure, a cap-2 structure, or any combination thereof. In certain embodiments, the polynucleotide comprises a pseudouridine, a N6-methyladenonsine, a 5-methylcytidine, a 2-thiouridine, or any combination thereof. In some embodiments, the pseudouridine comprises N1-methylpseudouridine. These features are known in the art and are discussed in, for example, Zhang et al. Front. Immunol., DOI=10.3389/fimmu.2019.00594 (2019); Eyler et al. PNAS 116 (46): 23068-23071; DOI: 10.1073/pnas. 1821754116 (2019); Nance and Meier, ACS Cent. Sci. 2021, 7, 5, 748-756; doi.org/10.1021/acscentsci.1c00197 (2021), and van Hoecke and Roose, J. Translational Med 17:54 (2019); https://doi.org/10.1186/s12967-019-1804-8, which modified nucleosides and mRNA features are incorporated herein by reference.

Variants of nucleic acid molecules of this disclosure are also contemplated. Variant nucleic acid molecules are at least 70%, 75%, 80%, 85%, 90%, and are preferably 95%, 96%, 97%, 98%, 99%, or 99.9% identical a nucleic acid molecule of a defined or reference polynucleotide as described herein, or that hybridize to a polynucleotide under stringent hybridization conditions of 0.015M sodium chloride, 0.0015M sodium citrate at about 65-68° C. or 0.015M sodium chloride, 0.0015M sodium citrate, and 50% formamide at about 42° C. Nucleic acid molecule variants retain the capacity to encode a fusion protein or a binding domain thereof having a functionality described herein, such as specifically binding a target molecule.

“Percent sequence identity” refers to a relationship between two or more sequences, as determined by comparing the sequences. Preferred methods to determine sequence identity are designed to give the best match between the sequences being compared. For example, the sequences are aligned for optimal comparison purposes (e.g., gaps can be introduced in one or both of a first and a second amino acid or nucleic acid sequence for optimal alignment). Further, non-homologous sequences may be disregarded for comparison purposes. The percent sequence identity referenced herein is calculated over the length of the reference sequence, unless indicated otherwise. Methods to determine sequence identity and similarity can be found in publicly available computer programs. Sequence alignments and percent identity calculations may be performed using a BLAST program (e.g., BLAST 2.0, BLASTP, BLASTN, or BLASTX). The mathematical algorithm used in the BLAST programs can be found in Altschul et al., Nucleic Acids Res. 25:3389-3402, 1997. Within the context of this disclosure, it will be understood that where sequence analysis software is used for analysis, the results of the analysis are based on the “default values” of the program referenced. “Default values” mean any set of values or parameters which originally load with the software when first initialized.

The term “isolated” means that the material is removed from its original environment (e.g., the natural environment if it is naturally occurring). For example, a naturally occurring nucleic acid or polypeptide present in a living animal is not isolated, but the same nucleic acid or polypeptide, separated from some or all of the co-existing materials in the natural system, is isolated. Such nucleic acid could be part of a vector and/or such nucleic acid or polypeptide could be part of a composition (e.g., a cell lysate), and still be isolated in that such vector or composition is not part of the natural environment for the nucleic acid or polypeptide. “Isolated” can, in some embodiments, also describe a polynucleotide, vector, host cell, or composition that is outside of a human body. In any of the presently disclosed embodiments, a polynucleotide, vector, polypeptide, or host cell can be “isolated.”

The term “gene” means the segment of DNA involved in producing a polypeptide chain; it includes regions preceding and following the coding region (“leader and trailer”) as well as intervening sequences (introns) between individual coding segments (exons).

A “functional variant” refers to a polypeptide or polynucleotide that is structurally similar or substantially structurally similar to a parent or reference compound of this disclosure, but differs slightly in composition (e.g., one base, atom or functional group is different, added, or removed), such that the polypeptide or encoded polypeptide is capable of performing at least one function of the parent polypeptide with at least 50% efficiency, preferably at least 55%, 60%, 70%, 75%, 80%, 85%, 90%, 95%, 96%, 97%, 98%, 99%, 99.9%, or 100% level of activity of the parent polypeptide. In other words, a functional variant of a polypeptide or encoded polypeptide of this disclosure has “similar binding,” “similar affinity” or “similar activity” when the functional variant displays no more than a 50% reduction in performance in a selected assay as compared to the parent or reference polypeptide, such as an assay for measuring binding affinity (e.g., Biacore® or tetramer staining measuring an association (Kₐ) or a dissociation (K_{D}) constant).

As used herein, a “functional portion” or “functional fragment” refers to a polypeptide or polynucleotide that comprises only a domain, portion or fragment of a parent or reference compound, and the polypeptide or encoded polypeptide retains at least 50% activity associated with the domain, portion or fragment of the parent or reference compound, preferably at least 55%, 60%, 70%, 75%, 80%, 85%, 90%, 95%, 96%, 97%, 98%, 99%, 99.9%, or 100% level of activity of the parent polypeptide, or provides a biological benefit (e.g., effector function). A “functional portion” or “functional fragment” of a polypeptide or encoded polypeptide of this disclosure has “similar binding” or “similar activity” when the functional portion or fragment displays no more than a 50% reduction in performance in a selected assay as compared to the parent or reference polypeptide (preferably no more than 20% or 10%, or no more than a log difference as compared to the parent or reference with regard to affinity), such as an assay for measuring binding affinity or measuring effector function (e.g., cytokine release). As discussed further herein, it will be understood that a functional portion, fragment, or variant of a parent or reference polypeptide preferably retains or substantially retains a native function such that the polypeptide (e.g. fusion protein) of the present disclosure that comprises the functional portion, fragment, or variant is capable of performing the function. By way of example, for a fusion protein that comprises a CD8β extracellular component and an intracellular component comprising a functional portion, fragment, or variant of a CD28 costimulatory domain, the fusion protein is functional to provide a CD28 costimulatory signal upon ligand/target binding; when e.g. the extracellular component binds to a MHC class I molecule. In some embodiments, the native function is preferably retained, substantially retained, or augmented.

As used herein, “heterologous” or “non-endogenous” or “exogenous” refers to any gene, protein, compound, nucleic acid molecule, or activity that is not native to a host cell or a subject, or any gene, protein, compound, nucleic acid molecule, or activity native to a host cell or a subject that has been altered. Heterologous, non-endogenous, or exogenous includes genes, proteins, compounds, or nucleic acid molecules that have been mutated or otherwise altered such that the structure, activity, or both is different as between the native and altered genes, proteins, compounds, or nucleic acid molecules. In certain embodiments, heterologous, non-endogenous, or exogenous genes, proteins, or nucleic acid molecules (e.g., receptors, ligands, etc.) may not be endogenous to a host cell or a subject, but instead nucleic acids encoding such genes, proteins, or nucleic acid molecules may have been added to a host cell by conjugation, transformation, transfection, electroporation, or the like, wherein the added nucleic acid molecule may integrate into a host cell genome or can exist as extra-chromosomal genetic material (e.g., as a plasmid or other self-replicating vector). The term “homologous” or “homolog” refers to a gene, protein, compound, nucleic acid molecule, or activity found in or derived from a host cell, species, or strain. For example, a heterologous or exogenous polynucleotide or gene encoding a polypeptide may be homologous to a native polynucleotide or gene and encode a homologous polypeptide or activity, but the polynucleotide or polypeptide may have an altered structure, sequence, expression level, or any combination thereof. A non-endogenous polynucleotide or gene, as well as the encoded polypeptide or activity, may be from the same species, a different species, or a combination thereof.

As used herein, the term “endogenous” or “native” refers to a polynucleotide, gene, protein, compound, molecule, or activity that is normally present in a host cell or a subject.

The term “expression”, as used herein, refers to the process by which a polypeptide is produced based on the encoding sequence of a nucleic acid molecule, such as a gene. The process may include transcription, post-transcriptional control, post-transcriptional modification, translation, post-translational control, post-translational modification, or any combination thereof. An expressed nucleic acid molecule is typically operably linked to an expression control sequence (e.g., a promoter).

The term “operably linked” refers to the association of two or more nucleic acid molecules on a single nucleic acid fragment so that the function of one is affected by the other. For example, a promoter is operably linked with a coding sequence when it is capable of affecting the expression of that coding sequence (i.e., the coding sequence is under the transcriptional control of the promoter). “Unlinked” means that the associated genetic elements are not closely associated with one another and the function of one does not affect the other.

As used herein, “expression vector” refers to a DNA construct containing a nucleic acid molecule that is operably linked to a suitable control sequence capable of effecting the expression of the nucleic acid molecule in a suitable host. Such control sequences include a promoter to effect transcription, an optional operator sequence to control such transcription, a sequence encoding suitable mRNA ribosome binding sites, and sequences which control termination of transcription and translation. The vector may be a plasmid, a phage particle, a virus, or simply a potential genomic insert. Once transformed into a suitable host, the vector may replicate and function independently of the host genome, or may, in some instances, integrate into the genome itself. In the present specification, “plasmid,” “expression plasmid,” “virus” and “vector” are often used interchangeably. Vectors are discussed further herein.

The term “introduced” in the context of inserting a nucleic acid molecule into a cell, means “transfection”, or “transformation” or “transduction” and includes reference to the incorporation of a nucleic acid molecule into a eukaryotic or prokaryotic cell wherein the nucleic acid molecule may be incorporated into the genome of a cell (e.g., chromosome, plasmid, plastid, or mitochondrial DNA), converted into an autonomous replicon, or transiently expressed (e.g., transfected mRNA). As used herein, the term “engineered,” “recombinant” or “non-natural” refers to an organism, microorganism, cell, nucleic acid molecule, or vector that includes at least one genetic alteration or has been modified by introduction of an exogenous nucleic acid molecule, wherein such alterations or modifications are introduced by genetic engineering (i.e., human intervention). Genetic alterations include, for example, modifications introducing expressible nucleic acid molecules encoding proteins, fusion proteins or enzymes, or other nucleic acid molecule additions, deletions, substitutions or other functional disruption of a cell's genetic material. Additional modifications include, for example, non-coding regulatory regions in which the modifications alter expression of a polynucleotide, gene or operon. In some embodiments, an “engineered” cell refers to a modified cell.

As described herein, more than one heterologous nucleic acid molecule can be introduced into a host cell as separate nucleic acid molecules, as a plurality of individually controlled genes, as a polycistronic nucleic acid molecule, as a single nucleic acid molecule encoding a fusion protein, or any combination thereof. When two or more heterologous nucleic acid molecules are introduced into a host cell, it is understood that the two or more heterologous nucleic acid molecules can be introduced as a single nucleic acid molecule (e.g., on a single vector), on separate vectors, integrated into the host chromosome at a single site or multiple sites, or any combination thereof. The number of referenced heterologous nucleic acid molecules or protein activities refers to the number of encoding nucleic acid molecules or the number of protein activities, not the number of separate nucleic acid molecules introduced into a host cell.

The term “construct” refers to any polynucleotide that contains a recombinant nucleic acid molecule (or, when the context clearly indicates, a (n e.g. fusion) protein of the present disclosure). A “transgene” or “transgene construct” refers to a construct that contains two or more genes operably linked in an arrangement that is not found in nature. A (polynucleotide) construct may be present in a vector (e.g., a bacterial vector, a viral vector) or may be integrated into a genome. A “vector” is a nucleic acid molecule that is capable of transporting another nucleic acid molecule. Vectors may be, for example, plasmids, cosmids, viruses, a RNA vector or a linear or circular DNA or RNA molecule that may include chromosomal, non-chromosomal, semi-synthetic or synthetic nucleic acid molecules. Vectors of the present disclosure also include transposon systems (e.g., Sleeping Beauty, see, e.g., Geurts et al., Mol. Ther. 8:108, 2003: Mátés et al., Nat. Genet. 41:753, 2009). Exemplary vectors are those capable of autonomous replication (episomal vector), capable of delivering a polynucleotide to a cell genome (e.g., viral vector), or capable of expressing nucleic acid molecules to which they are linked (expression vectors).

As used herein, the term “host” refers to a cell (e.g., T cell) or microorganism targeted for genetic modification with a heterologous nucleic acid molecule to produce a polypeptide of interest (e.g., a fusion protein of the present disclosure). In certain embodiments, a host cell may optionally already possess or be modified to include other genetic modifications that confer desired properties related or unrelated to, e.g., biosynthesis of the heterologous protein (e.g., inclusion of a detectable marker; deleted, altered or truncated endogenous TCR; or increased co-stimulatory factor expression).

“Antigen” or “Ag” as used herein refers to an immunogenic molecule that provokes an immune response. This immune response may involve, for example, antibody production, activation of specific immunologically-competent cells (e.g., T cells), or both. An antigen (immunogenic molecule) may be, for example, a peptide, glycopeptide, polypeptide, glycopolypeptide, polynucleotide, polysaccharide, lipid or the like. It is readily apparent that an antigen can be synthesized, produced recombinantly, or derived from a biological sample. Exemplary biological samples that can contain one or more antigens include tissue samples, tumor samples, cells, biological fluids, or combinations thereof. Antigens can be produced by cells that have been modified or genetically engineered to express an antigen. Antigens can be expressed at a cell surface or presented in complex with a MHC molecule.

The term “epitope” or “antigenic epitope” includes any molecule, structure, amino acid sequence or protein determinant that is recognized and specifically bound by a cognate binding molecule, such as an immunoglobulin, T cell receptor (TCR), chimeric antigen receptor, or other binding molecule, domain or protein. Epitopic determinants generally contain chemically active surface groupings of molecules, such as amino acids or sugar side chains, and can have specific three dimensional structural characteristics, as well as specific charge characteristics.

“T cell receptor” (TCR) refers to an immunoglobulin superfamily member (having a variable binding domain, a constant domain, a transmembrane region, and a short cytoplasmic tail; see, e.g., Janeway et al., Immunobiology: The Immune System in Health and Disease, 3^{rd} Ed., Current Biology Publications, p. 4:33, 1997) capable of specifically binding to an antigen peptide bound to a MHC receptor. A TCR can be found on the surface of a cell or in soluble form and generally is comprised of a heterodimer having α and β chains (also known as TCRα and TCRβ, respectively), or γ and δ chains (also known as TCRγ and TCRδ, respectively). Like immunoglobulins, the extracellular portion (protein extracellular portions or domains are also referred to herein as “ectodomains” herein, while protein intracellular or cytoplasmic portions or domains are also referred to herein as “endodomains” herein) of each TCR chain (e.g., α-chain, β-chain) contains two immunoglobulin domains: a variable domain (e.g., α-chain variable domain or V_{α}, β-chain variable domain or V_{β}; typically amino acids 1 to 116 based on Kabat numbering (Kabat et al., “Sequences of Proteins of Immunological Interest, US Dept. Health and Human Services, Public Health Service National Institutes of Health, 1991, 5^{th} ed.) at the N-terminus; and a constant domain (e.g., α-chain constant domain or C_{α}, typically amino acids 117 to 259 based on Kabat, β-chain constant domain or C_{β}, typically amino acids 117 to 295 based on Kabat) adjacent to the cell membrane. See also Lefranc et al., Dev. Comp. Immunol. 27:55, 2003. The variable domains contain complementary determining regions (CDRs) separated by framework regions (FRs) (see, e.g., Jores et al., Proc. Nat'l Acad. Sci. U.S.A. 87:9138, 1990; Chothia et al., EMBO J. 7:3745, 1988; see also Lefranc et al., Dev. Comp. Immunol. 27:55, 2003).

The term “variable region” or “variable domain” refers to the domain of a TCR α-chain or β-chain (or γ-chain and δ-chain for γδ TCRs), or of an antibody heavy or light chain, that is involved in binding to antigen. The variable domains of the α-chain and β-chain (Vα and Vβ, respectively) of a native TCR generally have similar structures, with each domain comprising four generally conserved framework regions (FRs) and three CDRs. Variable domains of antibody heavy (V_{H}) and light (V_{L}) chains each also generally comprise four generally conserved framework regions (FRs) and three CDRs.

The terms “complementarity determining region,” and “CDR,” are synonymous with “hypervariable region” or “HVR,” and are known in the art to refer to non-contiguous sequences of amino acids within TCR or antibody variable regions, which confer antigen specificity and/or binding affinity. In general, there are three CDRs in each variable region (i.e., three CDRs in each of the TCRα-chain and β-chain variable regions; 3 CDRs in each of the antibody heavy chain and light chain variable regions). In the case of TCRs, CDR3 is thought to be the main CDR responsible for recognizing processed antigen. CDR1 and CDR2 mainly interact with the MHC. Variable domain sequences can be aligned to a numbering scheme (e.g., Kabat, EU, International Immunogenetics Information System (IMGT) and Aho), which can allow equivalent residue positions to be annotated and for different molecules to be compared using Antigen receptor Numbering And Receptor Classification (ANARCI) software tool (2016, Bioinformatics 15:298-300).

In certain embodiments, a TCR is found on the surface of T cells (or T lymphocytes) and associates with the CD3 complex. The source of a TCR as used in the present disclosure may be from various animal species, such as a human, mouse, rat, rabbit or other mammal. “CD3” is a multi-protein complex of six chains that is involved in T cell signaling in response to antigen. (see, Abbas and Lichtman, 2003; Janeway et al., p. 172 and 178, 1999). In mammals, the complex generally comprises a CD3γ chain, a CD3δ chain, two CD3ε chains (each of which, in general, associates with a cognate CD3γ chain or CD3δ chain to form a dimer), and a homodimer of CD3ζ chains. The CD3γ, CD3δ, and CD3ε chains are related cell surface proteins of the immunoglobulin superfamily containing a single immunoglobulin domain. The transmembrane regions of the CD3γ, CD3δ, and CD3ε chains are negatively charged, which is thought to allow these chains to associate with positively charged regions of T cell receptor chains. The intracellular tails of the CD3γ, CD3δ, and CD3ε chains each contain a single conserved motif known as an immunoreceptor tyrosine-based activation motif or ITAM, whereas each CD3ζ chain has three ITAMS. Without wishing to be bound by theory, it is believed that ITAMs are important for the signaling capacity of a TCR complex. CD3 as used in the present disclosure may be from various animal species, including human, mouse, rat, or other mammals. Accordingly, it will be understood that a functional portion or a variant of a CD3 protein intracellular domain contains one or more ITAM and optionally other sequence features that are involved in signaling. Examples of sequences from human CD3 proteins are provided in SEQ ID NOs:69-77.

“Major histocompatibility complex molecules” (MHC molecules) refer to glycoproteins that deliver peptide antigens to a cell surface. MHC class I molecules are heterodimers consisting of a membrane spanning α chain (with three a domains) and a non-covalently associated β2 microglobulin. MHC class II molecules are composed of two transmembrane glycoproteins, α and β, both of which span the membrane. Each chain has two domains. MHC class I molecules deliver peptides originating in the cytosol to the cell surface, where a peptide: MHC complex is recognized by CD8⁺ T cells. MHC class II molecules deliver peptides originating in the vesicular system to the cell surface, where they are recognized by CD4⁺ T cells. An MHC molecule may be from various animal species, including human (i.e., HLA molecule), mouse, rat, cat, dog, goat, horse, or other mammals. HLAs corresponding to “class I” MHC present peptides from inside the cell and include, for example, HLA-A, HLA-B, and HLA-C. Alleles include, for example, HLA A*02:01; HLA-A*03:01; HLA-A*11:01; HLA-B*07:02; HLA-B*40:01; HLA-B*44:02; or HLA-B*44:03. HLAs corresponding to “class II” MHC present peptides from outside the cell and include, for example, HLA-DP, HLA-DM, HLA-DOA, HLA-DOB, HLA-DQ, and HLA-DR.

In some embodiments, a class I MHC comprises an HLA. In certain embodiments, the HLA comprises HLA-A, HLA-B, and/or HLA-C. In certain further embodiments, the HLA comprises HLA A*02:01; HLA-A*03:01; HLA-A*11:01; HLA-B*07:02; HLA-B*40:01; HLA-B*44:02; or HLA-B*44:03.

As used herein, the term “CD8 co-receptor” or “CD8” includes the cell surface glycoprotein CD8, which is sometimes expressed by T cells as a homodimer comprising two CD8α chains, or as a heterodimer comprising an α chain and a β chain. The CD8 co-receptor is believed to assist in the function of cytotoxic T cells (CD8⁺) and functions through signaling via its cytoplasmic tyrosine phosphorylation pathway (Gao and Jakobsen, Immunol. Today 21:630-636, 2000; Cole and Gao, Cell. Mol. Immunol. 1:81-88, 2004). In particular, and without wishing to be bound by theory, it is believed that the CD8 co-receptor binds to an MHC-I protein complex expressed on the surface of an antigen-expressing cell, and that this binding in the context of TCR: antigen-MHC binding initiates or assists in a T cell signaling pathway that produces an immune response (e.g., transcription and expression of cytokines, calcium secretion, cytolytic activity, or the like) against the antigen-expressing cell.

In humans, eight (8) different CD8 beta chain isoforms are known (“M1”-“M8”; see UniProtKB identifiers P10966-1, 2, 3, 4, 6, 7, 8, and 9); of these, isoforms 1, 2, 4, and 5 are thought to associate with the cell membrane in nature, while isoforms 3, 6, 7, and 8 are believed to associate with extracellular regions or be secreted. The amino acid sequences of these CD8 β-chain isoforms (including with and without leader (i.e., signal peptide) sequences) are incorporated by reference herein. Amino acid sequences from certain CD8 co-receptor β-chains of the present disclosure are shown in SEQ ID NOs:6-17. In certain embodiments, a (n e.g. fusion) protein of the present disclosure comprises an extracellular and/or transmembrane component from a CD8 co-receptor β-chain M1 isoform, or functional variant or portion thereof.

Also in humans, three CD8 alpha chain isoforms are known (see UniProtKB identifiers P01732-1, 2, and 3). The amino acid sequences of these CD8 α-chain isoforms (including with and without leader (i.e., signal peptide) sequences) are incorporated by reference herein. Amino acid sequences from certain CD8 co-receptor α-chains of the present disclosure are shown in SEQ ID NOs:1-5.

Reference to CD8α includes the “canonical” human CD8α protein (NP_001759.3) as well as splice isoform 2, which lacks an internal segment including the transmembrane domain resulting in a secreted protein (RefSeq NP_741969.1), and splice isoform 3, which uses an alternate promoter and 5′ UTR (RefSeq NP_001139345.1). Reference to CD8β includes the “canonical” human CD8β protein (RefSeq NP_004922) as well as isoforms 2-8, corresponding to RefSeq NP742099, RefSeq NP_742100, UniProt P10966-4, RefSeq NP_757362, Uniprot P10966-7, Uniprot P10966-8, and RefSeq NP_001171571.

It will be understood that the terms “CD8 polypeptide” and “CD8 co-receptor polypeptide” can be used interchangeably, including when the subject polypeptide functions as a protein monomer. A “CD8α” polypeptide can be a wild-type CD8α chain or a fragment thereof (of any isotype or species), as well as an engineered (e.g. fusion or chimeric) polypeptide that comprises at least some portion of a CD8α extracellular domain and is capable of binding to a Class I MHC molecule, such as in a manner that is at least substantially similar to the manner in which wild-type CD8α binds to the Class I MHC molecule. In some embodiments, a CD8α polypeptide further comprises CD8α transmembrane and/or intracellular amino acid sequence or features. In some embodiments, a CD8α polypeptide comprises an intracellular component that can associate with a Lck.

A “CD8β” polypeptide can be a wild-type CD8α chain or a fragment thereof (of any isotype or species), as well as an engineered (e.g. fusion or chimeric) polypeptide that comprises at least some portion of a CD8β extracellular domain and is capable of binding to a Class I MHC molecule, such as in a manner that is at least substantially similar to the manner in which wild-type CD8β binds to the Class I MHC molecule. In some embodiments, a CD8β polypeptide further comprises CD8β transmembrane and/or intracellular amino acid sequence or features.

“CD4” refers to an immunoglobulin co-receptor glycoprotein that assists the TCR in communicating with antigen-presenting cells (see, Campbell & Reece, Biology 909 (Benjamin Cummings, Sixth Ed., 2002); UniProtKB P01730). CD4 is found on the surface of immune cells such as T helper cells, monocytes, macrophages, and dendritic cells, and typically includes four immunoglobulin domains (D1 (comprising an Ig-like V-type domain), D2, D3, and D4 (respectively comprising Ig-like C2-type domains 1, 2, and 3)) that are expressed at the cell surface. During antigen presentation, CD4 is recruited, along with the TCR complex, to bind to different regions of the MHCII molecule (CD4 binds MHCII β2, while the TCR complex binds MHCII α1/β1). Without wishing to be bound by theory, it is believed that close proximity to the TCR complex allows CD4-associated kinase molecules to phosphorylate the immunoreceptor tyrosine activation motifs (ITAMs) present on the cytoplasmic domains of CD3. This activity is thought to amplify the signal generated by the activated TCR in order to produce various types of T helper cells. Examples of human CD4 amino acid sequences are disclosed in UniProt KB entry no. P01730; these amino acid sequences (including with and without leader (i.e., signal peptide) sequences) are incorporated by reference herein.

### Polypeptides, Polynucleotides, and Vectors

In one aspect, the present disclosure provides polypeptides, such as fusion proteins and other engineered proteins, that are useful to confer to, or improve, a desired activity of a host immune cell, such as an immune cell that targets a diseased or pathogenic cell (e.g. a cancer cell). Also provided are polynucleotides that encode any one or more of the polypeptides, and vectors that comprise a polynucleotide. In some contexts, a polynucleotide encodes two or more polypeptides that can function when associating as a multimer (e.g. as a dimer) at the surface of a host cell, such as a T cell. In any of the presently disclosed embodiments, a polypeptide can comprise a human amino acid sequence, or can be derived (e.g. engineered) from a human amino acid sequence. In any of the presently disclosed embodiments, a host cell can be a human cell.

In some embodiments, a polypeptide is provided that comprises: (i) an extracellular component that comprising a binding domain that is capable of binding to a MHC Class I molecule; (ii) a transmembrane domain; and (iii) an intracellular component comprising a signaling domain (e.g. a costimulatory domain) such as, for example, a CD28, 4-1BB, OX40, ICOS, or GITR signaling domain, or a functional portion or variant thereof. In some embodiments, the binding domain comprises an Ig V-like domain (e.g. from a CD8α, a CD8β, or a functional variant thereof), and/or the extracellular component comprises a length of about 170 amino acids, about 165 amino acids, about 160 amino acids, about 155 amino acids, about 150 amino acids, or about 145 amino acids, or of between 145 and 175 amino acids, or of between 145 and 170 amino acids, or of between 145 and 165 amino acids, or of between 145 and 160 amino acids, or of between 145 and 155 amino acids, or of between 145 and 155 amino acids, or of between 145 and 150 amino acids, or of between 150 and 175 amino acids, or of between 150 and 170 amino acids, or of between 150 and 165 amino acids, or of between 150 and 160 amino acids, or of between 150 and 155 amino acids, or of between 155 and 175 amino acids, or of between 155 and 170 amino acids, or of between 155 and 165 amino acids, or of between 155 and 160 amino acids, or of between 160 and 175 amino acids, or of between 160 and 170 amino acids, or of between 165 and 165 amino acids, or of between 165 and 175 amino acids, or of between 165 and 170 amino acids. In some embodiments, the extracellular component does not comprise a wild-type CD8α or wild-type CD8β extracellular component.

Certain embodiments include fusion (also referred-to herein as chimeric) CD8 co-receptor proteins, which can include an intracellular signaling domain from another protein (e.g., CD28, Lck, 4-1BB, ICOS, OX40, GITR) and can translate MHC-binding into a stimulatory signal in a host cell. Chimeric CD8 co-receptor proteins include those that may function advantageously with a cognate CD8 co-receptor protein (e.g., in some embodiments, a chimeric CD8β protein may function advantageously with a cognate CD8α protein, which itself may be wild-type or engineered (e.g. chimeric); a chimeric CD8α protein can function with a cognate CD8β (or CD8α) protein, which itself may be wild-type or engineered (e.g. chimeric). In some embodiments, two or more CD8 co-receptor proteins, one or more of which may be a chimeric CD8 co-receptor of the present disclosure, are encoded by a same polynucleotide or vector and/or are expressed in a same host cell. In some embodiments, a CD8 co-receptor protein (e.g. CD8α) and a chemokine receptor protein (e.g. CCR4 or CCR2b) are encoded by a same polynucleotide or vector and/or are expressed in a same host cell. In certain embodiments, two proteins of the present disclosure can be expressed co-ordinately, e.g. by use of an expression construct that drives expression of both proteins in a fusion construct, and the fusion comprises e.g. a cleavable or cleaving amino acid sequence so that the encoded component proteins separate from one another and can express as separate molecules at the host cell surface. In particular embodiments, the polynucleotide or vector further encodes a binding protein, such as a TCR, and/or the host cell further expresses a binding protein, such as a TCR. In some embodiments, a TCR is MHC I-restricted.

Also provided are engineered (e.g. fusion) polypeptides that can function advantageously without a cognate co-protein to perform a desired function in a host cell. In certain embodiments, a polypeptide comprises components (e.g. amino acid sequences or domains) from CD8α and CD8β (of any isoform or combination of isoforms), and, optionally, from one or more additional polypeptides, such as, for example, CD28. Such proteins can be referred to as “single-stalk” proteins. In certain other embodiments, a polypeptide comprises an extracellular domain from a CD3 complex protein (e.g. CD3ζ), an intracellular signaling domain or functional portion thereof from a costimulatory protein such as CD28 or 4-1BB, and a CD3 protein (e.g. CD3ζ) intracellular signaling (e.g. effector) domain.

In certain other embodiments, a polypeptide comprises an extracellular component from a C-type lectin-like receptor, such as NKG2D, and an intracellular component from a costimulatory protein such as, for example, CD28.

In certain other embodiments, an polypeptide can attenuate or prevent an undesired cell-suppressive signal from Fas:FasL binding and/or can translate such a binding into a desired (e.g. activating) signal to the host cell. For example, in some embodiments, a polypeptide comprises an extracellular component from Fas and comprises: no functional Fas intracellular signaling domain (e.g. the Fas intracellular signaling domain may be absent); an intracellular signaling domain from Lck (see e.g. Palacios and Weiss, Oncogene 23:7990-8000 (2004) and Rossy et al. Front. Immunol. doi.org/10.3389/fimmu.2012.00167 (2012)); an intracellular signaling domain from a CD8 co-receptor protein (e.g. CD8α); or an intracellular signaling domain from TRAF1 (see e.g. SEQ ID NO.:181 and e.g. Edilova et al. Front. Immunol. doi.org/10.3389/fimmu.2018.02969 (2018)).

Without being bound by theory, certain disclosed polypeptides may exert one or more effect by translating or, e.g. in the case of engineered non-signaling Fas polypeptides, failing to translate into downstream action, a signal that originates with an extracellular ligand- or target-binding event. Accordingly, certain presently disclosed polypeptides can bind to a (n e.g. cognate) target molecule. For example, in some embodiments, polypeptides comprising an extracellular binding domain from a CD8 protein can bind to a MHC Class I molecule (e.g. concurrent to TCR binding to antigen: MHC), in some embodiments, polypeptides comprising an extracellular binding domain from NKG2D can bind to a NKG2D ligand (discussed further herein), and in some embodiments, polypeptides comprising an extracellular binding domain from Fas can bind to FasL.

Binding to or associating with a target or cognate molecule can be assessed by known methods, such as, for example, peptide: MHC multimer/tetramer staining, Western blot, ELISA, analytical ultracentrifugation, spectroscopy, surface plasmon resonance (Biacore®) isothermal titration calorimetry, and biolayer interferometry (see, e.g., Dolton et al., Immunology 146:11-22, 2015, Scatchard et al., Ann. NY Acad. Sci. 51:660, 1949; Wilson, Science 20295:2103, 2002; Wolff et al., Cancer Res. 53:2560, 1993; and U.S. Pat. Nos. 5,283,173, 5,468,614, or the equivalent; all incorporated herein by reference). For example, a protein-protein binding interaction can be investigated by immobilizing one binding partner on a bead or plate, and passing the other binding partner thereover in solution, and detecting binding by, e.g., a refractive index or a wavelength shift. Flow cytometry and other cell sorting and imaging techniques may also be used to investigate binding by cell surface-expressed molecules.

It will be understood that CD8 co-receptor polypeptides of the present disclosure (and portions or domains or functional variants of same) retain the ability to bind to a MHC complex molecule, such as MHC Class I molecule. Briefly, CD8 co-receptors will typically include at least one immunoglobulin-like V-type domain that binds to a cognate MHC molecule. These V-type domains, and functional variants thereof, including those comprising one or more conservative or non-conservative amino acid substitutions relative to a parental or wild-type sequence, are contemplated herein. In certain embodiments, a functional variant or portion of a CD8α extracellular domain or a CD8β extracellular domain is capable of binding to a MHC Class I molecule. In further embodiments, a functional variant or portion of a CD8α or a CD8β extracellular domain comprises an Ig V-like domain, or a functional portion or variant thereof. Sequences comprising an Ig V-like domain can include, for example, amino acids 22-135 of SEQ ID NO:1 (see also amino acids 1-114 of SEQ ID NOS: 2 and 5), or amino acids 22-132 of SEQ ID NO:6 (see also amino acids 1-110 of SEQ ID NO:7), and are set forth in SEQ ID NOs:67 and 68. Also contemplated are engineered CD8 co-receptor polypeptides that comprise one or more mutations for enhanced or decreased binding to MHC (see e.g. Devine et al. J. Immunol 177 (6): 2006, which binding mutants are incorporated herein by reference). In some embodiments, a polypeptide comprising an Ig V-like domain comprises or consists of an amino acid sequence having at least 90%, at least 91%, at least 92%, at least 93%, at least 94%, at least 95%, at least 96%, at least 97%, at least 98%, or at least 99% identity to, or comprising or consisting of, the amino acid sequence set forth in SEQ ID NO.:67 or 68, or a portion or fragment thereof that is capable of binding to a MHC Class I molecule.

In some embodiments, a CD8 co-receptor polypeptides of the present disclosure comprises, in the extracellular component, a CD8 stalk region. A stalk region refers to the extracellular portion of a mature CD8α or CD8β chain that is not the Ig V-like domain. A stalk region typically comprises a length of from about 30 to about 50 amino acid residues and can, in some embodiments, contain one or more O-linked glycan. See e.g. Kern et al. Immunity 9 (4): 519-530 (1998). In some embodiments, a stalk region has at least about 90%, at least about 91%, at least about 92%, at least about 93%, at least about 94%, at least about 95%, at least about 96%, at least about 97%, at least about 98%, or at least about 99% identity to, or comprises or consists of, amino acids 112-149 of SEQ ID NO.:7 or amino acids 115-161 of SEQ ID NO.:2.

Moreover, in certain embodiments, a CD8 co-receptor polypeptide, or a variant or portion of the same (e.g., comprised in a presently disclosed fusion protein) comprises an extracellular portion, component, or domain of sufficient length to allow binding to MHC in the context of an immunological synapse; e.g., a synapse comprising a binding protein (e.g., a TCR) expressed by the CD8− (or fusion protein−) expressing cell in association with an antigen or antigen: MHC complex expressed by a target cell, and optionally further comprising, on the host cell, one or more native costimulatory polypeptide, and, on the target cell, one or more cognate ligand of the one or more costimulatory polypeptide.

Also provided are engineered Fas polypeptides. Briefly, Fas is expressed on the surface of some T cells and, in some contexts, binding of Fas to its ligand (FasL) expressed on a neighboring cell (e.g. a cancer cell) can suppress T cell function and lead to death of the T cell. Disclosed embodiments include a Fas polypeptide that lacks a functional Fas intracellular signaling domain (e.g., a truncated Fas polypeptide lacking an intracellular domain), as well as fusion proteins comprising a Fas extracellular component and an intracellular component from Lck, CD8α, or TRAF1, with the potential to convert Fas: FasL binding to a stimulatory signal.

Also provided are chemokine receptor polypeptides (e.g., CCR4, CCR2B, or a functional variant or portion thereof) that can provide a stimulatory signal when bound to a ligand. In some embodiments, a chemokine receptor polypeptide is co-expressed with a CD8 polypeptide of the present disclosure.

Without being bound by theory, engineered polypeptides comprising an extracellular domain from a CD3 protein (e.g., CD3ζ) and intracellular costimulatory (e.g., CD28, 4-1BB) and signaling/effector (e.g. CD3ζ) domains may exert one or more effect when part of a CD3 complex and/or in association with a TCR upon antigen-binding. In certain embodiments, such a polypeptide does not further comprise an extracellular spacer domain, an extracellular target (e.g. antigen or ligand)-binding domain, or any combination thereof. In some embodiments, a polypeptide comprises an extracellular component having a length of less than 110 amino acids, less than 100 amino acids, less than 90 amino acids, less than 80 amino acids, less than 70 amino acids, less than 60 amino acids, less than 50 amino acids, less than 40 amino acids, less than 30 amino acids, less than 20 amino acids, or less than 10 amino acids. In some embodiments, a polypeptide comprises an extracellular component having a length of about 9 amino acids. In some embodiments, an extracellular component of a polypeptide consists or consists essentially of the amino acid sequence set forth in any one of SEQ ID NOs.: 69, 71, 73, and 75.

An extracellular component and an intracellular component of a cell membrane-spanning polypeptide are connected by a transmembrane domain. In some embodiments, a polypeptide may lack, or may substantially lack, an intracellular component, but may comprise a transmembrane domain. A “transmembrane domain,” as used herein, is a portion of a transmembrane protein that can insert into or span a cell membrane. A transmembrane domain may also be referred-to as a “transmembrane component”. Transmembrane domains have a three-dimensional structure that is thermodynamically stable in a cell membrane and generally range in length from about 15 amino acids to about 30 amino acids. In some embodiments, a transmembrane domain has a length of 12, 13, 14, 15, 16, 17, 18, 19, 20, 21, 22, 23, 24, 25, 26, 27, 28, 29, 30, 31, 32, or 33 amino acids. The structure of a transmembrane domain may comprise an alpha helix, a beta barrel, a beta sheet, a beta helix, or any combination thereof. In certain embodiments, the transmembrane domain comprises or is derived from a known transmembrane protein (e.g., a CD4 transmembrane domain, a CD8α transmembrane domain, a CD8β transmembrane domain, a CD27 transmembrane domain, a CD28 transmembrane domain, a NKG2D transmembrane domain, a Fas transmembrane domain, a CCR4 transmembrane domain, a CCR2b transmembrane domain, a CD3 (zeta, delta, gamma, or epsilon) transmembrane domain, or any combination thereof).

In some embodiments, an amino acid sequence, polypeptide domain, or polypeptide component is “derived from” a source or parent polypeptide when it comprises no more than 5%, 10%, 15%, or 20% variation in amino acid sequence identity as compared to the source or parent polypeptide.

In certain embodiments, the extracellular component of a fusion protein further comprises a linker disposed between a binding component or domain (e.g., an Ig V-like domain) or receptor ectodomain and the transmembrane domain, or between the transmembrane domain and an intracellular component. As used herein when referring to a component of a fusion protein that connects two domains or components, a “linker” may be an amino acid sequence having from about two amino acids to about 500 amino acids, which can provide flexibility and room for conformational movement between two regions, domains, motifs, fragments, or modules connected by the linker. For example, a linker of the present disclosure can position a fusion protein or polypeptide away from the surface of a host cell expressing the fusion protein so as to enable proper contact between the host cell and a target cell, binding to MHC or other target or ligand, and subsequent signaling (Patel et al., Gene Therapy 6:412-419, 1999). Linker length in a fusion or binding protein of the present disclosure may be varied to maximize target (e.g. MHC) recognition based on the selected target molecule, selected binding epitope, or antigen binding domain size and affinity (see, e.g., Guest et al., J. Immunother. 28:203-11, 2005; PCT Publication No. WO 2014/031687). Examples of linkers include those having a glycine-serine amino acid chain having from one to about ten repeats of GlyₓSer_{y}, wherein x and y are each independently an integer from 0 to 10, provided that x and y are not both 0. Non-limiting examples of linkers are provided in SEQ ID NOs.: 182-184.

In some embodiments, a linker can have a length of up to and including 4, up to and including 6, up to and including 8, up to and including 10, up to and including 12, up to and including 15, up to and including 20, up to and including 30, up to and including 40, or up to and including 50 amino acids. In some embodiments, a linker can have a length of 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acids. In some embodiments, a linker can have a length of one to five amino acids, one to ten amino acids, one to fifteen amino acids, one to twenty amino acids, one to twenty-five amino acids, one to thirty amino acids, five to ten amino acids, five to fifteen amino acids, five to twenty amino acids, five to twenty-five amino acids, five to thirty amino acids, ten to fifteen amino acids, ten to twenty amino acids, ten to twenty-five amino acids, ten to thirty amino acids, fifteen to twenty amino acids, fifteen to twenty-five amino acids, fifteen to thirty amino acids, twenty to twenty-five amino acids, twenty to thirty amino acids, or twenty-five to thirty amino acids, less than thirty amino acids, less than twenty-five amino acids, less than twenty amino acids, less than fifteen amino acids, less than ten amino acids, or less than five amino acids.

Linkers of the present disclosure also include immunoglobulin constant regions (i.e., CH1, CH2, CH3, or CL, of any isotype) and portions thereof. In certain embodiments, the linker comprises a CH3 domain, a CH2 domain, or both. In certain embodiments, the linker comprises a CH2 domain and a CH3 domain. In further embodiments, the CH2 domain and the CH3 domain are each a same isotype. In particular embodiments, the CH2 domain and the CH3 domain are an IgG4 or IgG1 isotype. In other embodiments, the CH2 domain and the CH3 domain are each a different isotype. In specific embodiments, the CH2 comprises a N297Q mutation. Without wishing to be bound by theory, it is believed that CH2 domains with N297Q mutation do not bind FcγR (see, e.g., Sazinsky et al., PNAS 105 (51): 20167 (2008)). In certain embodiments, the linker comprises a human immunoglobulin constant region or a portion thereof. Additional linkers include extracellular domains (or portions thereof, such as hinge or stalk sequences) from CD27, CD28, CD8, CD4, or any combination thereof.

In any of the embodiments described herein, a linker may comprise a hinge region or a portion thereof. Hinge regions are flexible amino acid polymers of variable length and sequence (typically rich in proline and cysteine amino acids) and connect larger and less-flexible regions of immunoglobulin proteins. For example, hinge regions connect the Fc and Fab regions of antibodies and connect the constant and transmembrane regions of TCRs. In certain embodiments, the linker comprises an immunoglobulin constant region or a portion thereof and a hinge region or a portion thereof. In certain embodiments, the linker comprises a glycine-serine linker of the present disclosure. Hinge regions from CD proteins such as, for example, CD8α and CD8β are also contemplated.

In some embodiments, a polypeptide of the present disclosure is capable of providing a stimulatory signal to a host cell expressing the polypeptide. A stimulatory signal is typically provided by one or more sequences, domains, or motifs in an intracellular component of a polypeptide, though the extracellular component and/or transmembrane domain may, in some cases, also affect the stimulatory signal. A stimulatory signal is typically initiated by an interaction or association between a polypeptide of the present disclosure and one or more (e.g. extracellular) cognate polypeptides, target molecules, or ligands.

In certain contexts, “providing” a signal refers to facilitating, relaying, producing, and/or amplifying a signal. For example, a polypeptide can provide a stimulatory signal even if other biomolecules (e.g. other polypeptides) may play a further or downstream role in effecting the signal (e.g., via a signal transduction pathway or transcription of a gene). A portion of a polypeptide (e.g. a costimulatory or effector domain) may be said to “provide” a stimulatory signal though one or more other portions (e.g., an extracellular ligand-binding domain of the polypeptide) may also function in providing the stimulatory signal. In certain embodiments, a host cell is an immune cell such as, for example, a T cell (e.g. a CD4+ T cell, a CD8+ T cell, or both), and a stimulatory signal activates, or contributes to activation of, the immune cell. In some embodiments, a single stimulatory signal may at least partially activate an immune cell, or may contribute to at least partial activation. In some embodiments, two or more stimulatory signals (e.g., one stimulatory signal from a TCR complex, one co-stimulatory signal) are sufficient to activate an immune cell. Activation of an immune cell can comprise, for example, production of a cytokine, production of an antibody, a cytotoxic activity, a phagocytic activity, proliferation of the immune cell, intracellular mobilization of calcium, activation of a transcription factor, transcription of a gene, or the like, or any combination thereof. In some embodiments, a stimulatory signal can improve persistence of a host cell; e.g. of a T cell in an immunosuppressive (e.g. tumor micro-) environment.

For example, in some embodiments, a polypeptide of the present disclosure comprises an extracellular component from a CD8 polypeptide (e.g. CD8α or CD8β), or a functional portion or variant thereof, and a stimulatory signal is initiated by interaction of the extracellular component with a MHC Class I molecule. In certain other embodiments, a polypeptide of the present disclosure comprises an extracellular component from a NKG2D polypeptide, or a functional portion or variant thereof, and a stimulatory signal is initiated by interaction of the extracellular component with a NKG2D ligand. In certain other embodiments, a polypeptide of the present disclosure comprises an extracellular component from a Fas polypeptide, or a functional portion or variant thereof, and a stimulatory signal is initiated by interaction of the extracellular component with a Fas ligand (e.g. FasL). In certain other embodiments, a polypeptide of the present disclosure comprises an extracellular component from a PD-1 polypeptide, or a functional portion or variant thereof, and a stimulatory signal is initiated by interaction of the extracellular component with a PD-1 ligand (e.g. PD-L1).

In some embodiments, a polypeptide of the present disclosure comprises an extracellular component from a CD3 polypeptide (e.g. CD3ζ), intracellular sequence or effector domain from a CD3 polypeptide (e.g. CD3ζ) and an intracellular sequence or domain from a CD28 polypeptide, a 41BB polypeptide, a GITR polypeptide, an ICOS polypeptide, an OX40 polypeptide, a TRAF1 polypeptide, or a Lck polypeptide, and a stimulatory signal is initiated by association of the polypeptide into a CD3 complex or a TCR complex.

In some embodiments, a polypeptide of the present disclosure comprises a chemokine receptor polypeptide (e.g., CCR4, CCR2B, or a functional variant or portion thereof) and a stimulatory signal is initiated by binding of the chemokine receptor polypeptide to a ligand. In some embodiments, a chemokine receptor polypeptide is co-expressed with a CD8 polypeptide of the present disclosure.

In some embodiments, a stimulatory signal comprises an effector signal, wherein the effector signal directly or indirectly promotes an immunological response in a cell. An effector signal may be provided by, or to, an effector domain. As used herein, an “effector domain” is an intracellular portion, component, or domain of a polypeptide (e.g., fusion protein, receptor) that can directly or indirectly promote an immunological response in a cell when receiving an appropriate signal. In certain embodiments, an effector domain is from a protein or portion thereof or protein complex that receives a signal when bound, or when the protein or portion thereof or protein complex binds directly to a target molecule and triggers a signal from the effector domain. An effector domain may directly promote a cellular response when it contains one or more signaling domains or motifs, such as an Intracellular Tyrosine-based Activation Motif (ITAM), such as those found in costimulatory (also “co-stimulatory”, herein) molecules. Without wishing to be bound by theory, it is believed that ITAMs are important for T cell activation following ligand engagement by a T cell receptor or by a fusion protein comprising a T cell effector domain. In certain embodiments, the intracellular component or functional portion thereof comprises an ITAM. Exemplary effector domains include those from, CD3ε, CD3δ, CD3ζ, CD25, CD79A, CD79B, CARD11, DAP10, FcRα, FcRβ, FcRγ, Fyn, HVEM, ICOS, Lck, LAG3, LAT, LRP, NKG2D, NOTCHI, NOTCH2, NOTCH3, NOTCH4, Wnt, ROR2, Ryk, SLAMF1, Slp76, pTα, TCRα, TCRβ, TRIM, Zap70, PTCH2, or any combination thereof.

In some embodiments, a stimulatory signal comprises a costimulatory signal. In some contexts, a costimulatory signal is a secondary signal produced by a T cell costimulatory protein (e.g. CD28, ICOS, 4-1BB, OX40) and promotes e.g. T cell proliferation, survival, or one or more effector function, while a primary stimulatory signal is provided by the TCR complex to the T cell following engagement of the TCR with antigen: MHC. In some contexts, both TCR complex signaling and costimulatory signaling may be required for preferred activation of a T cell. In certain embodiments, a host cell is a T cell (e.g. a CD4+ T cell) and a primary stimulatory signal is provided by the TCR complex (e.g. comprising a heterologous MHC Class I-restricted TCR) while a costimulatory signal is provided by a polypeptide of the present disclosure, whether the polypeptide comprises a signaling portion or domain from CD28, ICOS, 4-1BB, OX40, or from a different polypeptide (e.g. from GITR, TRAF1, or Lck).

A costimulatory signal may be provided by a costimulatory domain, or a functional portion or variant thereof. In certain embodiments, the intracellular component of a polypeptide of the present disclosure comprises a costimulatory domain or a functional portion thereof selected from CD28, 4-1BB (CD137), OX40 (CD134), ICOS (CD278), CD27, CD2, CD5, ICAM-1 (CD54), LFA-1 (CD11a/CD18), GITR, CD30, CD40, BAFF-R, HVEM, LIGHT, NKG2C, SLAMF7, NKp80, CD160, B7-H3 CD94, DAP12, a ligand that specifically binds with CD83, or a functional variant thereof, or any combination thereof.

In certain embodiments, the intracellular component of a polypeptide (e.g. fusion protein) of the present disclosure comprises a costimulatory domain from CD28, or a functional portion or variant thereof (which may optionally include a non-leucine (e.g., glycine, serine, cysteine, alanine, valine, isoleucine, or the like) substitution at either one or both of positions 186-187 of the native CD28 protein (e.g., LL→GG; see Nguyen et al., Blood 102:4320, 2003)). Non-limiting examples of CD28 amino acid sequences are provided in SEQ ID NOs.: 18-20. In certain embodiments, a functional variant or portion of a CD28 costimulatory domain retains an ability to: (i) recruit and/or bind a SH2-domain-containing protein; and/or (ii) recruit and/or bind a SH3-domain-containing protein. In particular embodiments, a functional variant or portion of CD28 can bind to Lck, Tec, Itk, PI3K, Grb, Gads, or any combination thereof. In certain embodiments, a functional variant or portion of a CD28 costimulatory domain comprises a “PXXP” motif, wherein X can be any one or any two different amino acids (e.g., PRRP; PYAP; see SEQ ID NOS: 19 and 20). In certain embodiments, a functional variant or portion of a CD28 costimulatory domain comprises a tyrosine at a position corresponding to any one or more of positions 191, 206, 209, 218 of the native full-length human CD28 amino acid sequence. In certain embodiments, a functional variant or portion of a CD28 costimulatory domain comprises a tyrosine at positions corresponding to positions 206, 209, 218 of the native full-length human CD28 amino acid sequence. In certain embodiments, a functional variant or portion of a CD28 costimulatory domain comprises a “YXNX” motif, wherein X is any two same or different amino acids (e.g., YMNM). See Ogawa et al., Int. Immunol. 25 (12): 671 (2013) and Salter et al., Science Signaling 11: eaat6753 (2018).

In certain embodiments, a polypeptide of the present disclosure comprises a CD28 costimulatory domain comprising one or more amino acid mutations (e.g. substitution mutations) that modify (e.g. attenuate, diminish, boost, or increase) a signaling function by the CD28 costimulatory domain. An example of a signaling mutant CD28 sequence is provided in SEQ ID NO.:81 (such a mutant sequence is referred-to herein as a “partial” signaling mutant). Another example of a signaling mutant CD28 sequence is provided in SEQ ID NO.:108 (such a mutant sequence is referred-to herein as a “full” signaling mutant). In some contexts, attenuation of immune costimulatory or effector signaling may be desirable to, for example, decrease the risk of tonic signaling, undesired co-stimulation to endogenous TCR signaling, or the like.

It will be understood that “costimulatory domain” can refer to the portion of a protein that in the e.g. wild-type condition, is involved in producing a costimulatory signal, even if the subject protein contains alterations (e.g. mutations) that reduce or abrogate the signaling function.

In certain embodiments, the intracellular component of a polypeptide (e.g. fusion protein) of the present disclosure comprises a costimulatory domain from 4-1BB, or a functional portion or variant thereof. Non-limiting examples of 4-1BB amino acid sequences are provided in SEQ ID NOs: 21 and 22. In certain embodiments, a functional variant or portion of a 4-1BB costimulatory domain retains an ability to bind with TRAF1, TRAF2, and/or TRAF3, and/or to activate NF-κB. In certain embodiments, a functional variant or portion of a 4-1BB costimulatory domain comprises a motif “EED” at positions corresponding to positions 237-239 of the full-length human 4-1BB. In certain embodiments, a functional variant or portion of a 4-1BB costimulatory domain comprises a motif “EEE” at positions corresponding to positions 248-250 of the full-length human 4-1BB. See Jang et al., Biochem. Biophys. Res. Comm. 242:613 (1998).

In certain embodiments, the intracellular component of a polypeptide (e.g. fusion protein) of the present disclosure comprises a costimulatory domain from OX40, or a functional portion or variant thereof. Non-limiting examples of OX40 amino acid sequences are provided in SEQ ID NOs.: 23 and 24. In certain embodiments, a functional variant or portion of an OX40 costimulatory domain retains an ability to bind with: TRAF2; TRAF3; TRAF5; PI3K; or any combination thereof, and/or to activate NF-κB. In certain embodiments, a functional variant or portion of an OX40 costimulatory domain comprises a motif “GGSFRTPI” (SEQ ID NO.:187). See Kawamata et al., JBC, 273 (10): 5808 (1998).

In certain embodiments, the intracellular component of a polypeptide (e.g. fusion protein) of the present disclosure comprises a costimulatory domain from ICOS, or a functional portion or variant thereof. Non-limiting examples of ICOS amino acid sequences are provided in SEQ ID NOS.: 25 and 26. In certain embodiments, a functional variant or portion of an ICOS costimulatory domain retains an ability to bind with P50a subunit of PI3K. In certain embodiments, a functional variant or portion of an ICOS costimulatory domain comprises a “YXXM” motif, wherein X can be any two of a same or different amino acids. In certain embodiments, a functional variant or portion of an ICOS costimulatory domain comprises a “YMFM” motif. See Fos et al., J. Immunol. 181 (3): 1969 (2008).

In certain embodiments, the intracellular component of a polypeptide (e.g. fusion protein) of the present disclosure comprises a co-stimulatory domain from GITR, or a functional portion or variant thereof. Non-limiting examples of GITR amino acid sequences are provided in SEQ ID NOS.: 189 and 190. In certain embodiments, a functional variant or portion of a GITR co-stimulatory domain is capable of binding to TRAF1, TRAF2, and/or TRAF3.

In certain embodiments, the intracellular component of a polypeptide (e.g. fusion protein) of the present disclosure comprises an amino acid sequence from TRAF1 (SEQ ID NO.:181), or a functional variant or portion thereof that is capable of providing a stimulatory signal. In some embodiments, a polypeptide comprises, disposed between a transmembrane domain and a TRAF1 amino acid sequence, a linker, optionally having a length of from about 10 to about 30 amino acids, such as, for example, 10, 11, 12, 13, 14, 15, 16, 17, 18, 19, 20, 21, 22, 23, 24, 25, 26, 27, 28, 29, or 30 amino acids.

In certain embodiments, the intracellular component of a polypeptide (e.g. fusion protein) of the present disclosure comprises an amino acid sequence from Lck (SEQ ID NO.:188), or a functional variant or portion thereof that is capable of providing a stimulatory signal. In some embodiments, a polypeptide comprises the amino acid sequence set forth in SEQ ID NO.:102, or a functional variant or portion thereof. In some embodiments, a polypeptide comprises an amino acid sequence having at least 80%, at least 85%, at least 90%, at least 92%, at least 95%, at least 97%, or at least 99% identity to the amino acid sequence set forth in SEQ ID NO.:188 or 102.

It will be understood that any of the presently disclosed polypeptides (e.g. fusion proteins) can comprise any one or more (in any arrangement and in any combination) of the presently disclosed costimulatory domains or functional portions or variants thereof. Furthermore, any of the presently disclosed embodiments, a (n e.g. fusion) protein can comprise an extracellular and/or transmembrane component from a CD8 co-receptor β-chain, or a functional variant or portion thereof, from a CD8 co-receptor α-chain, or a functional variant or portion thereof.

In some embodiments, a stimulatory or costimulatory signal provided by a presently disclosed polypeptide to a host cell (e.g. T cell) expressing the same can comprise one or more effect, and/or substantially the same effect(s), as can result from an interaction between a native costimulatory polypeptide of the host cell and one or more cognate molecule; e.g., between CD28 and CD80, between CD28 and CD86, between ICOS and ICOS-L, between 4-1BB and 4-1BBL, or between OX40 and OX40L. In some embodiments, a stimulatory or costimulatory signal provided by a presently disclosed polypeptide to a host cell (e.g. T cell) expressing the same comprises one or more effect, or substantially the same effect(s), as can result from an interaction between GITR and GITR-L, between TRAF1 and TRAF2, cIAP2, cIAP1, MyD88, SHP-1, Src, or TNFR2, or any combination thereof, or between Lck and a CD3 polypeptide (e.g. CD3ζ) intracellular domain (e.g. containing an ITAM), ZAP-70, Fyn, PI3K, CD4, CD8, or any combination thereof. In some embodiments, a stimulatory or costimulatory signal provided by a presently disclosed polypeptide to a host cell (e.g. T cell) expressing the same comprises one or more effect, or substantially the same effect(s), as can result from: association of a CD3 polypeptide (e.g.) CD3ζ into a CD3 complex or a TCR complex, such as concurrent to antigen-binding by the TCR, binding of a chemokine receptor (e.g., CCR4 or CCR2B) to a ligand (e.g. to a chemokine such as CCL17 or CCL22 for CCR4, or CCL-2 (MCP-1), CCL77 (MCP-3), CCL8, CCL13 (MCP-4), or CCL16 for CCR2B. See e.g. Yoshie and Matushima, International Immunology, Volume 27, Issue 1, January 2015, Pages 11-20, doi.org/10.1093/intimm/dxu079;

Effects resulting from such native interactions are known in the art and can be assessed using known techniques.

In some embodiments, a stimulatory signal can be inferred or recognized by activation or function (e.g. cytotoxic activity, proliferation, or the like) of a host cell. In certain embodiments, a stimulatory signal can be inferred or recognized by an improved function or increased activation by a host cell (e.g. an immune cell, such as a T cell, for example a CD4+ T cell or a CD8+ T cell) as compared to a reference host cell that does not comprise the polypeptide or polypeptides. For example, a host cell and a reference host cell that each express an antigen-specific T cell receptor may be exposed to the antigen (e.g., pulsed with antigen-peptide, cultured with antigen-presenting cells or antigen-expressing target cells) and a stimulatory signal may be recognized by, for example, increased or improved (e.g. sustained) proliferation of the host cell comprising the polypeptide(s) of the present disclosure, increased or improved production of one or more cytokines (e.g. IFN-γ, TNFα, IL-2, or any combination thereof) by the host cell comprising the polypeptide(s) of the present disclosure, increased or improved cytotoxic activity against target cells by the host cell comprising the polypeptide(s) of the present disclosure, increased or improved expression of one or more activation markers, such as Nur77, or the like, as compared to the reference host cell. Accordingly, a polypeptide or plurality (e.g. a pair) of polypeptides of the present disclosure may be said to provide a stimulatory signal when a host cell expressing the same demonstrates one or more improved function or increased activation, as compared to a reference host cell.

In certain embodiments, a polypeptide may preform a recited function when in association with a further polypeptide. For example, in some embodiments, a polypeptide comprising an extracellular component from a CD8α is capable of binding to a MHC Class-I molecule when the polypeptide is comprised in a polypeptide multimer (e.g. homodimer or a heterodimer) with another CD8 polypeptide, such as a CD8β polypeptide or a CD8α polypeptide, including for example a chimeric co-receptor polypeptide of the present disclosure. In some contexts, preferred MHC Class-I-binding, preferred provision of a stimulatory signal, association with Lck, or any combination thereof, may occur when a CD8 polypeptide of the present disclosure is comprised in a dimer with a further CD8 polypeptide, which may be wild-type or may be engineered.

Certain functions of a disclosed polypeptide may be described with respect to a reference polypeptide. Unless the context provides otherwise, a reference polypeptide is identical to the subject polypeptide with the exception of the identified differences. In some embodiments, for example, a polypeptide comprises a variant of a CD28 costimulatory domain comprising one or more amino acid mutations (e.g. substitutions) to reduce (e.g. lower the intensity, duration, and/or frequency) of a stimulatory signal provided thereby, as compared to the stimulatory signal provided by a reference polypeptide comprising a wild-type CD28 costimulatory domain, or comprising a reference CD28 costimulatory domain that does not comprise the one or more amino acid mutations. A reference host cell is a host cell that is identical to (at least according to one or more criteria of interest), or is substantially identical to, the subject host cell with the exception of the identified differences. For example, a host cell may be a CD4+ T cell from a human subject, and a reference host cell may be a CD4+ T cell from the same human subject (such as, for example, a reference host cell may be obtained from the same blood sample as the host cell).

In certain embodiments, one or more of an extracellular component, a binding domain, a linker, a transmembrane domain, an intracellular component, or a costimulatory domain comprises one or more junction amino acids. “Junction amino acids” or “junction amino acid residues” refer to one or more (e.g., about 2-20) amino acid residues between two adjacent domains, motifs, regions, modules, or fragments of a protein, such as between a binding domain and an adjacent linker, between a transmembrane domain and an adjacent extracellular or intracellular domain, or on one or both ends of a linker that links two domains, motifs, regions, modules, or fragments (e.g., between a linker and an adjacent binding domain or between a linker and an adjacent hinge). Junction amino acids may result from the construct design of a fusion protein (e.g., amino acid residues resulting from the use of a restriction enzyme site or self-cleaving peptide sequences during the construction of a polynucleotide encoding a fusion protein). For example, a transmembrane domain of a fusion protein may have one or more junction amino acids at the amino-terminal end, carboxy-terminal end, or both.

In certain embodiments, the present disclosure provides fusion proteins that comprise: (i) an extracellular component comprising an extracellular domain from a CD8 co-receptor β-chain or a functional portion or variant thereof, or from a CD8 co-receptor α-chain or a functional portion or variant thereof, that is capable of binding to a MHC class I molecule; (ii) a transmembrane domain, provided that the transmembrane domain is not a transmembrane domain from a CD8 co-receptor α-chain when the extracellular component comprises a full length extracellular domain from the CD8 co-receptor α-chain; and (iii) an intracellular component comprising a co-stimulatory domain or a functional portion or variant thereof.

In certain embodiments, the extracellular component of a polypeptide or fusion protein comprises or is derived from a CD8 co-receptor β-chain, or a functional portion or variant thereof. In some embodiments, the CD8 co-receptor β-chain comprises a canonical β-chain, a M1 isoform, a M2 isoform, a M3 isoform, a M4 isoform, a M5 isoform, a M6 isoform, a M7 isoform, or a M8 isoform. In particular embodiments, the CD8 co-receptor β-chain is a M1 isoform. In some embodiments, the extracellular component comprises an amino acid sequence having at least 80%, at least 85%, at least 90%, at least 92%, at least 95%, at least 97%, or at least 99% identity to the amino acid sequence set forth in SEQ ID NO:7, or comprises or consists of the amino acid sequence set forth in SEQ ID NO:7.

In some embodiments, the transmembrane domain comprises or consists of a transmembrane domain from a CD4, a CD8β, a CD8α, a CD27, or a CD28, or a functional portion or variant thereof. In certain embodiments, the transmembrane domain comprises an amino acid sequence having at least 80%, at least 85%, at least 90%, at least 92%, at least 95%, at least 97%, or at least 99% identity to the amino acid sequence set forth in SEQ ID NO:8, or comprises or consists of the amino acid set forth in SEQ ID NO:8.

In some embodiments, the fusion protein further comprises an amino acid sequence having the amino acid sequence set forth in SEQ ID NO: 10, or a functional portion or variant thereof, disposed between the transmembrane domain and the intracellular component (e.g. costimulatory domain).

In particular embodiments, the extracellular component comprises the amino acid sequence set forth in SEQ ID NO:7 and the transmembrane domain comprises the amino acid sequence set forth in SEQ ID NO:8. In further embodiments, the intracellular component comprises the amino acid sequence set forth in SEQ ID NO.:10, optionally as set forth in SEQ ID NO.:9.

In certain embodiments, the extracellular component comprises or is derived from a CD8 co-receptor α-chain. The CD8 co-receptor α-chain may comprise or be derived from a canonical α-chain, isoform 2, or isoform 3.

In some embodiments, the co-stimulatory domain of a fusion protein comprises a co-stimulatory domain from one or more of CD28, 4-1BB (CD137), OX40 (CD134), ICOS (CD278), CD27, CD2, CD5, ICAM-1 (CD54), LFA-1 (CD11a/CD18), GITR, CD30, CD40, BAFF-R, HVEM, LIGHT, NKG2C, SLAMF7, NKp80, CD160, B7-H3, a ligand that specifically binds with CD83, CD94, DAP12, and/or comprises a functional variant of a co-stimulatory domain thereof.

In some embodiments, the co-stimulatory domain comprises a co-stimulatory domain from CD28, or a functional portion or variant thereof. In certain embodiments, the co-stimulatory domain comprises or consists of an amino acid sequence having at least 80% at least 85%, at least 90%, at least 92%, at least 95%, at least 97%, or at least 99% identity to the amino acid sequence shown in SEQ ID NO:19. In particular embodiments, the co-stimulatory domain comprises or consists of an amino acid sequence shown in SEQ ID NO:19. In other embodiments, the co-stimulatory domain comprises a variant of the amino acid sequence shown in SEQ ID NO: 19, wherein one or both of the leucine residues at positions 7 and 8 of SEQ ID NO: 19 is substituted for a different amino acid. In particular embodiments, the variant of the amino acid sequence shown in SEQ ID NO: 19 comprises a substitution of a glycine for one or both of the leucine residues at positions 7 and 8 of SEQ ID NO:19. In some embodiments, the co-stimulatory domain comprises or consists of an amino acid sequence having at least 80% identity to the amino acid sequence the amino acid sequence shown in SEQ ID NO: 20. In certain embodiments, the co-stimulatory domain comprises or consists of the amino acid sequence shown in SEQ ID NO:20. In some embodiments, the co-stimulatory domain comprises a co-stimulatory domain from 4-1BB, or a functional portion or variant thereof. In certain embodiments, the co-stimulatory domain comprises or consists of an amino acid sequence having at least 80% at least 85%, at least 90%, at least 92%, at least 95%, at least 97%, or at least 99% identity to the amino acid sequence shown in SEQ ID NO:22. In particular embodiments, the co-stimulatory domain comprises or consists of the amino acid sequence shown in SEQ ID NO:22. In some embodiments, the co-stimulatory domain comprises a co-stimulatory domain from OX40, or a functional portion or variant thereof. In certain embodiments, the co-stimulatory domain comprises or consists of an amino acid sequence having at least 80%, at least 85%, at least 90%, at least 92%, at least 95%, at least 97%, or at least 99% identity to the amino acid sequence shown in SEQ ID NO.:24. In particular embodiments, the co-stimulatory domain comprises or consists of the amino acid sequence shown in SEQ ID NO:24. In some embodiments, the co-stimulatory domain comprises a co-stimulatory domain from ICOS, or a functional portion or variant thereof. In certain embodiments, the co-stimulatory domain comprises or consists of an amino acid sequence having at least 80%, at least 85%, at least 90%, at least 92%, at least 95%, at least 97%, or at least 99% identity to the amino acid sequence shown in SEQ ID NO:26. In particular embodiments, the co-stimulatory domain comprises or consists of the amino acid sequence set forth in SEQ ID NO:26. In some embodiments, the co-stimulatory domain comprises a co-stimulatory domain from GITR, or a functional portion or variant thereof.

Also provided are fusion proteins that comprise: (i) an extracellular component comprising an extracellular domain from a CD8 co-receptor β-chain or a functional portion or variant thereof, or from a CD8 co-receptor α-chain or a functional portion or variant thereof, that is capable of binding to a MHC class I molecule; (ii) a transmembrane domain; and (iii) an intracellular component comprising a co-stimulatory domain from one, two, or three of: (a) a variant sequence of CD28 comprising or consisting of an amino acid sequence having at least 80% (i.e. at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%) identity to the amino acid sequence shown in SEQ ID NO:19 or 20, provided that: (1) no Tyr residue corresponding to position 12, 27, 30, or 39 of SEQ ID NO:19 is substituted with Phe when the extracellular component comprises a full length extracellular domain from a CD8 co-receptor α-chain and the transmembrane domain comprises a transmembrane domain from the CD8 co-receptor α-chain; and/or (2) one or both of the leucine residues corresponding to positions 7 and 8 of SEQ ID NO: 19 is substituted for a different amino acid, wherein the different amino acid optionally comprises glycine; (b) CD27, or a functional portion or variant thereof; (c) 4-1BB, or a functional portion or variant thereof; (d) ICOS, or a functional portion or variant thereof; (e) OX40, or a functional portion or variant thereof; (f) CD30, or a functional portion or variant thereof; (g) LFA-1, or a functional portion or variant thereof; (h) CD2, or a functional portion or variant thereof; (i) CD7, or a functional portion or variant thereof; (j) LIGHT, or a functional portion or variant thereof; (k) NKG2C, or a functional portion or variant thereof; (l) B7-H3, or a functional portion or variant thereof; (j) GITR, or a functional portion or variant thereof; (k) BAFF-R, or a functional portion or variant thereof; (l) CD5, or a functional portion or variant thereof; (m) HVEM, or a functional portion or variant thereof; (n) CD160, or a functional portion or variant thereof; (o) LFA-1, or a functional portion or variant thereof; (p) SLAMF7, or a functional portion or variant thereof; (q) NKp80, or a functional portion or variant thereof; (r) ICAM-1, or a functional portion or variant thereof; (s) CD94, or a functional portion or variant thereof; (t) DAP12, or a functional portion or variant thereof; or (u) a ligand that specifically binds with CD83.

In some embodiments, the extracellular component comprises or is derived from a CD8 co-receptor-chain, or a functional portion or variant thereof. In certain embodiments, the CD8 co-receptor β-chain comprises a canonical β-chain, a M1 isoform, a M2 isoform, a M3 isoform, a M4 isoform, a M5 isoform, a M6 isoform, a M7 isoform, or a M8 isoform. In some embodiments, fusion protein of claim 32, wherein the CD8 co-receptor β-chain is a M1 isoform. In particular embodiments, wherein the extracellular component comprises an amino acid sequence having at least 80% identity to the amino acid sequence set forth in SEQ ID NO:7, or comprises or consists of the amino acid sequence set forth in SEQ ID NO:7. In some embodiments, the transmembrane domain comprises or consists of a transmembrane domain from a CD4, a CD8β, a CD8α, a CD27, or a CD28, or a functional portion or variant thereof. In certain embodiments, the transmembrane domain comprises an amino acid sequence having at least 80% identity to the amino acid sequence set forth in SEQ ID NO:8, or comprises or consists of the amino acid set forth in SEQ ID NO:8. In some embodiments, the fusion protein further comprises the amino acid sequence set forth in SEQ ID NO:10, or a functional portion or variant thereof, disposed between the transmembrane domain and the intracellular component (e.g. costimulatory domain).

In particular embodiments, the extracellular component comprises the amino acid sequence set forth in SEQ ID NO:7 and the transmembrane domain comprises the amino acid sequence set forth in SEQ ID NO:8. In certain further embodiments, the intracellular component comprises the amino acid sequence set forth in SEQ ID NO.:10, optionally as set forth in SEQ ID NO.:9.

In some embodiments, the extracellular component comprises or is derived from a CD8 co-receptor α-chain. In certain embodiments, the CD8 co-receptor α-chain comprises a canonical α-chain, isoform2, or isoform 3. In particular embodiments, the extracellular component comprises or consists of an amino acid sequence having at least 80%, at least 85%, at least 90%, at least 92%, at least 95%, at least 97%, or at least 99% identity to the amino acid sequence set forth in set forth in SEQ ID NO: 2. In some embodiments, the transmembrane domain comprises an amino acid sequence having at least 80%, at least 85%, at least 90%, at least 92%, at least 95%, at least 97%, or at least 99% identity to the amino acid sequence set forth in SEQ ID NO:3, or comprises or consists of the amino acid sequence set forth in SEQ ID NO:3.

In some embodiments, the variant sequence of CD28 comprises a substitution of a glycine for one or both of the leucine residues corresponding to positions 7 and 8 of SEQ ID NO:19. In certain embodiments, the co-stimulatory domain comprises or consists an amino acid sequence having at least 80%, at least 85%, at least 90%, at least 92%, at least 95%, at least 97%, or at least 99% identity to the amino acid sequence shown in SEQ ID NO:20. In particular embodiments, the co-stimulatory domain comprises or consists of the amino acid sequence shown in SEQ ID NO:20.

In some embodiments, the co-stimulatory domain comprises a co-stimulatory domain from 4-1BB, or a functional portion or variant thereof. In certain embodiments, the co-stimulatory domain comprises or consists of an amino acid sequence having at least 80%, at least 85%, at least 90%, at least 92%, at least 95%, at least 97%, or at least 99% identity to the amino acid sequence shown in SEQ ID NO:22. In particular embodiments, the co-stimulatory domain comprises or consists of the amino acid sequence shown in SEQ ID NO:22. In some embodiments, the co-stimulatory domain comprises a co-stimulatory domain from OX40, or a functional portion or variant thereof. In certain embodiments, the co-stimulatory domain comprises or consists of an amino acid sequence having at least 80%, at least 85%, at least 90%, at least 92%, at least 95%, at least 97%, or at least 99% identity to the amino acid sequence shown in SEQ ID NO:24. In particular embodiments, the co-stimulatory domain comprises or consists of the amino acid sequence shown in SEQ ID NO:24. In some embodiments, the co-stimulatory domain comprises a co-stimulatory domain from ICOS, or a functional portion or variant thereof. In certain embodiments, the co-stimulatory domain comprises or consists of an amino acid sequence having at least 80%, at least 85%, at least 90%, at least 92%, at least 95%, at least 97%, or at least 99% identity to the amino acid sequence shown in SEQ ID NO:26. In particular embodiments, the co-stimulatory domain comprises or consists of the amino acid sequence set forth in SEQ ID NO: 26.

Also provided is a polypeptide that comprises: (i) an extracellular component from a CD8β; (ii) a transmembrane domain that is optionally from a CD8β; and (iii) an intracellular component comprising (iii)(1) a CD8β intracellular region amino acid sequence comprising or consisting of SEQ ID NO.:9 or SEQ ID NO.:10 and (iii)(2) a CD28 intracellular region amino acid sequence comprising a CD28 costimulatory domain and, optionally, a LL→GG mutation, wherein the polypeptide is capable of binding to a MHC Class I molecule.

Also provided is a polypeptide that comprises: (i) an extracellular component from a CD8β; (ii) a transmembrane domain that is optionally from a CD8β; and (iii) an intracellular component comprising a CD28 costimulatory domain and an optional LL→GG mutation, wherein the polypeptide is capable of binding to a MHC Class I molecule.

Also provided is a polypeptide that comprises: (i) an extracellular component from a CD8α; (ii) a transmembrane domain that is optionally from a CD8α; and (iii) an intracellular component comprising a CD28 costimulatory domain and an optional LL→GG mutation, wherein the polypeptide is capable of binding to a MHC Class I molecule.

Also provided is a polypeptide that comprises: (i) an extracellular component from a CD8β; (ii) a transmembrane domain that is optionally from a CD8β; and (iii) an intracellular component comprising (1) a CD28 costimulatory domain comprising the amino acid sequence DAMNMTARRAGPTRKHYQAYAAPRDFAAYRS (SEQ ID NO.:185) and (2)(1) an optional LL→GG mutation, wherein the polypeptide is capable of binding to a MHC Class I molecule.

Also provided is a polypeptide that comprises: (i) an extracellular component from a CD8α; (ii) a transmembrane domain that is optionally from a CD8α; and (iii) an intracellular component comprising (1) a CD28 costimulatory domain comprising the amino acid sequence DAMNMTARRAGPTRKHYQAYAAPRDFAAYRS (SEQ ID NO.:185) and (2) an optional LL→GG mutation, wherein the polypeptide is capable of binding to a MHC Class I molecule.

Also provided is a polypeptide that comprises: (i) an extracellular component from a CD8β; (ii) a transmembrane domain that is optionally from a CD8β; and (iii) an intracellular component comprising (iii)(1) a CD8β intracellular region amino acid sequence comprising or consisting of SEQ ID NO.:9 or SEQ ID NO.:10 and (iii)(2) a (n, optionally wild-type) CD28 costimulatory domain amino acid sequence, wherein the polypeptide is capable of binding to a MHC Class I molecule.

Also provided is a polypeptide that comprises: (i) an extracellular component from a CD8β; (ii) a transmembrane domain that is optionally from a CD8β; and (iii) an intracellular component comprising a costimulatory domain from (iii)(1) a 4-1BB, (iii)(2) an ICOS, (iii)(3), an OX40, or (iii)(4) a GITR, wherein the polypeptide is capable of binding to a MHC Class I molecule.

Also provided is a polypeptide that comprises: (i) an extracellular component from a CD8α; (ii) a transmembrane domain from a CD28; and (iii) an intracellular component comprising a CD28 costimulatory domain and, optionally, a LL→GG mutation, wherein the polypeptide is capable of binding to a MHC Class I molecule.

Also provided is a polypeptide that comprises: (i) an extracellular component from a CD8β; (ii) a transmembrane domain from a CD8β; and (iii) an intracellular component comprising (iii)(1) a CD8β intracellular region amino acid sequence (optionally comprising or consisting of SEQ ID NO.:9 or 10) and (iii)(2) a signaling domain from Lck, wherein the fusion protein is capable of binding to a MHC Class I molecule.

Also provided is a polypeptide that comprises: (i) an extracellular component from a CD8α; (ii) a transmembrane domain that is optionally from a CD8α; and (iii) an intracellular component comprising a CD28 costimulatory domain comprising the amino acid sequence DAMNMTARRAGPTRKHFQAFAAPRDFAAFRS (SEQ ID NO.:186) and optionally further comprising a LL→GG mutation, wherein the polypeptide is capable of binding to a MHC Class I molecule.

Also provided is a polypeptide that comprises: (i) an extracellular component from a CD8β; (ii) a transmembrane domain that is optionally from a CD8β; and (iii) an intracellular component comprising a CD28 costimulatory domain comprising the amino acid sequence DAMNMTARRAGPTRKHFQAFAAPRDFAAFRS (SEQ ID NO.X) and optionally further comprising a LL→GG mutation, wherein the polypeptide is capable of binding to a MHC Class I molecule.

Also provided is a polypeptide that comprises: (i) an extracellular component comprising (i)(1) a CD8α extracellular region amino acid sequence (e.g. comprising or consisting of a CD8α Ig V-like domain), (i)(2) a CD8β stalk region amino acid sequence, and (i)(3) a CD28 extracellular region amino acid sequence; (ii) a transmembrane domain from CD28; and (iii) an intracellular component comprising a CD28 costimulatory domain and an optional LL→GG mutation, wherein the polypeptide is capable of binding to a MHC Class I molecule. In certain embodiments, the CD28 extracellular region amino acid sequence of (i)(3) comprises or consists of amino acids 141-159 of SEQ ID NO.:141.

Also provided is a polypeptide that comprises: (i) an extracellular component comprising (i)(1) a CD8α extracellular region amino acid sequence (e.g. comprising or consisting of a CD8α Ig V-like domain), (i)(2) a CD8β stalk region amino acid sequence, and (i)(3) a CD28 extracellular region amino acid sequence; (ii) a transmembrane domain from CD28; and (iii) an intracellular component comprising (iii)(1) a CD28 costimulatory domain and an optional LL→GG mutation and (iii)(2) a CD8α intracellular signaling domain, wherein the polypeptide is capable of binding to a MHC Class I molecule. In certain embodiments, the CD28 extracellular region amino acid sequence of (i)(3) comprises or consists of amino acids 141-159 of SEQ ID NO.:141.

Also provided is a polypeptide that comprises (a) an extracellular component comprising an amino acid sequence having at least 80%, 85%, 90%, 91%, 92%, 93%, 94%, 95%, 96%, 97%, 98%, 99% identity, or more, to any one of SEQ ID NOs:69, 71, 73, or 75, (b) a transmembrane component comprising an amino acid sequence having at least 80%, 85%, 90%, 91%, 92%, 93%, 94%, 95%, 96%, 97%, 98%, 99% identity, or more, to any one of SEQ ID NOs:70, 72, 74, or 76, or a functional portion or variant thereof, and (c) an intracellular component comprising an intracellular domain from CD28, 4-1BB, GITR, ICOS, LCK, OX40, or a functional portion or variant thereof, and/or an intracellular signaling domain comprising an amino acid sequence having at least 90%, 91%, 92%, 93%, 94%, 95%, 96%, 97%, 98%, 99% identity, or more, to SEQ ID NO: 77. In certain embodiments, the intracellular component (c) of the polypeptide comprises an intracellular domain from CD28, 4-1BB, GITR, ICOS, LCK, OX40, or a functional portion or variant thereof, and an intracellular signaling component from CD3ζ or a functional portion or variant thereof. In certain embodiments, the intracellular domain from CD28 or 4-1BB is disposed between the intracellular signaling domain from CD3 and the transmembrane component. In certain embodiments, the extracellular component and the transmembrane component respectively comprise an extracellular domain and a transmembrane domain from a same CD3 protein; e.g., an extracellular domain and a transmembrane domain from CD3ε, an extracellular domain and a transmembrane domain from CD3ζ, an extracellular domain and a transmembrane domain from CD3γ, an extracellular domain and a transmembrane domain from CD3δ. In other embodiments, the extracellular domain and the transmembrane domain are each from a different CD3 protein.

In particular embodiments, a polypeptide comprises: (a) an extracellular component that has at least 90%, 91%, 92%, 93%, 94%, 95%, 96%, 97%, 98%, or 99% identity to, or comprises or consists of, the amino acid sequence according to SEQ ID NO.:69; (b) a transmembrane component that that has at least 90%, 91%, 92%, 93%, 94%, 95%, 96%, 97%, 98%, or 99% identity to, or comprises or consists of, the amino acid sequence according to SEQ ID NO.:70; and (c) an intracellular component that comprises (i) an amino acid sequence that has at least 90%, 91%, 92%, 93%, 94%, 95%, 96%, 97%, 98%, or 99% identity to, or comprises or consists of, the amino acid sequence of any one of SEQ ID NOs.: 19, 20, 22, 24, and 26, and (ii) an amino acid sequence that has at least 90%, 91%, 92%, 93%, 94%, 95%, 96%, 97%, 98%, or 99% identity to, or comprises or consists of, the amino acid sequence of SEQ ID NO.:77.

In particular embodiments, a polypeptide comprises: (a) an extracellular component that has at least 90%, 91%, 92%, 93%, 94%, 95%, 96%, 97%, 98%, or 99% identity to, or comprises or consists of, the amino acid sequence according to SEQ ID NO.:69; (b) a transmembrane component that that has at least 90%, 91%, 92%, 93%, 94%, 95%, 96%, 97%, 98%, or 99% identity to, or comprises or consists of, the amino acid sequence according to SEQ ID NO.:72; and (c) an intracellular component that comprises (i) an amino acid sequence that has at least 90%, 91%, 92%, 93%, 94%, 95%, 96%, 97%, 98%, or 99% identity to, or comprises or consists of, the amino acid sequence of any one of SEQ ID NOs.: 19, 20, 22, 24, and 26, and (ii) an amino acid sequence that has at least 90%, 91%, 92%, 93%, 94%, 95%, 96%, 97%, 98%, or 99% identity to, or comprises or consists of, the amino acid sequence of SEQ ID NO.:77.

In particular embodiments, a polypeptide comprises: (a) an extracellular component that has at least 90%, 91%, 92%, 93%, 94%, 95%, 96%, 97%, 98%, or 99% identity to, or comprises or consists of, the amino acid sequence according to SEQ ID NO.:69; (b) a transmembrane component that that has at least 90%, 91%, 92%, 93%, 94%, 95%, 96%, 97%, 98%, or 99% identity to, or comprises or consists of, the amino acid sequence according to SEQ ID NO.:74; and (c) an intracellular component that comprises (i) an amino acid sequence that has at least 90%, 91%, 92%, 93%, 94%, 95%, 96%, 97%, 98%, or 99% identity to, or comprises or consists of, the amino acid sequence of any one of SEQ ID NOs.: 19, 20, 22, 24, and 26, and (ii) an amino acid sequence that has at least 90%, 91%, 92%, 93%, 94%, 95%, 96%, 97%, 98%, or 99% identity to, or comprises or consists of, the amino acid sequence of SEQ ID NO.:77.

In particular embodiments, a polypeptide comprises: (a) an extracellular component that has at least 90%, 91%, 92%, 93%, 94%, 95%, 96%, 97%, 98%, or 99% identity to, or comprises or consists of, the amino acid sequence according to SEQ ID NO.:69; (b) a transmembrane component that that has at least 90%, 91%, 92%, 93%, 94%, 95%, 96%, 97%, 98%, or 99% identity to, or comprises or consists of, the amino acid sequence according to SEQ ID NO.:76; and (c) an intracellular component that comprises (i) an amino acid sequence that has at least 90%, 91%, 92%, 93%, 94%, 95%, 96%, 97%, 98%, or 99% identity to, or comprises or consists of, the amino acid sequence of any one of SEQ ID NOs.: 19, 20, 22, 24, and 26, and (ii) an amino acid sequence that has at least 90%, 91%, 92%, 93%, 94%, 95%, 96%, 97%, 98%, or 99% identity to, or comprises or consists of, the amino acid sequence of SEQ ID NO.:77.

In particular embodiments, a polypeptide comprises: (a) an extracellular component that has at least 90%, 91%, 92%, 93%, 94%, 95%, 96%, 97%, 98%, or 99% identity to, or comprises or consists of, the amino acid sequence according to SEQ ID NO.:71; (b) a transmembrane component that that has at least 90%, 91%, 92%, 93%, 94%, 95%, 96%, 97%, 98%, or 99% identity to, or comprises or consists of, the amino acid sequence according to SEQ ID NO.:70; and (c) an intracellular component that comprises (i) an amino acid sequence that has at least 90%, 91%, 92%, 93%, 94%, 95%, 96%, 97%, 98%, or 99% identity to, or comprises or consists of, the amino acid sequence of any one of SEQ ID NOs.: 19, 20, 22, 24, and 26, and (ii) an amino acid sequence that has at least 90%, 91%, 92%, 93%, 94%, 95%, 96%, 97%, 98%, or 99% identity to, or comprises or consists of, the amino acid sequence of SEQ ID NO.:77.

In particular embodiments, a polypeptide comprises: (a) an extracellular component that has at least 90%, 91%, 92%, 93%, 94%, 95%, 96%, 97%, 98%, or 99% identity to, or comprises or consists of, the amino acid sequence according to SEQ ID NO.:71; (b) a transmembrane component that that has at least 90%, 91%, 92%, 93%, 94%, 95%, 96%, 97%, 98%, or 99% identity to, or comprises or consists of, the amino acid sequence according to SEQ ID NO.:72; and (c) an intracellular component that comprises (i) an amino acid sequence that has at least 90%, 91%, 92%, 93%, 94%, 95%, 96%, 97%, 98%, or 99% identity to, or comprises or consists of, the amino acid sequence of any one of SEQ ID NOs.: 19, 20, 22, 24, and 26, and (ii) an amino acid sequence that has at least 90%, 91%, 92%, 93%, 94%, 95%, 96%, 97%, 98%, or 99% identity to, or comprises or consists of, the amino acid sequence of SEQ ID NO.:77.

In particular embodiments, a polypeptide comprises: (a) an extracellular component that has at least 90%, 91%, 92%, 93%, 94%, 95%, 96%, 97%, 98%, or 99% identity to, or comprises or consists of, the amino acid sequence according to SEQ ID NO.:71; (b) a transmembrane component that that has at least 90%, 91%, 92%, 93%, 94%, 95%, 96%, 97%, 98%, or 99% identity to, or comprises or consists of, the amino acid sequence according to SEQ ID NO.:74; and (c) an intracellular component that comprises (i) an amino acid sequence that has at least 90%, 91%, 92%, 93%, 94%, 95%, 96%, 97%, 98%, or 99% identity to, or comprises or consists of, the amino acid sequence of any one of SEQ ID NOs.: 19, 20, 22, 24, and 26, and (ii) an amino acid sequence that has at least 90%, 91%, 92%, 93%, 94%, 95%, 96%, 97%, 98%, or 99% identity to, or comprises or consists of, the amino acid sequence of SEQ ID NO.:77.

In particular embodiments, a polypeptide comprises: (a) an extracellular component that has at least 90%, 91%, 92%, 93%, 94%, 95%, 96%, 97%, 98%, or 99% identity to, or comprises or consists of, the amino acid sequence according to SEQ ID NO.:69; (b) a transmembrane component that that has at least 90%, 91%, 92%, 93%, 94%, 95%, 96%, 97%, 98%, or 99% identity to, or comprises or consists of, the amino acid sequence according to SEQ ID NO.:76; and (c) an intracellular component that comprises (i) an amino acid sequence that has at least 90%, 91%, 92%, 93%, 94%, 95%, 96%, 97%, 98%, or 99% identity to, or comprises or consists of, the amino acid sequence of any one of SEQ ID NOs.: 19, 20, 22, 24, and 26, and (ii) an amino acid sequence that has at least 90%, 91%, 92%, 93%, 94%, 95%, 96%, 97%, 98%, or 99% identity to, or comprises or consists of, the amino acid sequence of SEQ ID NO.:77.

In particular embodiments, a polypeptide comprises: (a) an extracellular component that has at least 90%, 91%, 92%, 93%, 94%, 95%, 96%, 97%, 98%, or 99% identity to, or comprises or consists of, the amino acid sequence according to SEQ ID NO.:73; (b) a transmembrane component that that has at least 90%, 91%, 92%, 93%, 94%, 95%, 96%, 97%, 98%, or 99% identity to, or comprises or consists of, the amino acid sequence according to SEQ ID NO.:70; and (c) an intracellular component that comprises (i) an amino acid sequence that has at least 90%, 91%, 92%, 93%, 94%, 95%, 96%, 97%, 98%, or 99% identity to, or comprises or consists of, the amino acid sequence of any one of SEQ ID NOs.: 19, 20, 22, 24, and 26, and (ii) an amino acid sequence that has at least 90%, 91%, 92%, 93%, 94%, 95%, 96%, 97%, 98%, or 99% identity to, or comprises or consists of, the amino acid sequence of SEQ ID NO.:77.

In particular embodiments, a polypeptide comprises: (a) an extracellular component that has at least 90%, 91%, 92%, 93%, 94%, 95%, 96%, 97%, 98%, or 99% identity to, or comprises or consists of, the amino acid sequence according to SEQ ID NO.:73; (b) a transmembrane component that that has at least 90%, 91%, 92%, 93%, 94%, 95%, 96%, 97%, 98%, or 99% identity to, or comprises or consists of, the amino acid sequence according to SEQ ID NO.:72; and (c) an intracellular component that comprises (i) an amino acid sequence that has at least 90%, 91%, 92%, 93%, 94%, 95%, 96%, 97%, 98%, or 99% identity to, or comprises or consists of, the amino acid sequence of any one of SEQ ID NOs.: 19, 20, 22, 24, and 26, and (ii) an amino acid sequence that has at least 90%, 91%, 92%, 93%, 94%, 95%, 96%, 97%, 98%, or 99% identity to, or comprises or consists of, the amino acid sequence of SEQ ID NO.:77.

In particular embodiments, a polypeptide comprises: (a) an extracellular component that has at least 90%, 91%, 92%, 93%, 94%, 95%, 96%, 97%, 98%, or 99% identity to, or comprises or consists of, the amino acid sequence according to SEQ ID NO.:73; (b) a transmembrane component that that has at least 90%, 91%, 92%, 93%, 94%, 95%, 96%, 97%, 98%, or 99% identity to, or comprises or consists of, the amino acid sequence according to SEQ ID NO.:74; and (c) an intracellular component that comprises (i) an amino acid sequence that has at least 90%, 91%, 92%, 93%, 94%, 95%, 96%, 97%, 98%, or 99% identity to, or comprises or consists of, the amino acid sequence of any one of SEQ ID NOs.: 19, 20, 22, 24, and 26, and (ii) an amino acid sequence that has at least 90%, 91%, 92%, 93%, 94%, 95%, 96%, 97%, 98%, or 99% identity to, or comprises or consists of, the amino acid sequence of SEQ ID NO.:77. In particular embodiments, a polypeptide comprises: (a) an extracellular component that has at least 90%, 91%, 92%, 93%, 94%, 95%, 96%, 97%, 98%, or 99% identity to, or comprises or consists of, the amino acid sequence according to SEQ ID NO.:73; (b) a transmembrane component that that has at least 90%, 91%, 92%, 93%, 94%, 95%, 96%, 97%, 98%, or 99% identity to, or comprises or consists of, the amino acid sequence according to SEQ ID NO.:76; and (c) an intracellular component that comprises (i) an amino acid sequence that has at least 90%, 91%, 92%, 93%, 94%, 95%, 96%, 97%, 98%, or 99% identity to, or comprises or consists of, the amino acid sequence of any one of SEQ ID NOs.: 19, 20, 22, 24, and 26, and (ii) an amino acid sequence that has at least 90%, 91%, 92%, 93%, 94%, 95%, 96%, 97%, 98%, or 99% identity to, or comprises or consists of, the amino acid sequence of SEQ ID NO.:77.

Also provided is a polypeptide that comprises: (i) an extracellular component from a CD3ζ; (ii) a transmembrane domain that is optionally from CD3ζ; and (iii) an intracellular component comprising (iii)(a) a CD28 costimulatory domain and (iii)(b) a CD3ζ effector domain. In some embodiments, (iii)(a) is disposed between (ii) and (iii)(b).

Also provided is a polypeptide that comprises: (i) an extracellular component from a CD3ζ; (ii) a transmembrane domain that is optionally from CD3; and (iii) an intracellular component comprising (iii)(a) a 4-1BB costimulatory domain and (iii)(b) a CD3ζ effector domain. In some embodiments, (iii)(a) is disposed between (ii) and (iii)(b).

In certain embodiments, any of the herein disclosed polypeptides comprising an extracellular component (e.g. ectodomain) from a CD3 protein (zeta, epsilon, gamma, or delta) does not further comprise an extracellular target-binding domain (e.g. an antigen-binding domain (e.g. an antibody variable domain)). In some embodiments, the extracellular component consists essentially of, or consists of, of the ectodomain from the CD3 protein.

Also provided is a polypeptide that comprises: (i) an extracellular component from a NKG2D; (ii) a transmembrane domain that is optionally from a NKG2D; and (iii) a CD28 costimulatory domain and an optional LL→GG mutation, wherein the polypeptide is capable of binding to a NKG2D ligand, wherein the NKG2D ligand optionally comprises a MIC family ligand, a ULBP family ligand, or both.

Also provided is a polypeptide that comprises a Fas extracellular component and a transmembrane domain that is optionally from Fas, and does not comprise a functional Fas intracellular signaling domain, wherein the polypeptide is capable of binding to a FasL, and wherein the polypeptide optionally comprises a truncated Fas protein that does not comprise a full-length Fas intracellular region, wherein the polypeptide is capable of binding to a FasL. In some embodiments, a functional Fas intracellular signaling domain facilitates one or more suppressive and/or apoptotic signal in a host cell expressing the Fas when the Fas binds to a FasL. In some embodiments, a full-length Fas intracellular region comprises the amino acid sequence

(SEQ ID NO.: 191)
KRKEVQKTCRKHRKENQGSHESPTLNPETVAINLSDVDLSKYITTIAGVM
TLSQVKGFVRKNGVNEAKIDEIKNDNVQDTAEQKVQLLRNWHQLHGKKEA
YDTLIKDLKKANLCTLAEKIQTIILKDITSDSENSNFRNEIQSLV.

Also provided is a polypeptide that comprises: (i) an extracellular component from a Fas; (ii) a transmembrane domain that is optionally from a Fas; and (iii) an intracellular component comprising a Lck intracellular signaling domain, wherein the polypeptide is capable of binding to a FasL.

Also provided is a polypeptide that comprises: (i) an extracellular component from a Fas; (ii) a transmembrane domain that is optionally from a Fas; and (iii) an intracellular component comprising a CD8α intracellular amino acid sequence, wherein the polypeptide is capable of binding to a FasL and, optionally, associating with a Lck.

Also provided is a polypeptide that comprises: (i) an extracellular component from a Fas; (ii) a transmembrane domain that is optionally from a Fas; (iii) an intracellular component comprising a TRAF1 intracellular signaling domain, and, optionally, (iv) a linker amino acid sequence disposed between and connecting the transmembrane domain and the TRAF1 intracellular signaling domain, wherein the polypeptide is capable of binding to a FasL.

It will be understood that a polypeptide (e.g., a fusion protein) comprising a referenced component from one source protein (e.g. a transmembrane domain, a costimulatory domain, a signaling domain, or the like) can comprise additional amino acid sequence or sequences from the said source protein; i.e., the extent to which amino acid sequence from the said source protein is present in the polypeptide or fusion protein may, in certain embodiments, surpass the specifically referenced component or portion thereof, unless the context clearly provides otherwise. It will also be understood that while certain (e.g. source) protein amino acid sequences, components, domains, or regions are provided herein and/or are known in the art, less than all of a specified amino acid sequence, domain, component, or region may be present in a protein of the present disclosure, provided that the protein can perform one or more functions as described herein. For example, in a source protein that contains a domain, motif, or site of interest, amino acids and other protein features that are not proximal to the domain, motif, or site of interest and/or will not or are not expected to impair functionality of the domain, motif, or site of interest will be recognized by those of ordinary skill in the art, and these may be present in or absent from an fusion protein of the present disclosure that contains the domain, motif, or site of interest. Moreover, any of the presently disclosed fusion proteins, components, or domains can comprise one or more junction amino acids.

Additionally, it will be understood that engineered CD8-containing polypeptides can comprise chimeric sequences or amino acid substitutions derived from different CD8 protein isoforms (e.g., a CD8β fusion protein may comprise amino acid sequences from two or more CD8β isoforms), and/or can comprise one or more amino acid sequence from a CD8β and one or more amino acid sequence from a CD8α. Furthermore, all two-protein combinations of a CD8β-containing protein with a cognate CD8α-containing protein (or of two CD8α-containing proteins) are encompassed, including combinations wherein one or neither of the two proteins is a (n optionally, full-length (with or without signal peptide)) wild-type CD8 or CD8α, respectively.

In certain embodiments, a polypeptide is provided that comprises an amino acid sequence having at least 90%, at least 91%, at least 92%, at least 93%, at least 94%, at least 95%, at least 96%, at least 97%, at least 98%, or at least 99% identity to, or comprising or consisting of, the amino acid sequence set forth in any one of SEQ ID NOs.: 36-42, 83-97, and 103-105. In certain embodiments, a polypeptide is provided that comprises or consists of two or more amino acid sequences, each of the two or more amino acid sequences independently having at least 90%, at least 91%, at least 92%, at least 93%, at least 94%, at least 95%, at least 96%, at least 97%, at least 98%, or at least 99% identity to, or comprising or consisting of, an amino acid sequence set forth in any one of SEQ ID NOs.: 36-42, 83-97, and 103-105. In certain embodiments, a polypeptide is provided that comprises an amino acid sequence having at least 90%, at least 91%, at least 92%, at least 93%, at least 94%, at least 95%, at least 96%, at least 97%, at least 98%, or at least 99% identity to, or comprising or consisting of, the amino acid sequence set forth in any one of SEQ ID NOs.: 113, 115-118, 120-167, and 176-178

In certain embodiments, a (n e.g. human) T cell that expresses (i) any one or more of the presently disclosed fusion proteins, polypeptides, or a combination of these and (ii) an antigen-specific T cell receptor (TCR) (e.g., a MHC-I-restricted TCR), is capable of any one or more of the following (1)-(7), as compared to a reference T cell that expresses (ii) and optionally expresses wild-type CD8αβ, but does not express (i): (1) increased proliferation (as determined by cell division) in the presence of antigen-expressing cells; (2) increased production of interferon-gamma (IFN-γ) in the presence of antigen, wherein the increased production is optionally an increase of at least 10%, at least 15%, at least 20%, at least 25%, at least 30%, at least 35%, at least 40%, at least 45%, at least 50%, at least 55%, at least 60%, at least 70%, at least 75%, at least 80%, at least 85%, at least 90%, at least 95%, at least 100%, at least 125%, at least 150%, at least 175%, at least 200%, at least 225%, at least 250%, at least 275%, or at least 300%; (3) increased production of TNFα; (4) increased production of IL-2; (5) increased activation, optionally as determined by Nur77 expression in the T cell after co-culture with antigen-presenting cells; (6) increased sensitivity to the presence of antigen, optionally as determined by activation of the T cell after co-culture with antigen-presenting cells, further optionally by determining Nur77 expression; (7) killing of antigen-presenting cells, optionally in vitro, over the course of 24, 48, 72, 96, 120, 144, 168, 216, 240, 264, and/or 288 hours.

In certain embodiments, the T cell is a CD4+ T cell. In certain embodiments, the T cell is a CD8+ T cell. In certain embodiments, the T cell is a primary human T cell obtained from a donor, such as a healthy donor.

Also provided are polynucleotides that encode any one or more (including, in some contexts, two or more) of the presently disclosed fusion proteins or polypeptides. A polynucleotide can comprise, or be comprised in, an expression construct wherein the polynucleotide is operably linked to an expression control sequence (e.g., a promoter). In some embodiments, a polynucleotide or expression construct encodes two or more polypeptides and the nucleic acid sequences encoding the two or more polypeptides may be separated by (or have disposed therebetween) nucleic acid sequences that encode a self-cleaving peptide, a furin cleavage sequence, an internal ribosomal skip element (IRES), or any combination thereof. Examples of polynucleotide sequences that encode 2A self-cleaving peptides are provided in SEQ ID NOs.: 50-54. Examples of 2A self-cleaving peptide amino acid sequences are provided in SEQ ID NOs.: 55-58.

In certain embodiments, the polynucleotide is codon-optimized for expression in a host cell. Codon optimization can be performed using known techniques and tools, e.g., using the GenScript® OptimiumGene™ tool; see also Scholten et al., Clin. Immunol. 119:135, 2006). Codon-optimized sequences include sequences that are partially codon-optimized (i.e., one or more codon is optimized for expression in the host cell) and those that are fully codon-optimized.

In some embodiments, an isolated polynucleotide comprises a polynucleotide that encodes a first fusion protein of the present disclosure, wherein the first encoded fusion protein comprises an extracellular domain from a CD8 co-receptor β-chain, or a functional portion or variant thereof. In further embodiments, the isolated polynucleotide further comprises a polynucleotide encoding a second protein, wherein the second encoded protein (which can be a fusion protein, such as a fusion protein of the present disclosure) comprises: (i) a CD8 co-receptor α-chain, or a functional portion or variant thereof; or (ii) an extracellular domain from a CD8 co-receptor α-chain, or a functional portion or variant thereof.

In other embodiments, an isolated polynucleotide comprises a polynucleotide that encodes a first fusion protein of the present disclosure, wherein the first encoded fusion protein comprises an extracellular domain from a CD8 co-receptor α-chain, or a functional portion or variant thereof. In further embodiments, the isolated polynucleotide further comprises a polynucleotide encoding a second protein (which can be a fusion protein, such as a fusion protein of the present disclosure), wherein the second encoded protein comprises: (i) a CD8 co-receptor α-chain, or a functional portion or variant thereof; (ii) an extracellular domain from a CD8 co-receptor α-chain or a functional portion or variant thereof; or (iii) a CD8 co-receptor β chain, or a functional portion or variant thereof; or (iv) an extracellular domain from a CD8 co-receptor β-chain, or a functional portion or variant thereof.

In certain embodiments, a polynucleotide (or vector or host cell, as disclosed herein) comprises a polynucleotide comprising: (a) a polynucleotide encoding a protein comprising at least an extracellular portion of a CD8 co-receptor α chain (which protein may be a wild-type CD8α or any polypeptide or fusion protein as described herein that comprises extracellular amino acid sequence from a CD8α); (b) a polynucleotide encoding a protein comprising at least an extracellular portion of a CD8 co-receptor β chain (which protein may be a wild-type CD8β or any polypeptide or fusion protein as described herein that comprises extracellular amino acid sequence from a CD8β); and (c) a polynucleotide encoding a self-cleaving peptide disposed between the polynucleotide of (a) and the polynucleotide of (b). In further embodiments, a polynucleotide comprises a polynucleotide that encodes a self-cleaving peptide and is disposed between: (1) a polynucleotide encoding a binding protein (e.g., TCR of the present disclosure) and a polynucleotide of (a); and/or (2) a polynucleotide encoding a binding protein and a polynucleotide of (b).

In still further embodiments, a polynucleotide can comprise, operably linked in-frame: (i) (pnCD8α)-(pnSCP1)-(pnCD8β)-(pnSCP2)-(pnTCR); (ii) (pnCD8β)-(pnSCP1)-(pnCD8α)-(pnSCP2)-(pnTCR); (iii) (pnTCR)-(pnSCP1)-(pnCD8α)-(pnSCP2)-(pnCD8β); (iv) (pnTCR)-(pnSCP1)-(pnCD8β)-(pnSCP2)-(pnCD8α); (v) (pnCD8α)-(pnSCP1)-(pnTCR)-(pnSCP2)-(pnCD8β); or (vi) (pnCD8β)-(pnSCP1)-(pnTCR)-(pnSCP2)-(pnCD8α), wherein pnCD8α is the polynucleotide of (a), wherein pnCD8β is the polynucleotide of (b), wherein pnTCR is the polynucleotide encoding a TCR, and wherein pnSCP1 and pnSCP2 are each independently a polynucleotide encoding a self-cleaving peptide, wherein the polynucleotides and/or the encoded self-cleaving peptides are optionally the same or different (e.g., P2A, T2A, F2A, E2A).

In some embodiments, the encoded TCR comprises a TCRα chain and a TCRβ chain, wherein the polynucleotide comprises a polynucleotide encoding a self-cleaving peptide disposed between the polynucleotide encoding a TCRα chain and the polynucleotide encoding a TCRβ chain. In some embodiments, the polynucleotide comprises, operably linked in-frame: (i) (pnCD8α)-(pnSCP1)-(pnCD8β)-(pnSCP2)-(pnTCRβ)-(pnSCP3)-(pnTCRα); (ii) (pnCD8β)-(pnSCP1)-(pnCD8α)-(pnSCP2)-(pnTCRβ)-(pnSCP3)-(pnTCRα); (iii) (pnCD8α)-(pnSCP1)-(pnCD8β)-(pnSCP2)-(pnTCRα)-(pnSCP3)-(pnTCRβ); (iv) (pnCD8β)-(pnSCP1)-(pnCD8α)-(pnSCP2)-(pnTCRα)-(pnSCP3)-(pnTCRβ); (v) (pnTCRβ)-(pnSCP1)-(pnTCRα)-(pnSCP2)-(pnCD8α)-(pnSCP3)-(pnCD8β); (vi) (pnTCRβ)-(pnSCP1)-(pnTCRα)-(pnSCP2)-(pnCD8β)-(pnSCP3)-(pnCD8α); (vii) (pnTCRα)-(pnSCP1)-(pnTCRβ)-(pnSCP2)-(pnCD8α)-(pnSCP3)-(pnCD8β); or (viii) (pnTCRα)-(pnSCP1)-(pnTCRβ)-(pnSCP2)-(pnCD8β)-(pnSCP3)-(pnCD8α), wherein pnCD8α is the polynucleotide of (a), wherein pnCD8β is the polynucleotide of (b), wherein pnTCRα is the polynucleotide encoding a TCR α chain, wherein pnTCRβ is the polynucleotide encoding a TCR β chain, and wherein pnSCP1, pnSCP2, and pnSCP3 are each independently a polynucleotide encoding a self-cleaving peptide, wherein the polynucleotides and/or the encoded self-cleaving peptides are optionally the same or different.

In further aspects, expression constructs are provided, wherein the expression constructs comprise a polynucleotide of the present disclosure operably linked to an expression control sequence (e.g., a promoter). In certain embodiments, the expression construct is comprised in a vector. An exemplary vector may comprise a polynucleotide capable of transporting another polynucleotide to which it has been linked, or which is capable of replication in a host organism. In certain embodiments, polynucleotides of the present disclosure may be operatively linked to certain elements of a vector. For example, polynucleotide sequences that are needed to effect the expression and processing of coding sequences to which they are ligated may be operatively linked. Expression control sequences may include appropriate transcription initiation, termination, promoter and enhancer sequences; efficient RNA processing signals such as splicing and polyadenylation signals; sequences that stabilize cytoplasmic mRNA; sequences that enhance translation efficiency (i.e., Kozak consensus sequences); sequences that enhance protein stability; and possibly sequences that enhance protein secretion. Expression control sequences may be operatively linked if they are contiguous with the gene of interest and expression control sequences that act in trans or at a distance to control the gene of interest.

Some examples of vectors include plasmids, viral vectors, cosmids, and others. Some vectors may be capable of autonomous replication in a host cell into which they are introduced (e.g. bacterial vectors having a bacterial origin of replication and episomal mammalian vectors), whereas other vectors may be integrated into the genome of a host cell or promote integration of the polynucleotide insert upon introduction into the host cell and thereby replicate along with the host genome (e.g., lentiviral vector, retroviral vector). Additionally, some vectors are capable of directing the expression of genes to which they are operatively linked (these vectors may be referred to as “expression vectors”). According to related embodiments, it is further understood that, if one or more agents (e.g., polynucleotides encoding proteins as described herein) are co-administered to a subject, that each agent may reside in separate or the same vectors, and multiple vectors (each containing a different agent or the same agent) may be introduced to a cell or cell population or administered to a subject.

In certain embodiments, the vector comprises a plasmid vector or a viral vector (e.g., a vector selected from lentiviral vector or a γ-retroviral vector). Viral vectors include retrovirus, adenovirus, parvovirus (e.g., adeno-associated viruses), coronavirus, negative strand RNA viruses such as ortho-myxovirus (e.g., influenza virus), rhabdovirus (e.g., rabies and vesicular stomatitis virus), paramyxovirus (e.g., measles and Sendai), positive strand RNA viruses such as picornavirus and alphavirus, and double-stranded DNA viruses including adenovirus, herpesvirus (e.g., Herpes Simplex virus types 1 and 2, Epstein-Barr virus, cytomegalovirus), and poxvirus (e.g., vaccinia, fowlpox and canarypox). Other viruses include Norwalk virus, togavirus, flavivirus, reoviruses, papovavirus, hepadnavirus, and hepatitis virus, for example. Examples of retroviruses include avian leukosis-sarcoma, mammalian C-type, B-type viruses, D type viruses, HTLV-BLV group, lentivirus, spumavirus (Coffin, J. M., Retroviridae: The viruses and their replication, In Fundamental Virology, Third Edition, B. N. Fields et al., Eds., Lippincott-Raven Publishers, Philadelphia, 1996).

“Retroviruses” are viruses having an RNA genome, which is reverse-transcribed into DNA using a reverse transcriptase enzyme, the reverse-transcribed DNA is then incorporated into the host cell genome. “Gammaretrovirus” refers to a genus of the retroviridae family. Examples of gammaretroviruses include mouse stem cell virus, murine leukemia virus, feline leukemia virus, feline sarcoma virus, and avian reticuloendotheliosis viruses.

“Lentiviral vector,” as used herein, means HIV-based lentiviral vectors for gene delivery, which can be integrative or non-integrative, have relatively large packaging capacity, and can transduce a range of different cell types. Lentiviral vectors are usually generated following transient transfection of three (packaging, envelope and transfer) or more plasmids into producer cells. Like HIV, lentiviral vectors enter the target cell through the interaction of viral surface glycoproteins with receptors on the cell surface. On entry, the viral RNA undergoes reverse transcription, which is mediated by the viral reverse transcriptase complex. The product of reverse transcription is a double-stranded linear viral DNA, which is the substrate for viral integration into the DNA of infected cells.

In certain embodiments, the viral vector can be a gammaretrovirus, e.g., Moloney murine leukemia virus (MLV)-derived vectors. In other embodiments, the viral vector can be a more complex retrovirus-derived vector, e.g., a lentivirus-derived vector. HIV-1-derived vectors belong to this category. Other examples include lentivirus vectors derived from HIV-2, FIV, equine infectious anemia virus, SIV, and Maedi-Visna virus (ovine lentivirus). Methods of using retroviral and lentiviral viral vectors and packaging cells for transducing mammalian host cells with viral particles containing binding protein (e.g. TCR) transgenes are known in the art and have been previous described, for example, in: U.S. Pat. No. 8,119,772; Walchli et al., PLOS One 6:327930, 2011; Zhao et al., J. Immunol. 174:4415, 2005; Engels et al., Hum. Gene Ther. 14:1155, 2003; Frecha et al., Mol. Ther. 18:1748, 2010; and Verhoeyen et al., Methods Mol. Biol. 506:97, 2009. Retroviral and lentiviral vector constructs and expression systems are also commercially available. Other viral vectors also can be used for polynucleotide delivery including DNA viral vectors, including, for example adenovirus-based vectors and adeno-associated virus (AAV)-based vectors; vectors derived from herpes simplex viruses (HSVs), including amplicon vectors, replication-defective HSV and attenuated HSV (Krisky et al., Gene Ther. 5:1517, 1998).

Other vectors can be used with the compositions and methods of this disclosure. Such vectors include those derived from baculoviruses and α-viruses. (Jolly, D J. 1999. Emerging Viral Vectors. pp 209-40 in Friedmann T. ed. The Development of Human Gene Therapy. New York: Cold Spring Harbor Lab), or plasmid vectors (such as sleeping beauty or other transposon vectors).

When a viral vector genome comprises a plurality of polynucleotides to be expressed in a host cell as separate transcripts, the viral vector may also comprise additional sequences between the two (or more) transcripts allowing for bicistronic or multicistronic expression. Examples of such sequences used in viral vectors include internal ribosome entry sites (IRES), furin cleavage sites, viral 2A peptide, or any combination thereof.

Construction of an expression vector that is used for genetically engineering and producing a (n e.g.) fusion protein of interest can be accomplished by using any suitable molecular biology engineering techniques known in the art. To obtain efficient transcription and translation, a polynucleotide in each recombinant expression construct includes at least one appropriate expression control sequence (also called a regulatory sequence), such as a leader sequence and particularly a promoter operably (i.e., operatively) linked to the nucleotide sequence encoding the polypeptide of interest.

Examples of polypeptide-encoding constructs (encoding one or two polypeptide) and encoded polypeptide of the present disclosure are described in FIGS. 9A-12C. Table 1 provides descriptions of these and certain other (one- or two-polypeptide) constructs of the present disclosure.

TABLE 1
Summary of Polypeptide Constructs A-AA

| | | Shorthand | Transgene (Tg) Design | | |
|---|---|---|---|---|---|
| Group | Constr. | Identifier | Tg1 | Tg2 | Detailed Design |
| CD8α/β | A | CD8aCD8b6*28 | EC: CD8a | EC: CD8b/ | Full CD8a P2A linked |
| chimeric | | | IC: CD8a | IC: 6 AA | to CD8b maintaining 6 |
| | | | | CD8b + CD28 | AA of IC region with |
| | | | | | CD28 intracellular |
| | | | | | region (GG mutation) |
| | B | CD8aCD8b*28 | EC: CD8a | EC: CD8b | Full CD8a P2A linked |
| | | | IC: CD8a | IC: CD28 | to CD8b with CD28 |
| | | | | | intracellular region |
| | | | | | (GG mutation) |
| | C | CD8a*28CD8b*28 | EC: CD8a | EC: CD8b | CD8a with CD28 |
| | | | IC: CD28 | IC: CD28 | intracellular region |
| | | | | | (GG mutation) P2A |
| | | | | | linked to CD8b with |
| | | | | | CD28 intracellular |
| | | | | | region (GG mutation) |
| | D | CD8a*28CD8b | EC: CD8a | EC: CD8b | CD8a with CD28 |
| | | | IC: CD28 | IC: CD8b | intracellular region |
| | | | | | (GG mutation) P2A |
| | | | | | linked to full CD8b |
| | E | CD8aCD8b6*28mt | EC: CD8a | EC: CD8b | Full CD8a P2A linked |
| | | | IC: CD8a | IC: 6 AA | to CD8b with CD28 |
| | | | | CD8b + CD28 | intracellular region |
| | | | | partial mutant | (GG mutation + partial |
| | | | | | signaling mutation) |
| | F | CD8a*28mtCD8b*28mt | EC: CD8a | EC: CD8b | CD8a with CD28 |
| | | | IC: CD28 | IC: CD28 | intracellular region |
| | | | partial mutant | partial mutant | (GG mutation + partial |
| | | | | | signaling mutation) |
| | | | | | P2A linked to CD8b |
| | | | | | with CD28 |
| | | | | | intracellular region |
| | | | | | (GG mutation + partial |
| | | | | | signaling mutation) |
| | G | CD8a_{—} | EC: CD8a | EC: CD8b | Full CD8a P2A linked |
| | | CD8-CD28 | IC: CD8a | IC: 6 AA | to CD8b maintaining 6 |
| | | | | CD8b + CD28wt | AA of IC region with |
| | | | | | CD28 intracellular |
| | | | | | region (wt LL) |
| | H | CD8a_CD8_{—} | EC: CD8a | EC: CD8b | Full CD8a P2A linked |
| | | 41BB | IC: CD8a | IC: 6 AA | to CD8b with 41BB |
| | | | | CD8b + 41BB | intracellular region |
| | I | CD8a_CD8_{—} | EC: CD8a | EC: CD8b | Full CD8a P2A linked |
| | | ICOS | IC: CD8a | IC: 6 AA | to CD8b with ICOS |
| | | | | CD8b + ICOS | intracellular region |
| | J | CD8a_CD8_{—} | EC: CD8a | EC: CD8b | Full CD8a P2A linked |
| | | OX40 | IC: CD8a | IC: 6 AA | to CD8b with OX40 |
| | | | | CD8b + OX40 | intracellular region |
| | K | CD8a_CD8_{—} | EC: CD8a | EC: CD8bIC: | Full CD8a P2A linked |
| | | GITR | IC: CD8a | 6 AA CD8b + | to CD8b with GITR |
| | | | | GITR | intracellular region |
| | L | CD8a_28Tm_{—} | EC: CD8a- | EC: CD8b | CD8a ex. cell- CD28 |
| | | 28IC_P2A_{—} | 28TM | IC: Lck | transmembrane with |
| | | CD8b_lck | IC: CD28 | | CD28 intracellular |
| | | | | | region (GG mutation) |
| | | | | | P2A linked to CD8b |
| | | | | | ex/tm/ic with 47-496 |
| | | | | | of Lck intracellular |
| | | | | | region |
| Single | M | CD8a_bSTALK_{—} | EC: CD8a- | — | CD8a/CD8b |
| Stalks | | 28cys_28Tm_{—} | CD8bstalk- | | stalk/CD28 up to |
| | | 28IC | 28cys-28tm | | cysteine in |
| | | | IC: CD28 | | extracellular region + |
| | | | | | CD28 transmembrane |
| | | | | | with CD28 |
| | | | | | intracellular region |
| | | | | | (GG mutation) |
| | N | CD8a_bSTALK_{—} | EC: CD8a- | — | CD8a/CD8b stalk + |
| | | 28tm_28IC | CD8bstalk- | | CD28 transmembrane |
| | | | 28tm | | with CD28 |
| | | | IC: CD28 | | intracellular region |
| | | | | | (GG mutation) |
| | O | CD8a_bSTALK_{—} | EC: CD8a- | — | CD8a/CD8b stalk + |
| | | 28Tm_28IC_{—} | CD8bstalk- | | CD28 transmembrane |
| | | aIC | 28tm | | with CD28 |
| | | | IC: CD28 + | | intracellular region |
| | | | CD8a | | (GG mutation) fused |
| | | | | | in front of CD8a |
| | | | | | intracellular region |
| | P | CD8a_bSTALK_{—} | EC: CD8a- | — | CD8a/CD8b stalk + |
| | | aTm_aIC | CD8bstalk- | | CD8a transmembrane |
| | | | CD8atm | | with CD8a |
| | | | IC: CD8a | | intracellular region |
| NKG2D | Q | hNKG2D_{—} | EC: NKG2D | — | NKG2D extracellular |
| IFP | | CD28 | IC: CD28 | | and transmembrane |
| | | | | | with CD28 |
| | | | | | intracellular (wt) |
| Chemo- | R | CD8aCCR4 | EC: CD8a | EC: CCR4 | Full CD8a P2A linked |
| kine | | | IC: CD8a | IC: CCR4 | to Full CCR4 |
| | S | CD8aCCR2b | EC: CD8a | EC: CCR2b | Full CD8a P2A linked |
| | | | IC: CD8a | IC: CCR2b | to Full CCR2b |
| CD8a/b | T | CD8-28 | EC: CD8a | EC: CD8b | CD8a with CD28 |
| chimeric | | full mutant | IC: CD28 | IC: CD28 | intracellular region |
| mutant | | | full mutant | full mutant | (GG mutation + full |
| | | | | | signaling mutation) |
| | | | | | P2A linked to CD8b |
| | | | | | with CD28 |
| | | | | | intracellular region |
| | | | | | (GG mutation + full |
| | | | | | signaling mutation) |
| Fas IFP | U | Fas no | EC: Fas | — | Fas extracellular + Fas |
| | | signal | IC: | | Tm truncated no |
| | | | truncated | | signaling domain |
| | | | Fas | | |
| | V | Fas_Lck | EC: Fas | — | Fas extracellular + |
| | | | IC: Lck | | FasTM + lck |
| | | | | | intracellular (47-496) |
| | W | Fas_CD8aIC | EC: Fas | — | Fas extracellular/TM + |
| | | | IC: CD8a | | CD8a intracellular |
| | X | Fas_TRAF1 | EC: Fas | — | Fas extracellular/TM + |
| | | | IC: TRAF1 | | linker (G/S/T 22 |
| | | | | | amino acid linker) + |
| | | | | | TRAF1 intracellular |
| PD-1 | Y | PD1_CD28 | EC: PD1 | — | PD-1 extracellular + |
| IFP | | | IC: CD28 | | CD28 extracellular to |
| | | | | | cysteine + CD28TM + |
| | | | | | CD28 intracellular |
| | | | | | region (GG mutation) |
| CD3ζ | Z | Zeta28z | EC: CD3z | — | CD3zEC-CD28IC- |
| fusion | | | IC: CD28/CD3z | | CD3zIC |
| | AA | ZetaBBz | EC: CD3z | — | CD3zEC-41BBIC- |
| | | | IC: 41BB/CD3z | | CD3zIC |

In Table 1: “EC” = Extracellular component; “IC” = Intracellular component; “P2A linked” = first and second protein-coding sequences being linked in a fusion construct by a sequence encoding a P2A self-cleaving peptide; “CD8a” = CD8α; “CD8b” = CD8β; “CD8b6” = CD8β extracellular component and transmembrane component, with 6 CD8β intracellular amino acids (HLCCRR; SEQ ID NO.: 10); “GG mutation” = LL-to-GG mutation in CD28 intracellular region; “partial signaling mutation” = CD28 intracellular region comprising DAMNMTARRAGPTRKHYQAYAAPRDFAAYRS (SEQ ID NO.: 185); “full signaling mutation” = CD28 intracellular region comprising DAMNMTARRAGPTRKHFQAFAAPRDFAAFRS (SEQ ID NO.: 186); “IFP” = Immunomodulatory Fusion Protein.

Table 2 provides amino acid sequences of components of these (one- or two-polypeptide) constructs.

TABLE 2
Amino Acid Sequences of Polypeptide Constructs A-AA

| | Polypeptide 1 | | | | Polypeptide 2 | | | |
|---|---|---|---|---|---|---|---|---|
| | Signal | EC | TM | IC | Signal | EC | TM | IC |
| | peptide | Component | domain | Component | peptide | Component | domain | Component |
| A | (SEQ ID | (SEQ ID | (SEQ ID | (SEQ ID | (SEQ ID | (SEQ ID | (SEQ ID | (SEQ ID |
| | NO.: 168) | NO.: 2) | NO.: 3) | NO.: 4) | NO.: 179) | NO.: 7) | NO.: 8) | NO.: 83) |
| B | (SEQ ID | (SEQ ID | (SEQ ID | (SEQ ID | (SEQ ID | (SEQ ID | (SEQ ID | (SEQ ID |
| | NO.: 168) | NO.: 2) | NO.: 3) | NO.: 4) | NO.: 179) | NO.: 7) | NO.: 8) | NO.: 20) |
| C | (SEQ ID | (SEQ ID | (SEQ ID | (SEQ ID | (SEQ ID | (SEQ ID | (SEQ ID | (SEQ ID |
| | NO.: 168) | NO.: 2) | NO.: 3) | NO.: 20) | NO.: 179) | NO.: 7) | NO.: 8) | NO.: 20) |
| D | (SEQ ID | (SEQ ID | (SEQ ID | (SEQ ID | (SEQ ID | (SEQ ID | (SEQ ID | (SEQ ID |
| | NO.: 168) | NO.: 2) | NO.: 3) | NO.: 20) | NO.: 179) | NO.: 7) | NO.: 8) | NO.: 9) |
| E | (SEQ ID | (SEQ ID | (SEQ ID | (SEQ ID | (SEQ ID | (SEQ ID | (SEQ ID | (SEQ ID |
| | NO.: 168) | NO.: 2) | NO.: 3) | NO.: 4) | NO.: 179) | NO.: 7) | NO.: 8) | NO.: 180) |
| F | (SEQ ID | (SEQ ID | (SEQ ID | (SEQ ID | (SEQ ID | (SEQ ID | (SEQ ID | (SEQ ID |
| | NO.: 168) | NO.: 2) | NO.: 3) | NO.: 81) | NO.: 179) | NO.: 7) | NO.: 8) | NO.: 81) |
| G | (SEQ ID | (SEQ ID | (SEQ ID | (SEQ ID | (SEQ ID | (SEQ ID | (SEQ ID | (SEQ ID |
| | NO.: 168) | NO.: 2) | NO.: 3) | NO.: 4) | NO.: 179) | NO.: 7) | NO.: 8) | NO.: 84) |
| H | (SEQ ID | (SEQ ID | (SEQ ID | (SEQ ID | (SEQ ID | (SEQ ID | (SEQ ID | (SEQ ID |
| | NO.: 168) | NO.: 2) | NO.: 3) | NO.: 4) | NO.: 179) | NO.: 7) | NO.: 8) | NO.: 85) |
| I | (SEQ ID | (SEQ ID | (SEQ ID | (SEQ ID | (SEQ ID | (SEQ ID | (SEQ ID | (SEQ ID |
| | NO.: 168) | NO.: 2) | NO.: 3) | NO.: 4) | NO.: 179) | NO.: 7) | NO.: 8) | NO.: 86) |
| J | (SEQ ID | (SEQ ID | (SEQ ID | (SEQ ID | (SEQ ID | (SEQ ID | (SEQ ID | (SEQ ID |
| | NO.: 168) | NO.: 2) | NO.: 3) | NO.: 4) | NO.: 179) | NO.: 7) | NO.: 8) | NO.: 87) |
| K | (SEQ ID | (SEQ ID | (SEQ ID | (SEQ ID | (SEQ ID | (SEQ ID | (SEQ ID | (SEQ ID |
| | NO.: 168) | NO.: 2) | NO.: 3) | NO.: 4) | NO.: 179) | NO.: 7) | NO.: 8) | NO.: 88) |
| L | (SEQ ID | (SEQ ID | (SEQ ID | (SEQ ID | (SEQ ID | (SEQ ID | (SEQ ID | (SEQ ID |
| | NO.: 168) | NO.: 173) | NO.: 80) | NO.: 20) | NO.: 179) | NO.: 7) | NO.: 8) | NO.: 89) |
| M | (SEQ ID | (SEQ ID | (SEQ ID | (SEQ ID | | | | |
| | NO.: 168) | NO.: 90) | NO.: 80) | NO.: 20) | | | | |
| N | (SEQ ID | (SEQ ID | (SEQ ID | (SEQ ID | | | | |
| | NO.: 168) | NO.: 92) | NO.: 80) | NO.: 20) | | | | |
| O | (SEQ ID | (SEQ ID | (SEQ ID | (SEQ ID | | | | |
| | NO.: 168) | NO.: 92) | NO.: 80) | NO.: 97) | | | | |
| P | (SEQ ID | (SEQ ID | (SEQ ID | (SEQ ID | | | | |
| | NO.: 168) | NO.: 94) | NO.: 3) | NO.: 4) | | | | |
| Q | (SEQ ID | (SEQ ID | (SEQ ID | (SEQ ID | | | | |
| | NO.: 169) | NO.: 174) | NO.: 80) | NO.: 19) | | | | |
| R | (SEQ ID | (SEQ ID | (SEQ ID | (SEQ ID | Polypeptide 2 Sequence | | | |
| | NO.: 168) | NO.: 2) | NO.: 3) | NO.: 4) | (SEQ ID NO.: 106) | | | |
| S | (SEQ ID | (SEQ ID | (SEQ ID | (SEQ ID | Polypeptide 2 Sequence | | | |
| | NO.: 168) | NO.: 2) | NO.: 3) | NO.: 4) | (SEQ ID NO.: 107) | | | |
| T | (SEQ ID | (SEQ ID | (SEQ ID | (SEQ ID | (SEQ ID | (SEQ ID | (SEQ ID | (SEQ ID |
| | NO.: 168) | NO.: 2) | NO.: 3) | NO.: 108) | NO.: 179) | NO.: 7) | NO.: 8) | NO.: 108) |
| U | (SEQ ID | (SEQ ID | (SEQ ID | (SEQ ID | | | | |
| | NO.: 170) | NO.: 98) | NO.: 100) | NO.: 101) | | | | |
| V | (SEQ ID | (SEQ ID | (SEQ ID | (SEQ ID | | | | |
| | NO.: 170) | NO.: 98) | NO.: 100) | NO.: 175) | | | | |
| W | (SEQ ID | (SEQ ID | (SEQ ID | (SEQ ID | | | | |
| | NO.: 170) | NO.: 98) | NO.: 100) | NO.: 4) | | | | |
| X | (SEQ ID | (SEQ ID | (SEQ ID | (SEQ ID | | | | |
| | NO.: 170) | NO.: 98) | NO.: 100) | NO.: 176) | | | | |
| Y | (SEQ ID | (SEQ ID | (SEQ ID | (SEQ ID | | | | |
| | NO.: 171) | NO.: 104) | NO.: 80) | NO.: 20) | | | | |
| Z | (SEQ ID | (SEQ ID | (SEQ ID | (SEQ ID | | | | |
| | NO.: 172) | NO.: 75) | NO.: 76) | NO.: 177) | | | | |
| AA | (SEQ ID | (SEQ ID | (SEQ ID | (SEQ ID | | | | |
| | NO.: 172) | NO.: 75) | NO.: 76) | NO.: 178) | | | | |
| WT | (SEQ ID | (SEQ ID | (SEQ ID | (SEQ ID | (SEQ ID | (SEQ ID | (SEQ ID | (SEQ ID |
| | NO.: 168) | NO.: 2) | NO.: 3) | NO.: 4) | NO.: 179) | NO.: 7) | NO.: 8) | NO.: 9) |

In some embodiments, a polypeptide is provided that comprises: (i) an extracellular component comprising an amino acid sequence having at least 90%, at least 91%, at least 92%, at least 93%, at least 94%, at least 95%, at least 96%, at least 97%, at least 98%, or at least 99% identity to, or comprising or consisting of, the amino acid sequence set forth in any one of SEQ ID NOs.: 2, 173, 90, 92, and 7; (ii) a transmembrane domain comprising an amino acid sequence having at least 90%, at least 91%, at least 92%, at least 93%, at least 94%, at least 95%, at least 96%, at least 97%, at least 98%, or at least 99% identity to, or comprising or consisting of, the amino acid sequence set forth in any one of SEQ ID NOs.: 3, 8, and 80; and (iii) an intracellular component comprising an amino acid sequence having at least 90%, at least 91%, at least 92%, at least 93%, at least 94%, at least 95%, at least 96%, at least 97%, at least 98%, or at least 99% identity to, or comprising or consisting of, the amino acid sequence set forth in any one of SEQ ID NOs.: 4, 83, 20, 9, 180, 81, 84, 85, 86, 87, 88, 89, 97, and 108. In some embodiments, a polypeptide is provided that comprises an extracellular component, a transmembrane domain, and an intracellular component according to Polypeptide 1 of any one of Constructs A-AA in Table 2. In certain other embodiments, a polypeptide is provided that comprises an extracellular component, a transmembrane domain, and an intracellular component according to Polypeptide 2 of any one of Constructs A-AA in Table 2. In certain further embodiments, a polynucleotide or vector that encodes the polypeptide, and/or a host cell that expresses and/or encodes the polypeptide, is provided. In some embodiments, a polynucleotide or vector encodes or comprise two or more such polypeptides that are different from one another. For example, in some embodiments, a polynucleotide or vector encodes: (i) a polypeptide that comprises an extracellular component, a transmembrane domain, and an intracellular component according to Polypeptide 1 of any one of Constructs A-AA in Table 2; and (ii) a polypeptide that comprises an extracellular component, a transmembrane domain, and an intracellular component according to Polypeptide 2 of any one of Constructs A-AA in Table 2. In certain embodiments, a polynucleotide or vector encodes the first polypeptide and the second polypeptide of any one of Constructs A-AA in Table 2, wherein, optionally, one or both of the encoded polypeptides may not comprise a signal peptide according to Table 2. Table 3 provides amino acid sequences of certain polypeptide constructs (Constructs A-AA (with or without signal peptide(s), where applicable)) of the present disclosure. Table 4 summarizes the amino acid SEQ ID NOs. of these polypeptide Constructs.

TABLE 3
Amino Acid Sequences of Constructs A-AA

| Construct | Polypeptide 1 | Polypeptide 2 |
|---|---|---|
| A | MALPVTALLLPLALLLHAARPSQFR | MRPRLWLLLAAQLTVLHGNSV |
| (with signal | VSPLDRTWNLGETVELKCQVLLSNP | LQQTPAYIKVQTNKMVMLSCE |
| peptides) | TSGCSWLFQPRGAAASPTFLLYLSQ | AKISLSNMRIYWLRQRQAPSSD |
| | NKPKAAEGLDTQRFSGKRLGDTFV | SHHEFLALWDSAKGTIHGEEVE |
| | LTLSDFRRENEGYYFCSALSNSIMYF | QEKIAVFRDASRFILNLTSVKPE |
| | SHFVPVFLPAKPTTTPAPRPPTPAPTI | DSGIYFCMIVGSPELTFGKGTQL |
| | ASQPLSLRPEACRPAAGGAVHTRGL | SVVDFLPTTAQPTKKSTLKKRV |
| | DFACDIYIWAPLAGTCGVLLLSLVIT | CRLPRPETQKGPLCSPITLGLLV |
| | LYCNHRNRRRVCKCPRPVVKSGDK | AGVLVLLVSLGVAIHLCCRRRS |
| | PSLSARYV | KRSRGGHSDYMNMTPRRPGPT |
| | (SEQ ID NO.: 1) | RKHYQPYAPPRDFAAYRS |
| | | (SEQ ID NO.: 113) |
| A (without | SQFRVSPLDRTWNLGETVELKCQVL | LQQTPAYIKVQTNKMVMLSCE |
| signal | LSNPTSGCSWLFQPRGAAASPTFLL | AKISLSNMRIYWLRQRQAPSSD |
| peptides) | YLSQNKPKAAEGLDTQRFSGKRLG | SHHEFLALWDSAKGTIHGEEVE |
| | DTFVLTLSDFRRENEGYYFCSALSN | QEKIAVFRDASRFILNLTSVKPE |
| | SIMYFSHFVPVFLPAKPTTTPAPRPP | DSGIYFCMIVGSPELTFGKGTQL |
| | TPAPTIASQPLSLRPEACRPAAGGAV | SVVDFLPTTAQPTKKSTLKKRV |
| | HTRGLDFACDIYIWAPLAGTCGVLL | CRLPRPETQKGPLCSPITLGLLV |
| | LSLVITLYCNHRNRRRVCKCPRPVV | AGVLVLLVSLGVAIHLCCRRRS |
| | KSGDKPSLSARYV | KRSRGGHSDYMNMTPRRPGPT |
| | (SEQ ID NO.: 114) | RKHYQPYAPPRDFAAYRS |
| | | (SEQ ID NO.: 115) |
| B (with | MALPVTALLLPLALLLHAARPSQFR | MRPRLWLLLAAQLTVLHGNSV |
| signal | VSPLDRTWNLGETVELKCQVLLSNP | LQQTPAYIKVQTNKMVMLSCE |
| peptides) | TSGCSWLFQPRGAAASPTFLLYLSQ | AKISLSNMRIYWLRQRQAPSSD |
| | NKPKAAEGLDTQRFSGKRLGDTFV | SHHEFLALWDSAKGTIHGEEVE |
| | LTLSDFRRENEGYYFCSALSNSIMYF | QEKIAVFRDASRFILNLTSVKPE |
| | SHFVPVFLPAKPTTTPAPRPPTPAPTI | DSGIYFCMIVGSPELTFGKGTQL |
| | ASQPLSLRPEACRPAAGGAVHTRGL | SVVDFLPTTAQPTKKSTLKKRV |
| | DFACDIYIWAPLAGTCGVLLLSLVIT | CRLPRPETQKGPLCSPITLGLLV |
| | LYCNHRNRRRVCKCPRPVVKSGDK | AGVLVLLVSLGVAIRSKRSRGG |
| | PSLSARYV | HSDYMNMTPRRPGPTRKHYQP |
| | (SEQ ID NO.: 1) | YAPPRDFAAYRS |
| | | (SEQ ID NO.: 116) |
| B (without | SQFRVSPLDRTWNLGETVELKCQVL | LQQTPAYIKVQTNKMVMLSCE |
| signal | LSNPTSGCSWLFQPRGAAASPTFLL | AKISLSNMRIYWLRQRQAPSSD |
| peptides) | YLSQNKPKAAEGLDTQRFSGKRLG | SHHEFLALWDSAKGTIHGEEVE |
| | DTFVLTLSDFRRENEGYYFCSALSN | QEKIAVFRDASRFILNLTSVKPE |
| | SIMYFSHFVPVFLPAKPTTTPAPRPP | DSGIYFCMIVGSPELTFGKGTQL |
| | TPAPTIASQPLSLRPEACRPAAGGAV | SVVDFLPTTAQPTKKSTLKKRV |
| | HTRGLDFACDIYIWAPLAGTCGVLL | CRLPRPETQKGPLCSPITLGLLV |
| | LSLVITLYCNHRNRRRVCKCPRPVV | AGVLVLLVSLGVAIRSKRSRGG |
| | KSGDKPSLSARYV | HSDYMNMTPRRPGPTRKHYQP |
| | (SEQ ID NO.: 114) | YAPPRDFAAYRS |
| | | (SEQ ID NO.: 117) |
| C (with | MALPVTALLLPLALLLHAARPSQFR | MRPRLWLLLAAQLTVLHGNSV |
| signal | VSPLDRTWNLGETVELKCQVLLSNP | LQQTPAYIKVQTNKMVMLSCE |
| peptides) | TSGCSWLFQPRGAAASPTFLLYLSQ | AKISLSNMRIYWLRQRQAPSSD |
| | NKPKAAEGLDTQRFSGKRLGDTFV | SHHEFLALWDSAKGTIHGEEVE |
| | LTLSDFRRENEGYYFCSALSNSIMYF | QEKIAVFRDASRFILNLTSVKPE |
| | SHFVPVFLPAKPTTTPAPRPPTPAPTI | DSGIYFCMIVGSPELTFGKGTQL |
| | ASQPLSLRPEACRPAAGGAVHTRGL | SVVDFLPTTAQPTKKSTLKKRV |
| | DFACDIYIWAPLAGTCGVLLLSLVIT | CRLPRPETQKGPLCSPITLGLLV |
| | RSKRSRGGHSDYMNMTPRRPGPTR | AGVLVLLVSLGVAIRSKRSRGG |
| | KHYQPYAPPRDFAAYRS | HSDYMNMTPRRPGPTRKHYQP |
| | (SEQ ID NO.: 118) | YAPPRDFAAYRS |
| | | (SEQ ID NO.: 116) |
| C (without | SQFRVSPLDRTWNLGETVELKCQVL | LQQTPAYIKVQTNKMVMLSCE |
| signal | LSNPTSGCSWLFQPRGAAASPTFLL | AKISLSNMRIYWLRQRQAPSSD |
| peptides) | YLSQNKPKAAEGLDTQRFSGKRLG | SHHEFLALWDSAKGTIHGEEVE |
| | DTFVLTLSDFRRENEGYYFCSALSN | QEKIAVFRDASRFILNLTSVKPE |
| | SIMYFSHFVPVFLPAKPTTTPAPRPP | DSGIYFCMIVGSPELTFGKGTQL |
| | TPAPTIASQPLSLRPEACRPAAGGAV | SVVDFLPTTAQPTKKSTLKKRV |
| | HTRGLDFACDIYIWAPLAGTCGVLL | CRLPRPETQKGPLCSPITLGLLV |
| | LSLVITRSKRSRGGHSDYMNMTPRR | AGVLVLLVSLGVAIRSKRSRGG |
| | PGPTRKHYQPYAPPRDFAAYRS | HSDYMNMTPRRPGPTRKHYQP |
| | (SEQ ID NO.: 39) | YAPPRDFAAYRS |
| | | (SEQ ID NO.: 117) |
| D (with | MALPVTALLLPLALLLHAARPSQFR | MRPRLWLLLAAQLTVLHGNSV |
| signal | VSPLDRTWNLGETVELKCQVLLSNP | LQQTPAYIKVQTNKMVMLSCE |
| peptides) | TSGCSWLFQPRGAAASPTFLLYLSQ | AKISLSNMRIYWLRQRQAPSSD |
| | NKPKAAEGLDTQRFSGKRLGDTFV | SHHEFLALWDSAKGTIHGEEVE |
| | LTLSDFRRENEGYYFCSALSNSIMYF | QEKIAVFRDASRFILNLTSVKPE |
| | SHFVPVFLPAKPTTTPAPRPPTPAPTI | DSGIYFCMIVGSPELTFGKGTQL |
| | ASQPLSLRPEACRPAAGGAVHTRGL | SVVDFLPTTAQPTKKSTLKKRV |
| | DFACDIYIWAPLAGTCGVLLLSLVIT | CRLPRPETQKGPLCSPITLGLLV |
| | RSKRSRGGHSDYMNMTPRRPGPTR | AGVLVLLVSLGVAIHLCCRRRR |
| | KHYQPYAPPRDFAAYRS | ARLRFMKQFYK |
| | (SEQ ID NO.: 118) | (SEQ ID NO.: 6) |
| D (without | SQFRVSPLDRTWNLGETVELKCQVL | LQQTPAYIKVQTNKMVMLSCE |
| signal | LSNPTSGCSWLFQPRGAAASPTFLL | AKISLSNMRIYWLRQRQAPSSD |
| peptides) | YLSQNKPKAAEGLDTQRFSGKRLG | SHHEFLALWDSAKGTIHGEEVE |
| | DTFVLTLSDFRRENEGYYFCSALSN | QEKIAVFRDASRFILNLTSVKPE |
| | SIMYFSHFVPVFLPAKPTTTPAPRPP | DSGIYFCMIVGSPELTFGKGTQL |
| | TPAPTIASQPLSLRPEACRPAAGGAV | SVVDFLPTTAQPTKKSTLKKRV |
| | HTRGLDFACDIYIWAPLAGTCGVLL | CRLPRPETQKGPLCSPITLGLLV |
| | LSLVITRSKRSRGGHSDYMNMTPRR | AGVLVLLVSLGVAIHLCCRRRR |
| | PGPTRKHYQPYAPPRDFAAYRS | ARLRFMKQFYK |
| | (SEQ ID NO.: 39) | (SEQ ID NO.: 119) |
| E (with | MALPVTALLLPLALLLHAARPSQFR | MRPRLWLLLAAQLTVLHGNSV |
| signal | VSPLDRTWNLGETVELKCQVLLSNP | LQQTPAYIKVQTNKMVMLSCE |
| peptides) | TSGCSWLFQPRGAAASPTFLLYLSQ | AKISLSNMRIYWLRQRQAPSSD |
| | NKPKAAEGLDTQRFSGKRLGDTFV | SHHEFLALWDSAKGTIHGEEVE |
| | LTLSDFRRENEGYYFCSALSNSIMYF | QEKIAVFRDASRFILNLTSVKPE |
| | SHFVPVFLPAKPTTTPAPRPPTPAPTI | DSGIYFCMIVGSPELTFGKGTQL |
| | ASQPLSLRPEACRPAAGGAVHTRGL | SVVDFLPTTAQPTKKSTLKKRV |
| | DFACDIYIWAPLAGTCGVLLLSLVIT | CRLPRPETQKGPLCSPITLGLLV |
| | LYCNHRNRRRVCKCPRPVVKSGDK | AGVLVLLVSLGVAIHLCCRRRS |
| | PSLSARYV | KRSRGGHSDAMNMTARRAGPT |
| | (SEQ ID NO.: 1) | RKHYQAYAAPRDFAAYRS |
| | | (SEQ ID NO.: 120) |
| E (without | SQFRVSPLDRTWNLGETVELKCQVL | LQQTPAYIKVQTNKMVMLSCE |
| signal | LSNPTSGCSWLFQPRGAAASPTFLL | AKISLSNMRIYWLRQRQAPSSD |
| peptides) | YLSQNKPKAAEGLDTQRFSGKRLG | SHHEFLALWDSAKGTIHGEEVE |
| | DTFVLTLSDFRRENEGYYFCSALSN | QEKIAVFRDASRFILNLTSVKPE |
| | SIMYFSHFVPVFLPAKPTTTPAPRPP | DSGIYFCMIVGSPELTFGKGTQL |
| | TPAPTIASQPLSLRPEACRPAAGGAV | SVVDFLPTTAQPTKKSTLKKRV |
| | HTRGLDFACDIYIWAPLAGTCGVLL | CRLPRPETQKGPLCSPITLGLLV |
| | LSLVITLYCNHRNRRRVCKCPRPVV | AGVLVLLVSLGVAIHLCCRRRS |
| | KSGDKPSLSARYV | KRSRGGHSDAMNMTARRAGPT |
| | (SEQ ID NO.: 114) | RKHYQAYAAPRDFAAYRS |
| | | (SEQ ID NO.: 121) |
| F (with | MALPVTALLLPLALLLHAARPSQFR | MRPRLWLLLAAQLTVLHGNSV |
| signal | VSPLDRTWNLGETVELKCQVLLSNP | LQQTPAYIKVQTNKMVMLSCE |
| peptides) | TSGCSWLFQPRGAAASPTFLLYLSQ | AKISLSNMRIYWLRQRQAPSSD |
| | NKPKAAEGLDTQRFSGKRLGDTFV | SHHEFLALWDSAKGTIHGEEVE |
| | LTLSDFRRENEGYYFCSALSNSIMYF | QEKIAVFRDASRFILNLTSVKPE |
| | SHFVPVFLPAKPTTTPAPRPPTPAPTI | DSGIYFCMIVGSPELTFGKGTQL |
| | ASQPLSLRPEACRPAAGGAVHTRGL | SVVDFLPTTAQPTKKSTLKKRV |
| | DFACDIYIWAPLAGTCGVLLLSLVIT | CRLPRPETQKGPLCSPITLGLLV |
| | RSKRSRGGHSDAMNMTARRAGPTR | AGVLVLLVSLGVAIRSKRSRGG |
| | KHYQAYAAPRDFAAYRS | HSDAMNMTARRAGPTRKHYQ |
| | (SEQ ID NO.: 122) | AYAAPRDFAAYRS |
| | | (SEQ ID NO.: 123) |
| F (without | SQFRVSPLDRTWNLGETVELKCQVL | LQQTPAYIKVQTNKMVMLSCE |
| signal | LSNPTSGCSWLFQPRGAAASPTFLL | AKISLSNMRIYWLRQRQAPSSD |
| peptides) | YLSQNKPKAAEGLDTQRFSGKRLG | SHHEFLALWDSAKGTIHGEEVE |
| | DTFVLTLSDFRRENEGYYFCSALSN | QEKIAVFRDASRFILNLTSVKPE |
| | SIMYFSHFVPVFLPAKPTTTPAPRPP | DSGIYFCMIVGSPELTFGKGTQL |
| | TPAPTIASQPLSLRPEACRPAAGGAV | SVVDFLPTTAQPTKKSTLKKRV |
| | HTRGLDFACDIYIWAPLAGTCGVLL | CRLPRPETQKGPLCSPITLGLLV |
| | LSLVITRSKRSRGGHSDAMNMTAR | AGVLVLLVSLGVAIRSKRSRGG |
| | RAGPTRKHYQAYAAPRDFAAYRS | HSDAMNMTARRAGPTRKHYQ |
| | (SEQ ID NO.: 124) | AYAAPRDFAAYRS |
| | | (SEQ ID NO.: 125) |
| G (with | MALPVTALLLPLALLLHAARPSQFR | MRPRLWLLLAAQLTVLHGNSV |
| signal | VSPLDRTWNLGETVELKCQVLLSNP | LQQTPAYIKVQTNKMVMLSCE |
| peptides) | TSGCSWLFQPRGAAASPTFLLYLSQ | AKISLSNMRIYWLRQRQAPSSD |
| | NKPKAAEGLDTQRFSGKRLGDTFV | SHHEFLALWDSAKGTIHGEEVE |
| | LTLSDFRRENEGYYFCSALSNSIMYF | QEKIAVFRDASRFILNLTSVKPE |
| | SHFVPVFLPAKPTTTPAPRPPTPAPTI | DSGIYFCMIVGSPELTFGKGTQL |
| | ASQPLSLRPEACRPAAGGAVHTRGL | SVVDFLPTTAQPTKKSTLKKRV |
| | DFACDIYIWAPLAGTCGVLLLSLVIT | CRLPRPETQKGPLCSPITLGLLV |
| | LYCNHRNRRRVCKCPRPVVKSGDK | AGVLVLLVSLGVAIHLCCRRRS |
| | PSLSARYV | KRSRLLHSDYMNMTPRRPGPTR |
| | (SEQ ID NO.: 1) | KHYQPYAPPRDFAAYRS |
| | | (SEQ ID NO.: 126) |
| G (without | SQFRVSPLDRTWNLGETVELKCQVL | LQQTPAYIKVQTNKMVMLSCE |
| signal | LSNPTSGCSWLFQPRGAAASPTFLL | AKISLSNMRIYWLRQRQAPSSD |
| peptides) | YLSQNKPKAAEGLDTQRFSGKRLG | SHHEFLALWDSAKGTIHGEEVE |
| | DTFVLTLSDFRRENEGYYFCSALSN | QEKIAVFRDASRFILNLTSVKPE |
| | SIMYFSHFVPVFLPAKPTTTPAPRPP | DSGIYFCMIVGSPELTFGKGTQL |
| | TPAPTIASQPLSLRPEACRPAAGGAV | SVVDFLPTTAQPTKKSTLKKRV |
| | HTRGLDFACDIYIWAPLAGTCGVLL | CRLPRPETQKGPLCSPITLGLLV |
| | LSLVITLYCNHRNRRRVCKCPRPVV | AGVLVLLVSLGVAIHLCCRRRS |
| | KSGDKPSLSARYV | KRSRLLHSDYMNMTPRRPGPTR |
| | (SEQ ID NO.: 114) | KHYQPYAPPRDFAAYRS |
| | | (SEQ ID NO.: 127) |
| H (with | MALPVTALLLPLALLLHAARPSQFR | MRPRLWLLLAAQLTVLHGNSV |
| signal | VSPLDRTWNLGETVELKCQVLLSNP | LQQTPAYIKVQTNKMVMLSCE |
| peptides) | TSGCSWLFQPRGAAASPTFLLYLSQ | AKISLSNMRIYWLRQRQAPSSD |
| | NKPKAAEGLDTQRFSGKRLGDTFV | SHHEFLALWDSAKGTIHGEEVE |
| | LTLSDFRRENEGYYFCSALSNSIMYF | QEKIAVFRDASRFILNLTSVKPE |
| | SHFVPVFLPAKPTTTPAPRPPTPAPTI | DSGIYFCMIVGSPELTFGKGTQL |
| | ASQPLSLRPEACRPAAGGAVHTRGL | SVVDFLPTTAQPTKKSTLKKRV |
| | DFACDIYIWAPLAGTCGVLLLSLVIT | CRLPRPETQKGPLCSPITLGLLV |
| | LYCNHRNRRRVCKCPRPVVKSGDK | AGVLVLLVSLGVAIHLCCRRKR |
| | PSLSARYV | GRKKLLYIFKQPFMRPVQTTQE |
| | (SEQ ID NO.: 1) | EDGCSCRFPEEEEGGCEL |
| | | (SEQ ID NO.: 128) |
| H (without | SQFRVSPLDRTWNLGETVELKCQVL | LQQTPAYIKVQTNKMVMLSCE |
| signal | LSNPTSGCSWLFQPRGAAASPTFLL | AKISLSNMRIYWLRQRQAPSSD |
| peptides) | YLSQNKPKAAEGLDTQRFSGKRLG | SHHEFLALWDSAKGTIHGEEVE |
| | DTFVLTLSDFRRENEGYYFCSALSN | QEKIAVFRDASRFILNLTSVKPE |
| | SIMYFSHFVPVFLPAKPTTTPAPRPP | DSGIYFCMIVGSPELTFGKGTQL |
| | TPAPTIASQPLSLRPEACRPAAGGAV | SVVDFLPTTAQPTKKSTLKKRV |
| | HTRGLDFACDIYIWAPLAGTCGVLL | CRLPRPETQKGPLCSPITLGLLV |
| | LSLVITLYCNHRNRRRVCKCPRPVV | AGVLVLLVSLGVAIHLCCRRKR |
| | KSGDKPSLSARYV | GRKKLLYIFKQPFMRPVQTTQE |
| | (SEQ ID NO.: 114) | EDGCSCRFPEEEEGGCEL |
| | | (SEQ ID NO.: 129) |
| I (with | MALPVTALLLPLALLLHAARPSQFR | MRPRLWLLLAAQLTVLHGNSV |
| signal | VSPLDRTWNLGETVELKCQVLLSNP | LQQTPAYIKVQTNKMVMLSCE |
| peptides) | TSGCSWLFQPRGAAASPTFLLYLSQ | AKISLSNMRIYWLRQRQAPSSD |
| | NKPKAAEGLDTQRFSGKRLGDTFV | SHHEFLALWDSAKGTIHGEEVE |
| | LTLSDFRRENEGYYFCSALSNSIMYF | QEKIAVFRDASRFILNLTSVKPE |
| | SHFVPVFLPAKPTTTPAPRPPTPAPTI | DSGIYFCMIVGSPELTFGKGTQL |
| | ASQPLSLRPEACRPAAGGAVHTRGL | SVVDFLPTTAQPTKKSTLKKRV |
| | DFACDIYIWAPLAGTCGVLLLSLVIT | CRLPRPETQKGPLCSPITLGLLV |
| | LYCNHRNRRRVCKCPRPVVKSGDK | AGVLVLLVSLGVAIHLCCRRCW |
| | PSLSARYV | LTKKKYSSSVHDPNGEYMFMR |
| | (SEQ ID NO.: 1) | AVNTAKKSRLTDVTL |
| | | (SEQ ID NO.: 130) |
| I (without | SQFRVSPLDRTWNLGETVELKCQVL | LQQTPAYIKVQTNKMVMLSCE |
| signal | LSNPTSGCSWLFQPRGAAASPTFLL | AKISLSNMRIYWLRQRQAPSSD |
| peptides) | YLSQNKPKAAEGLDTQRFSGKRLG | SHHEFLALWDSAKGTIHGEEVE |
| | DTFVLTLSDFRRENEGYYFCSALSN | QEKIAVFRDASRFILNLTSVKPE |
| | SIMYFSHFVPVFLPAKPTTTPAPRPP | DSGIYFCMIVGSPELTFGKGTQL |
| | TPAPTIASQPLSLRPEACRPAAGGAV | SVVDFLPTTAQPTKKSTLKKRV |
| | HTRGLDFACDIYIWAPLAGTCGVLL | CRLPRPETQKGPLCSPITLGLLV |
| | LSLVITLYCNHRNRRRVCKCPRPVV | AGVLVLLVSLGVAIHLCCRRCW |
| | KSGDKPSLSARYV | LTKKKYSSSVHDPNGEYMFMR |
| | (SEQ ID NO.: 114) | AVNTAKKSRLTDVTL |
| | | (SEQ ID NO.: 131) |
| J (with | MALPVTALLLPLALLLHAARPSQFR | MRPRLWLLLAAQLTVLHGNSV |
| signal | VSPLDRTWNLGETVELKCQVLLSNP | LQQTPAYIKVQTNKMVMLSCE |
| peptides) | TSGCSWLFQPRGAAASPTFLLYLSQ | AKISLSNMRIYWLRQRQAPSSD |
| | NKPKAAEGLDTQRFSGKRLGDTFV | SHHEFLALWDSAKGTIHGEEVE |
| | LTLSDFRRENEGYYFCSALSNSIMYF | QEKIAVFRDASRFILNLTSVKPE |
| | SHFVPVFLPAKPTTTPAPRPPTPAPTI | DSGIYFCMIVGSPELTFGKGTQL |
| | ASQPLSLRPEACRPAAGGAVHTRGL | SVVDFLPTTAQPTKKSTLKKRV |
| | DFACDIYIWAPLAGTCGVLLLSLVIT | CRLPRPETQKGPLCSPITLGLLV |
| | LYCNHRNRRRVCKCPRPVVKSGDK | AGVLVLLVSLGVAIHLCCRRAL |
| | PSLSARYV | YLLRRDQRLPPDAHKPPGGGSF |
| | (SEQ ID NO.: 1) | RTPIQEEQADAHSTLAKI |
| | | (SEQ ID NO.: 132) |
| J (without | SQFRVSPLDRTWNLGETVELKCQVL | LQQTPAYIKVQTNKMVMLSCE |
| signal | LSNPTSGCSWLFQPRGAAASPTFLL | AKISLSNMRIYWLRQRQAPSSD |
| peptides) | YLSQNKPKAAEGLDTQRFSGKRLG | SHHEFLALWDSAKGTIHGEEVE |
| | DTFVLTLSDFRRENEGYYFCSALSN | QEKIAVFRDASRFILNLTSVKPE |
| | SIMYFSHFVPVFLPAKPTTTPAPRPP | DSGIYFCMIVGSPELTFGKGTQL |
| | TPAPTIASQPLSLRPEACRPAAGGAV | SVVDFLPTTAQPTKKSTLKKRV |
| | HTRGLDFACDIYIWAPLAGTCGVLL | CRLPRPETQKGPLCSPITLGLLV |
| | LSLVITLYCNHRNRRRVCKCPRPVV | AGVLVLLVSLGVAIHLCCRRAL |
| | KSGDKPSLSARYV | YLLRRDQRLPPDAHKPPGGGSF |
| | (SEQ ID NO.: 114) | RTPIQEEQADAHSTLAKI |
| | | (SEQ ID NO.: 133) |
| K (with | MALPVTALLLPLALLLHAARPSQFR | MRPRLWLLLAAQLTVLHGNSV |
| signal | VSPLDRTWNLGETVELKCQVLLSNP | LQQTPAYIKVQTNKMVMLSCE |
| peptides) | TSGCSWLFQPRGAAASPTFLLYLSQ | AKISLSNMRIYWLRQRQAPSSD |
| | NKPKAAEGLDTQRFSGKRLGDTFV | SHHEFLALWDSAKGTIHGEEVE |
| | LTLSDFRRENEGYYFCSALSNSIMYF | QEKIAVFRDASRFILNLTSVKPE |
| | SHFVPVFLPAKPTTTPAPRPPTPAPTI | DSGIYFCMIVGSPELTFGKGTQL |
| | ASQPLSLRPEACRPAAGGAVHTRGL | SVVDFLPTTAQPTKKSTLKKRV |
| | DFACDIYIWAPLAGTCGVLLLSLVIT | CRLPRPETQKGPLCSPITLGLLV |
| | LYCNHRNRRRVCKCPRPVVKSGDK | AGVLVLLVSLGVAIHLCCRRQL |
| | PSLSARYV | GLHIWQLRSQCMWPRETQLLLE |
| | (SEQ ID NO.: 1) | VPPSTEDARSCQFPEEERGERSA |
| | | EEKGRLGDLWV |
| | | (SEQ ID NO.: 134) |
| K (without | SQFRVSPLDRTWNLGETVELKCQVL | LQQTPAYIKVQTNKMVMLSCE |
| signal | LSNPTSGCSWLFQPRGAAASPTFLL | AKISLSNMRIYWLRQRQAPSSD |
| peptides) | YLSQNKPKAAEGLDTQRFSGKRLG | SHHEFLALWDSAKGTIHGEEVE |
| | DTFVLTLSDFRRENEGYYFCSALSN | QEKIAVFRDASRFILNLTSVKPE |
| | SIMYFSHFVPVFLPAKPTTTPAPRPP | DSGIYFCMIVGSPELTFGKGTQL |
| | TPAPTIASQPLSLRPEACRPAAGGAV | SVVDFLPTTAQPTKKSTLKKRV |
| | HTRGLDFACDIYIWAPLAGTCGVLL | CRLPRPETQKGPLCSPITLGLLV |
| | LSLVITLYCNHRNRRRVCKCPRPVV | AGVLVLLVSLGVAIHLCCRRQL |
| | KSGDKPSLSARYV | GLHIWQLRSQCMWPRETQLLLE |
| | (SEQ ID NO.: 114) | VPPSTEDARSCQFPEEERGERSA |
| | | EEKGRLGDLWV |
| | | (SEQ ID NO.: 135) |
| L (with | MALPVTALLLPLALLLHAARPSQFR | MRPRLWLLLAAQLTVLHGNSV |
| signal | VSPLDRTWNLGETVELKCQVLLSNP | LQQTPAYIKVQTNKMVMLSCE |
| peptides) | TSGCSWLFQPRGAAASPTFLLYLSQ | AKISLSNMRIYWLRQRQAPSSD |
| | NKPKAAEGLDTQRFSGKRLGDTFV | SHHEFLALWDSAKGTIHGEEVE |
| | LTLSDFRRENEGYYFCSALSNSIMYF | QEKIAVFRDASRFILNLTSVKPE |
| | SHFVPVFLPAFWVLVVVGGVLACY | DSGIYFCMIVGSPELTFGKGTQL |
| | SLLVTVAFIIFWVRSKRSRGGHSDY | SVVDFLPTTAQPTKKSTLKKRV |
| | MNMTPRRPGPTRKHYQPYAPPRDF | CRLPRPETQKGPLCSPITLGLLV |
| | AAYRS | AGVLVLLVSLGVAIHLCCRRRR |
| | (SEQ ID NO.: 136) | ARLRFMKQFYKPLQDNLVIALH |
| | | SYEPSHDGDLGFEKGEQLRILEQ |
| | | SGEWWKAQSLTTGQEGFIPFNF |
| | | VAKANSLEPEPWFFKNLSRKDA |
| | | ERQLLAPGNTHGSFLIRESESTA |
| | | GSFSLSVRDFDQNQGEVVKHY |
| | | KIRNLDNGGFYISPRITFPGLHEL |
| | | VRHYTNASDGLCTRLSRPCQTQ |
| | | KPQKPWWEDEWEVPRETLKLV |
| | | ERLGAGQFGEVWMGYYNGHT |
| | | KVAVKSLKQGSMSPDAFLAEA |
| | | NLMKQLQHQRLVRLYAVVTQE |
| | | PIYIITEYMENGSLVDFLKTPSGI |
| | | KLTINKLLDMAAQIAEGMAFIE |
| | | ERNYIHRDLRAANILVSDTLSCK |
| | | IADFGLARLIEDNEYTAREGAKF |
| | | PIKWTAPEAINYGTFTIKSDVWS |
| | | FGILLTEIVTHGRIPYPGMTNPE |
| | | VIQNLERGYRMVRPDNCPEELY |
| | | QLMRLCWKERPEDRPTFDYLRS |
| | | VLEDFFTATEGQYQPQP |
| | | (SEQ ID NO.: 137) |
| L (without | SQFRVSPLDRTWNLGETVELKCQVL | LQQTPAYIKVQTNKMVMLSCE |
| signal | LSNPTSGCSWLFQPRGAAASPTFLL | AKISLSNMRIYWLRQRQAPSSD |
| peptides) | YLSQNKPKAAEGLDTQRFSGKRLG | SHHEFLALWDSAKGTIHGEEVE |
| | DTFVLTLSDFRRENEGYYFCSALSN | QEKIAVFRDASRFILNLTSVKPE |
| | SIMYFSHFVPVFLPAFWVLVVVGGV | DSGIYFCMIVGSPELTFGKGTQL |
| | LACYSLLVTVAFIIFWVRSKRSRGG | SVVDFLPTTAQPTKKSTLKKRV |
| | HSDYMNMTPRRPGPTRKHYQPYAP | CRLPRPETQKGPLCSPITLGLLV |
| | PRDFAAYRS | AGVLVLLVSLGVAIHLCCRRRR |
| | (SEQ ID NO.: 138) | ARLRFMKQFYKPLQDNLVIALH |
| | | SYEPSHDGDLGFEKGEQLRILEQ |
| | | SGEWWKAQSLTTGQEGFIPFNF |
| | | VAKANSLEPEPWFFKNLSRKDA |
| | | ERQLLAPGNTHGSFLIRESESTA |
| | | GSFSLSVRDFDQNQGEVVKHY |
| | | KIRNLDNGGFYISPRITFPGLHEL |
| | | VRHYTNASDGLCTRLSRPCQTQ |
| | | KPQKPWWEDEWEVPRETLKLV |
| | | ERLGAGQFGEVWMGYYNGHT |
| | | KVAVKSLKQGSMSPDAFLAEA |
| | | NLMKQLQHQRLVRLYAVVTQE |
| | | PIYIITEYMENGSLVDFLKTPSGI |
| | | KLTINKLLDMAAQIAEGMAFIE |
| | | ERNYIHRDLRAANILVSDTLSCK |
| | | IADFGLARLIEDNEYTAREGAKF |
| | | PIKWTAPEAINYGTFTIKSDVWS |
| | | FGILLTEIVTHGRIPYPGMTNPE |
| | | VIQNLERGYRMVRPDNCPEELY |
| | | QLMRLCWKERPEDRPTFDYLRS |
| | | VLEDFFTATEGQYQPQP |
| | | (SEQ ID NO.: 139) |
| M (with | MALPVTALLLPLALLLHAARPSQFR | |
| signal | VSPLDRTWNLGETVELKCQVLLSNP | |
| peptide) | TSGCSWLFQPRGAAASPTFLLYLSQ | |
| | NKPKAAEGLDTQRFSGKRLGDTFV | |
| | LTLSDFRRENEGYYFCSALSNSIMYF | |
| | SHFVPVFLPADFLPTTAQPTKKSTLK | |
| | KRVCRLPRPETQKGPLCCPSPLFPGP | |
| | SKPFWVLVVVGGVLACYSLLVTVA | |
| | FIIFWVRSKRSRGGHSDYMNMTPRR | |
| | PGPTRKHYQPYAPPRDFAAYRS | |
| | (SEQ ID NO.: 140) | |
| M (without | SQFRVSPLDRTWNLGETVELKCQVL | |
| signal | LSNPTSGCSWLFQPRGAAASPTFLL | |
| peptide) | YLSQNKPKAAEGLDTQRFSGKRLG | |
| | DTFVLTLSDFRRENEGYYFCSALSN | |
| | SIMYFSHFVPVFLPADFLPTTAQPTK | |
| | KSTLKKRVCRLPRPETQKGPLCCPS | |
| | PLFPGPSKPFWVLVVVGGVLACYSL | |
| | LVTVAFIIFWVRSKRSRGGHSDYMN | |
| | MTPRRPGPTRKHYQPYAPPRDFAA | |
| | YRS | |
| | (SEQ ID NO.: 141) | |
| N (with | MALPVTALLLPLALLLHAARPSQFR | |
| signal | VSPLDRTWNLGETVELKCQVLLSNP | |
| peptide) | TSGCSWLFQPRGAAASPTFLLYLSQ | |
| | NKPKAAEGLDTQRFSGKRLGDTFV | |
| | LTLSDFRRENEGYYFCSALSNSIMYF | |
| | SHFVPVFLPADFLPTTAQPTKKSTLK | |
| | KRVCRLPRPETQKGPLCFWVLVVV | |
| | GGVLACYSLLVTVAFIIFWVRSKRS | |
| | RGGHSDYMNMTPRRPGPTRKHYQP | |
| | YAPPRDFAAYRS | |
| | (SEQ ID NO.: 142) | |
| N (without | SQFRVSPLDRTWNLGETVELKCQVL | |
| signal | LSNPTSGCSWLFQPRGAAASPTFLL | |
| peptide) | YLSQNKPKAAEGLDTQRFSGKRLG | |
| | DTFVLTLSDFRRENEGYYFCSALSN | |
| | SIMYFSHFVPVFLPADFLPTTAQPTK | |
| | KSTLKKRVCRLPRPETQKGPLCFWV | |
| | LVVVGGVLACYSLLVTVAFIIFWVR | |
| | SKRSRGGHSDYMNMTPRRPGPTRK | |
| | HYQPYAPPRDFAAYRS | |
| | (SEQ ID NO.: 143) | |
| O (with | MALPVTALLLPLALLLHAARPSQFR | |
| signal | VSPLDRTWNLGETVELKCQVLLSNP | |
| peptide) | TSGCSWLFQPRGAAASPTFLLYLSQ | |
| | NKPKAAEGLDTQRFSGKRLGDTFV | |
| | LTLSDFRRENEGYYFCSALSNSIMYF | |
| | SHFVPVFLPADFLPTTAQPTKKSTLK | |
| | KRVCRLPRPETQKGPLCFWVLVVV | |
| | GGVLACYSLLVTVAFIIFWVRSKRS | |
| | RGGHSDYMNMTPRRPGPTRKHYQP | |
| | YAPPRDFAAYRSLYCNHRNRRRVC | |
| | KCPRPVVKSGDKPSLSARYV | |
| | (SEQ ID NO.: 144) | |
| O (without | SQFRVSPLDRTWNLGETVELKCQVL | |
| signal | LSNPTSGCSWLFQPRGAAASPTFLL | |
| peptide) | YLSQNKPKAAEGLDTQRFSGKRLG | |
| | DTFVLTLSDFRRENEGYYFCSALSN | |
| | SIMYFSHFVPVFLPADFLPTTAQPTK | |
| | KSTLKKRVCRLPRPETQKGPLCFWV | |
| | LVVVGGVLACYSLLVTVAFIIFWVR | |
| | SKRSRGGHSDYMNMTPRRPGPTRK | |
| | HYQPYAPPRDFAAYRSLYCNHRNR | |
| | RRVCKCPRPVVKSGDKPSLSARYV | |
| | (SEQ ID NO.: 145) | |
| P (with | MALPVTALLLPLALLLHAARPSQFR | |
| signal | VSPLDRTWNLGETVELKCQVLLSNP | |
| peptide) | TSGCSWLFQPRGAAASPTFLLYLSQ | |
| | NKPKAAEGLDTQRFSGKRLGDTFV | |
| | LTLSDFRRENEGYYFCSALSNSIMYF | |
| | SHFVPVFLPADFLPTTAQPTKKSTLK | |
| | KRVCRLPRPETQKGPLCDIYIWAPL | |
| | AGTCGVLLLSLVITLYCNHRNRRRV | |
| | CKCPRPVVKSGDKPSLSARYV | |
| | (SEQ ID NO.: 146) | |
| P (without | SQFRVSPLDRTWNLGETVELKCQVL | |
| signal | LSNPTSGCSWLFQPRGAAASPTFLL | |
| peptide) | YLSQNKPKAAEGLDTQRFSGKRLG | |
| | DTFVLTLSDFRRENEGYYFCSALSN | |
| | SIMYFSHFVPVFLPADFLPTTAQPTK | |
| | KSTLKKRVCRLPRPETQKGPLCDIYI | |
| | WAPLAGTCGVLLLSLVITLYCNHRN | |
| | RRRVCKCPRPVVKSGDKPSLSARYV | |
| | (SEQ ID NO.: 147) | |
| Q (with | MLRLLLALNLFPSIQVTGIPLTESYC | |
| signal | GPCPKNWICYKNNCYQFFDESKNW | |
| peptide) | YESQASCMSQNASLLKVYSKEDQD | |
| | LLKLVKSYHWMGLVHIPTNGSWQ | |
| | WEDGSILSPNLLTIIEMQKGDCALY | |
| | ASSFKGYIENCSTPNTYICMQRTVK | |
| | GKHLCPSPLFPGPSKPFWVLVVVGG | |
| | VLACYSLLVTVAFIIFWVRSKRSRLL | |
| | HSDYMNMTPRRPGPTRKHYQPYAP | |
| | PRDFAAYRS | |
| | (SEQ ID NO.: 148) | |
| Q (without | IPLTESYCGPCPKNWICYKNNCYQF | |
| signal | FDESKNWYESQASCMSQNASLLKV | |
| peptide) | YSKEDQDLLKLVKSYHWMGLVHIP | |
| | TNGSWQWEDGSILSPNLLTIIEMQK | |
| | GDCALYASSFKGYIENCSTPNTYIC | |
| | MQRTVKGKHLCPSPLFPGPSKPFWV | |
| | LVVVGGVLACYSLLVTVAFIIFWVR | |
| | SKRSRLLHSDYMNMTPRRPGPTRK | |
| | HYQPYAPPRDFAAYRS | |
| | (SEQ ID NO.: 149) | |
| R (with | MALPVTALLLPLALLLHAARPSQFR | |
| signal | VSPLDRTWNLGETVELKCQVLLSNP | |
| peptides) | TSGCSWLFQPRGAAASPTFLLYLSQ | |
| | NKPKAAEGLDTQRFSGKRLGDTFV | |
| | LTLSDFRRENEGYYFCSALSNSIMYF | |
| | SHFVPVFLPAKPTTTPAPRPPTPAPTI | |
| | ASQPLSLRPEACRPAAGGAVHTRGL | |
| | DFACDIYIWAPLAGTCGVLLLSLVIT | |
| | LYCNHRNRRRVCKCPRPVVKSGDK | |
| | PSLSARYV | |
| | (SEQ ID NO.: 1) | |
| R (without | SQFRVSPLDRTWNLGETVELKCQVL | MNPTDIADTTLDESIYSNYYLYE |
| signal | LSNPTSGCSWLFQPRGAAASPTFLL | SIPKPCTKEGIKAFGELFLPPLYS |
| peptide) | YLSQNKPKAAEGLDTQRFSGKRLG | LVFVFGLLGNSVVVLVLFKYKR |
| (Protein 2 | DTFVLTLSDFRRENEGYYFCSALSN | LRSMTDVYLLNLAISDLLFVFSL |
| (CCR4) | SIMYFSHFVPVFLPAKPTTTPAPRPP | PFWGYYAADQWVFGLGLCKMI |
| lacks a | TPAPTIASQPLSLRPEACRPAAGGAV | SWMYLVGFYSGIFFVMLMSIDR |
| signal | HTRGLDFACDIYIWAPLAGTCGVLL | YLAIVHAVFSLRARTLTYGVITS |
| peptide) | LSLVITLYCNHRNRRRVCKCPRPVV | LATWSVAVFASLPGFLFSTCYT |
| | KSGDKPSLSARYV | ERNHTYCKTKYSLNSTTWKVLS |
| | (SEQ ID NO.: 114) | SLEINILGLVIPLGIMLFCYSMIIR |
| | | TLQHCKNEKKNKAVKMIFAVV |
| | | VLFLGFWTPYNIVLFLETLVELE |
| | | VLQDCTFERYLDYAIQATETLA |
| | | FVHCCLNPIIYFFLGEKFRKYILQ |
| | | LFKTCRGLFVLCQYCGLLQIYS |
| | | ADTPSSSYTQSTMDHDLHDAL |
| | | (SEQ ID NO.: 106) |
| S (with | MALPVTALLLPLALLLHAARPSQFR | |
| signal | VSPLDRTWNLGETVELKCQVLLSNP | |
| peptide) | TSGCSWLFQPRGAAASPTFLLYLSQ | |
| | NKPKAAEGLDTQRFSGKRLGDTFV | |
| | LTLSDFRRENEGYYFCSALSNSIMYF | |
| | SHFVPVFLPAKPTTTPAPRPPTPAPTI | |
| | ASQPLSLRPEACRPAAGGAVHTRGL | |
| | DFACDIYIWAPLAGTCGVLLLSLVIT | |
| | LYCNHRNRRRVCKCPRPVVKSGDK | |
| | PSLSARYV | |
| | (SEQ ID NO.: 1) | |
| S (without | SQFRVSPLDRTWNLGETVELKCQVL | MLSTSRSRFIRNTNESGEEVTTF |
| signal | LSNPTSGCSWLFQPRGAAASPTFLL | FDYDYGAPCHKFDVKQIGAQLL |
| peptide | YLSQNKPKAAEGLDTQRFSGKRLG | PPLYSLVFIFGFVGNMLVVLILI |
| (Protein 2 | DTFVLTLSDFRRENEGYYFCSALSN | NCKKLKCLTDIYLLNLAISDLLF |
| (CCR2b) | SIMYFSHFVPVFLPAKPTTTPAPRPP | LITLPLWAHSAANEWVFGNAM |
| lacks a | TPAPTIASQPLSLRPEACRPAAGGAV | CKLFTGLYHIGYFGGIFFIILLTID |
| signal | HTRGLDFACDIYIWAPLAGTCGVLL | RYLAIVHAVFALKARTVTFGVV |
| peptide) | LSLVITLYCNHRNRRRVCKCPRPVV | TSVITWLVAVFASVPGIIFTKCQ |
| | KSGDKPSLSARYV | KEDSVYVCGPYFPRGWNNFHTI |
| | (SEQ ID NO.: 114) | MRNILGLVLPLLIMVICYSGILK |
| | | TLLRCRNEKKRHRAVRVIFTIMI |
| | | VYFLFWTPYNIVILLNTFQEFFG |
| | | LSNCESTSQLDQATQVTETLGM |
| | | THCCINPIIYAFVGEKFRRYLSV |
| | | FFRKHITKRFCKQCPVFYRETV |
| | | DGVTSTNTPSTGEQEVSAGL |
| | | (SEQ ID NO.: 107) |
| T (with | MALPVTALLLPLALLLHAARPSQFR | MRPRLWLLLAAQLTVLHGNSV |
| signal | VSPLDRTWNLGETVELKCQVLLSNP | LQQTPAYIKVQTNKMVMLSCE |
| peptides) | TSGCSWLFQPRGAAASPTFLLYLSQ | AKISLSNMRIYWLRQRQAPSSD |
| | NKPKAAEGLDTQRFSGKRLGDTFV | SHHEFLALWDSAKGTIHGEEVE |
| | LTLSDFRRENEGYYFCSALSNSIMYF | QEKIAVFRDASRFILNLTSVKPE |
| | SHFVPVFLPAKPTTTPAPRPPTPAPTI | DSGIYFCMIVGSPELTFGKGTQL |
| | ASQPLSLRPEACRPAAGGAVHTRGL | SVVDFLPTTAQPTKKSTLKKRV |
| | DFACDIYIWAPLAGTCGVLLLSLVIT | CRLPRPETQKGPLCSPITLGLLV |
| | RSKRSRGGHSDAMNMTARRAGPTR | AGVLVLLVSLGVAIRSKRSRGG |
| | KHFQAFAAPRDFAAFRS | HSDAMNMTARRAGPTRKHFQA |
| | (SEQ ID NO.: 150) | FAAPRDFAAFRS |
| | | (SEQ ID NO.: 151) |
| T (without | SQFRVSPLDRTWNLGETVELKCQVL | LQQTPAYIKVQTNKMVMLSCE |
| signal | LSNPTSGCSWLFQPRGAAASPTFLL | AKISLSNMRIYWLRQRQAPSSD |
| peptides) | YLSQNKPKAAEGLDTQRFSGKRLG | SHHEFLALWDSAKGTIHGEEVE |
| | DTFVLTLSDFRRENEGYYFCSALSN | QEKIAVFRDASRFILNLTSVKPE |
| | SIMYFSHFVPVFLPAKPTTTPAPRPP | DSGIYFCMIVGSPELTFGKGTQL |
| | TPAPTIASQPLSLRPEACRPAAGGAV | SVVDFLPTTAQPTKKSTLKKRV |
| | HTRGLDFACDIYIWAPLAGTCGVLL | CRLPRPETQKGPLCSPITLGLLV |
| | LSLVITRSKRSRGGHSDAMNMTAR | AGVLVLLVSLGVAIRSKRSRGG |
| | RAGPTRKHFQAFAAPRDFAAFRS | HSDAMNMTARRAGPTRKHFQA |
| | (SEQ ID NO.: 152) | FAAPRDFAAFRS |
| | | (SEQ ID NO.: 153) |
| U (with | MLGIWTLLPLVLTSVARLSSKSVNA | |
| signal | QVTDINSKGLELRKTVTTVETQNLE | |
| peptide) | GLHHDGQFCHKPCPPGERKARDCT | |
| | VNGDEPDCVPCQEGKEYTDKAHFS | |
| | SKCRRCRLCDEGHGLEVEINCTRTQ | |
| | NTKCRCKPNFFCNSTVCEHCDPCTK | |
| | CEHGIIKECTLTSNTKCKEEGSRSNL | |
| | GWLCLLLLPIPLIVWVK | |
| | (SEQ ID NO.: 154) | |
| U (without | QVTDINSKGLELRKTVTTVETQNLE | |
| signal | GLHHDGQFCHKPCPPGERKARDCT | |
| peptide) | VNGDEPDCVPCQEGKEYTDKAHFS | |
| | SKCRRCRLCDEGHGLEVEINCTRTQ | |
| | NTKCRCKPNFFCNSTVCEHCDPCTK | |
| | CEHGIIKECTLTSNTKCKEEGSRSNL | |
| | GWLCLLLLPIPLIVWVK | |
| | (SEQ ID NO.: 155) | |
| V (with | MLGIWTLLPLVLTSVARLSSKSVNA | |
| signal | QVTDINSKGLELRKTVTTVETQNLE | |
| peptide) | GLHHDGQFCHKPCPPGERKARDCT | |
| | VNGDEPDCVPCQEGKEYTDKAHFS | |
| | SKCRRCRLCDEGHGLEVEINCTRTQ | |
| | NTKCRCKPNFFCNSTVCEHCDPCTK | |
| | CEHGIIKECTLTSNTKCKEEGSRSNL | |
| | GWLCLLLLPIPLIVWVPLQDNLVIAL | |
| | HSYEPSHDGDLGFEKGEQLRILEQS | |
| | GEWWKAQSLTTGQEGFIPFNFVAK | |
| | ANSLEPEPWFFKNLSRKDAERQLLA | |
| | PGNTHGSFLIRESESTAGSFSLSVRD | |
| | FDQNQGEVVKHYKIRNLDNGGFYIS | |
| | PRITFPGLHELVRHYTNASDGLCTR | |
| | LSRPCQTQKPQKPWWEDEWEVPRE | |
| | TLKLVERLGAGQFGEVWMGYYNG | |
| | HTKVAVKSLKQGSMSPDAFLAEAN | |
| | LMKQLQHQRLVRLYAVVTQEPIYII | |
| | TEYMENGSLVDFLKTPSGIKLTINKL | |
| | LDMAAQIAEGMAFIEERNYIHRDLR | |
| | AANILVSDTLSCKIADFGLARLIEDN | |
| | EYTAREGAKFPIKWTAPEAINYGTF | |
| | TIKSDVWSFGILLTEIVTHGRIPYPG | |
| | MTNPEVIQNLERGYRMVRPDNCPE | |
| | ELYQLMRLCWKERPEDRPTFDYLR | |
| | SVLEDFFTATEGQYQPQP | |
| | (SEQ ID NO.: 156) | |
| V (without | QVTDINSKGLELRKTVTTVETQNLE | |
| signal | GLHHDGQFCHKPCPPGERKARDCT | |
| peptide) | VNGDEPDCVPCQEGKEYTDKAHFS | |
| | SKCRRCRLCDEGHGLEVEINCTRTQ | |
| | NTKCRCKPNFFCNSTVCEHCDPCTK | |
| | CEHGIIKECTLTSNTKCKEEGSRSNL | |
| | GWLCLLLLPIPLIVWVPLQDNLVIAL | |
| | HSYEPSHDGDLGFEKGEQLRILEQS | |
| | GEWWKAQSLTTGQEGFIPFNFVAK | |
| | ANSLEPEPWFFKNLSRKDAERQLLA | |
| | PGNTHGSFLIRESESTAGSFSLSVRD | |
| | FDQNQGEVVKHYKIRNLDNGGFYIS | |
| | PRITFPGLHELVRHYTNASDGLCTR | |
| | LSRPCQTQKPQKPWWEDEWEVPRE | |
| | TLKLVERLGAGQFGEVWMGYYNG | |
| | HTKVAVKSLKQGSMSPDAFLAEAN | |
| | LMKQLQHQRLVRLYAVVTQEPIYII | |
| | TEYMENGSLVDFLKTPSGIKLTINKL | |
| | LDMAAQIAEGMAFIEERNYIHRDLR | |
| | AANILVSDTLSCKIADFGLARLIEDN | |
| | EYTAREGAKFPIKWTAPEAINYGTF | |
| | TIKSDVWSFGILLTEIVTHGRIPYPG | |
| | MTNPEVIQNLERGYRMVRPDNCPE | |
| | ELYQLMRLCWKERPEDRPTFDYLR | |
| | SVLEDFFTATEGQYQPQP | |
| | (SEQ ID NO.: 157) | |
| W (with | MLGIWTLLPLVLTSVARLSSKSVNA | |
| signal | QVTDINSKGLELRKTVTTVETQNLE | |
| peptide) | GLHHDGQFCHKPCPPGERKARDCT | |
| | VNGDEPDCVPCQEGKEYTDKAHFS | |
| | SKCRRCRLCDEGHGLEVEINCTRTQ | |
| | NTKCRCKPNFFCNSTVCEHCDPCTK | |
| | CEHGIIKECTLTSNTKCKEEGSRSNL | |
| | GWLCLLLLPIPLIVWVLYCNHRNRR | |
| | RVCKCPRPVVKSGDKPSLSARYV | |
| | (SEQ ID NO.: 158) | |
| W (without | QVTDINSKGLELRKTVTTVETQNLE | |
| signal | GLHHDGQFCHKPCPPGERKARDCT | |
| peptide) | VNGDEPDCVPCQEGKEYTDKAHFS | |
| | SKCRRCRLCDEGHGLEVEINCTRTQ | |
| | NTKCRCKPNFFCNSTVCEHCDPCTK | |
| | CEHGIIKECTLTSNTKCKEEGSRSNL | |
| | GWLCLLLLPIPLIVWVLYCNHRNRR | |
| | RVCKCPRPVVKSGDKPSLSARYV | |
| | (SEQ ID NO.: 159) | |
| X (with | MLGIWTLLPLVLTSVARLSSKSVNA | |
| signal | QVTDINSKGLELRKTVTTVETQNLE | |
| peptide) | GLHHDGQFCHKPCPPGERKARDCT | |
| | VNGDEPDCVPCQEGKEYTDKAHFS | |
| | SKCRRCRLCDEGHGLEVEINCTRTQ | |
| | NTKCRCKPNFFCNSTVCEHCDPCTK | |
| | CEHGIIKECTLTSNTKCKEEGSRSNL | |
| | GWLCLLLLPIPLIVWVGSGGGTGGG | |
| | SGGSGGGTGGGSGMASSSGSSPRPA | |
| | PDENEFPFGCPPTVCQDPKEPRALC | |
| | CAGCLSENPRNGEDQICPKCRGEDL | |
| | QSISPGSRLRTQEKAHPEVAEAGIGC | |
| | PFAGVGCSFKGSPQSVQEHEVTSQT | |
| | SHLNLLLGFMKQWKARLGCGLESG | |
| | PMALEQNLSDLQLQAAVEVAGDLE | |
| | VDCYRAPCSESQEELALQHFMKEK | |
| | LLAELEGKLRVFENIVAVLNKEVEA | |
| | SHLALATSIHQSQLDRERILSLEQRV | |
| | VELQQTLAQKDQALGKLEQSLRLM | |
| | EEASFDGTFLWKITNVTRRCHESAC | |
| | GRTVSLFSPAFYTAKYGYKLCLRLY | |
| | LNGDGTGKRTHLSLFIVIMRGEYDA | |
| | LLPWPFRNKVTFMLLDQNNREHAI | |
| | DAFRPDLSSASFQRPQSETNVASGC | |
| | PLFFPLSKLQSPKHAYVKDDTMFLK | |
| | CIVETST | |
| | (SEQ ID NO.: 160) | |
| X (without | QVTDINSKGLELRKTVTTVETQNLE | |
| signal | GLHHDGQFCHKPCPPGERKARDCT | |
| peptide) | VNGDEPDCVPCQEGKEYTDKAHFS | |
| | SKCRRCRLCDEGHGLEVEINCTRTQ | |
| | NTKCRCKPNFFCNSTVCEHCDPCTK | |
| | CEHGIIKECTLTSNTKCKEEGSRSNL | |
| | GWLCLLLLPIPLIVWVGSGGGTGGG | |
| | SGGSGGGTGGGSGMASSSGSSPRPA | |
| | PDENEFPFGCPPTVCQDPKEPRALC | |
| | CAGCLSENPRNGEDQICPKCRGEDL | |
| | QSISPGSRLRTQEKAHPEVAEAGIGC | |
| | PFAGVGCSFKGSPQSVQEHEVTSQT | |
| | SHLNLLLGFMKQWKARLGCGLESG | |
| | PMALEQNLSDLQLQAAVEVAGDLE | |
| | VDCYRAPCSESQEELALQHFMKEK | |
| | LLAELEGKLRVFENIVAVLNKEVEA | |
| | SHLALATSIHQSQLDRERILSLEQRV | |
| | VELQQTLAQKDQALGKLEQSLRLM | |
| | EEASFDGTFLWKITNVTRRCHESAC | |
| | GRTVSLFSPAFYTAKYGYKLCLRLY | |
| | LNGDGTGKRTHLSLFIVIMRGEYDA | |
| | LLPWPFRNKVTFMLLDQNNREHAI | |
| | DAFRPDLSSASFQRPQSETNVASGC | |
| | PLFFPLSKLQSPKHAYVKDDTMFLK | |
| | CIVETST | |
| | (SEQ ID NO.: 161) | |
| Y (with | MQIPQAPWPVVWAVLQLGWRPGW | |
| signal | FLDSPDRPWNPPTFSPALLVVTEGD | |
| peptide) | NATFTCSFSNTSESFVLNWYRMSPS | |
| | NQTDKLAAFPEDRSQPGQDCRFRVT | |
| | QLPNGRDFHMSVVRARRNDSGTYL | |
| | CGAISLAPKAQIKESLRAELRVTERR | |
| | AEVPTAHPSPCPSPLFPGPSKPFWVL | |
| | VVVGGVLACYSLLVTVAFIIFWVRS | |
| | KRSRGGHSDYMNMTPRRPGPTRKH | |
| | YQPYAPPRDFAAYRS | |
| | (SEQ ID NO.: 162) | |
| Y (without | FLDSPDRPWNPPTFSPALLVVTEGD | |
| signal | NATFTCSFSNTSESFVLNWYRMSPS | |
| peptide) | NQTDKLAAFPEDRSQPGQDCRFRVT | |
| | QLPNGRDFHMSVVRARRNDSGTYL | |
| | CGAISLAPKAQIKESLRAELRVTERR | |
| | AEVPTAHPSPCPSPLFPGPSKPFWVL | |
| | VVVGGVLACYSLLVTVAFIIFWVRS | |
| | KRSRGGHSDYMNMTPRRPGPTRKH | |
| | YQPYAPPRDFAAYRS | |
| | (SEQ ID NO.: 163) | |
| Z (with | MKWKALFTAAILQAQLPITEAQSFG | |
| signal | LLDPKLCYLLDGILFIYGVILTALFL | |
| peptide) | RSKRSRGGHSDYMNMTPRRPGPTR | |
| | KHYQPYAPPRDFAAYRSRVKFSRSA | |
| | DAPAYQQGQNQLYNELNLGRREEY | |
| | DVLDKRRGRDPEMGGKPRRKNPQE | |
| | GLYNELQKDKMAEAYSEIGMKGER | |
| | RRGKGHDGLYQGLSTATKDTYDAL | |
| | HMQALPPR | |
| | (SEQ ID NO.: 164) | |
| Z (without | QSFGLLDPKLCYLLDGILFIYGVILT | |
| signal | ALFLRSKRSRGGHSDYMNMTPRRP | |
| peptide) | GPTRKHYQPYAPPRDFAAYRSRVKF | |
| | SRSADAPAYQQGQNQLYNELNLGR | |
| | REEYDVLDKRRGRDPEMGGKPRRK | |
| | NPQEGLYNELQKDKMAEAYSEIGM | |
| | KGERRRGKGHDGLYQGLSTATKDT | |
| | YDALHMQALPPR | |
| | (SEQ ID NO.: 165) | |
| AA (with | MKWKALFTAAILQAQLPITEAQSFG | |
| signal | LLDPKLCYLLDGILFIYGVILTALFL | |
| peptide) | RGRKKLLYIFKQPFMRPVQTTQEED | |
| | GCSCRFPEEEEGGCELRVKFSRSAD | |
| | APAYQQGQNQLYNELNLGRREEYD | |
| | VLDKRRGRDPEMGGKPRRKNPQEG | |
| | LYNELQKDKMAEAYSEIGMKGERR | |
| | RGKGHDGLYQGLSTATKDTYDALH | |
| | MQALPPR | |
| | (SEQ ID NO.: 166) | |
| AA | QSFGLLDPKLCYLLDGILFIYGVILT | |
| (without | ALFLRGRKKLLYIFKQPFMRPVQTT | |
| signal | QEEDGCSCRFPEEEEGGCELRVKFS | |
| peptide) | RSADAPAYQQGQNQLYNELNLGRR | |
| | EEYDVLDKRRGRDPEMGGKPRRKN | |
| | PQEGLYNELQKDKMAEAYSEIGMK | |
| | GERRRGKGHDGLYQGLSTATKDTY | |
| | DALHMQALPPR | |
| | (SEQ ID NO.: 167) | |

TABLE 4
Summary of Amino Acid SEQ ID NOs.
of Protein Constructs A-AA

| Construct | Polypeptide 1 | Polypeptide 2 |
|---|---|---|
| A (with signal peptides) | (SEQ ID NO.: 1) | (SEQ ID NO.: 113) |
| A (without signal peptides) | (SEQ ID NO.: 114) | (SEQ ID NO.: 115) |
| B (with signal peptides) | (SEQ ID NO.: 1) | (SEQ ID NO.: 116) |
| B (without signal peptides) | (SEQ ID NO.: 114) | (SEQ ID NO.: 117) |
| C (with signal peptides) | (SEQ ID NO.: 118) | (SEQ ID NO.: 116) |
| C (without signal peptides) | (SEQ ID NO.: 39) | (SEQ ID NO.: 117) |
| D (with signal peptides) | (SEQ ID NO.: 118) | (SEQ ID NO.: 6) |
| D (without signal peptides) | (SEQ ID NO.: 39) | (SEQ ID NO.: 119) |
| E (with signal peptides) | (SEQ ID NO.: 1) | (SEQ ID NO.: 120) |
| E (without signal peptides) | (SEQ ID NO.: 114) | (SEQ ID NO.: 121) |
| F (with signal peptides) | (SEQ ID NO.: 122) | (SEQ ID NO.: 123) |
| F (without signal peptides) | (SEQ ID NO.: 124) | (SEQ ID NO.: 125) |
| G (with signal peptides) | (SEQ ID NO.: 1) | (SEQ ID NO.: 126) |
| G (without signal peptides) | (SEQ ID NO.: 114) | (SEQ ID NO.: 127) |
| H (with signal peptides) | (SEQ ID NO.: 1) | (SEQ ID NO.: 128) |
| H (without signal peptides) | (SEQ ID NO.: 114) | (SEQ ID NO.: 129) |
| I (with signal peptides) | (SEQ ID NO.: 1) | (SEQ ID NO.: 130) |
| I (without signal peptides) | (SEQ ID NO.: 114) | (SEQ ID NO.: 131) |
| J (with signal peptides) | (SEQ ID NO.: 1) | (SEQ ID NO.: 132) |
| J (without signal peptides) | (SEQ ID NO.: 114) | (SEQ ID NO.: 133) |
| K (with signal peptides) | (SEQ ID NO.: 1) | (SEQ ID NO.: 134) |
| K (without signal peptides) | (SEQ ID NO.: 114) | (SEQ ID NO.: 135) |
| L (with signal peptides) | (SEQ ID NO.: 136) | (SEQ ID NO.: 137) |
| L (without signal peptides) | (SEQ ID NO.: 138) | (SEQ ID NO.: 139) |
| M (with signal peptide) | (SEQ ID NO.: 140) | n/a |
| M (without signal peptide) | (SEQ ID NO.: 141) | n/a |
| N (with signal peptide) | (SEQ ID NO.: 142) | n/a |
| N (without signal peptide) | (SEQ ID NO.: 143) | n/a |
| O (with signal peptide) | (SEQ ID NO.: 144) | n/a |
| O (without signal peptide) | (SEQ ID NO.: 145) | n/a |
| P (with signal peptide) | (SEQ ID NO.: 146) | n/a |
| P (without signal peptide) | (SEQ ID NO.: 147) | n/a |
| Q (with signal peptide) | (SEQ ID NO.: 148) | n/a |
| Q (without signal peptide) | (SEQ ID NO.: 149) | n/a |
| R (with signal peptides) | (SEQ ID NO.: 1) | n/a |
| R (without signal peptide) | (SEQ ID NO.: 114) | (SEQ ID NO.: 106) |
| S (with signal peptide) | (SEQ ID NO.: 1) | n/a |
| S (without signal peptide) | (SEQ ID NO.: 114) | (SEQ ID NO.: 107) |
| T (with signal peptides) | (SEQ ID NO.: 150) | (SEQ ID NO.: 151) |
| T (without signal peptides) | (SEQ ID NO.: 152) | (SEQ ID NO.: 153) |
| U (with signal peptide) | (SEQ ID NO.: 154) | n/a |
| U (without signal peptide) | (SEQ ID NO.: 155) | n/a |
| V (with signal peptide) | (SEQ ID NO.: 156) | n/a |
| V (without signal peptide) | (SEQ ID NO.: 157) | n/a |
| W (with signal peptide) | (SEQ ID NO.: 158) | n/a |
| W (without signal peptide) | (SEQ ID NO.: 159) | n/a |
| X (with signal peptide) | (SEQ ID NO.: 160) | n/a |
| X (without signal peptide) | (SEQ ID NO.: 161) | n/a |
| Y (with signal peptide) | (SEQ ID NO.: 162) | n/a |
| Y (without signal peptide) | (SEQ ID NO.: 163) | n/a |
| Z (with signal peptide) | (SEQ ID NO.: 164) | n/a |
| Z (without signal peptide) | (SEQ ID NO.: 165) | n/a |
| AA (with signal peptide) | (SEQ ID NO.: 166) | n/a |
| AA (without signal peptide) | (SEQ ID NO.: 167) | n/a |

Construct Y was used as a positive control and reference in certain experiments described in the Examples. Another construct was generated that encoded wild-type, full-length human CD8α and CD8β separated by a P2A coding sequence; this construct was also used as a control in certain experiments described in the Examples.

Table 5 provides certain additional embodiments of polypeptides (Polypeptide Types “A1”-“AA”) of the present disclosure.

TABLE 5
Polypeptide Types “A1”-“AA”

| Polypeptide Type A1 | Polypeptide Type A2: |
|---|---|
| A polypeptide comprising: | A polypeptide comprising: |
| (i) an extracellular component that | (i) an extracellular component that |
| comprises an amino acid sequence | comprises an amino acid sequence having |
| having at least 90%, at least 91%, at least | at least 90%, at least 91%, at least 92%, at |
| 92%, at least 93%, at least 94%, at least | least 93%, at least 94%, at least 95%, at |
| 95%, at least 96%, at least 97%, at least | least 96%, at least 97%, at least 98%, or |
| 98%, or at least 99% identity to, or | at least 99% identity to, or comprising or |
| comprising or consisting of, the amino | consisting of, the amino acid sequence set |
| acid sequence set forth in SEQ ID | forth in SEQ ID NO.: 7; |
| NO.: 2; | (ii) a transmembrane domain that |
| (ii) a transmembrane domain that | comprises an amino acid sequence having |
| comprises an amino acid sequence | at least 90%, at least 91%, at least 92%, at |
| having at least 90%, at least 91%, at least | least 93%, at least 94%, at least 95%, at |
| 92%, at least 93%, at least 94%, at least | least 96%, at least 97%, at least 98%, or |
| 95%, at least 96%, at least 97%, at least | at least 99% identity to, or comprising or |
| 98%, or at least 99% identity to, or | consisting of, the amino acid sequence set |
| comprising or consisting of, the amino | forth in SEQ ID NO.: 8; and |
| acid sequence set forth in SEQ ID | (iii) an intracellular component that |
| NO.: 3; and | comprises an amino acid sequence having |
| (iii) an intracellular component that | at least 90%, at least 91%, at least 92%, at |
| comprises an amino acid sequence | least 93%, at least 94%, at least 95%, at |
| having at least 90%, at least 91%, at least | least 96%, at least 97%, at least 98%, or |
| 92%, at least 93%, at least 94%, at least | at least 99% identity to, or comprising or |
| 95%, at least 96%, at least 97%, at least | consisting of, the amino acid sequence set |
| 98%, or at least 99% identity to, or | forth in SEQ ID NO.: 83, |
| comprising or consisting of, the amino | wherein the polypeptide is capable of |
| acid sequence set forth in SEQ ID NO.: 4, | binding to a MHC-Class I molecule and |
| wherein the polypeptide is capable of | providing a stimulatory signal. |
| binding to a MHC-Class I molecule and, | |
| optionally, associating with a Lck. | |

Polypeptide Type B
A polypeptide comprising:
(i) an extracellular component that comprises an amino acid sequence having at least
90%, at least 91%, at least 92%, at least 93%, at least 94%, at least 95%, at least
96%, at least 97%, at least 98%, or at least 99% identity to, or comprising or
consisting of, the amino acid sequence set forth in SEQ ID NO.: 7;
(ii) a transmembrane domain that comprises an amino acid sequence having at least
90%, at least 91%, at least 92%, at least 93%, at least 94%, at least 95%, at least
96%, at least 97%, at least 98%, or at least 99% identity to, or comprising or
consisting of, the amino acid sequence set forth in SEQ ID NO.: 8; and
(iii) an intracellular component that comprises an amino acid sequence having at least
90%, at least 91%, at least 92%, at least 93%, at least 94%, at least 95%, at least
96%, at least 97%, at least 98%, or at least 99% identity to, or comprising or
consisting of, the amino acid sequence set forth in SEQ ID NO.: 20,
wherein the polypeptide is capable of binding to a MHC-Class I molecule and
providing a stimulatory signal.

| | |
|---|---|
| Polypeptide Type C1 | Polypeptide Type C2 |
| A polypeptide comprising: | A polypeptide comprising: |
| (i) an extracellular component that | (i) an extracellular component that |
| comprises an amino acid sequence | comprises an amino acid sequence having |
| having at least 90%, at least 91%, at least | at least 90%, at least 91%, at least 92%, at |
| 92%, at least 93%, at least 94%, at least | least 93%, at least 94%, at least 95%, at |
| 95%, at least 96%, at least 97%, at least | least 96%, at least 97%, at least 98%, or |
| 98%, or at least 99% identity to, or | at least 99% identity to, or comprising or |
| comprising or consisting of, the amino | consisting of, the amino acid sequence set |
| acid sequence set forth in SEQ ID | forth in SEQ ID NO.: 7; |
| NO.: 2; | (ii) a transmembrane domain that |
| (ii) a transmembrane domain that | comprises an amino acid sequence having |
| comprises an amino acid sequence | at least 90%, at least 91%, at least 92%, at |
| having at least 90%, at least 91%, at least | least 93%, at least 94%, at least 95%, at |
| 92%, at least 93%, at least 94%, at least | least 96%, at least 97%, at least 98%, or |
| 95%, at least 96%, at least 97%, at least | at least 99% identity to, or comprising or |
| 98%, or at least 99% identity to, or | consisting of, the amino acid sequence set |
| comprising or consisting of, the amino | forth in SEQ ID NO.: 8; and |
| acid sequence set forth in SEQ ID | (iii) an intracellular component that |
| NO.: 3; and | comprises an amino acid sequence having |
| (iii) an intracellular component that | at least 90%, at least 91%, at least 92%, at |
| comprises an amino acid sequence | least 93%, at least 94%, at least 95%, at |
| having at least 90%, at least 91%, at least | least 96%, at least 97%, at least 98%, or |
| 92%, at least 93%, at least 94%, at least | at least 99% identity to, or comprising or |
| 95%, at least 96%, at least 97%, at least | consisting of, the amino acid sequence set |
| 98%, or at least 99% identity to, or | forth in SEQ ID NO.: 20, |
| comprising or consisting of, the amino | wherein the polypeptide is capable of |
| acid sequence set forth in SEQ ID | binding to a MHC-Class I molecule and |
| NO.: 20, | providing a stimulatory signal. |
| wherein the polypeptide is capable of | |
| binding to a MHC-Class I molecule and | |
| providing a stimulatory signal. | |
| Polypeptide Type D1 | Polypeptide Type D2 |
| A polypeptide comprising: | A polypeptide comprising: |
| (i) an extracellular component that | (i) an extracellular component that |
| comprises an amino acid sequence | comprises an amino acid sequence having |
| having at least 90%, at least 91%, at least | at least 90%, at least 91%, at least 92%, at |
| 92%, at least 93%, at least 94%, at least | least 93%, at least 94%, at least 95%, at |
| 95%, at least 96%, at least 97%, at least | least 96%, at least 97%, at least 98%, or |
| 98%, or at least 99% identity to, or | at least 99% identity to, or comprising or |
| comprising or consisting of, the amino | consisting of, the amino acid sequence set |
| acid sequence set forth in SEQ ID | forth in SEQ ID NO.: 7; |
| NO.: 2; | (ii) a transmembrane domain that |
| (ii) a transmembrane domain that | comprises an amino acid sequence having |
| comprises an amino acid sequence | at least 90%, at least 91%, at least 92%, at |
| having at least 90%, at least 91%, at least | least 93%, at least 94%, at least 95%, at |
| 92%, at least 93%, at least 94%, at least | least 96%, at least 97%, at least 98%, or |
| 95%, at least 96%, at least 97%, at least | at least 99% identity to, or comprising or |
| 98%, or at least 99% identity to, or | consisting of, the amino acid sequence set |
| comprising or consisting of, the amino | forth in SEQ ID NO.: 8; and |
| acid sequence set forth in SEQ ID | (iii) an intracellular component that |
| NO.: 3; and | comprises an amino acid sequence having |
| (iii) an intracellular component that | at least 90%, at least 91%, at least 92%, at |
| comprises an amino acid sequence | least 93%, at least 94%, at least 95%, at |
| having at least 90%, at least 91%, at least | least 96%, at least 97%, at least 98%, or |
| 92%, at least 93%, at least 94%, at least | at least 99% identity to, or comprising or |
| 95%, at least 96%, at least 97%, at least | consisting of, the amino acid sequence set |
| 98%, or at least 99% identity to, or | forth in SEQ ID NO.: 9, |
| comprising or consisting of, the amino | wherein the polypeptide is capable of |
| acid sequence set forth in SEQ ID | binding to a MHC-Class I molecule. |
| NO.: 20, | |
| wherein the polypeptide is capable of | |
| binding to a MHC-Class I molecule and | |
| providing a stimulatory signal. | |

Polypeptide Type E
A polypeptide comprising:
(i) an extracellular component that comprises an amino acid sequence having at least
90%, at least 91%, at least 92%, at least 93%, at least 94%, at least 95%, at least
96%, at least 97%, at least 98%, or at least 99% identity to, or comprising or
consisting of, the amino acid sequence set forth in SEQ ID NO.: 7;
(ii) a transmembrane domain that comprises an amino acid sequence having at least
90%, at least 91%, at least 92%, at least 93%, at least 94%, at least 95%, at least
96%, at least 97%, at least 98%, or at least 99% identity to, or comprising or
consisting of, the amino acid sequence set forth in SEQ ID NO.: 8; and
(iii) an intracellular component that comprises an amino acid sequence having at least
96%, at least 97%, at least 98%, or at least 99% identity to, or comprising or
90%, at least 91%, at least 92%, at least 93%, at least 94%, at least 95%, at least
96%, at least 97%, at least 98%, or at least 99% identity to, or comprising or
consisting of, the amino acid sequence set forth in SEQ ID NO.: 180,
wherein the polypeptide is capable of binding to a MHC-Class I molecule and
providing a stimulatory signal, wherein, optionally, the stimulatory signal is reduced
(e.g. lessened or attenuated) as compared to the stimulatory signal provided by a
reference polypeptide that comprises in the intracellular component thereof the amino
acid sequence set forth in any one of SEQ ID NOs .: 19, 20, 83, and 84.

| | |
|---|---|
| Polypeptide Type F1 | Polypeptide Type F2 |
| A polypeptide comprising: | A polypeptide comprising: |
| (i) an extracellular component that | (i) an extracellular component that |
| comprises an amino acid sequence | comprises an amino acid sequence having |
| having at least 90%, at least 91%, at least | at least 90%, at least 91%, at least 92%, at |
| 92%, at least 93%, at least 94%, at least | least 93%, at least 94%, at least 95%, at |
| 95%, at least 96%, at least 97%, at least | least 96%, at least 97%, at least 98%, or |
| 98%, or at least 99% identity to, or | at least 99% identity to, or comprising or |
| comprising or consisting of, the amino | consisting of, the amino acid sequence set |
| acid sequence set forth in SEQ ID | forth in SEQ ID NO.: 7; |
| NO.: 2; | (ii) a transmembrane domain that |
| (ii) a transmembrane domain that | comprises an amino acid sequence having |
| comprises an amino acid sequence | at least 90%, at least 91%, at least 92%, at |
| having at least 90%, at least 91%, at least | least 93%, at least 94%, at least 95%, at |
| 92%, at least 93%, at least 94%, at least | least 96%, at least 97%, at least 98%, or |
| 95%, at least 96%, at least 97%, at least | at least 99% identity to, or comprising or |
| 98%, or at least 99% identity to, or | consisting of, the amino acid sequence set |
| comprising or consisting of, the amino | forth in SEQ ID NO.: 8; and |
| acid sequence set forth in SEQ ID | (iii) an intracellular component that |
| NO.: 3; and | comprises an amino acid sequence having |
| (iii) an intracellular component that | at least 90%, at least 91%, at least 92%, at |
| comprises an amino acid sequence | least 93%, at least 94%, at least 95%, at |
| having at least 90%, at least 91%, at least | least 96%, at least 97%, at least 98%, or |
| 92%, at least 93%, at least 94%, at least | at least 99% identity to, or comprising or |
| 95%, at least 96%, at least 97%, at least | consisting of, the amino acid sequence set |
| 98%, or at least 99% identity to, or | forth in SEQ ID NO.: 81, |
| comprising or consisting of, the amino | wherein the polypeptide is capable of |
| acid sequence set forth in SEQ ID | binding to a MHC-Class I molecule and |
| NO.: 81, | providing a stimulatory signal, wherein, |
| wherein the polypeptide is capable of | optionally, the stimulatory signal is |
| binding to a MHC-Class I molecule and | reduced as compared to the stimulatory |
| providing a stimulatory signal, wherein, | signal provided by a reference |
| optionally, the stimulatory signal is | polypeptide that comprises in the |
| reduced as compared to the stimulatory | intracellular component thereof the amino |
| signal provided by a reference | acid sequence set forth in any one of SEQ |
| polypeptide that comprises in the | ID NOs .: 19, 20, 83, and 84. |
| intracellular component thereof the | |
| amino acid sequence set forth in SEQ ID | |
| NO.: 19 or 20. | |

Polypeptide Type G
A polypeptide comprising:
(i) an extracellular component that comprises an amino acid sequence having at least
90%, at least 91%, at least 92%, at least 93%, at least 94%, at least 95%, at least
96%, at least 97%, at least 98%, or at least 99% identity to, or comprising or
consisting of, the amino acid sequence set forth in SEQ ID NO.: 7;
(ii) a transmembrane domain that comprises an amino acid sequence having at least
90%, at least 91%, at least 92%, at least 93%, at least 94%, at least 95%, at least
96%, at least 97%, at least 98%, or at least 99% identity to, or comprising or
consisting of, the amino acid sequence set forth in SEQ ID NO.: 8; and
(iii) an intracellular component that comprises an amino acid sequence having at least
90%, at least 91%, at least 92%, at least 93%, at least 94%, at least 95%, at least
96%, at least 97%, at least 98%, or at least 99% identity to, or comprising or
consisting of, the amino acid sequence set forth in SEQ ID NO.: 84,
wherein the polypeptide is capable of binding to a MHC-Class I molecule and
providing a stimulatory signal.
Polypeptide Type H
A polypeptide comprising:
(i) an extracellular component that comprises an amino acid sequence having at least
90%, at least 91%, at least 92%, at least 93%, at least 94%, at least 95%, at least
96%, at least 97%, at least 98%, or at least 99% identity to, or comprising or
consisting of, the amino acid sequence set forth in SEQ ID NO.: 7;
(ii) a transmembrane domain that comprises an amino acid sequence having at least
90%, at least 91%, at least 92%, at least 93%, at least 94%, at least 95%, at least
96%, at least 97%, at least 98%, or at least 99% identity to, or comprising or
consisting of, the amino acid sequence set forth in SEQ ID NO.: 8; and
(iii) an intracellular component that comprises an amino acid sequence having at least
90%, at least 91%, at least 92%, at least 93%, at least 94%, at least 95%, at least
96%, at least 97%, at least 98%, or at least 99% identity to, or comprising or
consisting of, the amino acid sequence set forth in SEQ ID NO.: 85,
wherein the polypeptide is capable of binding to a MHC-Class I molecule and
providing a stimulatory signal.
Polypeptide Type I
A polypeptide comprising:
(i) an extracellular component that comprises an amino acid sequence having at least
90%, at least 91%, at least 92%, at least 93%, at least 94%, at least 95%, at least
96%, at least 97%, at least 98%, or at least 99% identity to, or comprising or
consisting of, the amino acid sequence set forth in SEQ ID NO.: 7;
(ii) a transmembrane domain that comprises an amino acid sequence having at least
90%, at least 91%, at least 92%, at least 93%, at least 94%, at least 95%, at least
96%, at least 97%, at least 98%, or at least 99% identity to, or comprising or
consisting of, the amino acid sequence set forth in SEQ ID NO.: 8; and
(iii) an intracellular component that comprises an amino acid sequence having at least
90%, at least 91%, at least 92%, at least 93%, at least 94%, at least 95%, at least
96%, at least 97%, at least 98%, or at least 99% identity to, or comprising or
consisting of, the amino acid sequence set forth in SEQ ID NO.: 86,
wherein the polypeptide is capable of binding to a MHC-Class I molecule and
providing a stimulatory signal.
Polypeptide Type J
A polypeptide comprising:
(i) an extracellular component that comprises an amino acid sequence that has at least
90%, at least 91%, at least 92%, at least 93%, at least 94%, at least 95%, at least
96%, at least 97%, at least 98%, or at least 99% identity to, or that comprises or
consists of, the amino acid sequence set forth in SEQ ID NO.: 7;
(ii) a transmembrane domain that comprises an amino acid sequence that has at least
90%, at least 91%, at least 92%, at least 93%, at least 94%, at least 95%, at least
96%, at least 97%, at least 98%, or at least 99% identity to, or that comprises or
consists of, the amino acid sequence set forth in SEQ ID NO.: 8; and
(iii) an intracellular component that comprises an amino acid sequence that has at
least 90%, at least 91%, at least 92%, at least 93%, at least 94%, at least 95%, at least
96%, at least 97%, at least 98%, or at least 99% identity to, or that comprises or
consists of, the amino acid sequence set forth in SEQ ID NO.: 87,
wherein the polypeptide is capable of binding to a MHC-Class I molecule and
providing a stimulatory signal.
Polypeptide Type K
A polypeptide comprising:
(i) an extracellular component that comprises an amino acid sequence having at least
90%, at least 91%, at least 92%, at least 93%, at least 94%, at least 95%, at least
96%, at least 97%, at least 98%, or at least 99% identity to, or comprising or
consisting of, the amino acid sequence set forth in SEQ ID NO.: 7;
(ii) a transmembrane domain that comprises an amino acid sequence having at least
90%, at least 91%, at least 92%, at least 93%, at least 94%, at least 95%, at least
96%, at least 97%, at least 98%, or at least 99% identity to, or comprising or
consisting of, the amino acid sequence set forth in SEQ ID NO.: 8; and
(iii) an intracellular component that comprises an amino acid sequence having at least
90%, at least 91%, at least 92%, at least 93%, at least 94%, at least 95%, at least
96%, at least 97%, at least 98%, or at least 99% identity to, or comprising or
consisting of, the amino acid sequence set forth in SEQ ID NO.: 88,
wherein the polypeptide is capable of binding to a MHC-Class I molecule and
providing a stimulatory signal.

| | |
|---|---|
| Polypeptide Type L1 | Polypeptide Type L2 |
| A polypeptide comprising: | A polypeptide comprising: |
| (i) an extracellular component that | (i) an extracellular component that |
| comprises an amino acid sequence | comprises an amino acid sequence having |
| having at least 90%, at least 91%, at least | at least 90%, at least 91%, at least 92%, at |
| 92%, at least 93%, at least 94%, at least | least 93%, at least 94%, at least 95%, at |
| 95%, at least 96%, at least 97%, at least | least 96%, at least 97%, at least 98%, or |
| 98%, or at least 99% identity to, or | at least 99% identity to, or comprising or |
| comprising or consisting of, the amino | consisting of, the amino acid sequence set |
| acid sequence set forth in SEQ ID | forth in SEQ ID NO.: 7; |
| NO.: 173; | (ii) a transmembrane domain that |
| (ii) a transmembrane domain that | comprises an amino acid sequence having |
| comprises an amino acid sequence | at least 90%, at least 91%, at least 92%, at |
| having at least 90%, at least 91%, at least | least 93%, at least 94%, at least 95%, at |
| 92%, at least 93%, at least 94%, at least | least 96%, at least 97%, at least 98%, or |
| 95%, at least 96%, at least 97%, at least | at least 99% identity to, or comprising or |
| 98%, or at least 99% identity to, or | consisting of, the amino acid sequence set |
| comprising or consisting of, the amino | forth in SEQ ID NO.: 8; and |
| acid sequence set forth in SEQ ID | (iii) an intracellular component that |
| NO.: 80; and | comprises an amino acid sequence having |
| (iii) an intracellular component that | at least 90%, at least 91%, at least 92%, at |
| comprises an amino acid sequence | least 93%, at least 94%, at least 95%, at |
| having at least 90%, at least 91%, at least | least 96%, at least 97%, at least 98%, or |
| 92%, at least 93%, at least 94%, at least | at least 99% identity to, or comprising or |
| 95%, at least 96%, at least 97%, at least | consisting of, the amino acid sequence set |
| 98%, or at least 99% identity to, or | forth in SEQ ID NO.: 89, |
| comprising or consisting of, the amino | wherein the polypeptide is capable of |
| acid sequence set forth in SEQ ID | binding to a MHC-Class I molecule and |
| NO.: 20, | providing a stimulatory signal. |
| wherein the polypeptide is capable of | |
| binding to a MHC-Class I molecule and | |
| providing a stimulatory signal. | |

Polypeptide Type M
A polypeptide comprising:
(i) an extracellular component that comprises an amino acid sequence having at least
90%, at least 91%, at least 92%, at least 93%, at least 94%, at least 95%, at least
96%, at least 97%, at least 98%, or at least 99% identity to, or comprising or
consisting of, the amino acid sequence set forth in SEQ ID NO.: 90;
(ii) a transmembrane domain that comprises an amino acid sequence having at least
90%, at least 91%, at least 92%, at least 93%, at least 94%, at least 95%, at least
96%, at least 97%, at least 98%, or at least 99% identity to, or comprising or
consisting of, the amino acid sequence set forth in SEQ ID NO.: 80; and
(iii) an intracellular component that comprises an amino acid sequence having at least
90%, at least 91%, at least 92%, at least 93%, at least 94%, at least 95%, at least
96%, at least 97%, at least 98%, or at least 99% identity to, or comprising or
consisting of, the amino acid sequence set forth in SEQ ID NO.: 20,
wherein the polypeptide is capable of binding to a MHC-Class I molecule and
providing a stimulatory signal.
Polypeptide Type N
A polypeptide comprising:
(i) an extracellular component that comprises an amino acid sequence having at least
90%, at least 91%, at least 92%, at least 93%, at least 94%, at least 95%, at least
96%, at least 97%, at least 98%, or at least 99% identity to, or comprising or
consisting of, the amino acid sequence set forth in SEQ ID NO.: 92;
(ii) a transmembrane domain that comprises an amino acid sequence having at least
90%, at least 91%, at least 92%, at least 93%, at least 94%, at least 95%, at least
96%, at least 97%, at least 98%, or at least 99% identity to, or comprising or
consisting of, the amino acid sequence set forth in SEQ ID NO.: 80; and
(iii) an intracellular component that comprises an amino acid sequence having at least
90%, at least 91%, at least 92%, at least 93%, at least 94%, at least 95%, at least
96%, at least 97%, at least 98%, or at least 99% identity to, or comprising or
consisting of, the amino acid sequence set forth in SEQ ID NO.: 20,
wherein the polypeptide is capable of binding to a MHC-Class I molecule and
providing a stimulatory signal.
Polypeptide Type O
A polypeptide comprising:
(i) an extracellular component that comprises an amino acid sequence having at least
90%, at least 91%, at least 92%, at least 93%, at least 94%, at least 95%, at least
96%, at least 97%, at least 98%, or at least 99% identity to, or comprising or
consisting of, the amino acid sequence set forth in SEQ ID NO.: 92;
(ii) a transmembrane domain that comprises an amino acid sequence having at least
90%, at least 91%, at least 92%, at least 93%, at least 94%, at least 95%, at least
96%, at least 97%, at least 98%, or at least 99% identity to, or comprising or
consisting of, the amino acid sequence set forth in SEQ ID NO.: 80; and
(iii) an intracellular component that comprises an amino acid sequence having at least
90%, at least 91%, at least 92%, at least 93%, at least 94%, at least 95%, at least
96%, at least 97%, at least 98%, or at least 99% identity to, or comprising or
consisting of, the amino acid sequence set forth in SEQ ID NO.: 97,
wherein the polypeptide is capable of binding to a MHC-Class I molecule, providing
a stimulatory signal, and, optionally, associating with a Lck.
Polypeptide Type P
A polypeptide comprising:
(i) an extracellular component that comprises an amino acid sequence having at least
90%, at least 91%, at least 92%, at least 93%, at least 94%, at least 95%, at least
96%, at least 97%, at least 98%, or at least 99% identity to, or comprising or
consisting of, the amino acid sequence set forth in SEQ ID NO.: 94;
(ii) a transmembrane domain that comprises an amino acid sequence having at least
90%, at least 91%, at least 92%, at least 93%, at least 94%, at least 95%, at least
96%, at least 97%, at least 98%, or at least 99% identity to, or comprising or
consisting of, the amino acid sequence set forth in SEQ ID NO.: 3; and
(iii) an intracellular component that comprises an amino acid sequence having at least
90%, at least 91%, at least 92%, at least 93%, at least 94%, at least 95%, at least
96%, at least 97%, at least 98%, or at least 99% identity to, or comprising or
consisting of, the amino acid sequence set forth in SEQ ID NO.: 4,
wherein the polypeptide is capable of binding to a MHC-Class I molecule, providing
a stimulatory signal, and, optionally, associating with a Lck.
Polypeptide Type Q
A polypeptide comprising:
(i) an extracellular component that comprises an amino acid sequence having at least
90%, at least 91%, at least 92%, at least 93%, at least 94%, at least 95%, at least
96%, at least 97%, at least 98%, or at least 99% identity to, or comprising or
consisting of, the amino acid sequence set forth in SEQ ID NO.: 174;
(ii) a transmembrane domain that comprises an amino acid sequence having at least
90%, at least 91%, at least 92%, at least 93%, at least 94%, at least 95%, at least
96%, at least 97%, at least 98%, or at least 99% identity to, or comprising or
consisting of, the amino acid sequence set forth in SEQ ID NO.: 80; and
(iii) an intracellular component that comprises an amino acid sequence having at least
90%, at least 91%, at least 92%, at least 93%, at least 94%, at least 95%, at least
96%, at least 97%, at least 98%, or at least 99% identity to, or comprising or
consisting of, the amino acid sequence set forth in SEQ ID NO.: 19;
wherein the polypeptide is capable of binding to a NKG2D ligand and providing a
stimulatory signal.
Polypeptide Type R
A polypeptide comprising an amino acid sequence that has at least 90%, at least 91%,
at least 92%, at least 93%, at least 94%, at least 95%, at least 96%, at least 97%, at
least 98%, or at least 99% identity to, or that comprises or consists of, the amino acid
sequence set forth in SEQ ID NO.: 106, wherein the polypeptide is capable of binding
to a CCR4 ligand and providing a stimulatory signal.
Polypeptide Type S
A polypeptide comprising an amino acid sequence that has at least 90%, at least 91%,
at least 92%, at least 93%, at least 94%, at least 95%, at least 96%, at least 97%, at
least 98%, or at least 99% identity to, or that comprises or consists of, the amino acid
sequence set forth in SEQ ID NO.: 107,
wherein the polypeptide is capable of binding to a CCR2B ligand and providing a
stimulatory signal.

| | |
|---|---|
| Polypeptide Type T1 | Polypeptide Type T2 |
| A polypeptide comprising: | A polypeptide comprising: |
| (i) an extracellular component that | (i) an extracellular component that |
| comprises an amino acid sequence | comprises an amino acid sequence having |
| having at least 90%, at least 91%, at least | at least 90%, at least 91%, at least 92%, at |
| 92%, at least 93%, at least 94%, at least | least 93%, at least 94%, at least 95%, at |
| 95%, at least 96%, at least 97%, at least | least 96%, at least 97%, at least 98%, or |
| 98%, or at least 99% identity to, or | at least 99% identity to, or comprising or |
| comprising or consisting of, the amino | consisting of, the amino acid sequence set |
| acid sequence set forth in SEQ ID | forth in SEQ ID NO.: 7; |
| NO.: 2; | (ii) a transmembrane domain that |
| (ii) a transmembrane domain that | comprises an amino acid sequence having |
| comprises an amino acid sequence | at least 90%, at least 91%, at least 92%, at |
| having at least 90%, at least 91%, at least | least 93%, at least 94%, at least 95%, at |
| 92%, at least 93%, at least 94%, at least | least 96%, at least 97%, at least 98%, or |
| 95%, at least 96%, at least 97%, at least | at least 99% identity to, or comprising or |
| 98%, or at least 99% identity to, or | consisting of, the amino acid sequence set |
| comprising or consisting of, the amino | forth in SEQ ID NO.: 8; and |
| acid sequence set forth in SEQ ID | (iii) an intracellular component that |
| NO.: 3; and | comprises an amino acid sequence having |
| (iii) an intracellular component that | at least 90%, at least 91%, at least 92%, at |
| comprises an amino acid sequence | least 93%, at least 94%, at least 95%, at |
| having at least 90%, at least 91%, at least | least 96%, at least 97%, at least 98%, or |
| 92%, at least 93%, at least 94%, at least | at least 99% identity to, or comprising or |
| 95%, at least 96%, at least 97%, at least | consisting of, the amino acid sequence set |
| 98%, or at least 99% identity to, or | forth in SEQ ID NO.: 108, |
| comprising or consisting of, the amino | wherein the polypeptide is capable of |
| acid sequence set forth in SEQ ID | binding to a MHC-Class I molecule and |
| NO.: 108, wherein the polypeptide is | providing a stimulatory signal, wherein, |
| capable of binding to a MHC-Class I | optionally, the stimulatory signal is |
| molecule and providing a stimulatory | reduced as compared to the stimulatory |
| signal, wherein, optionally, the | signal provided by a reference |
| stimulatory signal is reduced as | polypeptide that comprises in the |
| compared to the stimulatory signal | intracellular component thereof the amino |
| provided by a reference polypeptide that | acid sequence set forth in any one of SEQ |
| comprises in the intracellular component | ID NOs .: 19, 20, 83, and 84. |
| thereof the amino acid sequence set forth | |
| in set forth in SEQ ID NO.: 19 or 20. | |

Polypeptide Type U
A polypeptide comprising:
(i) an extracellular component that comprises an amino acid sequence having at least
90%, at least 91%, at least 92%, at least 93%, at least 94%, at least 95%, at least
96%, at least 97%, at least 98%, or at least 99% identity to, or comprising or
consisting of, the amino acid sequence set forth in SEQ ID NO.: 98;
(ii) a transmembrane domain that comprises an amino acid sequence having at least
90%, at least 91%, at least 92%, at least 93%, at least 94%, at least 95%, at least
96%, at least 97%, at least 98%, or at least 99% identity to, or comprising or
consisting of, the amino acid sequence set forth in SEQ ID NO.: 100; and
(iii) an optional intracellular component that does not comprise a functional Fas
signaling domain and optionally comprises or consists of not more than 50 amino
acids, not more than 40 amino acids, not more than 30 amino acids, not more than 20
amino acids, not more than 10 amino acids, or not more than 5 amino acids, and
further optionally comprises or consists of a single lysine amino acid,
wherein the polypeptide is capable of binding to a Fas ligand and does not provide a
Fas:FasL signal (e.g. does not provide or promote a suppressive and/or apoptotic
signal in a host cell).
Polypeptide Type V
A polypeptide comprising:
(i) an extracellular component that comprises an amino acid sequence having at least
90%, at least 91%, at least 92%, at least 93%, at least 94%, at least 95%, at least
96%, at least 97%, at least 98%, or at least 99% identity to, or comprising or
consisting of, the amino acid sequence set forth in SEQ ID NO.: 98;
(ii) a transmembrane domain that comprises an amino acid sequence having at least
90%, at least 91%, at least 92%, at least 93%, at least 94%, at least 95%, at least
96%, at least 97%, at least 98%, or at least 99% identity to, or comprising or
consisting of, the amino acid sequence set forth in SEQ ID NO.: 100; and
(iii) an intracellular component that comprises an amino acid sequence having at least
90%, at least 91%, at least 92%, at least 93%, at least 94%, at least 95%, at least
96%, at least 97%, at least 98%, or at least 99% identity to, or comprising or
consisting of, the amino acid sequence set forth in SEQ ID NO.: 175;
wherein the polypeptide is capable of binding to a Fas ligand and providing a
stimulatory signal.
Polypeptide Type W
A polypeptide comprising:
(i) an extracellular component that comprises an amino acid sequence having at least
90%, at least 91%, at least 92%, at least 93%, at least 94%, at least 95%, at least
96%, at least 97%, at least 98%, or at least 99% identity to, or comprising or
consisting of, the amino acid sequence set forth in SEQ ID NO.: 98;
(ii) a transmembrane domain that comprises an amino acid sequence having at least
90%, at least 91%, at least 92%, at least 93%, at least 94%, at least 95%, at least
96%, at least 97%, at least 98%, or at least 99% identity to, or comprising or
consisting of, the amino acid sequence set forth in SEQ ID NO.: 100; and
(iii) an intracellular component that comprises an amino acid sequence having at least
90%, at least 91%, at least 92%, at least 93%, at least 94%, at least 95%, at least
96%, at least 97%, at least 98%, or at least 99% identity to, or comprising or
consisting of, the amino acid sequence set forth in SEQ ID NO.: 4,
wherein the polypeptide is capable of binding to a Fas ligand and, optionally,
associating with a Lck.
Polypeptide Type X
A polypeptide comprising:
(i) an extracellular component that comprises an amino acid sequence having at least
90%, at least 91%, at least 92%, at least 93%, at least 94%, at least 95%, at least
96%, at least 97%, at least 98%, or at least 99% identity to, or comprising or
consisting of, the amino acid sequence set forth in SEQ ID NO.: 98;
(ii) a transmembrane domain that comprises an amino acid sequence having at least
90%, at least 91%, at least 92%, at least 93%, at least 94%, at least 95%, at least
96%, at least 97%, at least 98%, or at least 99% identity to, or comprising or
consisting of, the amino acid sequence set forth in SEQ ID NO.: 100; and
(iii) an intracellular component that comprises an amino acid sequence having at least
90%, at least 91%, at least 92%, at least 93%, at least 94%, at least 95%, at least
96%, at least 97%, at least 98%, or at least 99% identity to, or comprising or
consisting of, the amino acid sequence set forth in SEQ ID NO.: 176,
wherein the polypeptide is capable of binding to a Fas ligand and providing a
stimulatory signal.
Polypeptide Type Y
A polypeptide comprising:
(i) an extracellular component that comprises an amino acid sequence having at least
90%, at least 91%, at least 92%, at least 93%, at least 94%, at least 95%, at least
96%, at least 97%, at least 98%, or at least 99% identity to, or comprising or
consisting of, the amino acid sequence set forth in SEQ ID NO.: 104;
(ii) a transmembrane domain that comprises an amino acid sequence having at least
90%, at least 91%, at least 92%, at least 93%, at least 94%, at least 95%, at least
96%, at least 97%, at least 98%, or at least 99% identity to, or comprising or
consisting of, the amino acid sequence set forth in SEQ ID NO.: 80; and
(iii) an intracellular component that comprises an amino acid sequence having at least
90%, at least 91%, at least 92%, at least 93%, at least 94%, at least 95%, at least
96%, at least 97%, at least 98%, or at least 99% identity to, or comprising or
consisting of, the amino acid sequence set forth in SEQ ID NO.: 20,
wherein the polypeptide is capable of binding to a PD-1 ligand and providing a
stimulatory signal.
Polypeptide Type Z
A polypeptide comprising:
(i) an extracellular component that comprises an amino acid sequence having at least
90%, at least 91%, at least 92%, at least 93%, at least 94%, at least 95%, at least
96%, at least 97%, at least 98%, or at least 99% identity to, or comprising or
consisting of, the amino acid sequence set forth in SEQ ID NO.: 75;
(ii) a transmembrane domain that comprises an amino acid sequence having at least
90%, at least 91%, at least 92%, at least 93%, at least 94%, at least 95%, at least
96%, at least 97%, at least 98%, or at least 99% identity to, or comprising or
consisting of, the amino acid sequence set forth in SEQ ID NO.: 76; and
(iii) an intracellular component that comprises an amino acid sequence having at least
90%, at least 91%, at least 92%, at least 93%, at least 94%, at least 95%, at least
96%, at least 97%, at least 98%, or at least 99% identity to, or comprising or
consisting of, the amino acid sequence set forth in SEQ ID NO.: 177,
wherein the polypeptide is capable of providing a stimulatory signal and an effector
signal.
Polypeptide Type AA
A polypeptide comprising:
(i) an extracellular component that comprises an amino acid sequence having at least
90%, at least 91%, at least 92%, at least 93%, at least 94%, at least 95%, at least
96%, at least 97%, at least 98%, or at least 99% identity to, or comprising or
consisting of, the amino acid sequence set forth in SEQ ID NO.: 75;
(ii) a transmembrane domain that comprises an amino acid sequence having at least
90%, at least 91%, at least 92%, at least 93%, at least 94%, at least 95%, at least
96%, at least 97%, at least 98%, or at least 99% identity to, or comprising or
consisting of, the amino acid sequence set forth in SEQ ID NO.: 76; and
(iii) an intracellular component that comprises an amino acid sequence having at least
90%, at least 91%, at least 92%, at least 93%, at least 94%, at least 95%, at least
96%, at least 97%, at least 98%, or at least 99% identity to, or comprising or
consisting of, the amino acid sequence set forth in SEQ ID NO.: 178,
wherein the polypeptide is capable of providing a stimulatory signal and an effector
signal

Accordingly, in some embodiments, a polypeptide of Type A1, of Type A2, of Type B, of Type C1, of Type C2, of Type D1, of Type D2, of Type E, of Type F1, of Type F2, of Type G, of Type H, of Type I, of Type J, of Type K, of Type L1, of Type L2, of Type M, of Type N, of Type O, of Type P, of Type Q, of Type R, of Type S, of Type T1, of Type T2, of Type U, of Type V, of Type W, of Type X, of Type Y, of Type Z, or of Type AA, in accordance with Table 5, is provided. Also provided is a polynucleotide that encodes the polypeptide, a vector that comprises the polynucleotide, and a host cell (e.g., an immune cell such as a T cell, for example a CD4+ T cell or CD8+ T cell) that expresses the polypeptide and/or comprises the polynucleotide or vector. Also provided are host cell compositions and methods of using a polypeptide, polynucleotide, vector, host cell, or host cell composition to treat a disease in a subject, such as a cancer, for example a cancer associated with expression of a MHC Class I-restricted antigen.

In certain embodiments, two or more polypeptides selected from any of Types A1-AA in Table 5 are provided. For example, in some embodiments, a polynucleotide or vector is provided that encodes, and/or a host cell is provided that expresses and/or encodes any two or more polypeptides selected from Types A1-AA in Table 5. In certain embodiments, two polypeptides are according to the following Types: A1 and A2, respectively; A1 and B, respectively; A1 and C2, respectively; A1 and D2, respectively; A1 and E, respectively; A1 and F2, respectively; A1 and G, respectively; A1 and H, respectively; A1 and I, respectively; A1 and J, respectively; A1 and K, respectively; A1 and L2, respectively; A1 and T2, respectively; A1 and M, respectively; A1 and N, respectively; A1 and O, respectively; A1 and P, respectively; A1 and R, respectively; A1 and S, respectively; C1 and A2, respectively; Cl and B, respectively; C1 and C2, respectively; C1 and D2, respectively; Cl and E, respectively; C1 and F2, respectively; C1 and G, respectively; C1 and H, respectively; C1 and I, respectively; C1 and J, respectively; Cl and K, respectively; C1 and L2, respectively; C1 and T2, respectively; C1 and M, respectively; C1 and N, respectively; C1 and O, respectively; C1 and P, respectively; C1 and R, respectively; C1 and S, respectively; D1 and A2, respectively; D1 and B, respectively; D1 and C2, respectively; D1 and D2, respectively; D1 and E, respectively; D1 and F2, respectively; D1 and G, respectively; D1 and H, respectively; D1 and I, respectively; DI and J, respectively; DI and K, respectively; D1 and L2, respectively; D1 and T2, respectively; D1 and M, respectively; D1 and N, respectively; D1 and O, respectively; D1 and P, respectively; D1 and R, respectively; D1 and S, respectively; F1 and A2, respectively; F1 and B, respectively; F1 and C2, respectively; F1 and D2, respectively; F1 and E, respectively; F1 and F2, respectively; F1 and G, respectively; F1 and H, respectively; F1 and I, respectively; F1 and J, respectively; F1 and K, respectively; F1 and L2, respectively; F1 and T2, respectively; F1 and M, respectively; F1 and N, respectively; F1 and O, respectively; F1 and P, respectively; F1 and R, respectively; F1 and S, respectively; L1 and A2, respectively; L1 and B, respectively; L1 and C2, respectively; L1 and D2, respectively; L1 and E, respectively; L1 and F2, respectively; L1 and G, respectively; L1 and H, respectively; L1 and I, respectively; L1 and J, respectively; L1 and K, respectively; L1 and L2, respectively; L1 and T2, respectively; L1 and M, respectively; L1 and N, respectively; L1 and O, respectively; L1 and P, respectively; L1 and R, respectively; L1 and S, respectively; T1 and A2, respectively; Tl and B, respectively; T1 and C2, respectively; T1 and D2, respectively; Tl and E, respectively; T1 and F2, respectively; Tl and G, respectively; Tl and H, respectively; Tl and I, respectively; Tl and J, respectively; T1 and K, respectively; T1 and L2, respectively; T1 and T2, respectively; Tl and M, respectively; Tl and N, respectively; Tl and O, respectively; Tl and P, respectively; T1 and R, respectively; Tl and S, respectively; A2 and M, N, O, or P, respectively; B and M, N, O, or P, respectively; C2 and M, N, O, or P, respectively; D2 and M, N, O, or P, respectively; E and M, N, O, or P, respectively; F2 and M, N, O, or P, respectively; G and M, N, O, or P, respectively; H and M, N, O, or P, respectively; H and M, N, O, or P, respectively; I and M, N, O, or P, respectively; J and M, N, O, or P, respectively; K and M, N, O, or P, respectively; L2 and M, N, O, or P, respectively; or T2 and M, N, O, or P, respectively.

In some embodiments, a polypeptide is provided, wherein the polypeptide is Polypeptide 1 or Polypeptide 2 of any one of Costructs A-AA in Table 3 or Table 4. In some embodiments, a polynucleotide is provided that encodes, and/or a host cell is provided that expresses, the polypeptide. In some embodiments, a polynucleotide encodes, and/or a host cell expresses, Polypeptide 1 of any one of Constructs A-AA in Table 3 or Table 4 and Polypeptide 2 of any one of Constructs A-AA in Table 3 or Table 4. In certain embodiments, a polynucleotide encodes, and/or a host cell expresses, Polypeptide 1 and Polypeptide 2 of any one of Costructs A-AA in Table 3 or Table 4.

In certain embodiments, a protein of the present disclosure comprises a protein tag. Protein tags are unique peptide sequences that are affixed or genetically fused to, or are a part of, a protein of interest and can be recognized or bound by, for example, a heterologous or non-endogenous cognate binding molecule or a substrate (e.g., receptor, ligand, antibody, carbohydrate, or metal matrix) or a protein of this disclosure. Protein tags can be useful for detecting, identifying, isolating, tracking, purifying, enriching for, targeting, or biologically or chemically modifying tagged proteins of interest, particularly when a tagged protein is part of a heterogeneous population of cell proteins or cells (e.g., a biological sample like peripheral blood). In tagged cell surface proteins, the ability of the tag(s) to be specifically bound by a cognate binding molecule or a fusion protein or engineered protein of this disclosure is distinct from, or is in addition to, the ability of binding domain(s) contained by the cell surface protein (e.g., fusion protein, TCR) to specifically bind target molecule(s). In certain embodiments, a protein tag of a protein of this disclosure comprises a Myc tag, His tag, Flag tag, Xpress tag, Avi tag, Calmodulin tag, Polyglutamate tag, HA tag, Nus tag, S tag, SBP tag, Softag. V5 tag, CBP, GST, MBP, GFP, Thioredoxin tag, Strep® tag, or any combination thereof.

### Host Cells

A polynucleotide encoding any (e.g. fusion) protein (or any two or more of these) of this disclosure can, for example, be inserted into an appropriate vector (e.g., viral vector or non-viral plasmid vector) for introduction into a host cell of interest (e.g., an immune cell, such as a T cell). In certain embodiments, a polynucleotide or polynucleotides of the present disclosure is/are used to transfect/transduce a host cell (e.g., a T cell) for use in adoptive transfer therapy (e.g., targeting a cancer antigen. Methods for transfecting/transducing T cells with desired nucleic acids have been described (e.g., U.S. Patent Application Pub. No. US 2004/0087025) as have adoptive transfer procedures using T cells of desired target-specificity (e.g., Schmitt et al., Hum. Gen. 20:1240, 2009; Dossett et al., Mol. Ther. 17:742, 2009; Till et al., Blood 112:2261, 2008; Wang et al., Hum. Gene Ther. 18:712, 2007; Kuball et al., Blood 109:2331, 2007; US 2011/0243972; US 2011/0189141; Leen et al., Ann. Rev. Immunol. 25:243, 2007), such that adaptation of these methodologies to the presently disclosed embodiments is contemplated, based on the teachings herein, including those directed to proteins of the present disclosure. Accordingly, in another aspect, host cells are provided that comprise a polynucleotide or vector of the present disclosure and can express the encoded protein or proteins.

In certain embodiments, markers can be used to identify, monitor or isolate a host cell transduced with a heterologous polynucleotide encoding a (n e.g. fusion) protein as provided herein. Exemplary markers include green fluorescent protein, an extracellular domain of human CD2, a truncated human EGFR (huEGFRt, (see Wang et al., Blood 118:1255, 2011), a truncated human CD19 (huCD19t); a truncated human CD34 (huCD34t); or a truncated human NGFR (huNGFRt). In certain embodiments, an encoded marker comprises EGFRt, CD19t, CD34t, or NGFRt.

In certain embodiments, the host cell is a hematopoietic progenitor cell or a human immune system cell. A “hematopoietic progenitor cell”, as referred to herein, is a cell that can be derived from hematopoietic stem cells or fetal tissue and is capable of further differentiation into mature cells types (e.g., immune system cells). Exemplary hematopoietic progenitor cells include those with a CD24^{Lo} Lin⁻CD117⁺ phenotype or those found in the thymus (referred to as progenitor thymocytes).

As used herein, an “immune system cell” means any cell of the immune system that originates from a hematopoietic stem cell in the bone marrow, which gives rise to two major lineages, a myeloid progenitor cell (which give rise to myeloid cells such as monocytes, macrophages, dendritic cells, megakaryocytes and granulocytes) and a lymphoid progenitor cell (which give rise to lymphoid cells such as T cells, B cells, natural killer (NK) cells, and NK-T cells). Exemplary immune system cells include a CD4⁺ T cell, a CD8⁺ T cell, a CD4⁻ CD8⁻ double negative T cell, a γδ T cell, a regulatory T cell, a stem cell memory T cell, a natural killer cell (e.g., a NK cell or a NK-T cell), a B cell, and a dendritic cell. Macrophages and dendritic cells may be referred to as “antigen presenting cells” or “APCs,” which are specialized cells that can activate T cells when a major histocompatibility complex (MHC) receptor on the surface of the APC complexed with a peptide interacts with a TCR on the surface of a T cell.

A “T cell” or “T lymphocyte” is an immune system cell that matures in the thymus and produces T cell receptors (TCRs). T cells can be naïve (not exposed to antigen; increased expression of CD62L, CCR7, CD28, CD3, CD127, and CD45RA, and decreased expression of CD45RO as compared to T_{CM}), memory T cells (T_{M}) (antigen-experienced and long-lived), and effector cells (antigen-experienced, cytotoxic). T_{M} can be further divided into subsets of central memory T cells (T_{CM}, increased expression of CD62L, CCR7, CD28, CD127, CD45RO, and CD95, and decreased expression of CD54RA as compared to naïve T cells) and effector memory T cells (T_{EM}, decreased expression of CD62L, CCR7, CD28, CD45RA, and increased expression of CD127 as compared to naïve T cells or T_{CM}).

Effector T cells (T_{E}) refers to antigen-experienced CD8⁺ cytotoxic T lymphocytes that have decreased expression of CD62L, CCR7, CD28, and are positive for granzyme and perforin as compared to T_{CM}. Helper T cells (T_{H}) are CD4⁺ cells that influence the activity of other immune cells by releasing cytokines. CD4⁺ T cells can activate and suppress an adaptive immune response, and which of those two functions is induced will depend on presence of other cells and signals. T cells can be collected using known techniques, and the various subpopulations or combinations thereof can be enriched or depleted by known techniques, such as by affinity binding to antibodies, flow cytometry, or immunomagnetic selection. Other exemplary T cells include regulatory T cells, such as CD4⁺ CD25⁺ (Foxp3⁺) regulatory T cells and Treg17 cells, as well as Tr1, Th3, CD8⁺ CD28⁻, and Qa-1 restricted T cells.

As used herein, “enriched” or “depleted” with respect to amounts of cell types in a mixture refers to an increase in the number of the “enriched” type, a decrease in the number of the “depleted” cells, or both, in a mixture of cells resulting from one or more enriching or depleting processes or steps. Thus, depending upon the source of an original population of cells subjected to an enriching process, a mixture or composition may contain 30%, 35%, 40%, 45%, 50%, 55%, 60%, 65%, 70%, 75%, 80%, 85%, 90%, 91%, 92%, 93%, 94%, 95%, 96%, 97%, 98%, or 99% or more (in number or count) of the “enriched” cells. Cells subjected to a depleting process can result in a mixture or composition containing 50%, 45%, 40%, 35%, 30%, 25%, 20%, 15%, 10%, 9%, 8%, 7%, 6%, 5%, 4%, 3%, 2%, or 1% percent or less (in number or count) of the “depleted” cells. In certain embodiments, amounts of a certain cell type in a mixture will be enriched and amounts of a different cell type will be depleted, such as enriching for CD4⁺ cells while depleting CD8⁺ cells, or enriching for CD62L⁺ cells while depleting CD62L⁻ cells, or combinations thereof.

“Cells of T cell lineage” refer to cells that show at least one phenotypic characteristic of a T cell, or a precursor or progenitor thereof that distinguishes the cells from other lymphoid cells, and cells of the erythroid or myeloid lineages. Such phenotypic characteristics can include expression of one or more proteins specific for T cells (e.g., CD3⁺, CD4⁺, CD8⁺), or a physiological, morphological, functional, or immunological feature specific for a T cell. For example, cells of the T cell lineage may be progenitor or precursor cells committed to the T cell lineage; CD25⁺ immature and inactivated T cells; cells that have undergone CD4 or CD8 linage commitment; thymocyte progenitor cells that are CD4⁺ CD8⁺ double positive; single positive CD4⁺ or CD8⁺; TCRαβ or TCR γδ; or mature and functional or activated T cells.

In certain embodiments, the immune system cell is a CD4+ T cell, a CD8+ T cell, a CD4− CD8− double negative T cell, a γδ T cell, a natural killer cell (e.g., NK cell or NK-T cell), a dendritic cell, a B cell, or any combination thereof. In certain embodiments, the immune system cell is a CD4+ T cell. In certain embodiments, the T cell is a naïve T cell, a central memory T cell, an effector memory T cell, a stem cell memory T cell, or any combination thereof.

A host cell may include any individual cell or cell culture which may receive a vector or the incorporation of nucleic acids or express proteins. The term also encompasses progeny of the host cell, whether genetically or phenotypically the same or different. Suitable host cells may depend on the vector and may include mammalian cells, animal cells, human cells, simian cells, insect cells, yeast cells, and bacterial cells. These cells may be induced to incorporate the vector or other material by use of a viral vector, transformation via calcium phosphate precipitation, DEAE-dextran, electroporation, microinjection, or other methods. See, for example, Sambrook et al., Molecular Cloning: A Laboratory Manual 2d ed. (Cold Spring Harbor Laboratory, 1989). In a related aspect, methods are provided for preparing a cell, wherein the method comprises introducing into the cell a polynucleotide or vector encoding any protein in accordance with the present disclosure. In certain embodiments, the vector further comprises a polynucleotide encoding a binding protein. In other embodiments, the cell is modified to comprise, or comprises, a heterologous polynucleotide encoding a binding protein.

In certain embodiments, a host cell of the present disclosure can comprise a heterologous polynucleotide that encodes (i) a fusion protein comprising one or more sequences from (or derived from) a CD8 co-receptor α-chain; (ii) a fusion protein comprising one or more sequences from (or derived from) a CD8 co-receptor β-chain; (iii) a CD8 co-receptor α-chain, or a functional variant or portion thereof; (vi) a CD8 co-receptor β-chain, or a functional variant or portion thereof, or (vii) any combination of (i)-(vi). In any of the herein disclosed embodiments, a fusion protein of the present disclosure can associate with a cognate CD8 co-receptor chain (e.g. having a wild-type amino acid sequence), or with a second fusion protein that comprises one or more domains or portions of a cognate CD8 co-receptor, to form a homodimer or a heterodimer when expressed at a host cell surface.

Accordingly, in certain embodiments, a polynucleotide of the present disclosure encodes: (i) a fusion protein comprising one or more sequences from (or derived from) a CD8 co-receptor α-chain; and (ii) a fusion protein comprising one or more sequences from (or derived from) a CD8 co-receptor β-chain.

In some embodiments, a polynucleotide of the present disclosure encodes: (i) a fusion protein comprising one or more sequences from (or derived from) a CD8 co-receptor α-chain; and (ii) a fusion protein comprising one or more sequences from (or derived from) a CD8 co-receptor α-chain.

In certain embodiments, a polynucleotide of the present disclosure encodes (i) a fusion protein comprising one or more sequences from (or derived from) a CD8 co-receptor α-chain; and (ii) a CD8 co-receptor β-chain, or a functional variant or portion thereof.

In certain embodiments, a polynucleotide of the present disclosure encodes (i) a fusion protein comprising one or more sequences from (or derived from) a CD8 co-receptor β-chain; and (ii) a CD8 co-receptor α-chain, or a functional variant or portion thereof.

In further embodiments, one or both substituent proteins of a co-receptor protein pair comprising (i) a fusion protein of the present disclosure and (ii) a cognate CD8 co-receptor protein or a functional variant or portion of the same (or a second fusion protein comprising one or more (native or variant) sequences from a cognate CD8 co-receptor) is modified to enhance association of the co-receptor pair when the substituent proteins are expressed at a cell surface. For example, in certain embodiments, a cysteine amino acid is introduced at one or more position in one or both of the substituent proteins such that the substituent proteins can form a cysteine-cysteine bond therebetween.

In any of the presently disclosed embodiments, a host cell can further comprise a polynucleotide (endogenous, heterologous, or both) that encodes a binding protein that is capable of specifically binding to an antigen. In certain embodiments, a binding protein comprises a TCR, which can, in some embodiments, be a MHC-I-restricted TCR or a MHC-II-restricted TCR. In certain embodiments, a binding protein comprises a CAR.

In any of the presently disclosed embodiments, polynucleotide encoding a first protein (e.g., fusion protein, CD8 co-receptor protein, binding protein, or marker) can be separated from a polynucleotide encoding a second protein (e.g., fusion protein, CD8 co-receptor protein, binding protein, or marker) by a polynucleotide that encodes a self-cleaving polypeptide. In certain embodiments, an encoded self-cleaving polypeptide comprises a 2A peptide from porcine teschovirus-1 (P2A), Thoseaasigna virus (T2A), equine rhinitis A virus (E2A), or foot-and-mouth disease virus (F2A)). Further exemplary nucleic acid and amino acid sequences of 2A peptides are set forth in, for example, Kim et al. (PLOS One 6: e18556, 2011, which 2A nucleic acid and amino acid sequences are incorporated herein by reference in their entirety).

“Chimeric antigen receptor” (CAR) refers to a binding protein that is engineered to contain two or more naturally occurring amino acid sequences linked together in a way that does not occur naturally or does not occur naturally in a host cell, which fusion protein can function as a receptor when present on a surface of a cell. CARs of the present disclosure include an extracellular portion comprising an antigen binding domain (i.e., obtained or derived from an immunoglobulin or immunoglobulin-like molecule, such as a scFv or scTCR derived from an antibody or TCR specific for a cancer antigen, or an antigen-binding domain derived or obtained from a killer immunoreceptor from an NK cell) linked to a transmembrane domain and one or more intracellular signaling domains (optionally containing co-stimulatory domain(s)) (see, e.g., Sadelain et al., Cancer Discov., 3 (4): 388 (2013); see also Harris and Kranz, Trends Pharmacol. Sci., 37 (3): 220 (2016); Stone et al., Cancer Immunol. Immunother., 63 (11): 1163 (2014)).

In certain embodiments, a binding protein comprises a CAR comprising an antigen-specific TCR binding domain (see, e.g., Walseng et al., Scientific Reports 7:10713, 2017; the TCR CAR constructs and methods of which are hereby incorporated by reference in their entirety), which can be a MHC-I-restricted TCR binding domain, a MHCII-restricted TCR binding domain, or both.

In some embodiments, a binding protein (e.g. a TCR) specifically binds to a tumor-associated antigen, an antigen associated with an infectious disease, an antigen associated with an autoimmune disease, an antigen associate with a neurodegenerative disease, or the like.

In certain embodiments, a binding protein of the instant disclosure specifically binds to a tumor-associated antigen. In particular embodiments, the tumor-associated antigen is selected from ROR1, EGFR, EGFRvIII, EGP-2, EGP-40, GD2, GD3, HPV E6, HPV E7, Her2, L1-CAM, Lewis A, Lewis Y, MUC1, MUC16, PSCA, PSMA, CD19, CD20, CD22, CD56, CD23, CD24, CD30, CD33, CD37, CD44v7/8, CD38, CD56, CD123, CA125, c-MET, FcRH5, WT1, folate receptor α, VEGF-α, VEGFR1, VEGFR2, IL-13Rα2, IL-11Rα, HER2, MAGE-A1, MAGE-A3, MAGE-A4, SSX-2, PRAME, KRAS (e.g. G12V, G12C, or G12D), Merkel Cell polyomavirus T antigen, Core Binding Factor protein, HA-1^{H}, PSA, ephrin A2, ephrin B2, an NKG2D, NY-ESO-1, TAG-72, mesothelin, NY-ESO, 5T4, BCMA, FAP, Carbonic anhydrase 9, ERBB2, a BRAF antigen such as a BRAF^{V600E} antigen, and CEA. In some embodiments, a tumor-associated antigen is selected from BCMA, CD3, CEACAM6, c-Met, EGFR, EGFRVIII, ErbB2, ErbB3, ErbB4, EphA2, IGFIR, GD2, O-acetyl GD2, O-acetyl GD3, GHRHR, GHR, FLT1, KDR, FLT4, CD44v6, CD151, CA125, CEA, CTLA-4, GITR, BTLA, TGFBR2, TGFBR1, IL6R, gp130, Lewis A, Lewis Y, TNFR1, TNFR2, PD1, PD-L1, PD-L2, HVEM, MAGE-A (e.g., including MAGE-A1, MAGE-A3, and MAGE-A4), HER2, mesothelin, NY-ESO-1, KRAS (e.g. G12V, G12C, or G12D), PSMA, RANK, ROR1, TNFRSF4, CD40, CD137, TWEAK-R, HLA, tumor- or pathogen-associated peptide bound to HLA, hTERT peptide bound to HLA, tyrosinase peptide bound to HLA, WT-1 peptide bound to HLA, LTβR, LIFRβ, LRP5, MUC1, OSMRβ, TCRα, TCRβ, CD19, CD20, CD22, CD25, CD28, CD30, CD33, CD52, CD56, CD79a, CD79b, CD80, CD81, CD86, CD123, CD171, CD276, B7H4, TLR7, TLR9, PTCH1, WT-1, HA¹-H, Robo1, α-fetoprotein (AFP), Frizzled, OX40, PRAME, and SSX-2. In any of the herein disclosed embodiments, a binding protein can comprise a binding domain disclosed in any one of PCT Publication Nos.: WO 2016/022400; WO 2018/170338; WO 2018/090057; WO 2017/112944; WO 2017/193104; WO 2018/058002; or WO 2013/071154; the CDR, framework, variable, and TCR sequences of which are incorporated herein by reference.

A “binding domain” (also referred to as a “binding region” or “binding moiety”), as used herein (e.g. with regard to a binding protein such as a TCR), refers to a molecule or portion thereof (e.g., peptide, oligopeptide, polypeptide, protein (e.g., a fusion protein)) that possesses the ability to specifically and non-covalently associate, unite, or combine with a target. A binding domain includes any naturally occurring, synthetic, semi-synthetic, or recombinantly produced binding partner for a biological molecule, a molecular complex (i.e., complex comprising two or more biological molecules), or other target of interest. Examples of binding domains include, in general, single chain immunoglobulin variable regions (e.g., scTCR, scFv, Fab, TCR variable regions), receptor ectodomains, ligands (e.g., cytokines, chemokines), or synthetic polypeptides selected for their specific ability to bind to a biological molecule, a molecular complex or other target of interest. In certain embodiments, the binding domain of a binding protein is a scFv, scTCR, or ligand. In certain embodiments, the binding domain is chimeric, human, or humanized.

As used herein, “specifically binds” or “specific for” refers to an association or union of a binding protein (e.g., a T cell receptor or a chimeric antigen receptor) or a binding domain (or fusion protein thereof) to a target molecule with an affinity or Kₐ (i.e., an equilibrium association constant of a particular binding interaction with units of 1/M) equal to or greater than 10⁵ M⁻¹ (which equals the ratio of the on-rate [Kₒₙ] to the off rate [K_{off}] for this association reaction), while not significantly associating or uniting with any other molecules or components in a sample. Binding proteins or binding domains may be classified as “high-affinity” binding proteins or binding domains or as “low-affinity” binding proteins or binding domains. “High-affinity” binding proteins or binding domains refer to those binding proteins or binding domains having a Kₐ of at least 10⁷ M⁻¹, at least 10⁸ M⁻¹, at least 10⁹ M¹, at least 10¹⁰ M⁻¹, at least 10¹¹ M⁻¹, at least 10¹² M⁻¹, or at least 10¹³ M⁻¹. “Low-affinity” binding proteins or binding domains refer to those binding proteins or binding domains having a Kₐ of up to 10⁷ M⁻¹, up to 10⁶ M⁻¹, or up to 10⁵ M⁻¹. Alternatively, affinity may be defined as an equilibrium dissociation constant (Kd) of a particular binding interaction with units of M (e.g., 10⁻⁵ M to 10⁻¹³ M)

In certain embodiments, a receptor or binding domain may have “enhanced affinity,” which refers to selected or engineered receptors or binding domains with stronger binding to a target antigen than a wild type (or parent) binding domain. For example, enhanced affinity may be due to a Kₐ (equilibrium association constant) for the target antigen that is higher than the wild type binding domain, due to a K_{d} (dissociation constant) for the target antigen that is less than that of the wild type binding domain, due to an off-rate (k_{off}) for the target antigen that is less than that of the wild type binding domain, or a combination thereof. In certain embodiments, fusion proteins may be codon-optimized to enhance expression in a particular host cell, such as T cells (Scholten et al., Clin. Immunol. 119:135, 2006).

A variety of assays are known for identifying binding domains of the present disclosure that specifically bind a particular target, as well as determining binding domain or fusion protein affinities, such as Western blot, ELISA, analytical ultracentrifugation, spectroscopy and surface plasmon resonance (Biacore®) analysis (see, e.g., Scatchard et al., Ann. N.Y. Acad. Sci. 51:660, 1949; Wilson, Science 295:2103, 2002; Wolff et al., Cancer Res. 53:2560, 1993; and U.S. Pat. Nos. 5,283,173, 5,468,614, or the equivalent). Assays for assessing affinity or apparent affinity or relative affinity are also known. In certain examples, apparent affinity for a fusion protein is measured by assessing binding to various concentrations of tetramers, for example, by flow cytometry using labeled tetramers. In some examples, apparent K_{D} of a fusion protein is measured using 2-fold dilutions of labeled tetramers at a range of concentrations, followed by determination of binding curves by non-linear regression, apparent K_{D} being determined as the concentration of ligand that yielded half-maximal binding.

Also provided are host cells that comprise:
(i) a heterologous polynucleotide that encodes a fusion protein, wherein the encoded fusion protein comprises: (a) an extracellular component comprising an extracellular domain from a CD8 co-receptor α-chain; (b) a transmembrane domain from a CD8 co-receptor α-chain; and (c) an intracellular component comprising a co stimulatory domain from CD28, or a functional portion or variant thereof; and
(ii) a heterologous polynucleotide encoding a binding protein that specifically binds to an antigen or an antigen: MHC complex.

In any of the presently disclosed embodiments, a binding protein encoded by a host cell (e.g., CD4 T cell) of the present disclosure can comprise a binding domain (e.g., a CAR or a TCR) that specifically binds to a MHC-I: antigen complex.

In any of the foregoing embodiments, a host cell (e.g., an immune cell) may modified to reduce or eliminate expression of one or more endogenous genes that encode a polypeptide involved in immune signaling or other related activities. Exemplary gene knockouts include those that encode PD-1, LAG-3, CTLA4, TIM3, TIGIT, FasL, an HLA molecule, a TCR molecule, or the like. Without wishing to be bound by theory, certain endogenously expressed immune cell proteins may be recognized as foreign by an allogeneic host receiving the modified immune cells, which may result in elimination of the modified immune cells (e.g., an HLA allele), or may downregulate the immune activity of the modified immune cells (e.g., PD-1, LAG-3, CTLA4, FasL, Fas, TIGIT, TIM3), or may interfere with, suppress, or counter the activity of a heterologously expressed protein of the present disclosure.

Accordingly, decreasing or eliminating expression or activity of such endogenous genes or proteins can improve the activity, tolerance, or persistence of the modified cells in an autologous or allogeneic host setting, and may allow for universal administration of the cells (e.g., to any recipient regardless of HLA type). In certain embodiments, a modified cell is a donor cell (e.g., allogeneic) or an autologous cell. In certain embodiments, a modified cell of this disclosure comprises a chromosomal gene knockout of one or more of a gene that encodes PD-1, LAG-3, CTLA4, TIM3, TIGIT, FasL, Fas, an HLA component (e.g., a gene that encodes an α1 macroglobulin, an α2 macroglobulin, an α3 macroglobulin, a β1 microglobulin, or a β2 microglobulin), or a TCR component (e.g., a gene that encodes a TCR variable region or a TCR constant region) (see, e.g., Torikai et al., Nature Sci. Rep. 6:21757 (2016); Torikai et al., Blood 119 (24): 5697 (2012); and Torikai et al., Blood 122 (8): 1341 (2013), the gene-editing techniques, compositions, and adoptive cell therapies of which are herein incorporated by reference in their entirety).

As used herein, the term “chromosomal gene knockout” refers to a genetic alteration or introduced inhibitory agent in a host cell that prevents (e.g., reduces, delays, suppresses, or abrogates) production, by the host cell, of a functionally active endogenous polypeptide product. Alterations resulting in a chromosomal gene knockout can include, for example, introduced nonsense mutations (including the formation of premature stop codons), missense mutations, gene deletion, and strand breaks, as well as the heterologous expression of inhibitory nucleic acid molecules that inhibit endogenous gene expression in the host cell.

In certain embodiments, a chromosomal gene knock-out or gene knock-in is made by chromosomal editing of a host cell. Chromosomal editing can be performed using, for example, endonucleases. As used herein “endonuclease” refers to an enzyme capable of catalyzing cleavage of a phosphodiester bond within a polynucleotide chain. In certain embodiments, an endonuclease is capable of cleaving a targeted gene thereby inactivating or “knocking out” the targeted gene. An endonuclease may be a naturally occurring, recombinant, genetically modified, or fusion endonuclease. The nucleic acid strand breaks caused by the endonuclease are commonly repaired through the distinct mechanisms of homologous recombination or non-homologous end joining (NHEJ). During homologous recombination, a donor nucleic acid molecule may be used for a donor gene “knock-in”, for target gene “knock-out”, and optionally to inactivate a target gene through a donor gene knock in or target gene knock out event. NHEJ is an error-prone repair process that often results in changes to the DNA sequence at the site of the cleavage, e.g., a substitution, deletion, or addition of at least one nucleotide. NHEJ may be used to “knock-out” a target gene. Examples of endonucleases include zinc finger nucleases, TALE-nucleases, CRISPR-Cas nucleases, meganucleases, and megaTALs.

As used herein, a “zinc finger nuclease” (ZFN) refers to a fusion protein comprising a zinc finger DNA-binding domain fused to a non-specific DNA cleavage domain, such as a Fokl endonuclease. Each zinc finger motif of about 30 amino acids binds to about 3 base pairs of DNA, and amino acids at certain residues can be changed to alter triplet sequence specificity (see, e.g., Desjarlais et al., Proc. Natl. Acad. Sci. 90:2256-2260, 1993; Wolfe et al., J. Mol. Biol. 285:1917-1934, 1999). Multiple zinc finger motifs can be linked in tandem to create binding specificity to desired DNA sequences, such as regions having a length ranging from about 9 to about 18 base pairs. By way of background, ZFNs mediate genome editing by catalyzing the formation of a site-specific DNA double strand break (DSB) in the genome, and targeted integration of a transgene comprising flanking sequences homologous to the genome at the site of DSB is facilitated by homology directed repair. Alternatively, a DSB generated by a ZFN can result in knock out of target gene via repair by non-homologous end joining (NHEJ), which is an error-prone cellular repair pathway that results in the insertion or deletion of nucleotides at the cleavage site. In certain embodiments, a gene knockout comprises an insertion, a deletion, a mutation or a combination thereof, made using a ZFN molecule.

As used herein, a “transcription activator-like effector nuclease” (TALEN) refers to a fusion protein comprising a TALE DNA-binding domain and a DNA cleavage domain, such as a FokI endonuclease. A “TALE DNA binding domain” or “TALE” is composed of one or more TALE repeat domains/units, each generally having a highly conserved 33-35 amino acid sequence with divergent 12th and 13th amino acids. The TALE repeat domains are involved in binding of the TALE to a target DNA sequence. The divergent amino acid residues, referred to as the Repeat Variable Diresidue (RVD), correlate with specific nucleotide recognition. The natural (canonical) code for DNA recognition of these TALEs has been determined such that an HD (histine-aspartic acid) sequence at positions 12 and 13 of the TALE leads to the TALE binding to cytosine (C), NG (asparagine-glycine) binds to a T nucleotide, NI (asparagine-isoleucine) to A, NN (asparagine-asparagine) binds to a G or A nucleotide, and NG (asparagine-glycine) binds to a T nucleotide. Non-canonical (atypical) RVDs are also known (see, e.g., U.S. Patent Publication No. US 2011/0301073, which atypical RVDs are incorporated by reference herein in their entirety). TALENs can be used to direct site-specific double-strand breaks (DSB) in the genome of T cells. Non-homologous end joining (NHEJ) ligates DNA from both sides of a double-strand break in which there is little or no sequence overlap for annealing, thereby introducing errors that knock out gene expression. Alternatively, homology directed repair can introduce a transgene at the site of DSB providing homologous flanking sequences are present in the transgene. In certain embodiments, a gene knockout comprises an insertion, a deletion, a mutation or a combination thereof, and made using a TALEN molecule.

As used herein, a “clustered regularly interspaced short palindromic repeats/Cas” (CRISPR/Cas) nuclease system refers to a system that employs a CRISPR RNA (crRNA)-guided Cas nuclease to recognize target sites within a genome (known as protospacers) via base-pairing complementarity and then to cleave the DNA if a short, conserved protospacer associated motif (PAM) immediately follows 3′ of the complementary target sequence. CRISPR/Cas systems are classified into three types (i.e., type I, type II, and type III) based on the sequence and structure of the Cas nucleases. The crRNA-guided surveillance complexes in types I and III need multiple Cas subunits. Type II system, the most studied, comprises at least three components: an RNA-guided Cas9 nuclease, a crRNA, and a trans-acting crRNA (tracrRNA). The tracrRNA comprises a duplex forming region. A crRNA and a tracrRNA form a duplex that is capable of interacting with a Cas9 nuclease and guiding the Cas9/crRNA: tracrRNA complex to a specific site on the target DNA via Watson-Crick base-pairing between the spacer on the crRNA and the protospacer on the target DNA upstream from a PAM. Cas9 nuclease cleaves a double-stranded break within a region defined by the crRNA spacer. Repair by NHEJ results in insertions and/or deletions which disrupt expression of the targeted locus. Alternatively, a transgene with homologous flanking sequences can be introduced at the site of DSB via homology directed repair. The crRNA and tracrRNA can be engineered into a single guide RNA (sgRNA or gRNA) (see, e.g., Jinek et al., Science 337:816-21, 2012). Further, the region of the guide RNA complementary to the target site can be altered or programed to target a desired sequence (Xie et al., PLOS One 9: e100448, 2014; U.S. Pat. Appl. Pub. No. US 2014/0068797, U.S. Pat. Appl. Pub. No. US 2014/0186843; U.S. Pat. No. 8,697,359, and PCT Publication No. WO 2015/071474; each of which is incorporated by reference). In certain embodiments, a gene knockout comprises an insertion, a deletion, a mutation or a combination thereof, and made using a CRISPR/Cas nuclease system.

Exemplary gRNA sequences and methods of using the same to knock out endogenous genes that encode immune cell proteins include those described in Ren et al., Clin. Cancer Res. 23 (9): 2255-2266 (2017), the gRNAs, CAS9 DNAs, vectors, and gene knockout techniques of which are hereby incorporated by reference in their entirety.

Alternative Cas nucleases may be used, including but not limited to, Cas 12, Cas 13, and Cas 14 nucleases, and variants thereof. For example, Cas nucleases disclosed in WO 2019/178427, which is hereby incorporated by reference in its entirety (including the Cas nucleases, CRISPR-Cas systems, and related methods disclosed therein), may be utilized.

As used herein, a “meganuclease,” also referred to as a “homing endonuclease,” refers to an endodeoxyribonuclease characterized by a large recognition site (double stranded DNA sequences of about 12 to about 40 base pairs). Meganucleases can be divided into five families based on sequence and structure motifs: LAGLIDADG, GIY-YIG, HNH, His-Cys box and PD-(D/E) XK. Exemplary meganucleases include I-SceI, I-CeuI, PI-PspI, PI-Sce, I-SceIV, I-CsmI, I-PanI, I-SceII, I-PpoI, I-SceIII, I-CreI, I-TevI, I-TevII and I-TevIII, whose recognition sequences are known (see, e.g., U.S. Pat. Nos. 5,420,032 and 6,833,252; Belfort et al., Nucleic Acids Res. 25:3379-3388, 1997; Dujon et al., Gene 82:115-118, 1989; Perler et al., Nucleic Acids Res. 22:1125-1127, 1994; Jasin, Trends Genet. 12:224-228, 1996; Gimble et al., J. Mol. Biol. 263:163-180, 1996; Argast et al., J. Mol. Biol. 280:345-353, 1998).

In certain embodiments, naturally occurring meganucleases may be used to promote site-specific genome modification of a target selected from PD-1, LAG3, TIM3, CTLA4, TIGIT, FasL, an HLA-encoding gene, or a TCR component-encoding gene. In other embodiments, an engineered meganuclease having a novel binding specificity for a target gene is used for site-specific genome modification (see, e.g., Porteus et al., Nat. Biotechnol. 23:967-73, 2005; Sussman et al., J. Mol. Biol. 342:31-41, 2004; Epinat et al., Nucleic Acids Res. 31:2952-62, 2003; Chevalier et al., Molec. Cell 10:895-905, 2002; Ashworth et al., Nature 441:656-659, 2006; Paques et al., Curr. Gene Ther. 7:49-66, 2007; U.S. Patent Publication Nos. US 2007/0117128; US 2006/0206949; US 2006/0153826; US 2006/0078552; and US 2004/0002092). In further embodiments, a chromosomal gene knockout is generated using a homing endonuclease that has been modified with modular DNA binding domains of TALENs to make a fusion protein known as a megaTAL. MegaTALs can be utilized to not only knock-out one or more target genes, but to also introduce (knock in) heterologous or exogenous polynucleotides when used in combination with an exogenous donor template encoding a polypeptide of interest.

In certain embodiments, a chromosomal gene knockout comprises an inhibitory nucleic acid molecule that is introduced into a host cell (e.g., an immune cell) comprising a heterologous polynucleotide encoding an antigen-specific receptor that specifically binds to a tumor associated antigen, wherein the inhibitory nucleic acid molecule encodes a target-specific inhibitor and wherein the encoded target-specific inhibitor inhibits endogenous gene expression (e.g., of PD-1, TIM3, LAG3, CTLA4,

TIGIT, Fas, FasL, an HLA component, or a TCR component, or any combination thereof) in the host cell.

A chromosomal gene knockout can be confirmed directly by DNA sequencing of the host immune cell following use of the knockout procedure or agent. Chromosomal gene knockouts can also be inferred from the absence of gene expression (e.g., the absence of an mRNA or polypeptide product encoded by the gene) following the knockout.

In certain embodiments, a chromosomal gene knockout comprises a knockout of an HLA component gene selected from an α1 macroglobulin gene, an α2 macroglobulin gene, an α3 macroglobulin gene, a β1 microglobulin gene, or a β2 microglobulin gene. In certain embodiments, a chromosomal gene knockout comprises a knockout of a TCR component gene selected from a TCR α variable region gene, a TCR β variable region gene, a TCR constant region gene, or a combination thereof.

Any of the foregoing gene-editing techniques can be used to introduce a polynucleotide of the present disclosure (e.g., encoding a binding protein and/or a protein such as a CD8 co-receptor polypeptide) into a host cell genome. In some embodiments, a heterologous polynucleotide is introduced into a locus encoding an endogenous TCR component, HLA component, PD-1, LAG-3, CTLA4, TIM3, or TIGIT, or a safe harbor locus such as Rosa26, AAVS1, CCR5, or the like. In certain embodiments, a heterologous polynucleotide encoding a binding protein and/or encoding a CD8 co-receptor polypeptide is introduced into a host cell TRAC locus. In further embodiments, a chromosomal knockout of a host cell TRBC locus is introduced.

Accordingly, in certain embodiments, a host cell (e.g., modified immune cell) is provided that comprises, in an endogenous TRAC locus, a heterologous polynucleotide encoding a binding protein of the present disclosure, a CD8 co-receptor of the present disclosure, or both. In further embodiments, the host cell comprises a chromosomal knockout of an endogenous TRBC locus.

### Uses

The present disclosure also provides methods of treating a disease or disorder, wherein the methods comprise administering a host (e.g. immune) cell of the present disclosure or a composition comprising the same. Briefly, it will be understood that when discussing treatment of a subject, the term “host cell” refers to a cell modified to comprise a presently disclosed protein, polynucleotide, or vector, irrespective of whether the host cell is autologous to the subject receiving treatment. In other words, “host” in this context describes the relationship between the cell and the heterologous protein, molecule, or vector, and not the relationship between the cell and the subject receiving treatment.

“Treat” or “treatment” or “ameliorate” refers to medical management of a disease, disorder, or condition of a subject (e.g., a human or non-human mammal, such as a primate, horse, cat, dog, goat, mouse, or rat). In general, an appropriate dose or treatment regimen comprising a host cell expressing a polypeptide (e.g. fusion protein) of the present disclosure or multiple such polypeptides, and optionally an adjuvant, is administered in an amount sufficient to elicit a therapeutic or prophylactic benefit. Therapeutic or prophylactic/preventive benefit includes improved clinical outcome; lessening or alleviation of symptoms associated with a disease; decreased occurrence of symptoms; improved quality of life; longer disease-free status; diminishment of extent of disease; stabilization of disease state; delay of disease progression; remission; survival; prolonged survival; or any combination thereof.

A “therapeutically effective amount” or “effective amount” of a polypeptide, fusion protein or host cell expressing the same refers to an amount of polypeptides or fusion proteins or host cells sufficient to result in a therapeutic effect, including improved clinical outcome; lessening or alleviation of symptoms associated with a disease; decreased occurrence of symptoms; improved quality of life; longer disease-free status; diminishment of extent of disease, stabilization of disease state; delay of disease progression; remission; survival; or prolonged survival in a statistically significant manner. The same applies to a polynucleotide or vector that encodes a protein of this disclosure.

When referring to an individual active ingredient or a cell expressing a single active ingredient, administered alone, a therapeutically effective amount refers to the effects of that ingredient or cell expressing that ingredient alone. When referring to a combination, a therapeutically effective amount refers to the combined amounts of active ingredients or combined adjunctive active ingredient with a cell expressing an active ingredient that results in a therapeutic effect, whether administered serially or simultaneously. A combination may also be a cell expressing more than one active ingredient.

The term “pharmaceutically acceptable excipient or carrier” or “physiologically acceptable excipient or carrier” refer to biologically compatible vehicles, e.g., physiological saline, which are described in greater detail herein, that are suitable for administration to a human or other non-human mammalian subject and generally recognized as safe or not causing a serious adverse event.

As used herein, “statistically significant” refers to a p-value of 0.050 or less when calculated using the Student's t-test and indicates that it is unlikely that a particular event or result being measured has arisen by chance.

As used herein, the term “adoptive immune therapy” or “adoptive immunotherapy” refers to administration of naturally occurring or genetically engineered, disease-antigen-specific immune cells (e.g., T cells). Adoptive cellular immunotherapy may be autologous (immune cells are from the recipient), allogeneic (immune cells are from a donor of the same species) or syngeneic (immune cells are from a donor genetically identical to the recipient).

In some embodiments, the host cell expresses at its cell surface (i) a polypeptide (e.g. a fusion protein) of the present disclosure and (ii) a binding protein specific for an antigen that is associated with or expressed by the disease or condition. In certain embodiments, the host cell expresses at its cell surface (i) a co-receptor pair comprising (a) a polypeptide (e.g. a fusion protein) and (b) a cognate co-receptor protein or fusion protein comprising a cognate co-receptor domain; and (ii) a binding protein that specifically binds to an antigen that associates with a MHC molecule.

In further embodiments, a host cell expressing (i) a polypeptide (e.g. a fusion protein) of the present disclosure and (ii) a binding protein is administered as part of a cellular immunotherapy that comprises (e.g., in a same composition or unit dose, or in separate compositions or unit doses) an effector (e.g. immune) cell that expresses at its cell surface a binding protein (e.g., CAR or TCR) that specifically binds to an antigen expressed by or otherwise associated with the disease or condition.

In certain embodiments, the effector (e.g immune) cell specifically binds to the same antigen as the host (e.g. immune) cell.

In other embodiments, the effector (e.g. immune) cell specifically binds to a different antigen as the host (e.g. immune) cell, provided that the different antigen is also expressed by or otherwise associated with the disease or condition.

In some embodiments, modified CD4+ T cells are administered to the subject. In some embodiments, modified CD8+ T cells are administered to the subject. In some embodiments, modified CD4+ T cells and modified CD8+ T cells.

In further embodiments, the host cell and/or effector cell are administered to treat a hyperproliferative disorder. As used herein, “hyperproliferative disorder” refers to excessive growth or proliferation as compared to a normal or undiseased cell. Exemplary hyperproliferative disorders include tumors, cancers, neoplastic tissue, carcinoma, sarcoma, malignant cells, pre-malignant cells, as well as non-neoplastic or non-malignant hyperproliferative disorders (e.g., adenoma, fibroma, lipoma, leiomyoma, hemangioma, fibrosis, restenosis, as well as autoimmune diseases such as rheumatoid arthritis, osteoarthritis, psoriasis, inflammatory bowel disease, or the like). Certain diseases that involve abnormal or excessive growth that occurs more slowly than in the context of a hyperproliferative disease can be referred to as “proliferative diseases”, and include certain tumors, cancers, neoplastic tissue, carcinoma, sarcoma, malignant cells, pre malignant cells, as well as non-neoplastic or non-malignant disorders.

Furthermore, “cancer” may refer to any accelerated proliferation of cells, including solid tumors, ascites tumors, blood or lymph or other malignancies; connective tissue malignancies; metastatic disease; minimal residual disease following transplantation of organs or stem cells; multi-drug resistant cancers, primary or secondary malignancies, angiogenesis related to malignancy, or other forms of cancer.

In certain embodiments, a cancer treatable according to the presently disclosed methods and uses comprises a carcinoma, a sarcoma, a glioma, a lymphoma, a leukemia, a myeloma, or any combination thereof. In certain embodiments, cancer comprises a cancer of the head or neck, melanoma, pancreatic cancer, cholangiocarcinoma, hepatocellular cancer, breast cancer including triple-negative breast cancer (TNBC), gastric cancer, non-small-cell lung cancer, prostate cancer, esophageal cancer, mesothelioma, small-cell lung cancer, colorectal cancer, glioblastoma, or any combination thereof.

In certain embodiments, a cancer comprises Askin's tumor, sarcoma botryoides, chondrosarcoma, Ewing's sarcoma, PNET, malignant hemangioendothelioma, malignant schwannoma, osteosarcoma, alveolar soft part sarcoma, angiosarcoma, cystosarcoma phyllodes, dermatofibrosarcoma protuberans (DFSP), desmoid tumor, desmoplastic small round cell tumor, epithelioid sarcoma, extraskeletal chondrosarcoma, extraskeletal osteosarcoma, fibrosarcoma, gastrointestinal stromal tumor (GIST), hemangiopericytoma, hemangiosarcoma, Kaposi's sarcoma, leiomyosarcoma, liposarcoma, lymphangiosarcoma, lymphosarcoma, undifferentiated pleomorphic sarcoma, malignant peripheral nerve sheath tumor (MPNST), neurofibrosarcoma, rhabdomyosarcoma, synovial sarcoma, undifferentiated pleomorphic sarcoma, squamous cell carcinoma, basal cell carcinoma, adenocarcinoma, linitis plastic, vipoma, cholangiocarcinoma, hepatocellular carcinoma, adenoid cystic carcinoma, renal cell carcinoma, Grawitz tumor, ependymoma, astrocytoma, oligodendroglioma, brainstem glioma, optice nerve glioma, a mixed glioma, Hodgkin's lymphoma, a B-cell lymphoma, non-Hodgkin's lymphoma (NHL), Burkitt's lymphoma, small lymphocytic lymphoma (SLL), diffuse large B-cell lymphoma, follicular lymphoma, immunoblastic large cell lymphoma, precursor B-lymphoblastic lymphoma, and mantle cell lymphoma, Waldenström's macroglobulinemia, CD37+ dendritic cell lymphoma, lymphoplasmacytic lymphoma, splenic marginal zone lymphoma, extra-nodal marginal zone B-cell lymphoma of mucosa-associated (MALT) lymphoid tissue, nodal marginal zone B-cell lymphoma, mediastinal (thymic) large B-cell lymphoma, intravascular large B-cell lymphoma, primary effusion lymphoma, adult T-cell lymphoma, extranodal NK/T-cell lymphoma, nasal type, enteropathy-associated T-cell lymphoma, hepatosplenic T-cell lymphoma, blastic NK cell lymphoma, Sezary syndrome, angioimmunoblastic T cell lymphoma, anaplastic large cell lymphoma, or any combination thereof.

In certain embodiments, the cancer comprises a solid tumor. In some embodiments, the solid tumor is a sarcoma or a carcinoma. In certain embodiments, the solid tumor is selected from: chondrosarcoma; fibrosarcoma (fibroblastic sarcoma); Dermatofibrosarcoma protuberans (DFSP); osteosarcoma; rhabdomyosarcoma; Ewing's sarcoma; a gastrointestinal stromal tumor; Leiomyosarcoma; angiosarcoma (vascular sarcoma); Kaposi's sarcoma; liposarcoma; pleomorphic sarcoma; or synovial sarcoma.

In certain embodiments, the solid tumor is selected from a lung carcinoma (e.g., Adenocarcinoma, Squamous Cell Carcinoma (Epidermoid Carcinoma); Squamous cell carcinoma; Adenocarcinoma; Adenosquamous carcinoma; anaplastic carcinoma; Large cell carcinoma; Small cell carcinoma; a breast carcinoma (e.g., Ductal Carcinoma in situ (non-invasive), Lobular carcinoma in situ (non-invasive), Invasive Ductal Carcinoma, Invasive lobular carcinoma, Non-invasive Carcinoma); a liver carcinoma (e.g., Hepatocellular Carcinoma, Cholangiocarcinomas or Bile Duct Cancer); Large-cell undifferentiated carcinoma, Bronchioalveolar carcinoma); an ovarian carcinoma (e.g., Surface epithelial-stromal tumor (Adenocarcinoma) or ovarian epithelial carcinoma (which includes serous tumor, endometrioid tumor and mucinous cystadenocarcinoma), Epidermoid (Squamous cell carcinoma), Embryonal carcinoma and choriocarcinoma (germ cell tumors)); a kidney carcinoma (e.g., Renal adenocarcinoma, hypernephroma, Transitional cell carcinoma (renal pelvis), Squamous cell carcinoma, Bellini duct carcinoma, Clear cell adenocarcinoma, Transitional cell carcinoma, Carcinoid tumor of the renal pelvis); an adrenal carcinoma (e.g., Adrenocortical carcinoma), a carcinoma of the testis (e.g., Germ cell carcinoma (Seminoma, Choriocarcinoma, Embryonal carciroma, Teratocarcinoma), Serous carcinoma); Gastric carcinoma (e.g., Adenocarcinoma); an intestinal carcinoma (e.g., Adenocarcinoma of the duodenum); a colorectal carcinoma; or a skin carcinoma (e.g., Basal cell carcinoma, Squamous cell carcinoma). In certain embodiments, the solid tumor is an ovarian carcinoma, an ovarian epithelial carcinoma, a cervical adenocarcinoma or small cell carcinoma, a pancreatic carcinoma, a colorectal carcinoma (e.g., an adenocarcinoma or squamous cell carcinoma), a lung carcinoma, a breast ductal carcinoma, or an adenocarcinoma of the prostate.

In any of the presently disclosed embodiments, the host cell is an allogeneic cell, a syngeneic cell, or an autologous cell. Typically, the host cell will further express or encode an antigen-binding protein such as, for example, a TCR.

Subjects that can be treated by the present invention are, in general, human and other primate subjects, such as monkeys and apes for veterinary medicine purposes. In any of the aforementioned embodiments, the subject may be a human subject. The subjects can be male or female and can be any suitable age, including infant, juvenile, adolescent, adult, and geriatric subjects. Cells according to the present disclosure may be administered in a manner appropriate to the disease, condition, or disorder to be treated as determined by persons skilled in the medical art. In any of the above embodiments, a cell comprising a cell as described herein is administered intravenously, intraperitoneally, intratumorally, into the bone marrow, into a lymph node, or into the cerebrospinal fluid. An appropriate dose, suitable duration, and frequency of administration of the compositions will be determined by such factors as the age, size, gender, and condition of the patient; the type and severity of the disease, condition, or disorder; the particular form of the active ingredient; and the method of administration.

In any of the above embodiments, methods of the present disclosure comprise administering a host cell of the present disclosure. The amount of cells in a composition is at least one cell (for example, one fusion protein-modified CD8⁺ T cell subpopulation; one fusion protein-modified CD4⁺ T cell subpopulation) or is more typically greater than 10² cells, for example, up to 10⁶, up to 10⁷, up to 10⁸ cells, up to 10⁹ cells, or more than 10¹⁰ cells. In certain embodiments, the cells are administered in a range from about 10⁶ to about 10¹⁰ cells/m², preferably in a range of about 10⁵ to about 10⁹ cells/m². The number of cells will depend upon the ultimate use for which the composition is intended as well the type of cells included therein. For example, in certain embodiments, cells modified to contain a fusion protein and a binding protein specific for a particular antigen will comprise a cell population containing at least 30%, 35%, 40%, 45%, 50%, 55%, 60%, 65%, 70%, 75%, 80%, 85%, 90%, 95% or more of such cells. For uses provided herein, cells are generally in a volume of a liter or less, 500 mls or less, 250 mls or less, or 100 mls or less. In embodiments, the density of the desired cells is typically greater than 10⁴ cells/ml and generally is greater than 10⁷ cells/ml, generally 10⁸ cells/ml or greater. The cells may be administered as a single infusion or in multiple infusions over a range of time. A clinically relevant number of immune cells can be apportioned into multiple infusions that cumulatively equal or exceed 10⁶, 10⁷, 10⁸, 10⁹, 10¹⁰, or 10¹¹ cells.

Unit doses are also provided herein which comprise a host cell or host cell composition of this disclosure. In certain embodiments, a unit dose comprises a host cell (i.e., expressing a fusion protein and a binding protein) and an effector immune cell, wherein the host cell and the effector immune cell can each be a CD4⁺ T cell, a CD8⁺ T cell, or both.

In certain embodiments, a unit dose comprises: (i) CD4⁺ T cells that express at their cell surface: (a) a co-receptor pair comprising one or more CD8-derived fusion protein of the present disclosure; and (b) MHC-I-restricted binding protein; and (ii) CD8⁺ effector immune cells that express at their cell surface a binding protein.

In certain embodiments, a unit dose comprises: (i) CD4⁺ T cells that express at their cell surface: (a) one or more polypeptide as set forth in any one of Tables 1-5; and (b) MHC-I-restricted binding protein; and (ii) CD8⁺ effector immune cells that express at their cell surface a binding protein.

In certain embodiments, a unit dose comprises: (i) CD4⁺ T cells that express at their cell surface: (a) fusion or engineered protein according to the present disclosure; and (b) MHC-I-restricted binding protein; and optionally, (ii) CD8⁺ effector immune cells that express at their cell surface a binding protein.

In certain embodiments, the MHC-I-restricted binding protein and the binding protein of the CD8+ effector immune cells each specifically bind to an (e.g. the same or a different) epitope from the same antigen.

In certain embodiments, a unit dose comprises (i) a composition comprising at least about 30%, at least about 40%, at least about 50%, at least about 60%, at least about 70%, at least about 80%, at least about 85%, at least about 90%, or at least about 95% modified or unmodified CD4⁺ T cells, combined with (ii) a composition comprising at least about 30%, at least about 40%, at least about 50%, at least about 60%, at least about 70%, at least about 80%, at least about 85%, at least about 90%, or at least about 95% modified or unmodified CD8⁺ T cells, in about a 1:1 ratio, wherein the unit dose contains a reduced amount or substantially no naïve T cells (i.e., has less than about 50%, less than about 40%, less than about 30%, less than about 20%, less than about 10%, less than about 5%, or less then about 1% the population of naïve T cells present in a unit dose as compared to a patient sample having a comparable number of PBMCs).

In some embodiments, a unit dose comprises (i) a composition comprising at least about 50% modified or unmodified CD4⁺ T cells, combined with (ii) a composition comprising at least about 50% modified or unmodified CD8⁺ T cells, in about a 1:1 ratio, wherein the unit dose contains a reduced amount or substantially no naïve T cells. In further embodiments, a unit dose comprises (i) a composition comprising at least about 60% modified or unmodified CD4⁺ T cells, combined with (ii) a composition comprising at least about 60% modified or unmodified CD8⁺ T cells, in about a 1:1 ratio, wherein the unit dose contains a reduced amount or substantially no naïve T cells. In still further embodiments, a unit dose comprises (i) a composition comprising at least about 70% modified or unmodified CD4⁺ T cells, combined with (ii) a composition comprising at least about 70% modified or unmodified CD8⁺ T cells, in about a 1:1 ratio, wherein the unit dose contains a reduced amount or substantially no naïve T cells. In some embodiments, a unit dose comprises (i) a composition comprising at least about 80% modified or unmodified CD4⁺ T cells, combined with (ii) a composition comprising at least about 80% modified or unmodified CD8⁺ T cells, in about a 1:1 ratio, wherein the unit dose contains a reduced amount or substantially no naïve T cells. In some embodiments, a unit dose comprises (i) a composition comprising at least about 85% modified or unmodified CD4⁺ T cells, combined with (ii) a composition comprising at least about 85% modified or unmodified CD8⁺ T cells, in about a 1:1 ratio, wherein the unit dose contains a reduced amount or substantially no naïve T cells. In some embodiments, a unit dose comprises (i) a composition comprising at least about 90% modified or unmodified CD4⁺ T cells, combined with (ii) a composition comprising at least about 90% modified or unmodified CD8⁺ T cells, in about a 1:1 ratio, wherein the unit dose contains a reduced amount or substantially no naïve T cells.

In any of the embodiments described herein, a unit dose comprises equal, or approximately equal numbers of modified or unmodified CD45RA⁻ CD3⁺ CD8⁺ and modified or unmodified CD45RA⁻ CD3⁺ CD4⁺ T_{M} cells.

Also contemplated are pharmaceutical compositions that comprise (e.g. fusion) proteins or cells expressing the (e.g. fusion) proteins as disclosed herein and a pharmaceutically acceptable carrier, diluents, or excipient. Suitable excipients include water, saline, dextrose, glycerol, or the like and combinations thereof. In embodiments, compositions comprising fusion proteins or host cells as disclosed herein further comprise a suitable infusion media. Suitable infusion media can be any isotonic medium formulation, typically normal saline, Normosol R (Abbott) or Plasma-Lyte A (Baxter), 5% dextrose in water, Ringer's lactate can be utilized. An infusion medium can be supplemented with human serum albumin or other human serum components.

Pharmaceutical compositions may be administered in a manner appropriate to the disease or condition to be treated (or prevented) as determined by persons skilled in the medical art. An appropriate dose and a suitable duration and frequency of administration of the compositions will be determined by such factors as the health condition of the patient, size of the patient (i.e., weight, mass, or body area), the type and severity of the patient's condition, the undesired type or level or activity of the tagged cells, the particular form of the active ingredient, and the method of administration. In general, an appropriate dose and treatment regimen provide the composition(s) in an amount sufficient to provide therapeutic and/or prophylactic benefit (such as described herein, including an improved clinical outcome, such as more frequent complete or partial remissions, or longer disease-free and/or overall survival, or a lessening of symptom severity). For prophylactic use, a dose should be sufficient to prevent, delay the onset of, or diminish the severity of a disease associated with disease or disorder. Prophylactic benefit of the immunogenic compositions administered according to the methods described herein can be determined by performing pre-clinical (including in vitro and in vivo animal studies) and clinical studies and analyzing data obtained therefrom by appropriate statistical, biological, and clinical methods and techniques, all of which can readily be practiced by a person skilled in the art.

Certain methods of treatment or prevention contemplated herein include administering a host cell (which may be autologous, allogeneic or syngeneic) comprising a desired polynucleotide as described herein that is stably integrated into the chromosome of the cell. For example, such a cellular composition may be generated ex vivo using autologous, allogeneic or syngeneic immune system cells (e.g., T cells, antigen-presenting cells, natural killer cells) in order to administer a desired, fusion protein-expressing T-cell composition to a subject as an adoptive immunotherapy. In certain embodiments, the host cell is a hematopoietic progenitor cell or a human immune cell. In certain embodiments, the immune system cell is a CD4⁺ T cell, a CD8+ T cell, a CD4⁻ CD8⁻ double-negative T cell, a γδ T cell, a natural killer cell, a dendritic cell, or any combination thereof. In certain embodiments, the immune system cell is a naïve T cell, a central memory T cell, a stem cell memory T cell, an effector memory T cell, or any combination thereof. In particular embodiments, the cell is a CD4⁺ T cell. In particular embodiments, the cell is a CD8⁺ T cell.

As used herein, administration of a composition refers to delivering the same to a subject, regardless of the route or mode of delivery. Administration may be effected continuously or intermittently, and parenterally. Administration may be for treating a subject already confirmed as having a recognized condition, disease or disease state, or for treating a subject susceptible to or at risk of developing such a condition, disease or disease state. Co-administration with an adjunctive therapy may include simultaneous and/or sequential delivery of multiple agents in any order and on any dosing schedule (e.g., fusion protein-expressing recombinant (i.e., engineered) host cells with one or more cytokines; immunosuppressive therapy such as calcineurin inhibitors, corticosteroids, microtubule inhibitors, low dose of a mycophenolic acid prodrug, or any combination thereof).

In certain embodiments, a plurality of doses of a recombinant host cell as described herein is administered to the subject, which may be administered at intervals between administrations of about two to about four or more weeks.

In still further embodiments, the subject being treated is further receiving immunosuppressive therapy, such as calcineurin inhibitors, corticosteroids, microtubule inhibitors, low dose of a mycophenolic acid prodrug, or any combination thereof. In yet further embodiments, the subject being treated has received a non-myeloablative or a myeloablative hematopoietic cell transplant, wherein the treatment may be administered at least two to at least three months after the non-myeloablative hematopoietic cell transplant.

An effective amount of a pharmaceutical composition refers to an amount sufficient, at dosages and for periods of time needed, to achieve the desired clinical results or beneficial treatment, as described herein. An effective amount may be delivered in one or more administrations. If the administration is to a subject already known or confirmed to have a disease or disease-state, the term “therapeutic amount” may be used in reference to treatment, whereas “prophylactically effective amount” may be used to describe administrating an effective amount to a subject that is susceptible or at risk of developing a disease or disease-state (e.g., recurrence) as a preventative course.

The level of a CTL immune response may be determined by any one of numerous immunological methods described herein and routinely practiced in the art. The level of a CTL immune response may be determined prior to and following administration of any one of the herein described fusion proteins expressed by, for example, a T cell. Cytotoxicity assays for determining CTL activity may be performed using any one of several techniques and methods routinely practiced in the art (see, e.g., Henkart et al., “Cytotoxic T-Lymphocytes” in Fundamental Immunology, Paul (ed.) (2003 Lippincott Williams & Wilkins, Philadelphia, PA), pages 1127-50, and references cited therein).

Antigen-specific T cell responses are typically determined by comparisons of observed T cell responses according to any of the herein described T cell functional parameters (e.g., proliferation, cytokine release, CTL activity, altered cell surface marker phenotype, etc.) that may be made between T cells that are exposed to a cognate antigen in an appropriate context (e.g., the antigen used to prime or activate the T cells, when presented by immunocompatible antigen-presenting cells) and T cells from the same source population that are exposed instead to a structurally distinct or irrelevant control antigen. A response to the cognate antigen that is greater, with statistical significance, than the response to the control antigen signifies antigen-specificity.

A biological sample may be obtained from a subject for determining the presence and level of an immune response to a tagged protein or cell as described herein. A “biological sample” as used herein may be a blood sample (from which serum or plasma may be prepared), biopsy specimen, body fluids (e.g., lung lavage, ascites, mucosal washings, synovial fluid), bone marrow, lymph nodes, tissue explant, organ culture, or any other tissue or cell preparation from the subject or a biological source. Biological samples may also be obtained from the subject prior to receiving any immunogenic composition, which biological sample is useful as a control for establishing baseline (i.e., pre-immunization) data.

The pharmaceutical compositions described herein may be presented in unit-dose or multi-dose containers, such as sealed ampoules or vials. Such containers may be frozen to preserve the stability of the formulation until. In certain embodiments, a unit dose comprises a recombinant host cell as described herein at a dose of about 10⁷ cells/m² to about 10¹¹ cells/m². The development of suitable dosing and treatment regimens for using the particular compositions described herein in a variety of treatment regimens, including e.g., parenteral or intravenous administration or formulation.

If the subject composition is administered parenterally, the composition may also include sterile aqueous or oleaginous solution or suspension. Suitable non-toxic parenterally acceptable diluents or solvents include water, Ringer's solution, isotonic salt solution, 1,3-butanediol, ethanol, propylene glycol or polythethylene glycols in mixtures with water. Aqueous solutions or suspensions may further comprise one or more buffering agents, such as sodium acetate, sodium citrate, sodium borate or sodium tartrate. Of course, any material used in preparing any dosage unit formulation should be pharmaceutically pure and substantially non-toxic in the amounts employed. In addition, the active compounds may be incorporated into sustained-release preparation and formulations. Dosage unit form, as used herein, refers to physically discrete units suited as unitary dosages for the subject to be treated; each unit may contain a predetermined quantity of recombinant cells or active compound calculated to produce the desired effect in association with an appropriate pharmaceutical carrier.

In general, an appropriate dosage and treatment regimen provides the active molecules or cells in an amount sufficient to provide therapeutic or prophylactic benefit. Such a response can be monitored by establishing an improved clinical outcome (e.g., more frequent remissions, complete or partial, or longer disease-free survival) in treated subjects as compared to non-treated subjects. Increases in preexisting immune responses to a tumor protein generally correlate with an improved clinical outcome. Such immune responses may generally be evaluated using standard proliferation, cytotoxicity or cytokine assays, which are routine in the art and may be performed using samples obtained from a subject before and after treatment.

Methods according to this disclosure may further include administering one or more additional agents to treat the disease or disorder in a combination therapy. For example, in certain embodiments, a combination therapy comprises administering a fusion protein (or an engineered host cell expressing the same) with (concurrently, simultaneously, or sequentially) an immune checkpoint inhibitor. In some embodiments, a combination therapy comprises administering fusion protein of the present disclosure (or an engineered host cell expressing the same) with an agonist of a stimulatory immune checkpoint agent. In further embodiments, a combination therapy comprises administering a fusion protein of the present disclosure (or an engineered host cell expressing the same) with a secondary therapy, such as chemotherapeutic agent, a radiation therapy, a surgery, an antibody, or any combination thereof.

As used herein, the term “immune suppression agent” or “immunosuppression agent” refers to one or more cells, proteins, molecules, compounds or complexes providing inhibitory signals to assist in controlling or suppressing an immune response. For example, immune suppression agents include those molecules that partially or totally block immune stimulation; decrease, prevent or delay immune activation; or increase, activate, or up regulate immune suppression. Exemplary immunosuppression agents to target (e.g., with an immune checkpoint inhibitor) include PD-1, PD-L1, PD-L2, LAG3, CTLA4, B7-H3, B7-H4, CD244/2B4, HVEM, BTLA, CD160, TIM3, GAL9, KIR, PVR1G (CD112R), PVRL2, adenosine, A2aR, immunosuppressive cytokines (e.g., IL-10, IL-4, IL-1RA, IL-35), IDO, arginase, VISTA, TIGIT, LAIR1, CEACAM-1, CEACAM-3, CEACAM-5, Treg cells, or any combination thereof.

An immune suppression agent inhibitor (also referred to as an immune checkpoint inhibitor) may be a compound, an antibody, an antibody fragment or fusion polypeptide (e.g., Fc fusion, such as CTLA4-Fc or LAG3-Fc), an antisense molecule, a ribozyme or RNAi molecule, or a low molecular weight organic molecule. In any of the embodiments disclosed herein, a method may comprise administering an engineered host (e.g. immune) cell of the present disclosure with one or more inhibitor of any one of the following immune suppression components, singly or in any combination.

In certain embodiments, a modified cell is used in combination with a PD-1 inhibitor, for example a PD-1-specific antibody or binding fragment thereof, such as pidilizumab, nivolumab (Keytruda, formerly MDX-1106), pembrolizumab (Opdivo, formerly MK-3475), MEDI0680 (formerly AMP-514), AMP-224, BMS-936558 or any combination thereof. In further embodiments, a modified cell of the present disclosure is used in combination with a PD-L1 specific antibody or binding fragment thereof, such as BMS-936559, durvalumab (MEDI4736), atezolizumab (RG7446), avelumab (MSB0010718C), MPDL3280A, or any combination thereof. In certain embodiments, a modified cell of the present disclosure is used in combination with a LAG3 inhibitor, such as LAG525, IMP321, IMP701, 9H12, BMS-986016, or any combination thereof. In certain embodiments, a modified cell is used in combination with an inhibitor of CTLA4. In particular embodiments, a modified cell of the present disclosure (is used in combination with a CTLA4 specific antibody or binding fragment thereof, such as ipilimumab, tremelimumab, CTLA4-Ig fusion proteins (e.g., abatacept, belatacept), or any combination thereof. In certain embodiments, a modified cell of the present disclosure is used in combination with a B7-H3 specific antibody or binding fragment thereof, such as enoblituzumab (MGA271), 376.96, or both. A B7-H4 antibody binding fragment may be a scFv or fusion protein thereof, as described in, for example, Dangaj et al., Cancer Res. 73:4820, 2013, as well as those described in U.S. Pat. No. 9,574,000 and PCT Patent Publication Nos. WO/201640724A1 and WO 2013/025779A1. In certain embodiments, a modified cell of the present disclosure is used in combination with an inhibitor of CD244. In certain embodiments, a modified cell of the present disclosure is used in combination with an inhibitor of BLTA, HVEM, CD160, or any combination thereof. Anti CD-160 antibodies are described in, for example, PCT Publication No. WO 2010/084158. In certain embodiments, a modified cell of the present disclosure is used in combination with an inhibitor of TIM3. In certain embodiments, a modified cell of the present disclosure is used in combination with an inhibitor of Gal9. In certain embodiments, a modified cell of the present disclosure is used in combination with an inhibitor of adenosine signaling, such as a decoy adenosine receptor. In certain embodiments, a modified cell of the present disclosure is used in combination with an inhibitor of A2aR. In certain embodiments, a modified cell of the present disclosure is used in combination with an inhibitor of KIR, such as lirilumab (BMS-986015). In certain embodiments, a modified cell of the present disclosure is used in combination with an inhibitor of an inhibitory cytokine (typically, a cytokine other than TGFβ) or Treg development or activity. In certain embodiments, a modified cell of the present disclosure is used in combination with an IDO inhibitor, such as levo-1-methyl tryptophan, epacadostat (INCB024360; Liu et al., Blood 115:3520-30, 2010), ebselen (Terentis et al., Biochem. 49:591-600, 2010), indoximod, NLG919 (Mautino et al., American Association for Cancer Research 104th Annual Meeting 2013; Apr. 6-10, 2013), 1-methyl-tryptophan (1-MT)-tira-pazamine, or any combination thereof. In certain embodiments, a modified cell of the present disclosure is used in combination with an arginase inhibitor, such as N (omega)-Nitro-L-arginine methyl ester (L-NAME), N-omega-hydroxy-nor-l-arginine (nor-NOHA), L-NOHA, 2 (S)-amino-6-boronohexanoic acid (ABH), S-(2-boronoethyl)-L-cysteine (BEC), or any combination thereof. In certain embodiments, a modified cell of the present disclosure is used in combination with an inhibitor of VISTA, such as CA-170 (Curis, Lexington, Mass.). In certain embodiments, a modified cell of the present disclosure is used in combination with an inhibitor of TIGIT such as, for example, COM902 (Compugen, Toronto, Ontario Canada), an inhibitor of CD155, such as, for example, COM701 (Compugen), or both. In certain embodiments, a modified cell of the present disclosure is used in combination with an inhibitor of PVRIG, PVRL2, or both. Anti-PVRIG antibodies are described in, for example, PCT Publication No. WO 2016/134333. Anti-PVRL2 antibodies are described in, for example, PCT Publication No. WO 2017/021526. In certain embodiments, a modified cell of the present disclosure is used in combination with a LAIR 1 inhibitor. In certain embodiments, a modified cell of the present disclosure is used in combination with an inhibitor of CEACAM-1, CEACAM-3, CEACAM-5, or any combination thereof. In certain embodiments, a modified cell of the present disclosure is used in combination with an agent that increases the activity (i.e., is an agonist) of a stimulatory immune checkpoint molecule. For example, a modified cell of the present disclosure can be used in combination with a CD137 (4-1BB) agonist (such as, for example, urelumab), a CD134 (OX-40) agonist (such as, for example, MEDI6469, MEDI6383, or MEDI0562), lenalidomide, pomalidomide, a CD27 agonist (such as, for example, CDX-1127), a CD28 agonist (such as, for example, TGN1412, CD80, or CD86), a CD40 agonist (such as, for example, CP-870,893, rhuCD40L, or SGN-40), a CD122 agonist (such as, for example, IL-2) an agonist of GITR (such as, for example, humanized monoclonal antibodies described in PCT Patent Publication No. WO 2016/054638), an agonist of ICOS (CD278) (such as, for example, GSK3359609, mAb 88.2, JTX-2011, Icos 145-1, Icos 314-8, or any combination thereof). In any of the embodiments disclosed herein, a method may comprise administering a modified cell of the present disclosure with one or more agonist of a stimulatory immune checkpoint molecule, including any of the foregoing, singly or in any combination. In certain embodiments, a combination therapy comprises a modified cell of the present disclosure and a secondary therapy comprising one or more of: an antibody or antigen binding-fragment thereof that is specific for a cancer antigen expressed by the non-inflamed solid tumor, a radiation treatment, a surgery, a chemotherapeutic agent, a cytokine, RNAi, a further adoptive cell therapy, or any combination thereof. In certain embodiments, a combination therapy method comprises administering a modified cell and further administering a radiation treatment or a surgery. Radiation therapy is well-known in the art and includes X-ray therapies, such as gamma-irradiation, and radiopharmaceutical therapies. Surgeries and surgical techniques appropriate to treating a given cancer or tumor in a subject are known to those of ordinary skill in the art.

In certain embodiments, a combination therapy method comprises administering a modified (e.g immune) cell of the present disclosure and further administering a chemotherapeutic agent. A chemotherapeutic agent includes, but is not limited to, an inhibitor of chromatin function, a topoisomerase inhibitor, a microtubule inhibiting drug, a DNA damaging agent, an antimetabolite (such as folate antagonists, pyrimidine analogs, purine analogs, and sugar-modified analogs), a DNA synthesis inhibitor, a DNA interactive agent (such as an intercalating agent), and a DNA repair inhibitor. Illustrative chemotherapeutic agents include, without limitation, the following groups: anti-metabolites/anti-cancer agents, such as pyrimidine analogs (5-fluorouracil, floxuridine, capecitabine, gemcitabine and cytarabine) and purine analogs, folate antagonists and related inhibitors (mercaptopurine, thioguanine, pentostatin and 2-chlorodeoxyadenosine (cladribine)); antiproliferative/antimitotic agents including natural products such as vinca alkaloids (vinblastine, vincristine, and vinorelbine), microtubule disruptors such as taxane (paclitaxel, docetaxel), vincristin, vinblastin, nocodazole, epothilones and navelbine, epidipodophyllotoxins (etoposide, teniposide), DNA damaging agents (actinomycin, amsacrine, anthracyclines, bleomycin, busulfan, camptothecin, carboplatin, chlorambucil, cisplatin, cyclophosphamide, Cytoxan, dactinomycin, daunorubicin, doxorubicin, epirubicin, hexamethylmelamineoxaliplatin, iphosphamide, melphalan, merchlorehtamine, mitomycin, mitoxantrone, nitrosourea, plicamycin, procarbazine, taxol, taxotere, temozolamide, teniposide, triethylenethiophosphoramide and etoposide (VP 16)); antibiotics such as dactinomycin (actinomycin D), daunorubicin, doxorubicin (adriamycin), idarubicin, anthracyclines, mitoxantrone, bleomycins, plicamycin (mithramycin) and mitomycin; enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nitrosoureas (carmustine (BCNU) and analogs, streptozocin), trazenes-dacarbazinine (DTIC); antiproliferative/antimitotic antimetabolites such as folic acid analogs (methotrexate); platinum coordination complexes (cisplatin, carboplatin), procarbazine, hydroxyurea, mitotane, aminoglutethimide; hormones, hormone analogs (estrogen, tamoxifen, goserelin, bicalutamide, nilutamide) and aromatase inhibitors (letrozole, anastrozole); anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory agents; antisecretory agents (breveldin); immunosuppressives (cyclosporine, tacrolimus (FK-506), sirolimus (rapamycin), azathioprine, mycophenolate mofetil); anti-angiogenic compounds (TNP470, genistein) and growth factor inhibitors (vascular endothelial growth factor (VEGF) inhibitors, fibroblast growth factor (FGF) inhibitors); angiotensin receptor blocker; nitric oxide donors; anti-sense oligonucleotides; antibodies (trastuzumab, rituximab); chimeric antigen receptors; cell cycle inhibitors and differentiation inducers (tretinoin); mTOR inhibitors, topoisomerase inhibitors (doxorubicin (adriamycin), amsacrine, camptothecin, daunorubicin, dactinomycin, eniposide, epirubicin, etoposide, idarubicin, irinotecan (CPT-11) and mitoxantrone, topotecan, irinotecan), corticosteroids (cortisone, dexamethasone, hydrocortisone, methylpednisolone, prednisone, and prenisolone); growth factor signal transduction kinase inhibitors; mitochondrial dysfunction inducers, toxins such as Cholera toxin, ricin, Pseudomonas exotoxin, Bordetella pertussis adenylate cyclase toxin, or diphtheria toxin, and caspase activators; and chromatin disruptors.

Cytokines are used to manipulate host immune response towards anticancer activity. See, e.g., Floros & Tarhini, Semin. Oncol. 42 (4): 539-548, 2015. Cytokines useful for promoting immune anticancer or antitumor response include, for example, IFN-α, IL-2, IL-3, IL-4, IL-10, IL-12, IL-13, IL-15, IL-16, IL-17, IL-18, IL-21, IL-24, and GM-CSF, singly or in any combination with the cells or other compositions of this disclosure.

In further embodiments, the subject had previously received lymphodepleting chemotherapy prior to receiving the composition or HCT. In certain embodiments, a lymphodepleting chemotherapy comprises a conditioning regimen comprising cyclophosphamide, fludarabine, anti-thymocyte globulin, or a combination thereof.

The present disclosure also provides the following non-limiting enumerated Embodiments:

Embodiment 1. A fusion protein comprising: (i) an extracellular component comprising an extracellular domain from a CD8 co-receptor β-chain or a functional portion or variant thereof, or from a CD8 co-receptor α-chain or a functional portion or variant thereof, that is capable of binding to a MHC class I molecule; (ii) a transmembrane domain, provided that the transmembrane domain is not a transmembrane domain from a CD8 co-receptor α-chain when the extracellular component comprises a full length extracellular domain from the CD8 co-receptor α-chain; and (ii) an intracellular component comprising a co-stimulatory domain or a functional portion or variant thereof.

Embodiment 2. The fusion protein of Embodiment 1, wherein the extracellular component comprises or is derived from a CD8 co-receptor β-chain, or a functional portion or variant thereof.

Embodiment 3. The fusion protein of Embodiment 2, wherein the CD8 co-receptor β-chain comprises a canonical β-chain, a M1 isoform, a M2 isoform, a M3 isoform, a M4 isoform, a M5 isoform, a M6 isoform, a M7 isoform, or a M8 isoform.

Embodiment 4. The fusion protein of Embodiment 3, wherein the CD8 co-receptor β-chain is a M1 isoform.

Embodiment 5. The fusion protein of any one of Embodiments 1-4, wherein the extracellular component comprises an amino acid sequence having at least 80% identity to the amino acid sequence set forth in SEQ ID NO:7, or comprises or consists of the amino acid sequence set forth in SEQ ID NO:7.

Embodiment 6. The fusion protein of any one of Embodiments 1-4, wherein the transmembrane domain comprises or consists of a transmembrane domain from a CD4, a CD8β, a CD8α, a CD27, or a CD28, or a functional portion or variant thereof.

Embodiment 7. The fusion protein of any one of Embodiments 1-5, wherein the transmembrane domain comprises an amino acid sequence having at least 80% identity to the amino acid sequence set forth in SEQ ID NO:8, or comprises or consists of the amino acid set forth in SEQ ID NO:8.

Embodiment 8. The fusion protein of any one of Embodiments 1-7, further comprising an amino acid sequence having the amino acid sequence set forth in SEQ ID NO: 10, or a functional portion or variant thereof, disposed between the transmembrane domain and the intracellular component.

Embodiment 9. The fusion protein of any one of Embodiments 1-8, wherein the extracellular component comprises the amino acid sequence set forth in SEQ ID NO:7 and the transmembrane domain comprises the amino acid sequence set forth in SEQ ID NO: 8.

Embodiment 10. The fusion protein of Embodiment 1, wherein the extracellular component comprises or is derived from a CD8 co-receptor α-chain.

Embodiment 11. The fusion protein of Embodiment 10, wherein the CD8 co-receptor α-chain comprises a canonical α-chain, an isoform 2, or an isoform 3.

Embodiment 12. The fusion protein of any one of Embodiments 1-11, wherein the co-stimulatory domain comprises a co-stimulatory domain from one or more of CD28, 4-1BB (CD137), OX40 (CD134), ICOS (CD278), CD27, CD2, CD5, ICAM-1 (CD54), LFA-1 (CD11a/CD18), GITR, CD30, CD40, BAFF-R, HVEM, LIGHT, NKG2C, SLAMF7, NKp80, CD160, B7-H3, a ligand that specifically binds with CD83, CD94, DAP12, and/or comprises a functional variant of a co-stimulatory domain thereof.

Embodiment 13. The fusion protein of Embodiment 12, wherein the co-stimulatory domain comprises a co-stimulatory domain from CD28, or a functional portion or variant thereof.

Embodiment 14. The fusion protein of Embodiment 13, wherein the co-stimulatory domain comprises or consists of an amino acid sequence having at least 80% identity to the amino acid sequence shown in SEQ ID NO: 19.

Embodiment 15. The fusion protein of Embodiment 13 or 14, wherein the co-stimulatory domain comprises or consists of an amino acid sequence shown in SEQ ID NO: 19.

Embodiment 16. The fusion protein of Embodiment 13 or 14, wherein the co-stimulatory domain comprises a variant of the amino acid sequence shown in SEQ ID NO: 19, wherein one or both of the leucine residues at positions 7 and 8 of SEQ ID NO: 19 is substituted for a different amino acid.

Embodiment 17. The fusion protein of Embodiment 16, wherein the variant of the amino acid sequence shown in SEQ ID NO: 19 comprises a substitution of a glycine for one or both of the leucine residues at positions 7 and 8 of SEQ ID NO: 19.

Embodiment 18. The fusion protein of Embodiment 17, wherein the co-stimulatory domain comprises or consists of an amino acid sequence having at least 80% identity to the amino acid sequence the amino acid sequence shown in SEQ ID NO: 20.

Embodiment 19. The fusion protein of Embodiment 17 or 18, wherein the co-stimulatory domain comprises or consists of the amino acid sequence shown in SEQ ID NO: 20.

Embodiment 20. The fusion protein of any one of Embodiments 12-19, wherein the co-stimulatory domain comprises a co-stimulatory domain from 4-1BB, or a functional portion or variant thereof.

Embodiment 21. The fusion protein of Embodiment 20, wherein the co-stimulatory domain comprises or consists of an amino acid sequence having at least 80% identity to the amino acid sequence shown in SEQ ID NO:22.

Embodiment 22. The fusion protein of Embodiment 20 or 21, wherein the co-stimulatory domain comprises or consists of the amino acid sequence shown in SEQ ID NO: 22

Embodiment 23. The fusion protein of any one of Embodiments 12-22, wherein the co-stimulatory domain comprises a co-stimulatory domain from OX40, or a functional portion or variant thereof.

Embodiment 24. The fusion protein of Embodiment 23, wherein the co-stimulatory domain comprises or consists of an amino acid sequence having at least 80% identity to the amino acid sequence shown in SEQ ID NO:24.

Embodiment 25. The fusion protein of Embodiment 22 or 23, wherein the co-stimulatory domain comprises or consists of the amino acid sequence shown in SEQ ID NO: 24.

Embodiment 26. The fusion protein of any one of Embodiments 12-22, wherein the co-stimulatory domain comprises a co-stimulatory domain from ICOS, or a functional portion or variant thereof.

Embodiment 27. The fusion protein of Embodiment 26, wherein the co-stimulatory domain comprises or consists of an amino acid sequence having at least 80% identity to the amino acid sequence shown in SEQ ID NO:26.

Embodiment 28. The fusion protein of Embodiment 26 or 27, wherein the co-stimulatory domain comprises or consists of the amino acid sequence set forth in SEQ ID NO: 26.

Embodiment 29. The fusion protein of any one of Embodiments 1-28, further comprising a junction amino acid.

Embodiment 30. A fusion protein comprising: (i) an extracellular component comprising an extracellular domain from a CD8 co-receptor β-chain or a functional portion or variant thereof, or from a CD8 co-receptor α-chain or a functional portion or variant thereof, that is capable of binding to a MHC class I molecule; (ii) a transmembrane domain; and (iii) an intracellular component comprising a co-stimulatory domain from one, two, or three of: (a) a variant sequence of CD28 comprising or consisting of an amino acid sequence having at least 80% identity to the amino acid sequence shown in SEQ ID NO: 19 or 20, provided that: (1) no Tyr residue corresponding to position 12, 27, 30, or 39 of SEQ ID NO: 19 is substituted with Phe when the extracellular component comprises a full length extracellular domain from a CD8 co-receptor α-chain and the transmembrane domain comprises a transmembrane domain from the CD8 co-receptor α-chain; and/or (2) one or both of the leucine residues corresponding to positions 7 and 8 of SEQ ID NO: 19 is substituted for a different amino acid, wherein the different amino acid optionally comprises glycine; (b) CD27, or a functional portion or variant thereof; (c) 4-1BB, or a functional portion or variant thereof; (d) ICOS, or a functional portion or variant thereof; (e) OX40, or a functional portion or variant thereof; (f) CD30, or a functional portion or variant thereof; (g) LFA-1, or a functional portion or variant thereof; (h) CD2, or a functional portion or variant thereof; (i) CD7, or a functional portion or variant thereof; (j) LIGHT, or a functional portion or variant thereof; (k) NKG2C, or a functional portion or variant thereof; (l) B7-H3, or a functional portion or variant thereof; (j) GITR, or a functional portion or variant thereof; (k) BAFF-R, or a functional portion or variant thereof; (l) CD5, or a functional portion or variant thereof; (m) HVEM, or a functional portion or variant thereof; (n) CD160, or a functional portion or variant thereof; (o) LFA-1, or a functional portion or variant thereof; (p) SLAMF7, or a functional portion or variant thereof; (q) NKp80, or a functional portion or variant thereof; (r) ICAM-1, or a functional portion or variant thereof; (s) CD94, or a functional portion or variant thereof; (t) DAP12, or a functional portion or variant thereof; or (u) a ligand that specifically binds with CD83.

Embodiment 31. The fusion protein of Embodiment 30, wherein the extracellular component comprises or is derived from a CD8 co-receptor β-chain, or a functional portion or variant thereof.

Embodiment 32. The fusion protein of Embodiment 31, wherein the CD8 co-receptor β-chain comprises a canonical β-chain, a M1 isoform, a M2 isoform, a M3 isoform, a M4 isoform, a M5 isoform, a M6 isoform, a M7 isoform, or a M8 isoform. Embodiment 33. The fusion protein of Embodiment 32, wherein the CD8 co-receptor β-chain is a M1 isoform.

Embodiment 34. The fusion protein of any one of Embodiments 30-33, wherein the extracellular component comprises an amino acid sequence having at least 80% identity to the amino acid sequence set forth in SEQ ID NO:7, or comprises or consists of the amino acid sequence set forth in SEQ ID NO:7.

Embodiment 35. The fusion protein of any one of Embodiments 30-34, wherein the transmembrane domain comprises or consists of a transmembrane domain from a CD4, a CD8β, a CD8α, a CD27, or a CD28, or a functional portion or variant thereof.

Embodiment 36. The fusion protein of any one of Embodiments 30-35, wherein the transmembrane domain comprises an amino acid sequence having at least 80% identity to the amino acid sequence set forth in SEQ ID NO:8, or comprises or consists of the amino acid set forth in SEQ ID NO:8.

Embodiment 37. The fusion protein of any one of Embodiments 30-36, further comprising an amino acid sequence having the amino acid sequence set forth in SEQ ID NO: 10, or a functional portion or variant thereof, disposed between the transmembrane domain and the intracellular component.

Embodiment 38. The fusion protein of any one of Embodiments 30-37, wherein the extracellular component comprises the amino acid sequence set forth in SEQ ID NO: 7 and the transmembrane domain comprises the amino acid sequence set forth in SEQ ID NO:8.

Embodiment 39. The fusion protein of Embodiment 30, wherein the extracellular component comprises or is derived from a CD8 co-receptor α-chain.

Embodiment 40. The fusion protein of Embodiment 39, wherein the CD8 co-receptor α-chain comprises a canonical α-chain, isoform2, or isoform 3.

Embodiment 41. The fusion protein of Embodiment 39 or 40, wherein the extracellular component comprises or consists of an amino acid sequence having at least 80% identity to the amino acid sequence set forth in set forth in SEQ ID NO: 2.

Embodiment 42. The fusion protein of Embodiment any one of Embodiments 35-41, wherein the transmembrane component comprises an amino acid sequence having at least 80% identity to the amino acid sequence set forth in SEQ ID NO:3, or comprises or consists of the amino acid sequence set forth in SEQ ID NO:3.

Embodiment 43. The fusion protein of any one of Embodiments 30-42, wherein the variant sequence of CD28 comprises a substitution of a glycine for one or both of the leucine residues corresponding to positions 7 and 8 of SEQ ID NO:19.

Embodiment 44. The fusion protein of Embodiment 43, wherein the co-stimulatory domain comprises or consists an amino acid sequence having at least 80% identity to the amino acid sequence shown in SEQ ID NO:20.

Embodiment 45. The fusion protein of Embodiment 43 or 44, wherein the co-stimulatory domain comprises or consists of the amino acid sequence shown in SEQ ID NO: 20.

Embodiment 46. The fusion protein of any one of Embodiments 30-45, wherein the co-stimulatory domain comprises a co-stimulatory domain from 4-1BB, or a functional portion or variant thereof.

Embodiment 47. The fusion protein of Embodiment 46, wherein the co-stimulatory domain comprises or consists of an amino acid sequence having at least 80% identity to the amino acid sequence shown in SEQ ID NO:22.

Embodiment 48. The fusion protein of any one of Embodiments 46 or 47, wherein the co-stimulatory domain comprises or consists of the amino acid sequence shown in SEQ ID NO:22

Embodiment 49. The fusion protein of any one of Embodiments 30-48, wherein the co-stimulatory domain comprises a co-stimulatory domain from OX40, or a functional portion or variant thereof.

Embodiment 50. The fusion protein of Embodiment 49, wherein the co-stimulatory domain comprises or consists of an amino acid sequence having at least 80% identity to the amino acid sequence shown in SEQ ID NO:24.

Embodiment 51. The fusion protein of Embodiment 50, wherein the co-stimulatory domain comprises or consists of the amino acid sequence shown in SEQ ID NO: 24.

Embodiment 52. The fusion protein of any one of Embodiments 30-51, wherein the co-stimulatory domain comprises a co-stimulatory domain from ICOS, or a functional portion or variant thereof.

Embodiment 53. The fusion protein of Embodiment 52, wherein the co-stimulatory domain comprises or consists of an amino acid sequence having at least 80% identity to the amino acid sequence shown in SEQ ID NO:26.

Embodiment 54. The fusion protein of Embodiment 52 or 53, wherein the co-stimulatory domain comprises or consists of the amino acid sequence set forth in SEQ ID NO: 26.

Embodiment 55. The fusion protein of any one of Embodiments 30-54, further comprising a junction amino acid.

Embodiment 56. An isolated polynucleotide comprising a polynucleotide that encodes a first fusion protein of any one of Embodiments 1-55, wherein the first encoded fusion protein comprises an extracellular domain from a CD8 co-receptor α-chain, or a functional portion or variant thereof.

Embodiment 57. The isolated polynucleotide of Embodiment 56, further comprising a polynucleotide encoding a second protein, wherein the second encoded protein comprises: (i) a CD8 co-receptor α-chain, or a functional portion or variant thereof; or (ii) an extracellular domain from a CD8 co-receptor α-chain, or a functional portion or variant thereof.

Embodiment 58. An isolated polynucleotide comprising a polynucleotide that encodes a first fusion protein of any one of Embodiments 1-55, wherein the first encoded fusion protein comprises an extracellular domain from a CD8 co-receptor α-chain, or a functional portion or variant thereof.

Embodiment 59. The isolated polynucleotide of Embodiment 58, further comprising a polynucleotide encoding a second protein, wherein the second encoded protein comprises: (i) a CD8 co-receptor α-chain, or a functional portion or variant thereof; (ii) an extracellular domain from a CD8 co-receptor α-chain or a functional portion or variant thereof; (iii) a CD8 co-receptor β chain, or a functional portion or variant thereof; or (iv) an extracellular domain from a CD8 co-receptor β-chain, or a functional portion or variant thereof.

Embodiment 60. The isolated polynucleotide Embodiment 57 or 59, further comprising a polynucleotide encoding a self-cleaving peptide disposed between the first fusion protein and the second protein.

Embodiment 61. The isolated polynucleotide of Embodiment 59 or 60, wherein the encoded self-cleaving peptide comprises or consists of the amino acid sequence shown in any one of SEQ ID NOs:55-58.

Embodiment 62. The isolate polynucleotide of any one of Embodiments 56-61, wherein the first or second encoded fusion protein comprises or consists of the amino acid sequence shown in any one of SEQ ID NOs:36-42.

Embodiment 63. The isolated polynucleotide of any one of Embodiments 56-62, wherein any one or more of the polynucleotides encoding the first or second fusion protein is codon-optimized for expression by a host cell, wherein the host cell is optionally a T cell, preferably a CD4⁺ T cell.

Embodiment 64. The isolated polynucleotide of any one of Embodiments 56-63, wherein the polynucleotide comprises the nucleic acid sequence shown in any one of SEQ ID NOS: 27-35, 43-54, and 59-66.

Embodiment 65. The isolated polynucleotide of Embodiment 64, wherein the polynucleotide consists of the nucleotide sequence shown in any one of SEQ ID NOs:27-35 and 59-86.

Embodiment 66. An expression vector comprising the isolated polynucleotide of any one of Embodiments 56-65 operably linked to an expression control sequence.

Embodiment 67. The expression vector of Embodiment 66, wherein the vector is capable of delivering the polynucleotide to a host cell.

Embodiment 68. The expression vector of Embodiment 67, wherein the host cell is a hematopoietic progenitor cell or a human immune system cell.

Embodiment 69. The expression vector of Embodiment 68, wherein the human immune system cell is a CD4⁺ T cell, a CD8⁺ T cell, a CD4⁻ CD8⁻ double negative T cell, a γδ T cell, a natural killer cell, a natural killer T cell, a dendritic cell, or any combination thereof.

Embodiment 70. The expression vector of Embodiment 68 or 69, wherein the human immune system cell is a naïve T cell, a central memory T cell, a stem cell memory T cell, an effector memory T cell, or any combination thereof.

Embodiment 71. The expression vector of any one of Embodiments 66-70, wherein the vector is a viral vector.

Embodiment 72. The expression vector of Embodiment 71, wherein the viral vector is a lentiviral vector or a γ-retroviral vector.

Embodiment 73. A host cell comprising the polynucleotide of any one of Embodiments 56-65.

Embodiment 74. A host cell expressing at its cell surface the fusion protein of any one of Embodiments 1-55.

Embodiment 75. The host cell of Embodiment 73 or 74, wherein the host cell is a human immune system cell.

Embodiment 76. The host cell of Embodiment 75, wherein the human immune system cell is a CD4+ T cell, a CD8+ T cell, a CD4− CD8− double negative T cell, a γδ T cell, a natural killer cell, a natural killer T cell, a dendritic cell, or any combination thereof.

Embodiment 77. The host cell of Embodiment 75 or 76, wherein the human immune system cell is a CD4⁺ T cell.

Embodiment 78. The host cell of any one of Embodiments 75-77, wherein the human immune system cell is a naïve T cell, a central memory T cell, a stem cell memory T cell, an effector memory T cell, or any combination thereof.

Embodiment 79. The host cell of any one of Embodiments 75-78, further comprising a polynucleotide encoding a binding protein that specifically binds to an antigen or an antigen: MHC complex, wherein the polynucleotide encoding a binding protein is optionally heterologous to the host cell.

Embodiment 80. A host cell comprising: (i) a heterologous polynucleotide that encodes a fusion protein, wherein the encoded fusion protein comprises: (a) an extracellular component comprising an extracellular domain from a CD8 co-receptor α-chain; (b) a transmembrane domain from a CD8 co-receptor α-chain; and (c) an intracellular component comprising a co stimulatory domain from CD28, or a functional portion or variant thereof; and (ii) a heterologous polynucleotide encoding a binding protein that specifically binds to an antigen or an antigen: MHC complex.

Embodiment 81. The host cell of Embodiment 80, wherein the host cell comprises a human immune system cell.

Embodiment 82. The host cell of Embodiment 81, wherein the human immune system cell comprises a CD4⁺ T cell, a CD8⁺ T cell, a CD4 CD8-double negative T cell, a γδ T cell, a natural killer cell, a natural killer T cell, a dendritic cell, or any combination thereof.

Embodiment 83. The host cell of Embodiment 81, wherein the human immune system cell comprises a CD4⁺ T cell.

Embodiment 84. The host cell of any one of Embodiments 79-83, wherein the encoded binding protein comprises a TCR or a CAR.

Embodiment 85. The host cell of any one of Embodiments 79-84, wherein the binding protein comprises a binding domain from a MHC-I-restricted TCR, or a functional variant or portion thereof.

Embodiment 86. The host cell of any one of Embodiments 79-85, wherein the binding protein specifically binds to an antigen or antigen: MHC complex that is expressed by or associated with a cancer.

Embodiment 87. The host cell of Embodiment 86, wherein the antigen is selected from a ROR1, EGFR, EGFRVIII, EGP-2, EGP-40, GD2, GD3, HPV E6, HPV E7, Her2, L1-CAM, Lewis A, Lewis Y, MUC1, MUC16, PSCA, PSMA, CD19, CD20, CD22, CD56, CD23, CD24, CD30, CD33, CD37, CD44v7/8, CD38, CD56, CD123, CA125, c-MET, FcRH5, WT1, folate receptor α, VEGF-α, VEGFR1, VEGFR2, IL-13Rα2, IL-11Rα, MAGE-A1, PSA, ephrin A2, ephrin B2, NKG2D, NY-ESO-1, TAG-72, mesothelin, NY-ESO, 5T4, BCMA, FAP, Core Binding Factor protein; Cyclin-A1; Carbonic anhydrase 9, ERBB2, a BRAF antigen such as BRAFV600E, MAGE-A3, MAGE-A4, SSX-2, PRAME, HA-1, KRAS (e.g. G12V, G12C, or G12D), or CEA antigen.

Embodiment 88. The host cell of any one of Embodiments 79-87, comprising a chromosomal gene knockout or a mutation of a PD-1 gene; a LAG3 gene; a TIM3 gene; a CTLA4 gene; an HLA component gene; a TCR component gene, or any combination thereof.

Embodiment 89. A composition comprising a fusion protein of any one of Embodiments 1-55 and a pharmaceutically acceptable carrier, excipient, or diluent.

Embodiment 90. A composition comprising a host cell of any one of Embodiments 79-88, and a pharmaceutically acceptable carrier, excipient, or diluent.

Embodiment 91. A unit dose, comprising an effective amount of the host cell of any one of Embodiments 79-88, or of the host cell composition of Embodiment 90.

Embodiment 92. The unit dose of Embodiment 91, comprising (i) a composition comprising at least about 30% CD4⁺ T host cells, combined with (ii) a composition comprising at least about 30% CD8⁺ T cells, in about a 1:1 ratio.

Embodiment 93. The unit dose of Embodiment 92, comprising an effective amount of an effector immune cell comprising a polynucleotide that encodes a binding protein that is capable of specifically binding to an antigen or an antigen: MHC complex.

Embodiment 94. The unit dose of Embodiment 93, wherein the effector immune cell is a T cell, optionally a CD8⁺ T cell.

Embodiment 95. The unit dose of Embodiment 93 or 94, wherein the binding protein encoded by the effector immune cell comprises a TCR or a CAR.

Embodiment 96. The unit dose of any one of Embodiments 93-95, wherein the binding protein encoded by the effector immune cell is specific for the same or a different antigen as compared to a binding protein encoded by the host cell.

Embodiment 97. A method of treating a disease or condition in a subject, the method comprising administering to the subject an effective amount of: (i) a host cell of any one of Embodiments 79-88; and/or (ii) a composition of Embodiment 90; and/or (iii) a unit dose of any one of Embodiments 91-96, wherein the disease or condition is characterized by: (a) the presence of the antigen bound by the encoded binding protein of the host cell; and/or (b) the presence of the antigen bound by the encoded binding protein of the effector immune cell.

Embodiment 98. The method of Embodiment 97, wherein the disease or condition is a cancer.

Embodiment 99. The method of Embodiment 98, wherein the cancer comprises a carcinoma, a sarcoma, a glioma, a lymphoma, a leukemia, a myeloma, or any combination thereof.

Embodiment 100. The method of Embodiment 98 or 99, wherein the cancer comprises a cancer of the head or neck, melanoma, pancreatic cancer, cholangiocarcinoma, hepatocellular cancer, breast cancer including triple-negative breast cancer (TNBC), gastric cancer, non-small-cell lung cancer, prostate cancer, esophageal cancer, mesothelioma, small-cell lung cancer, colorectal cancer, glioblastoma, or any combination thereof.

Embodiment 101. The method of any one of Embodiments 98-100, wherein the cancer comprises Askin's tumor, sarcoma botryoides, chondrosarcoma, Ewing's sarcoma, PNET, malignant hemangioendothelioma, malignant schwannoma, osteosarcoma, alveolar soft part sarcoma, angiosarcoma, cystosarcoma phyllodes, dermatofibrosarcoma protuberans (DFSP), desmoid tumor, desmoplastic small round cell tumor, epithelioid sarcoma, extraskeletal chondrosarcoma, extraskeletal osteosarcoma, fibrosarcoma, gastrointestinal stromal tumor (GIST), hemangiopericytoma, hemangiosarcoma, Kaposi's sarcoma, leiomyosarcoma, liposarcoma, lymphangiosarcoma, lymphosarcoma, undifferentiated pleomorphic sarcoma, malignant peripheral nerve sheath tumor (MPNST), neurofibrosarcoma, rhabdomyosarcoma, synovial sarcoma, undifferentiated pleomorphic sarcoma, squamous cell carcinoma, basal cell carcinoma, adenocarcinoma, linitis plastic, vipoma, cholangiocarcinoma, hepatocellular carcinoma, adenoid cystic carcinoma, renal cell carcinoma, Grawitz tumor, ependymoma, astrocytoma, oligodendroglioma, brainstem glioma, optice nerve glioma, a mixed glioma, Hodgkin's lymphoma, a B-cell lymphoma, non-Hodgkin's lymphoma (NHL), Burkitt's lymphoma, small lymphocytic lymphoma (SLL), diffuse large B-cell lymphoma, follicular lymphoma, immunoblastic large cell lymphoma, precursor B-lymphoblastic lymphoma, and mantle cell lymphoma, Waldenström's macroglobulinemia, CD37⁺ dendritic cell lymphoma, lymphoplasmacytic lymphoma, splenic marginal zone lymphoma, extra-nodal marginal zone B-cell lymphoma of mucosa-associated (MALT) lymphoid tissue, nodal marginal zone B-cell lymphoma, mediastinal (thymic) large B-cell lymphoma, intravascular large B-cell lymphoma, primary effusion lymphoma, adult T-cell lymphoma, extranodal NK/T-cell lymphoma, nasal type, enteropathy-associated T-cell lymphoma, hepatosplenic T-cell lymphoma, blastic NK cell lymphoma, Sezary syndrome, angioimmunoblastic T cell lymphoma, anaplastic large cell lymphoma, or any combination thereof.

Embodiment 102. The method of any one of Embodiments 98-101, wherein the cancer comprises a solid tumor.

Embodiment 103. The method of Embodiment 102, wherein the solid tumor is a sarcoma or a carcinoma.

Embodiment 104. The method of Embodiment 102 or 103, wherein the solid tumor is selected from: chondrosarcoma; fibrosarcoma (fibroblastic sarcoma); Dermatofibrosarcoma protuberans (DFSP); osteosarcoma; rhabdomyosarcoma; Ewing's sarcoma; a gastrointestinal stromal tumor; Leiomyosarcoma; angiosarcoma (vascular sarcoma); Kaposi's sarcoma; liposarcoma; pleomorphic sarcoma; or synovial sarcoma.

Embodiment 105. The method of Embodiment 102 or 103, wherein the solid tumor is selected from a lung carcinoma (e.g., Adenocarcinoma, Squamous Cell Carcinoma (Epidermoid Carcinoma); Squamous cell carcinoma; Adenocarcinoma; Adenosquamous carcinoma; anaplastic carcinoma; Large cell carcinoma; Small cell carcinoma; a breast carcinoma (e.g., Ductal Carcinoma in situ (non-invasive), Lobular carcinoma in situ (non-invasive), Invasive Ductal Carcinoma, Invasive lobular carcinoma, Non-invasive Carcinoma); a liver carcinoma (e.g., Hepatocellular Carcinoma, Cholangiocarcinomas or Bile Duct Cancer); Large-cell undifferentiated carcinoma, Bronchioalveolar carcinoma); an ovarian carcinoma (e.g., Surface epithelial-stromal tumor (Adenocarcinoma) or ovarian epithelial carcinoma (which includes serous tumor, endometrioid tumor and mucinous cystadenocarcinoma), Epidermoid (Squamous cell carcinoma), Embryonal carcinoma and choriocarcinoma (germ cell tumors)); a kidney carcinoma (e.g., Renal adenocarcinoma, hypernephroma, Transitional cell carcinoma (renal pelvis), Squamous cell carcinoma, Bellini duct carcinoma, Clear cell adenocarcinoma, Transitional cell carcinoma, Carcinoid tumor of the renal pelvis); an adrenal carcinoma (e.g., Adrenocortical carcinoma), a carcinoma of the testis (e.g., Germ cell carcinoma (Seminoma, Choriocarcinoma, Embryonal carciroma, Teratocarcinoma), Serous carcinoma); Gastric carcinoma (e.g., Adenocarcinoma); an intestinal carcinoma (e.g., Adenocarcinoma of the duodenum); a colorectal carcinoma; or a skin carcinoma (e.g., Basal cell carcinoma, Squamous cell carcinoma).

Embodiment 106. The method of Embodiment 102 or 103, wherein the solid tumor is an ovarian carcinoma, an ovarian epithelial carcinoma, a cervical adenocarcinoma or small cell carcinoma, a pancreatic carcinoma, a colorectal carcinoma (e.g., an adenocarcinoma or squamous cell carcinoma), a lung carcinoma, a breast ductal carcinoma, or an adenocarcinoma of the prostate.

Embodiment 107. The method of any one of Embodiments 97-106, wherein the host cell is allogeneic, syngeneic, or autologous to the subject.

Embodiment 108. The method of any one of Embodiments 97-107, comprising administering a plurality of unit doses to the subject.

Embodiment 109. The method of Embodiment 108, wherein the plurality of unit doses are administered at intervals between administrations of about two, three, four, five, six, seven, eight, or more weeks.

Embodiment 110. The method according to any one of Embodiments 97-109, wherein the unit dose comprises about 10⁵ cells/m² to about 10¹¹ cells/m².

Embodiment 111. The method of any one of Embodiments 97-110, wherein the subject further receives an adjunctive therapy comprising: (i) chemotherapy; (ii) radiation therapy; (iii) an inhibitor of an immune suppression component; (iv) an agonist of a stimulatory immune checkpoint agent; (v) RNAi; (vi) a cytokine; (vii) a surgery; (viii) a monoclonal antibody and/or an antibody-drug conjugate; or (ix) any combination of (i)-(viii), in any order.

Embodiment 112. The method of Embodiment 111, wherein the adjunctive therapy is administered to the subject before, concurrently with, or after being administered the host cells or composition.

TABLE OF
SEQUENCES

| SEQ ID | | |
|---|---|---|
| NO | DESCRIPTION | SEQUENCE |
| 1 | CD8 co-receptor α | MALPVTALLLPLALLLHAARPSQFRVSPLDRTWNLG |
| | chain precursor, | ETVELKCQVLLSNPTSGCSWLFQPRGAAASPTFLLYL |
| | isoform 1 (human) | SQNKPKAAEGLDTQRFSGKRLGDTFVLTLSDFRREN |
| | | EGYYFCSALSNSIMYFSHFVPVFLPAKPTTTPAPRPPT |
| | | PAPTIASQPLSLRPEACRPAAGGAVHTRGLDFACDIYI |
| | | WAPLAGTCGVLLLSLVITLYCNHRNRRRVCKCPRPV |
| | | VKSGDKPSLSARYV |
| 2 | CD8 co-receptor α | SQFRVSPLDRTWNLGETVELKCQVLLSNPTSGCSWL |
| | chain extracellular | FQPRGAAASPTFLLYLSQNKPKAAEGLDTQRFSGKR |
| | domain | LGDTFVLTLSDFRRENEGYYFCSALSNSIMYFSHFVP |
| | | VFLPAKPTTTPAPRPPTPAPTIASQPLSLRPEACRPAA |
| | | GGAVHTRGLDFACD |
| 3 | CD8 co-receptor α | IYIWAPLAGTCGVLLLSLVIT |
| | chain transmembrane | |
| | domain | |
| 4 | CD8 co-receptor α | LYCNHRNRRRVCKCPRPVVKSGDKPSLSARYV |
| | chain intracellular | |
| | domain | |
| 5 | CD8 co-receptor α | SQFRVSPLDRTWNLGETVELKCQVLLSNPTSGCSWL |
| | chain extracellular | FQPRGAAASPTFLLYLSQNKPKAAEGLDTQRFSGKR |
| | and transmembrane | LGDTFVLTLSDFRRENEGYYFCSALSNSIMYFSHFVP |
| | domains | VFLPAKPTTTPAPRPPTPAPTIASQPLSLRPEACRPAA |
| | | GGAVHTRGLDFACDIYIWAPLAGTCGVLLLSLVIT |
| 6 | CD8 co-receptor β | MRPRLWLLLAAQLTVLHGNSVLQQTPAYIKVQTNK |
| | chain precursor, | MVMLSCEAKISLSNMRIYWLRQRQAPSSDSHHEFLA |
| | isoform 1 (human) | LWDSAKGTIHGEEVEQEKIAVFRDASRFILNLTSVKP |
| | | EDSGIYFCMIVGSPELTFGKGTQLSVVDFLPTTAQPT |
| | | KKSTLKKRVCRLPRPETQKGPLCSPITLGLLVAGVLV |
| | | LLVSLGVAIHLCCRRRRARLRFMKQFYK |
| 7 | CD8 co-receptor β | LQQTPAYIKVQTNKMVMLSCEAKISLSNMRIYWLRQ |
| | chain, isoform 1, | RQAPSSDSHHEFLALWDSAKGTIHGEEVEQEKIAVFR |
| | extracellular domain | DASRFILNLTSVKPEDSGIYFCMIVGSPELTFGKGTQL |
| | | SVVDFLPTTAQPTKKSTLKKRVCRLPRPETQKGPLCS |
| | | P |
| 8 | CD8 co-receptor β | ITLGLLVAGVLVLLVSLGVAI |
| | chain, isoform 1, | |
| | transmembrane | |
| | domain | |
| 9 | CD8 co-receptor β | HLCCRRRRARLRFMKQFYK |
| | chain, isoform 1, | |
| | intracellular domain | |
| 10 | CD8 co-receptor β | HLCCRR |
| | chain, isoform 1, | |
| | intracellular domain | |
| | sequence | |
| 11 | CD8 co-receptor β | MRPRLWLLLAAQLTVLHGNSVLQQTPAYIKVQTNK |
| | chain precursor, | MVMLSCEAKISLSNMRIYWLRQRQAPSSDSHHEFLA |
| | isoform 2 (human) | LWDSAKGTIHGEEVEQEKIAVFRDASRFILNLTSVKP |
| | | EDSGIYFCMIVGSPELTFGKGTQLSVVDFLPTTAQPT |
| | | KKSTLKKRVCRLPRPETQKGPLCSPITLGLLVAGVLV |
| | | LLVSLGVAIHLCCRRRRARLRFMKQLRLHPLEKCSR |
| | | MDY |
| 12 | CD8 co-receptor β | MRPRLWLLLAAQLTVLHGNSVLQQTPAYIKVQTNK |
| | chain precursor, | MVMLSCEAKISLSNMRIYWLRQRQAPSSDSHHEFLA |
| | isoform 3 (human) | LWDSAKGTIHGEEVEQEKIAVFRDASRFILNLTSVKP |
| | | EDSGIYFCMIVGSPELTFGKGTQLSVVDFLPTTAQPT |
| | | KKSTLKKRVCRLPRPETQKGRRRRARLRFMKQPQGE |
| | | GISGTFVPQCLHGYYSNTTTSQKLLNPWILKT |
| 13 | CD8 co-receptor β | MRPRLWLLLAAQLTVLHGNSVLQQTPAYIKVQTNK |
| | chain precursor, | MVMLSCEAKISLSNMRIYWLRQRQAPSSDSHHEFLA |
| | isoform 4 (human) | LWDSAKGTIHGEEVEQEKIAVFRDASRFILNLTSVKP |
| | | EDSGIYFCMIVGSPELTFGKGTQLSVVDFLPTTAQPT |
| | | KKSTLKKRVCRLPRPETQKGPLCSPITLGLLVAGVLV |
| | | LLVSLGVAIHLCCRRRRARLRFMKQKFNIVCLKISGF |
| | | TTCCCFQILQISREYGFGVLLQKDIGQ |
| 14 | CD8 co-receptor β | MRPRLWLLLAAQLTVLHGNSVLQQTPAYIKVQTNK |
| | chain precursor, | MVMLSCEAKISLSNMRIYWLRQRQAPSSDSHHEFLA |
| | isoform 5 (human) | LWDSAKGTIHGEEVEQEKIAVFRDASRFILNLTSVKP |
| | | EDSGIYFCMIVGSPELTFGKGTQLSVVDFLPTTAQPT |
| | | KKSTLKKRVCRLPRPETQKGPLCSPITLGLLVAGVLV |
| | | LLVSLGVAIHLCCRRRRARLRFMKQPQGEGISGTFVP |
| | | QCLHGYYSNTTTSQKLLNPWILKT |
| 15 | CD8 co-receptor β | MRPRLWLLLAAQLTVLHGNSVLQQTPAYIKVQTNK |
| | chain precursor, | MVMLSCEAKISLSNMRIYWLRQRQAPSSDSHHEFLA |
| | isoform 6 (human) | LWDSAKGTIHGEEVEQEKIAVFRDASRFILNLTSVKP |
| | | EDSGIYFCMIVGSPELTFGKGTQLSVVDFLPTTAQPT |
| | | KKSTLKKRVCRLPRPETQKGRRRRARLRFMKQFYK |
| 16 | CD8 co-receptor β | MRPRLWLLLAAQLTVLHGNSVLQQTPAYIKVQTNK |
| | chain precursor, | MVMLSCEAKISLSNMRIYWLRQRQAPSSDSHHEFLA |
| | isoform 7 (human) | LWDSAKGTIHGEEVEQEKIAVFRDASRFILNLTSVKP |
| | | EDSGIYFCMIVGSPELTFGKGTQLSVVDFLPTTAQPT |
| | | KKSTLKKRVCRLPRPETQKDFTNKQRIGFWCPATKR |
| | | HRSVMSTMWKNERRDTFNPGEFNGC |
| 17 | CD8 co-receptor β | MRPRLWLLLAAQLTVLHGNSVLQQTPAYIKVQTNK |
| | chain precursor, | MVMLSCEAKISLSNMRIYWLRQRQAPSSDSHHEFLA |
| | isoform 8 (human) | LWDSAKGTIHGEEVEQEKIAVFRDASRFILNLTSVKP |
| | | EDSGIYFCMIVGSPELTFGKGTQLSVVDFLPTTAQPT |
| | | KKSTLKKRVCRLPRPETQKGLKGKVYQEPLSPNACM |
| | | DTTAILQPHRSCLTHGS |
| 18 | CD28 precursor | MLRLLLALNLFPSIQVTGNKILVKQSPMLVAYDNAV |
| | (human) | NLSCKYSYNLFSREFRASLHKGLDSAVEVCVVYGNY |
| | | SQQLQVYSKTGFNCDGKLGNESVTFYLQNLYVNQT |
| | | DIYFCKIEVMYPPPYLDNEKSNGTIIHVKGKHLCPSPL |
| | | FPGPSKPFWVLVVVGGVLACYSLLVTVAFIIFWVRSK |
| | | RSRLLHSDYMNMTPRRPGPTRKHYQPYAPPRDFAAY |
| | | RS |
| 19 | CD28 intracellular | RSKRSRLLHSDYMNMTPRRPGPTRKHYQPYAPPRDF |
| | domain | AAYRS |
| 20 | CD28 intracellular | RSKRSRGGHSDYMNMTPRRPGPTRKHYQPYAPPRDF |
| | domain (LL to GG) | AAYRS |
| 21 | 4-1BB precursor | MGNSCYNIVATLLLVLNFERTRSLQDPCSNCPAGTFC |
| | (human) | DNNRNQICSPCPPNSFSSAGGQRTCDICRQCKGVFRT |
| | | RKECSSTSNAECDCTPGFHCLGAGCSMCEQDCKQGQ |
| | | ELTKKGCKDCCFGTFNDQKRGICRPWTNCSLDGKSV |
| | | LVNGTKERDVVCGPSPADLSPGASSVTPPAPAREPGH |
| | | SPQIISFFLALTSTALLFLLFFLTLRFSVVKRGRKKLLY |
| | | IFKQPFMRPVQTTQEEDGCSCRFPEEEEGGCEL |
| 22 | 4-1BB intracellular | KRGRKKLLYIFKQPFMRPVQTTQEEDGCSCRFPEEEE |
| | domain | GGCEL |
| 23 | OX40 precursor | MCVGARRLGRGPCAALLLLGLGLSTVTGLHCVGDT |
| | (human) | YPSNDRCCHECRPGNGMVSRCSRSQNTVCRPCGPGF |
| | | YNDVVSSKPKPCTWCNLRSGSERKQLCTATQDTVCR |
| | | CRAGTQPLDSYKPGVDCAPCPPGHFSPGDNQACKPW |
| | | TNCTLAGKHTLQPASNSDAICEDRDPPATQPQETQGP |
| | | PARPITVQPTEAWPRTSQGPSTRPVEVPGGRAVAAIL |
| | | GLGLVLGLLGPLAILLALYLLRRDQRLPPDAHKPPGG |
| | | GSFRPIQEEQADAHSTLAKI |
| 24 | OX40 intracellular | ALYLLRRDQRLPPDAHKPPGGGSFRTPIQEEQADAHS |
| | domain | TLAKI |
| 25 | ICOS precursor | MKSGLWYFFLFCLRIKVLTGEINGSANYEMFIFHNGG |
| | (human) | VQILCKYPDIVQQFKMQLLKGGQILCDLTKTKGSGN |
| | | TVSIKSLKFCHSQLSNNSVSFFLYNLDHSHANYYFCN |
| | | LSIFDPPPFKVTLTGGYLHIYESQLCCQLKFWLPIGCA |
| | | AFVVVCILGCILICWLTKKKYSSSVHDPNGEYMFMR |
| | | AVNTAKKSRLTDVTL |
| 26 | ICOS intracellular | CWLTKKKYSSSVHDPNGEYMFMRAVNTAKKSRLTD |
| | domain | VTL |
| 27 | CD8 co-receptor α | ATGGCTCTGCCTGTGACAGCTCTGCTGCTGCCTCTG |
| | chain (codon- | GCTCTGCTTCTGCATGCCGCTAGACCCAGCCAGTT |
| | optimized nucleotide) | CAGAGTGTCCCCTCTGGACAGAACCTGGAACCTGG |
| | | GCGAGACAGTGGAACTGAAGTGCCAGGTGCTGCT |
| | | GAGCAATCCTACCAGCGGCTGCAGCTGGCTGTTTC |
| | | AGCCTAGAGGTGCTGCCGCCTCTCCTACCTTTCTGC |
| | | TGTACCTGAGCCAGAACAAGCCCAAGGCCGCCGA |
| | | AGGACTGGACACCCAGAGATTCAGCGGCAAGAGA |
| | | CTGGGCGACACCTTCGTGCTGACCCTGAGCGACTT |
| | | CAGAAGAGAGAACGAGGGCTACTACTTCTGCAGC |
| | | GCCCTGAGCAACAGCATCATGTACTTCAGCCACTT |
| | | CGTGCCCGTGTTTCTGCCCGCCAAGCCTACAACAA |
| | | CCCCTGCTCCTAGACCTCCTACACCAGCTCCTACA |
| | | ATCGCCAGCCAGCCTCTGTCTCTGAGGCCAGAAGC |
| | | TTGTAGACCTGCTGCTGGCGGAGCCGTGCATACAA |
| | | GAGGACTGGATTTCGCCTGCGACATCTACATCTGG |
| | | GCCCCTCTGGCTGGAACATGTGGCGTGCTGCTGCT |
| | | GTCCCTGGTCATCACCCTGTACTGCAACCACCGGA |
| | | ACAGGCGGAGAGTGTGCAAGTGCCCTAGACCTGT |
| | | GGTCAAGAGCGGCGACAAGCCTAGCCTGAGCGCC |
| | | AGATATGTT |
| 28 | CD8 co-receptor β | ATGAGGCCTAGACTGTGGCTGCTGCTGGCTGCTCA |
| | chain (codon- | GCTGACAGTGCTGCACGGCAATTCTGTCCTGCAGC |
| | optimized nucleotide) | AGACCCCTGCCTACATCAAGGTGCAGACCAACAA |
| | | GATGGTCATGCTGAGCTGCGAGGCCAAGATCAGCC |
| | | TGAGCAACATGCGGATCTACTGGCTGCGGCAGAG |
| | | ACAGGCCCCTAGCTCTGATAGCCACCACGAGTTTC |
| | | TGGCCCTGTGGGATTCTGCCAAGGGCACCATTCAC |
| | | GGCGAGGAAGTGGAACAAGAGAAGATCGCCGTGT |
| | | TCCGGGACGCCAGCAGATTCATCCTGAACCTGACC |
| | | AGCGTGAAGCCCGAGGACAGCGGCATCTATTTCTG |
| | | CATGATCGTGGGCAGCCCCGAGCTGACATTTGGCA |
| | | AGGGAACACAGCTGAGCGTGGTGGACTTCCTGCCT |
| | | ACTACAGCCCAGCCTACCAAGAAGTCTACCCTGAA |
| | | GAAAAGAGTGTGCAGACTGCCCAGACCTGAGACA |
| | | CAGAAAGGCCCTCTGTGCAGCCCTATCACACTGGG |
| | | ATTGCTGGTGGCTGGCGTGCTGGTGCTGCTGGTTT |
| | | CTCTGGGAGTTGCCATCCACCTGTGCTGTAGACGG |
| | | CGGAGAGCCCGGCTGCGGTTCATGAAGCAGTTCTA |
| | | CAAGtga |
| 29 | CD8 co-receptor β | ATGAGGCCTAGACTGTGGCTGCTGCTGGCTGCTCA |
| | chain extracellular | GCTGACAGTGCTGCACGGCAATTCTGTCCTGCAGC |
| | and transmembrane | AGACCCCTGCCTACATCAAGGTGCAGACCAACAA |
| | domains (codon- | GATGGTCATGCTGAGCTGCGAGGCCAAGATCAGCC |
| | optimized nucleotide) | TGAGCAACATGCGGATCTACTGGCTGCGGCAGAG |
| | | ACAGGCCCCTAGCTCTGATAGCCACCACGAGTTTC |
| | | TGGCCCTGTGGGATTCTGCCAAGGGCACCATTCAC |
| | | GGCGAGGAAGTGGAACAAGAGAAGATCGCCGTGT |
| | | TCCGGGACGCCAGCAGATTCATCCTGAACCTGACC |
| | | AGCGTGAAGCCCGAGGACAGCGGCATCTATTTCTG |
| | | CATGATCGTGGGCAGCCCCGAGCTGACATTTGGCA |
| | | AGGGAACACAGCTGAGCGTGGTGGACTTCCTGCCT |
| | | ACTACAGCCCAGCCTACCAAGAAGTCTACCCTGAA |
| | | GAAAAGAGTGTGCAGACTGCCCAGACCTGAGACA |
| | | CAGAAAGGCCCTCTGTGCAGCCCTATCACACTGGG |
| | | ATTGCTGGTGGCTGGCGTGCTGGTGCTGCTGGTTT |
| | | CTCTGGGAGTTGCCATC |
| 30 | CD8 co-receptor β | ATGAGGCCTAGACTGTGGCTGCTGCTGGCTGCTCA |
| | chain extracellular | GCTGACAGTGCTGCACGGCAATTCTGTCCTGCAGC |
| | and transmembrane | AGACCCCTGCCTACATCAAGGTGCAGACCAACAA |
| | domains with | GATGGTCATGCTGAGCTGCGAGGCCAAGATCAGCC |
| | HLCCRR (codon- | TGAGCAACATGCGGATCTACTGGCTGCGGCAGAG |
| | optimized nucleotide) | ACAGGCCCCTAGCTCTGATAGCCACCACGAGTTTC |
| | | TGGCCCTGTGGGATTCTGCCAAGGGCACCATTCAC |
| | | GGCGAGGAAGTGGAACAAGAGAAGATCGCCGTGT |
| | | TCCGGGACGCCAGCAGATTCATCCTGAACCTGACC |
| | | AGCGTGAAGCCCGAGGACAGCGGCATCTATTTCTG |
| | | CATGATCGTGGGCAGCCCCGAGCTGACATTTGGCA |
| | | AGGGAACACAGCTGAGCGTGGTGGACTTCCTGCCT |
| | | ACTACAGCCCAGCCTACCAAGAAGTCTACCCTGAA |
| | | GAAAAGAGTGTGCAGACTGCCCAGACCTGAGACA |
| | | CAGAAAGGCCCTCTGTGCAGCCCTATCACACTGGG |
| | | ATTGCTGGTGGCTGGCGTGCTGGTGCTGCTGGTTT |
| | | CTCTGGGAGTTGCCATCCACCTGTGCTGCAGAAGA |
| 31 | CD28 intracellular | CGGAGCAAGAGAAGCAGACTGCTGCACAGCGACT |
| | domain (nucleotide, | ACATGAACATGACCCCTAGACGGCCCGGACCTACC |
| | codon-optimized) | AGAAAGCACTACCAGCCTTACGCTCCTCCTAGAGA |
| | | CTTCGCCGCCTACAGATCTtga |
| 32 | CD28 intracellular | CGCAGCAAGCGGAGCAGAGGCGGCCACAGCGACT |
| | domain (LL to GG) | ACATGAACATGACCCCTAGACGGCCTGGCCCCACC |
| | (nucleotide, codon- | AGAAAGCACTACCAGCCCTACGCCCCTCCCCGGGA |
| | optimized) | CTTTGCCGCCTACAGAAGCtga |
| 33 | 4-1BB intracellular | AAGCGGGGCAGAAAGAAGCTGCTGTACATCTTCA |
| | domain (nucleotide, | AGCAGCCCTTCATGCGGCCCGTGCAGACCACACAA |
| | codon-optimized) | GAGGAAGATGGCTGCTCCTGCAGATTCCCCGAGGA |
| | | AGAAGAAGGCGGCTGCGAACTTtga |
| 34 | ICOS intracellular | TGCTGGCTGACCAAGAAAAAGTACAGCAGCAGCG |
| | domain (nucleotide, | TGCACGACCCCAACGGCGAGTACATGTTCATGAGA |
| | codon-optimized) | GCCGTGAACACCGCCAAGAAGTCCAGACTGACCG |
| | | ACGTGACACTGtga |
| 35 | OX40 intracellular | GCCCTGTATCTGCTGAGAAGGGACCAGAGACTGCC |
| | domain (nucleotide, | TCCTGACGCTCACAAACCTCCAGGCGGCGGAAGCT |
| | codon-optimized) | TCAGAACCCCTATCCAAGAGGAACAGGCTGACGC |
| | | CCACAGCACCCTGGCCAAAATTtga |
| 36 | Partial CD8 β-chain | QQTPAYIKVQTNKMVMLSCEAKISLSNMRIYWLRQR |
| | extracellular domain- | QAPSSDSHHEFLALWDSAKGTIHGEEVEQEKIAVFRD |
| | CD8 β-chain | ASRFILNLTSVKPEDSGIYFCMIVGSPELTFGKGTQLS |
| | transmembrane | VVDFLPTTAQPTKKSTLKKRVCRLPRPETQKGPLCSP |
| | domain-(HLCCRR)- | ITLGLLVAGVLVLLVSLGVAIHLCCRRRSKRSRLLHS |
| | CD28 intracellular | DYMNMTPRRPGPTRKHYQPYAPPRDFAAYRS |
| | domain (LL) (amino | |
| | acid) | |
| 37 | Partial CD8 β-chain | QQTPAYIKVQTNKMVMLSCEAKISLSNMRIYWLRQR |
| | extracellular domain- | QAPSSDSHHEFLALWDSAKGTIHGEEVEQEKIAVFRD |
| | CD8 β-chain | ASRFILNLTSVKPEDSGIYFCMIVGSPELTFGKGTQLS |
| | transmembrane | VVDFLPTTAQPTKKSTLKKRVCRLPRPETQKGPLCSP |
| | domain-(HLCCRR)- | ITLGLLVAGVLVLLVSLGVAIHLCCRRRSKRSRGGHS |
| | CD28 intracellular | DYMNMTPRRPGPTRKHYQPYAPPRDFAAYRS |
| | domain (GG) (amino | |
| | acid) | |
| 38 | Partial CD8 β-chain | QQTPAYIKVQTNKMVMLSCEAKISLSNMRIYWLRQR |
| | extracellular domain- | QAPSSDSHHEFLALWDSAKGTIHGEEVEQEKIAVFRD |
| | CD8 β-chain | ASRFILNLTSVKPEDSGIYFCMIVGSPELTFGKGTQLS |
| | transmembrane | VVDFLPTTAQPTKKSTLKKRVCRLPRPETQKGPLCSP |
| | domain-CD28 | ITLGLLVAGVLVLLVSLGVAIRSKRSRGGHSDYMNM |
| | intracellular domain | TPRRPGPTRKHYQPYAPPRDFAAYRS |
| | (GG) (amino acid) | |
| 39 | CD8 α-chain | SQFRVSPLDRTWNLGETVELKCQVLLSNPTSGCSWL |
| | extracellular domain- | FQPRGAAASPTFLLYLSQNKPKAAEGLDTQRFSGKR |
| | CD8 α-chain | LGDTFVLTLSDFRRENEGYYFCSALSNSIMYFSHFVP |
| | transmembrane | VFLPAKPTTTPAPRPPTPAPTIASQPLSLRPEACRPAA |
| | domain-CD28 | GGAVHTRGLDFACDIYIWAPLAGTCGVLLLSLVITRS |
| | intracellular domain | KRSRGGHSDYMNMTPRRPGPTRKHYQPYAPPRDFA |
| | (GG) (amino acid) | AYRS |
| 40 | Partial CD8 β-chain | QQTPAYIKVQTNKMVMLSCEAKISLSNMRIYWLRQR |
| | extracellular domain- | QAPSSDSHHEFLALWDSAKGTIHGEEVEQEKIAVFRD |
| | CD8 β-chain | ASRFILNLTSVKPEDSGIYFCMIVGSPELTFGKGTQLS |
| | transmembrane | VVDFLPTTAQPTKKSTLKKRVCRLPRPETQKGPLCSP |
| | domain-(HLCCRR)- | ITLGLLVAGVLVLLVSLGVAIHLCCRRKRGRKKLLYI |
| | 4-1BB intracellular | FKQPFMRPVQTTQEEDGCCRFPEEEEGGCEL |
| | domain (amino acid) | |
| 41 | Partial CD8 β-chain | QQTPAYIKVQTNKMVMLSCEAKISLSNMRIYWLRQR |
| | extracellular domain- | QAPSSDSHHEFLALWDSAKGTIHGEEVEQEKIAVFRD |
| | CD8 β-chain | ASRFILNLTSVKPEDSGIYFCMIVGSPELTFGKGTQLS |
| | transmembrane | VVDFLPTTAQPTKKSTLKKRVCRLPRPETQKGPLCSP |
| | domain-(HLCCRR)- | ITLGLLVAGVLVLLVSLGVAIHLCCRRCWLTKKKYS |
| | ICOS intracellular | SSVHDPNGEYMFMRAVNTAKKSRLTDVTL |
| | domain (amino acid) | |
| 42 | Partial CD8 β-chain | QQTPAYIKVQTNKMVMLSCEAKISLSNMRIYWLRQR |
| | extracellular domain- | QAPSSDSHHEFLALWDSAKGTIHGEEVEQEKIAVFRD |
| | CD8 β-chain | ASRFILNLTSVKPEDSGIYFCMIVGSPELTFGKGTQLS |
| | transmembrane | VVDFLPTTAQPTKKSTLKKRVCRLPRPETQKGPLCSP |
| | domain-(HLCCRR)- | ITLGLLVAGVLVLLVSLGVAIHLCCRRALYLLRRDQR |
| | OX40 intracellular | LPPDAHKPPGGGSFRTPIQEEQADAHSTLAKI |
| | domain (amino acid) | |
| 43 | CD8 β-chain | ATGAGGCCTAGACTGTGGCTGCTGCTGGCTGCTCA |
| | extracellular domain- | GCTGACAGTGCTGCACGGCAATTCTGTCCTGCAGC |
| | CD8 β-chain | AGACCCCTGCCTACATCAAGGTGCAGACCAACAA |
| | transmembrane | GATGGTCATGCTGAGCTGCGAGGCCAAGATCAGCC |
| | domain-(HLCCRR)- | TGAGCAACATGCGGATCTACTGGCTGCGGCAGAG |
| | CD28 intracellular | ACAGGCCCCTAGCTCTGATAGCCACCACGAGTTTC |
| | domain (LL) (codon- | TGGCCCTGTGGGATTCTGCCAAGGGCACCATTCAC |
| | optimized nucleotide) | GGCGAGGAAGTGGAACAAGAGAAGATCGCCGTGT |
| | | TCCGGGACGCCAGCAGATTCATCCTGAACCTGACC |
| | | AGCGTGAAGCCCGAGGACAGCGGCATCTATTTCTG |
| | | CATGATCGTGGGCAGCCCCGAGCTGACATTTGGCA |
| | | AGGGAACACAGCTGAGCGTGGTGGACTTCCTGCCT |
| | | ACTACAGCCCAGCCTACCAAGAAGTCTACCCTGAA |
| | | GAAAAGAGTGTGCAGACTGCCCAGACCTGAGACA |
| | | CAGAAAGGCCCTCTGTGCAGCCCTATCACACTGGG |
| | | ATTGCTGGTGGCTGGCGTGCTGGTGCTGCTGGTTT |
| | | CTCTGGGAGTTGCCATCCACCTGTGCTGCAGAAGA |
| | | CGGAGCAAGAGAAGCAGACTGCTGCACAGCGACT |
| | | ACATGAACATGACCCCTAGACGGCCCGGACCTACC |
| | | AGAAAGCACTACCAGCCTTACGCTCCTCCTAGAGA |
| | | CTTCGCCGCCTACAGATCTtga |
| 44 | CD8 β-chain | ATGAGGCCTAGACTGTGGCTGCTGCTGGCTGCTCA |
| | extracellular domain- | GCTGACAGTGCTGCACGGCAATTCTGTCCTGCAGC |
| | CD8 β-chain | AGACCCCTGCCTACATCAAGGTGCAGACCAACAA |
| | transmembrane | GATGGTCATGCTGAGCTGCGAGGCCAAGATCAGCC |
| | domain-(HLCCRR)- | TGAGCAACATGCGGATCTACTGGCTGCGGCAGAG |
| | CD28 intracellular | ACAGGCCCCTAGCTCTGATAGCCACCACGAGTTTC |
| | domain (GG) (codon- | TGGCCCTGTGGGATTCTGCCAAGGGCACCATTCAC |
| | optimized nucleotide) | GGCGAGGAAGTGGAACAAGAGAAGATCGCCGTGT |
| | | TCCGGGACGCCAGCAGATTCATCCTGAACCTGACC |
| | | AGCGTGAAGCCCGAGGACAGCGGCATCTATTTCTG |
| | | CATGATCGTGGGCAGCCCCGAGCTGACATTTGGCA |
| | | AGGGAACACAGCTGAGCGTGGTGGACTTCCTGCCT |
| | | ACTACAGCCCAGCCTACCAAGAAGTCTACCCTGAA |
| | | GAAAAGAGTGTGCAGACTGCCCAGACCTGAGACA |
| | | CAGAAAGGCCCTCTGTGCAGCCCTATCACACTGGG |
| | | ATTGCTGGTGGCTGGCGTGCTGGTGCTGCTGGTTT |
| | | CTCTGGGAGTTGCCATCCGCAGCAAGCGGAGCAG |
| | | AGGCGGCCACAGCGACTACATGAACATGACCCCT |
| | | AGACGGCCTGGCCCCACCAGAAAGCACTACCAGC |
| | | CCTACGCCCCTCCCCGGGACTTTGCCGCCTACAGA |
| | | AGCtga |
| 45 | CD8 β-chain | ATGAGGCCTAGACTGTGGCTGCTGCTGGCTGCTCA |
| | extracellular domain- | GCTGACAGTGCTGCACGGCAATTCTGTCCTGCAGC |
| | CD8 β-chain | AGACCCCTGCCTACATCAAGGTGCAGACCAACAA |
| | transmembrane | GATGGTCATGCTGAGCTGCGAGGCCAAGATCAGCC |
| | domain-CD28 | TGAGCAACATGCGGATCTACTGGCTGCGGCAGAG |
| | intracellular domain | ACAGGCCCCTAGCTCTGATAGCCACCACGAGTTTC |
| | (GG) (codon- | TGGCCCTGTGGGATTCTGCCAAGGGCACCATTCAC |
| | optimized nucleotide) | GGCGAGGAAGTGGAACAAGAGAAGATCGCCGTGT |
| | | TCCGGGACGCCAGCAGATTCATCCTGAACCTGACC |
| | | AGCGTGAAGCCCGAGGACAGCGGCATCTATTTCTG |
| | | CATGATCGTGGGCAGCCCCGAGCTGACATTTGGCA |
| | | AGGGAACACAGCTGAGCGTGGTGGACTTCCTGCCT |
| | | ACTACAGCCCAGCCTACCAAGAAGTCTACCCTGAA |
| | | GAAAAGAGTGTGCAGACTGCCCAGACCTGAGACA |
| | | CAGAAAGGCCCTCTGTGCAGCCCTATCACACTGGG |
| | | ATTGCTGGTGGCTGGCGTGCTGGTGCTGCTGGTTT |
| | | CTCTGGGAGTTGCCATCCGCAGCAAGCGGAGCAG |
| | | AGGCGGCCACAGCGACTACATGAACATGACCCCT |
| | | AGACGGCCTGGCCCCACCAGAAAGCACTACCAGC |
| | | CCTACGCCCCTCCCCGGGACTTTGCCGCCTACAGA |
| | | AGCtga |
| 46 | CD8 α-chain | ATGGCTCTGCCTGTGACAGCTCTGCTGCTGCCTCTG |
| | extracellular domain- | GCTCTGCTTCTGCATGCCGCTAGACCCAGCCAGTT |
| | CD8 α-chain | CAGAGTGTCCCCTCTGGACAGAACCTGGAACCTGG |
| | transmembrane | GCGAGACAGTGGAACTGAAGTGCCAGGTGCTGCT |
| | domain-CD28 | GAGCAATCCTACCAGCGGCTGCAGCTGGCTGTTTC |
| | intracellular domain | AGCCTAGAGGTGCTGCCGCCTCTCCTACCTTTCTGC |
| | (GG) (codon- | TGTACCTGAGCCAGAACAAGCCCAAGGCCGCCGA |
| | optimized nucleotide) | AGGACTGGACACCCAGAGATTCAGCGGCAAGAGA |
| | | CTGGGCGACACCTTCGTGCTGACCCTGAGCGACTT |
| | | CAGAAGAGAGAACGAGGGCTACTACTTCTGCAGC |
| | | GCCCTGAGCAACAGCATCATGTACTTCAGCCACTT |
| | | CGTGCCCGTGTTTCTGCCCGCCAAGCCTACAACAA |
| | | CCCCTGCTCCTAGACCTCCTACACCAGCTCCTACA |
| | | ATCGCCAGCCAGCCTCTGTCTCTGAGGCCAGAAGC |
| | | TTGTAGACCTGCTGCTGGCGGAGCCGTGCATACAA |
| | | GAGGACTGGATTTCGCCTGCGACATCTACATCTGG |
| | | GCCCCTCTGGCTGGAACATGTGGCGTGCTGCTGCT |
| | | GTCCCTGGTCATCACCCGCAGCAAGCGGAGCAGA |
| | | GGCGGCCACAGCGACTACATGAACATGACCCCTA |
| | | GACGGCCTGGCCCCACCAGAAAGCACTACCAGCC |
| | | CTACGCCCCTCCCCGGGACTTTGCCGCCTACAGAA |
| | | GCG |
| 47 | CD8 β-chain | ATGAGGCCTAGACTGTGGCTGCTGCTGGCTGCTCA |
| | extracellular domain- | GCTGACAGTGCTGCACGGCAATTCTGTCCTGCAGC |
| | CD8 β-chain | AGACCCCTGCCTACATCAAGGTGCAGACCAACAA |
| | transmembrane | GATGGTCATGCTGAGCTGCGAGGCCAAGATCAGCC |
| | domain-(HLCCRR)- | TGAGCAACATGCGGATCTACTGGCTGCGGCAGAG |
| | 4-1BB intracellular | ACAGGCCCCTAGCTCTGATAGCCACCACGAGTTTC |
| | domain (codon- | TGGCCCTGTGGGATTCTGCCAAGGGCACCATTCAC |
| | optimized nucleotide) | GGCGAGGAAGTGGAACAAGAGAAGATCGCCGTGT |
| | | TCCGGGACGCCAGCAGATTCATCCTGAACCTGACC |
| | | AGCGTGAAGCCCGAGGACAGCGGCATCTATTTCTG |
| | | CATGATCGTGGGCAGCCCCGAGCTGACATTTGGCA |
| | | AGGGAACACAGCTGAGCGTGGTGGACTTCCTGCCT |
| | | ACTACAGCCCAGCCTACCAAGAAGTCTACCCTGAA |
| | | GAAAAGAGTGTGCAGACTGCCCAGACCTGAGACA |
| | | CAGAAAGGCCCTCTGTGCAGCCCTATCACACTGGG |
| | | ATTGCTGGTGGCTGGCGTGCTGGTGCTGCTGGTTT |
| | | CTCTGGGAGTTGCCATCCACCTGTGCTGCAGAAGA |
| | | AAGCGGGGCAGAAAGAAGCTGCTGTACATCTTCA |
| | | AGCAGCCCTTCATGCGGCCCGTGCAGACCACACAA |
| | | GAGGAAGATGGCTGCTCCTGCAGATTCCCCGAGGA |
| | | AGAAGAAGGCGGCTGCGAACTTtga |
| 48 | CD8 β-chain | ATGAGGCCTAGACTGTGGCTGCTGCTGGCTGCTCA |
| | extracellular domain- | GCTGACAGTGCTGCACGGCAATTCTGTCCTGCAGC |
| | CD8 β-chain | AGACCCCTGCCTACATCAAGGTGCAGACCAACAA |
| | transmembrane | GATGGTCATGCTGAGCTGCGAGGCCAAGATCAGCC |
| | domain-(HLCCRR)- | TGAGCAACATGCGGATCTACTGGCTGCGGCAGAG |
| | ICOS intracellular | ACAGGCCCCTAGCTCTGATAGCCACCACGAGTTTC |
| | domain (codon- | TGGCCCTGTGGGATTCTGCCAAGGGCACCATTCAC |
| | optimized nucleotide) | GGCGAGGAAGTGGAACAAGAGAAGATCGCCGTGT |
| | | TCCGGGACGCCAGCAGATTCATCCTGAACCTGACC |
| | | AGCGTGAAGCCCGAGGACAGCGGCATCTATTTCTG |
| | | CATGATCGTGGGCAGCCCCGAGCTGACATTTGGCA |
| | | AGGGAACACAGCTGAGCGTGGTGGACTTCCTGCCT |
| | | ACTACAGCCCAGCCTACCAAGAAGTCTACCCTGAA |
| | | GAAAAGAGTGTGCAGACTGCCCAGACCTGAGACA |
| | | CAGAAAGGCCCTCTGTGCAGCCCTATCACACTGGG |
| | | ATTGCTGGTGGCTGGCGTGCTGGTGCTGCTGGTTT |
| | | CTCTGGGAGTTGCCATCCACCTGTGCTGCAGAAGA |
| | | TGCTGGCTGACCAAGAAAAAGTACAGCAGCAGCG |
| | | TGCACGACCCCAACGGCGAGTACATGTTCATGAGA |
| | | GCCGTGAACACCGCCAAGAAGTCCAGACTGACCG |
| | | ACGTGACACTGtga |
| 49 | CD8 β-chain | ATGAGGCCTAGACTGTGGCTGCTGCTGGCTGCTCA |
| | extracellular domain- | GCTGACAGTGCTGCACGGCAATTCTGTCCTGCAGC |
| | CD8 β-chain | AGACCCCTGCCTACATCAAGGTGCAGACCAACAA |
| | transmembrane | GATGGTCATGCTGAGCTGCGAGGCCAAGATCAGCC |
| | domain-(HLCCRR)- | TGAGCAACATGCGGATCTACTGGCTGCGGCAGAG |
| | OX40 intracellular | ACAGGCCCCTAGCTCTGATAGCCACCACGAGTTTC |
| | domain (codon- | TGGCCCTGTGGGATTCTGCCAAGGGCACCATTCAC |
| | optimized nucleotide) | GGCGAGGAAGTGGAACAAGAGAAGATCGCCGTGT |
| | | TCCGGGACGCCAGCAGATTCATCCTGAACCTGACC |
| | | AGCGTGAAGCCCGAGGACAGCGGCATCTATTTCTG |
| | | CATGATCGTGGGCAGCCCCGAGCTGACATTTGGCA |
| | | AGGGAACACAGCTGAGCGTGGTGGACTTCCTGCCT |
| | | ACTACAGCCCAGCCTACCAAGAAGTCTACCCTGAA |
| | | GAAAAGAGTGTGCAGACTGCCCAGACCTGAGACA |
| | | CAGAAAGGCCCTCTGTGCAGCCCTATCACACTGGG |
| | | ATTGCTGGTGGCTGGCGTGCTGGTGCTGCTGGTTT |
| | | CTCTGGGAGTTGCCATCCACCTGTGCTGCAGAAGA |
| | | GCCCTGTATCTGCTGAGAAGGGACCAGAGACTGCC |
| | | TCCTGACGCTCACAAACCTCCAGGCGGCGGAAGCT |
| | | TCAGAACCCCTATCCAAGAGGAACAGGCTGACGC |
| | | CCACAGCACCCTGGCCAAAATTtga |
| 50 | Porcine teschovirus-1 | GGTTCCGGAGCCACGAACTTCTCTCTGTTAAAGCA |
| | 2A (P2A) peptide | AGCAGGAGACGTGGAAGAAAACCCCGGTCCC |
| | (nucleotide, codon- | |
| | optimized) | |
| 51 | Porcine teschovirus-1 | GGAAGCGGAGCTACTAACTTCAGCCTGCTGAAGCA |
| | 2A (P2A) peptide | GGCTGGAGACGTGGAGGAGAACCCTGGACCT |
| | (nucleotide) | |
| 52 | Thoseaasigna virus | GGAAGCGGAGAGGGCAGAGGAAGTCTGCTAACAT |
| | 2A (T2A) peptide | GCGGTGACGTCGAGGAGAATCCTGGACCT |
| | (nucleotide) | |
| 53 | Equine rhinitis A | GGAAGCGGACAGTGTACTAATTATGCTCTCTTGAA |
| | virus (ERAV) 2A | ATTGGCTGGAGATGTTGAGAGCAACCCTGGACCT |
| | (E2A) peptide | |
| | (nucleotide) | |
| 54 | Foot-and-Mouth | GGAAGCGGAGTGAAACAGACTTTGAATTTTGACCT |
| | disease virus 2A | TCTCAAGTTGGCGGGAGACGTGGAGTCCAACCCTG |
| | (F2A) peptide | GACCT |
| | (nucleotide) | |
| 55 | Porcine teschovirus-1 | GSGATNFSLLKQAGDVEENPGP |
| | 2A (P2A) peptide | |
| | (amino acid) | |
| 56 | Thoseaasigna virus | LEGGGEGRGSLLTCGDVEENPGPR |
| | 2A (T2A) peptide | |
| | (amino acid) | |
| 57 | Equine rhinitis A | QCTNYALLKLAGDVESNPGP |
| | virus (ERAV) 2A | |
| | (E2A) peptide (amino | |
| | acid) | |
| 58 | Foot-and-Mouth | GSGVKQTLNFDLLKLAGDVESNPGP |
| | disease virus 2A | |
| | (F2A) peptide (amino | |
| | acid) | |
| 59 | CD8α-P2A-CD8β | ATGGCTCTGCCTGTGACAGCTCTGCTGCTGCCTCTG |
| | (EC-TM- | GCTCTGCTTCTGCATGCCGCTAGACCCAGCCAGTT |
| | (HLCCRR))- | CAGAGTGTCCCCTCTGGACAGAACCTGGAACCTGG |
| | CD28(IC(LL)) | GCGAGACAGTGGAACTGAAGTGCCAGGTGCTGCT |
| | codon-optimized | GAGCAATCCTACCAGCGGCTGCAGCTGGCTGTTTC |
| | nucleotide) | AGCCTAGAGGTGCTGCCGCCTCTCCTACCTTTCTGC |
| | | TGTACCTGAGCCAGAACAAGCCCAAGGCCGCCGA |
| | | AGGACTGGACACCCAGAGATTCAGCGGCAAGAGA |
| | | CTGGGCGACACCTTCGTGCTGACCCTGAGCGACTT |
| | | CAGAAGAGAGAACGAGGGCTACTACTTCTGCAGC |
| | | GCCCTGAGCAACAGCATCATGTACTTCAGCCACTT |
| | | CGTGCCCGTGTTTCTGCCCGCCAAGCCTACAACAA |
| | | CCCCTGCTCCTAGACCTCCTACACCAGCTCCTACA |
| | | ATCGCCAGCCAGCCTCTGTCTCTGAGGCCAGAAGC |
| | | TTGTAGACCTGCTGCTGGCGGAGCCGTGCATACAA |
| | | GAGGACTGGATTTCGCCTGCGACATCTACATCTGG |
| | | GCCCCTCTGGCTGGAACATGTGGCGTGCTGCTGCT |
| | | GTCCCTGGTCATCACCCTGTACTGCAACCACCGGA |
| | | ACAGGCGGAGAGTGTGCAAGTGCCCTAGACCTGT |
| | | GGTCAAGAGCGGCGACAAGCCTAGCCTGAGCGCC |
| | | AGATATGTTGGTTCCGGAGCCACGAACTTCTCTCT |
| | | GTTAAAGCAAGCAGGAGACGTGGAAGAAAACCCC |
| | | GGTCCCATGAGGCCTAGACTGTGGCTGCTGCTGGC |
| | | TGCTCAGCTGACAGTGCTGCACGGCAATTCTGTCC |
| | | TGCAGCAGACCCCTGCCTACATCAAGGTGCAGACC |
| | | AACAAGATGGTCATGCTGAGCTGCGAGGCCAAGA |
| | | TCAGCCTGAGCAACATGCGGATCTACTGGCTGCGG |
| | | CAGAGACAGGCCCCTAGCTCTGATAGCCACCACGA |
| | | GTTTCTGGCCCTGTGGGATTCTGCCAAGGGCACCA |
| | | TTCACGGCGAGGAAGTGGAACAAGAGAAGATCGC |
| | | CGTGTTCCGGGACGCCAGCAGATTCATCCTGAACC |
| | | TGACCAGCGTGAAGCCCGAGGACAGCGGCATCTA |
| | | TTTCTGCATGATCGTGGGCAGCCCCGAGCTGACAT |
| | | TTGGCAAGGGAACACAGCTGAGCGTGGTGGACTTC |
| | | CTGCCTACTACAGCCCAGCCTACCAAGAAGTCTAC |
| | | CCTGAAGAAAAGAGTGTGCAGACTGCCCAGACCT |
| | | GAGACACAGAAAGGCCCTCTGTGCAGCCCTATCAC |
| | | ACTGGGATTGCTGGTGGCTGGCGTGCTGGTGCTGC |
| | | TGGTTTCTCTGGGAGTTGCCATCCACCTGTGCTGCA |
| | | GAAGACGGAGCAAGAGAAGCAGACTGCTGCACAG |
| | | CGACTACATGAACATGACCCCTAGACGGCCCGGAC |
| | | CTACCAGAAAGCACTACCAGCCTTACGCTCCTCCT |
| | | AGAGACTTCGCCGCCTACAGATCTtga |
| 60 | CD8α-P2A-CD8β | ATGGCTCTGCCTGTGACAGCTCTGCTGCTGCCTCTG |
| | (EC-TM-HLCCRR)- | GCTCTGCTTCTGCATGCCGCTAGACCCAGCCAGTT |
| | CD28(IC(GG)) | CAGAGTGTCCCCTCTGGACAGAACCTGGAACCTGG |
| | (codon-optimized | GCGAGACAGTGGAACTGAAGTGCCAGGTGCTGCT |
| | nucleotide) | GAGCAATCCTACCAGCGGCTGCAGCTGGCTGTTTC |
| | | AGCCTAGAGGTGCTGCCGCCTCTCCTACCTTTCTGC |
| | | TGTACCTGAGCCAGAACAAGCCCAAGGCCGCCGA |
| | | AGGACTGGACACCCAGAGATTCAGGGCAAGAGA |
| | | CTGGGCGACACCTTCGTGCTGACCCTGAGCGACTT |
| | | CAGAAGAGAGAACGAGGGCTACTACTTCTGCAGC |
| | | GCCCTGAGCAACAGCATCATGTACTTCAGCCACTT |
| | | CGTGCCCGTGTTTCTGCCCGCCAAGCCTACAACAA |
| | | CCCCTGCTCCTAGACCTCCTACACCAGCTCCTACA |
| | | ATCGCCAGCCAGCCTCTGTCTCTGAGGCCAGAAGC |
| | | TTGTAGACCTGCTGCTGGCGGAGCCGTGCATACAA |
| | | GAGGACTGGATTTCGCCTGCGACATCTACATCTGG |
| | | GCCCCTCTGGCTGGAACATGTGGCGTGCTGCTGCT |
| | | GTCCCTGGTCATCACCCTGTACTGCAACCACCGGA |
| | | ACAGGCGGAGAGTGTGCAAGTGCCCTAGACCTGT |
| | | GGTCAAGAGCGGCGACAAGCCTAGCCTGAGCGCC |
| | | AGATATGTTGGTTCCGGAGCCACGAACTTCTCTCT |
| | | GTTAAAGCAAGCAGGAGACGTGGAAGAAAACCCC |
| | | GGTCCCATGAGGCCTAGACTGTGGCTGCTGCTGGC |
| | | TGCTCAGCTGACAGTGCTGCACGGCAATTCTGTCC |
| | | TGCAGCAGACCCCTGCCTACATCAAGGTGCAGACC |
| | | AACAAGATGGTCATGCTGAGCTGCGAGGCCAAGA |
| | | TCAGCCTGAGCAACATGCGGATCTACTGGCTGCGG |
| | | CAGAGACAGGCCCCTAGCTCTGATAGCCACCACGA |
| | | GTTTCTGGCCCTGTGGGATTCTGCCAAGGGCACCA |
| | | TTCACGGCGAGGAAGTGGAACAAGAGAAGATCGC |
| | | CGTGTTCCGGGACGCCAGCAGATTCATCCTGAACC |
| | | TGACCAGCGTGAAGCCCGAGGACAGCGGCATCTA |
| | | TTTCTGCATGATCGTGGGCAGCCCCGAGCTGACAT |
| | | TTGGCAAGGGAACACAGCTGAGCGTGGTGGACTTC |
| | | CTGCCTACTACAGCCCAGCCTACCAAGAAGTCTAC |
| | | CCTGAAGAAAAGAGTGTGCAGACTGCCCAGACCT |
| | | GAGACACAGAAAGGCCCTCTGTGCAGCCCTATCAC |
| | | ACTGGGATTGCTGGTGGCTGGCGTGCTGGTGCTGC |
| | | TGGTTTCTCTGGGAGTTGCCATCCACCTGTGCTGCA |
| | | GAAGACGCAGCAAGCGGAGCAGAGGCGGCCACAG |
| | | CGACTACATGAACATGACCCCTAGACGGCCTGGCC |
| | | CCACCAGAAAGCACTACCAGCCCTACGCCCCTCCC |
| | | CGGGACTTTGCCGCCTACAGAAGCtga |
| 61 | CD8α-P2A- | ATGGCTCTGCCTGTGACAGCTCTGCTGCTGCCTCTG |
| | CD8β(EC-TM)- | GCTCTGCTTCTGCATGCCGCTAGACCCAGCCAGTT |
| | CD28(IC(GG)) | CAGAGTGTCCCCTCTGGACAGAACCTGGAACCTGG |
| | (codon-optimized | GCGAGACAGTGGAACTGAAGTGCCAGGTGCTGCT |
| | nucleotide) | GAGCAATCCTACCAGCGGCTGCAGCTGGCTGTTTC |
| | | AGCCTAGAGGTGCTGCCGCCTCTCCTACCTTTCTGC |
| | | TGTACCTGAGCCAGAACAAGCCCAAGGCCGCCGA |
| | | AGGACTGGACACCCAGAGATTCAGCGGCAAGAGA |
| | | CTGGGCGACACCTTCGTGCTGACCCTGAGCGACTT |
| | | CAGAAGAGAGAACGAGGGCTACTACTTCTGCAGC |
| | | GCCCTGAGCAACAGCATCATGTACTTCAGCCACTT |
| | | CGTGCCCGTGTTTCTGCCCGCCAAGCCTACAACAA |
| | | CCCCTGCTCCTAGACCTCCTACACCAGCTCCTACA |
| | | ATCGCCAGCCAGCCTCTGTCTCTGAGGCCAGAAGC |
| | | TTGTAGACCTGCTGCTGGCGGAGCCGTGCATACAA |
| | | GAGGACTGGATTTCGCCTGCGACATCTACATCTGG |
| | | GCCCCTCTGGCTGGAACATGTGGCGTGCTGCTGCT |
| | | GTCCCTGGTCATCACCCTGTACTGCAACCACCGGA |
| | | ACAGGCGGAGAGTGTGCAAGTGCCCTAGACCTGT |
| | | GGTCAAGAGCGGCGACAAGCCTAGCCTGAGCGCC |
| | | AGATATGTTGGTTCCGGAGCCACGAACTTCTCTCT |
| | | GTTAAAGCAAGCAGGAGACGTGGAAGAAAACCCC |
| | | GGTCCCATGAGGCCTAGACTGTGGCTGCTGCTGGC |
| | | TGCTCAGCTGACAGTGCTGCACGGCAATTCTGTCC |
| | | TGCAGCAGACCCCTGCCTACATCAAGGTGCAGACC |
| | | AACAAGATGGTCATGCTGAGCTGCGAGGCCAAGA |
| | | TCAGCCTGAGCAACATGCGGATCTACTGGCTGCGG |
| | | CAGAGACAGGCCCCTAGCTCTGATAGCCACCACGA |
| | | GTTTCTGGCCCTGTGGGATTCTGCCAAGGGCACCA |
| | | TTCACGGCGAGGAAGTGGAACAAGAGAAGATCGC |
| | | CGTGTTCCGGGACGCCAGCAGATTCATCCTGAACC |
| | | TGACCAGCGTGAAGCCCGAGGACAGCGGCATCTA |
| | | TTTCTGCATGATCGTGGGCAGCCCCGAGCTGACAT |
| | | TTGGCAAGGGAACACAGCTGAGCGTGGTGGACTTC |
| | | CTGCCTACTACAGCCCAGCCTACCAAGAAGTCTAC |
| | | CCTGAAGAAAAGAGTGTGCAGACTGCCCAGACCT |
| | | GAGACACAGAAAGGCCCTCTGTGCAGCCCTATCAC |
| | | ACTGGGATTGCTGGTGGCTGGCGTGCTGGTGCTGC |
| | | TGGTTTCTCTGGGAGTTGCCATCCGCAGCAAGCGG |
| | | AGCAGAGGCGGCCACAGCGACTACATGAACATGA |
| | | CCCCTAGACGGCCTGGCCCCACCAGAAAGCACTAC |
| | | CAGCCCTACGCCCCTCCCCGGGACTTTGCCGCCTA |
| | | CAGAAGCtga |
| 62 | CD8α(EC-TM)- | ATGGCTCTGCCTGTGACAGCTCTGCTGCTGCCTCTG |
| | CD28(IC(GG))-P2A- | GCTCTGCTTCTGCATGCCGCTAGACCCAGCCAGTT |
| | CD8β(EC-TM)- | CAGAGTGTCCCCTCTGGACAGAACCTGGAACCTGG |
| | CD28(IC(GG)) | GCGAGACAGTGGAACTGAAGTGCCAGGTGCTGCT |
| | (codon-optimized | GAGCAATCCTACCAGCGGCTGCAGCTGGCTGTTTC |
| | nucleotide) | AGCCTAGAGGTGCTGCCGCCTCTCCTACCTTTCTGC |
| | | TGTACCTGAGCCAGAACAAGCCCAAGGCCGCCGA |
| | | AGGACTGGACACCCAGAGATTCAGCGGCAAGAGA |
| | | CTGGGCGACACCTTCGTGCTGACCCTGAGCGACTT |
| | | CAGAAGAGAGAACGAGGGCTACTACTTCTGCAGC |
| | | GCCCTGAGCAACAGCATCATGTACTTCAGCCACTT |
| | | CGTGCCCGTGTTTCTGCCCGCCAAGCCTACAACAA |
| | | CCCCTGCTCCTAGACCTCCTACACCAGCTCCTACA |
| | | ATCGCCAGCCAGCCTCTGTCTCTGAGGCCAGAAGC |
| | | TTGTAGACCTGCTGCTGGCGGAGCCGTGCATACAA |
| | | GAGGACTGGATTTCGCCTGCGACATCTACATCTGG |
| | | GCCCCTCTGGCTGGAACATGTGGCGTGCTGCTGCT |
| | | GTCCCTGGTCATCACCCGCAGCAAGCGGAGCAGA |
| | | GGCGGCCACAGCGACTACATGAACATGACCCCTA |
| | | GACGGCCTGGCCCCACCAGAAAGCACTACCAGCC |
| | | CTACGCCCCTCCCCGGGACTTTGCCGCCTACAGAA |
| | | GCGGTTCCGGAGCCACGAACTTCTCTCTGTTAAAG |
| | | CAAGCAGGAGACGTGGAAGAAAACCCCGGTCCCA |
| | | TGAGGCCTAGACTGTGGCTGCTGCTGGCTGCTCAG |
| | | CTGACAGTGCTGCACGGCAATTCTGTCCTGCAGCA |
| | | GACCCCTGCCTACATCAAGGTGCAGACCAACAAG |
| | | ATGGTCATGCTGAGCTGCGAGGCCAAGATCAGCCT |
| | | GAGCAACATGCGGATCTACTGGCTGCGGCAGAGA |
| | | CAGGCCCCTAGCTCTGATAGCCACCACGAGTTTCT |
| | | GGCCCTGTGGGATTCTGCCAAGGGCACCATTCACG |
| | | GCGAGGAAGTGGAACAAGAGAAGATCGCCGTGTT |
| | | CCGGGACGCCAGCAGATTCATCCTGAACCTGACCA |
| | | GCGTGAAGCCCGAGGACAGCGGCATCTATTTCTGC |
| | | ATGATCGTGGGCAGCCCCGAGCTGACATTTGGCAA |
| | | GGGAACACAGCTGAGCGTGGTGGACTTCCTGCCTA |
| | | CTACAGCCCAGCCTACCAAGAAGTCTACCCTGAAG |
| | | AAAAGAGTGTGCAGACTGCCCAGACCTGAGACAC |
| | | AGAAAGGCCCTCTGTGCAGCCCTATCACACTGGGA |
| | | TTGCTGGTGGCTGGCGTGCTGGTGCTGCTGGTTTCT |
| | | CTGGGAGTTGCCATCCGCAGCAAGCGGAGCAGAG |
| | | GCGGCCACAGCGACTACATGAACATGACCCCTAG |
| | | ACGGCCTGGCCCCACCAGAAAGCACTACCAGCCCT |
| | | ACGCCCCTCCCCGGGACTTTGCCGCCTACAGAAGCt |
| | | ga |
| 63 | CD8α(EC-TM)- | ATGGCTCTGCCTGTGACAGCTCTGCTGCTGCCTCTG |
| | CD28(IC(GG))-P2A- | GCTCTGCTTCTGCATGCCGCTAGACCCAGCCAGTT |
| | CD8β(codon- | CAGAGTGTCCCCTCTGGACAGAACCTGGAACCTGG |
| | optimized nucleotide) | GCGAGACAGTGGAACTGAAGTGCCAGGTGCTGCT |
| | | GAGCAATCCTACCAGCGGCTGCAGCTGGCTGTTTC |
| | | AGCCTAGAGGTGCTGCCGCCTCTCCTACCTTTCTGC |
| | | TGTACCTGAGCCAGAACAAGCCCAAGGCCGCCGA |
| | | AGGACTGGACACCCAGAGATTCAGCGGCAAGAGA |
| | | CTGGGCGACACCTTCGTGCTGACCCTGAGCGACTT |
| | | CAGAAGAGAGAACGAGGGCTACTACTTCTGCAGC |
| | | GCCCTGAGCAACAGCATCATGTACTTCAGCCACTT |
| | | CGTGCCCGTGTTTCTGCCCGCCAAGCCTACAACAA |
| | | CCCCTGCTCCTAGACCTCCTACACCAGCTCCTACA |
| | | ATCGCCAGCCAGCCTCTGTCTCTGAGGCCAGAAGC |
| | | TTGTAGACCTGCTGCTGGCGGAGCCGTGCATACAA |
| | | GAGGACTGGATTTCGCCTGCGACATCTACATCTGG |
| | | GCCCCTCTGGCTGGAACATGTGGCGTGCTGCTGCT |
| | | GTCCCTGGTCATCACCCGCAGCAAGCGGAGCAGA |
| | | GGCGGCCACAGCGACTACATGAACATGACCCCTA |
| | | GACGGCCTGGCCCCACCAGAAAGCACTACCAGCC |
| | | CTACGCCCCTCCCCGGGACTTTGCCGCCTACAGAA |
| | | GCGGTTCCGGAGCCACGAACTTCTCTCTGTTAAAG |
| | | CAAGCAGGAGACGTGGAAGAAAACCCCGGTCCCA |
| | | TGAGGCCTAGACTGTGGCTGCTGCTGGCTGCTCAG |
| | | CTGACAGTGCTGCACGGCAATTCTGTCCTGCAGCA |
| | | GACCCCTGCCTACATCAAGGTGCAGACCAACAAG |
| | | ATGGTCATGCTGAGCTGCGAGGCCAAGATCAGCCT |
| | | GAGCAACATGCGGATCTACTGGCTGCGGCAGAGA |
| | | CAGGCCCCTAGCTCTGATAGCCACCACGAGTTTCT |
| | | GGCCCTGTGGGATTCTGCCAAGGGCACCATTCACG |
| | | GCGAGGAAGTGGAACAAGAGAAGATCGCCGTGTT |
| | | CCGGGACGCCAGCAGATTCATCCTGAACCTGACCA |
| | | GCGTGAAGCCCGAGGACAGCGGCATCTATTTCTGC |
| | | ATGATCGTGGGCAGCCCCGAGCTGACATTTGGCAA |
| | | GGGAACACAGCTGAGCGTGGTGGACTTCCTGCCTA |
| | | CTACAGCCCAGCCTACCAAGAAGTCTACCCTGAAG |
| | | AAAAGAGTGTGCAGACTGCCCAGACCTGAGACAC |
| | | AGAAAGGCCCTCTGTGCAGCCCTATCACACTGGGA |
| | | TTGCTGGTGGCTGGCGTGCTGGTGCTGCTGGTTTCT |
| | | CTGGGAGTTGCCATCCACCTGTGCTGTAGACGGCG |
| | | GAGAGCCCGGCTGCGGTTCATGAAGCAGTTCTACA |
| | | AGtga |
| 64 | CD8α-P2A- | ATGGCTCTGCCTGTGACAGCTCTGCTGCTGCCTCTG |
| | CD8β(EC-TM- | GCTCTGCTTCTGCATGCCGCTAGACCCAGCCAGTT |
| | HCCLRR)-4- | CAGAGTGTCCCCTCTGGACAGAACCTGGAACCTGG |
| | 1BB(IC)(codon- | GCGAGACAGTGGAACTGAAGTGCCAGGTGCTGCT |
| | optimized nucleotide) | GAGCAATCCTACCAGCGGCTGCAGCTGGCTGTTTC |
| | | AGCCTAGAGGTGCTGCCGCCTCTCCTACCTTTCTGC |
| | | TGTACCTGAGCCAGAACAAGCCCAAGGCCGCCGA |
| | | AGGACTGGACACCCAGAGATTCAGCGGCAAGAGA |
| | | CTGGGCGACACCTTCGTGCTGACCCTGAGCGACTT |
| | | CAGAAGAGAGAACGAGGGCTACTACTTCTGCAGC |
| | | GCCCTGAGCAACAGCATCATGTACTTCAGCCACTT |
| | | CGTGCCCGTGTTTCTGCCCGCCAAGCCTACAACAA |
| | | CCCCTGCTCCTAGACCTCCTACACCAGCTCCTACA |
| | | ATCGCCAGCCAGCCTCTGTCTCTGAGGCCAGAAGC |
| | | TTGTAGACCTGCTGCTGGCGGAGCCGTGCATACAA |
| | | GAGGACTGGATTTCGCCTGCGACATCTACATCTGG |
| | | GCCCCTCTGGCTGGAACATGTGGCGTGCTGCTGCT |
| | | GTCCCTGGTCATCACCCTGTACTGCAACCACCGGA |
| | | ACAGGCGGAGAGTGTGCAAGTGCCCTAGACCTGT |
| | | GGTCAAGAGCGGCGACAAGCCTAGCCTGAGCGCC |
| | | AGATATGTTGGTTCCGGAGCCACGAACTTCTCTCT |
| | | GTTAAAGCAAGCAGGAGACGTGGAAGAAAACCCC |
| | | GGTCCCATGAGGCCTAGACTGTGGCTGCTGCTGGC |
| | | TGCTCAGCTGACAGTGCTGCACGGCAATTCTGTCC |
| | | TGCAGCAGACCCCTGCCTACATCAAGGTGCAGACC |
| | | AACAAGATGGTCATGCTGAGCTGCGAGGCCAAGA |
| | | TCAGCCTGAGCAACATGCGGATCTACTGGCTGCGG |
| | | CAGAGACAGGCCCCTAGCTCTGATAGCCACCACGA |
| | | GTTTCTGGCCCTGTGGGATTCTGCCAAGGGCACCA |
| | | TTCACGGCGAGGAAGTGGAACAAGAGAAGATCGC |
| | | CGTGTTCCGGGACGCCAGCAGATTCATCCTGAACC |
| | | TGACCAGCGTGAAGCCCGAGGACAGCGGCATCTA |
| | | TTTCTGCATGATCGTGGGCAGCCCCGAGCTGACAT |
| | | TTGGCAAGGGAACACAGCTGAGCGTGGTGGACTTC |
| | | CTGCCTACTACAGCCCAGCCTACCAAGAAGTCTAC |
| | | CCTGAAGAAAAGAGTGTGCAGACTGCCCAGACCT |
| | | GAGACACAGAAAGGCCCTCTGTGCAGCCCTATCAC |
| | | ACTGGGATTGCTGGTGGCTGGCGTGCTGGTGCTGC |
| | | TGGTTTCTCTGGGAGTTGCCATCCACCTGTGCTGCA |
| | | GAAGAAAGCGGGGCAGAAAGAAGCTGCTGTACAT |
| | | CTTCAAGCAGCCCTTCATGCGGCCCGTGCAGACCA |
| | | CACAAGAGGAAGATGGCTGCTCCTGCAGATTCCCC |
| | | GAGGAAGAAGAAGGCGGCTGCGAACTTtga |
| 65 | CD8α-P2A- | ATGGCTCTGCCTGTGACAGCTCTGCTGCTGCCTCTG |
| | CD8β(EC-TM- | GCTCTGCTTCTGCATGCCGCTAGACCCAGCCAGTT |
| | HCCLRR)-ICOS(IC) | CAGAGTGTCCCCTCTGGACAGAACCTGGAACCTGG |
| | (codon-optimized | GCGAGACAGTGGAACTGAAGTGCCAGGTGCTGCT |
| | nucleotide) | GAGCAATCCTACCAGCGGCTGCAGCTGGCTGTTTC |
| | | AGCCTAGAGGTGCTGCCGCCTCTCCTACCTTTCTGC |
| | | TGTACCTGAGCCAGAACAAGCCCAAGGCCGCCGA |
| | | AGGACTGGACACCCAGAGATTCAGCGGCAAGAGA |
| | | CTGGGCGACACCTTCGTGCTGACCCTGAGCGACTT |
| | | CAGAAGAGAGAACGAGGGCTACTACTTCTGCAGC |
| | | GCCCTGAGCAACAGCATCATGTACTTCAGCCACTT |
| | | CGTGCCCGTGTTTCTGCCCGCCAAGCCTACAACAA |
| | | CCCCTGCTCCTAGACCTCCTACACCAGCTCCTACA |
| | | ATCGCCAGCCAGCCTCTGTCTCTGAGGCCAGAAGC |
| | | TTGTAGACCTGCTGCTGGCGGAGCCGTGCATACAA |
| | | GAGGACTGGATTTCGCCTGCGACATCTACATCTGG |
| | | GCCCCTCTGGCTGGAACATGTGGCGTGCTGCTGCT |
| | | GTCCCTGGTCATCACCCTGTACTGCAACCACCGGA |
| | | ACAGGCGGAGAGTGTGCAAGTGCCCTAGACCTGT |
| | | GGTCAAGAGCGGCGACAAGCCTAGCCTGAGCGCC |
| | | AGATATGTTGGTTCCGGAGCCACGAACTTCTCTCT |
| | | GTTAAAGCAAGCAGGAGACGTGGAAGAAAACCCC |
| | | GGTCCCATGAGGCCTAGACTGTGGCTGCTGCTGGC |
| | | TGCTCAGCTGACAGTGCTGCACGGCAATTCTGTCC |
| | | TGCAGCAGACCCCTGCCTACATCAAGGTGCAGACC |
| | | AACAAGATGGTCATGCTGAGCTGCGAGGCCAAGA |
| | | TCAGCCTGAGCAACATGCGGATCTACTGGCTGCGG |
| | | CAGAGACAGGCCCCTAGCTCTGATAGCCACCACGA |
| | | GTTTCTGGCCCTGTGGGATTCTGCCAAGGGCACCA |
| | | TTCACGGCGAGGAAGTGGAACAAGAGAAGATCGC |
| | | CGTGTTCCGGGACGCCAGCAGATTCATCCTGAACC |
| | | TGACCAGCGTGAAGCCCGAGGACAGCGGCATCTA |
| | | TTTCTGCATGATCGTGGGCAGCCCCGAGCTGACAT |
| | | TTGGCAAGGGAACACAGCTGAGCGTGGTGGACTTC |
| | | CTGCCTACTACAGCCCAGCCTACCAAGAAGTCTAC |
| | | CCTGAAGAAAAGAGTGTGCAGACTGCCCAGACCT |
| | | GAGACACAGAAAGGCCCTCTGTGCAGCCCTATCAC |
| | | ACTGGGATTGCTGGTGGCTGGCGTGCTGGTGCTGC |
| | | TGGTTTCTCTGGGAGTTGCCATCCACCTGTGCTGCA |
| | | GAAGATGCTGGCTGACCAAGAAAAAGTACAGCAG |
| | | CAGCGTGCACGACCCCAACGGCGAGTACATGTTCA |
| | | TGAGAGCCGTGAACACCGCCAAGAAGTCCAGACT |
| | | GACCGACGTGACACTGtga |
| 66 | CD8α-P2A- | ATGGCTCTGCCTGTGACAGCTCTGCTGCTGCCTCTG |
| | CD8β(EC-TM- | GCTCTGCTTCTGCATGCCGCTAGACCCAGCCAGTT |
| | HCCLRR)-OX40(IC) | CAGAGTGTCCCCTCTGGACAGAACCTGGAACCTGG |
| | (codon-optimized | GCGAGACAGTGGAACTGAAGTGCCAGGTGCTGCT |
| | nucleotide) | GAGCAATCCTACCAGCGGCTGCAGCTGGCTGTTTC |
| | | AGCCTAGAGGTGCTGCCGCCTCTCCTACCTTTCTGC |
| | | TGTACCTGAGCCAGAACAAGCCCAAGGCCGCCGA |
| | | AGGACTGGACACCCAGAGATTCAGCGGCAAGAGA |
| | | CTGGGCGACACCTTCGTGCTGACCCTGAGCGACTT |
| | | CAGAAGAGAGAACGAGGGCTACTACTTCTGCAGC |
| | | GCCCTGAGCAACAGCATCATGTACTTCAGCCACTT |
| | | CGTGCCCGTGTTTCTGCCCGCCAAGCCTACAACAA |
| | | CCCCTGCTCCTAGACCTCCTACACCAGCTCCTACA |
| | | ATCGCCAGCCAGCCTCTGTCTCTGAGGCCAGAAGC |
| | | TTGTAGACCTGCTGCTGGCGGAGCCGTGCATACAA |
| | | GAGGACTGGATTTCGCCTGCGACATCTACATCTGG |
| | | GCCCCTCTGGCTGGAACATGTGGCGTGCTGCTGCT |
| | | GTCCCTGGTCATCACCCTGTACTGCAACCACCGGA |
| | | ACAGGCGGAGAGTGTGCAAGTGCCCTAGACCTGT |
| | | GGTCAAGAGCGGCGACAAGCCTAGCCTGAGCGCC |
| | | AGATATGTTGGTTCCGGAGCCACGAACTTCTCTCT |
| | | GTTAAAGCAAGCAGGAGACGTGGAAGAAAACCCC |
| | | GGTCCCATGAGGCCTAGACTGTGGCTGCTGCTGGC |
| | | TGCTCAGCTGACAGTGCTGCACGGCAATTCTGTCC |
| | | TGCAGCAGACCCCTGCCTACATCAAGGTGCAGACC |
| | | AACAAGATGGTCATGCTGAGCTGCGAGGCCAAGA |
| | | TCAGCCTGAGCAACATGCGGATCTACTGGCTGCGG |
| | | CAGAGACAGGCCCCTAGCTCTGATAGCCACCACGA |
| | | GTTTCTGGCCCTGTGGGATTCTGCCAAGGGCACCA |
| | | TTCACGGCGAGGAAGTGGAACAAGAGAAGATCGC |
| | | CGTGTTCCGGGACGCCAGCAGATTCATCCTGAACC |
| | | TGACCAGCGTGAAGCCCGAGGACAGCGGCATCTA |
| | | TTTCTGCATGATCGTGGGCAGCCCCGAGCTGACAT |
| | | TTGGCAAGGGAACACAGCTGAGCGTGGTGGACTTC |
| | | CTGCCTACTACAGCCCAGCCTACCAAGAAGTCTAC |
| | | CCTGAAGAAAAGAGTGTGCAGACTGCCCAGACCT |
| | | GAGACACAGAAAGGCCCTCTGTGCAGCCCTATCAC |
| | | ACTGGGATTGCTGGTGGCTGGCGTGCTGGTGCTGC |
| | | TGGTTTCTCTGGGAGTTGCCATCCACCTGTGCTGCA |
| | | GAAGAGCCCTGTATCTGCTGAGAAGGGACCAGAG |
| | | ACTGCCTCCTGACGCTCACAAACCTCCAGGCGGCG |
| | | GAAGCTTCAGAACCCCTATCCAAGAGGAACAGGC |
| | | TGACGCCCACAGCACCCTGGCCAAAATTtga |
| 67 | CD8α IgV-like | SQFRVSPLDRTWNLGETVELKCQVLLSNPTSGCSWL |
| | domain | FQPRGAAASPTFLLYLSQNKPKAAEGLDTQRFSGKR |
| | | LGDTFVLTLSDFRRENEGYYFCSALSNSIMYFSHFVP |
| | | VFLPA |
| 68 | CD8β IgV-like | LQQTPAYIKVQTNKMVMLSCEAKISLSNMRIYWLRQ |
| | domain | RQAPSSDSHHEFLALWDSAKGTIHGEEVEQEKIA VFR |
| | | DASRFILNLTSVKPEDSGIYFCMIVGSPELTFGKGTQL |
| 69 | CD3ϵ ectodomain | DGNEEMGGITQTPYKVSISGTTVILTCPQYPGSEILWQ |
| | (without signal | HNDKNIGGDEDDKNIGSDEDHLSLKEFSELEQSGYY |
| | peptide) | VCYPRGSKPEDANFYLYLRARVCENCMEMD |
| 70 | CD3ϵ transmembrane | VMSVATIVIVDICITGGLLLLVYYWS |
| | domain | |
| 71 | CD3δ ectodomain | FKIPIEELEDRVFVNCNTSITWVEGTVGTLLSDITRLD |
| | (without signal | LGKRILDPRGIYRCNGTDIYKDKESTVQVHYRMCQS |
| | peptide) | CVELDPATVA |
| 72 | CD3δ transmembrane | GIIVTDVIATLLLALGVFCFA |
| | domain | |
| 73 | CD3γ ectodomain | QSIKGNHLVKVYDYQEDGSVLLTCDAEAKNITWFKD |
| | (without signal | GKMIGFLTEDKKKWNLGSNAKDPRGMYQCKGSQN |
| | peptide) | KSKPLQVYYRMCQNCIELNAATIS |
| 74 | CD3γ transmembrane | GFLFAEIVSIFVLAVGVYFIA |
| | domain | |
| 75 | CD3ζ | QSFGLLDPK |
| | ectodomain (without | |
| | signal peptide) | |
| 76 | CD3ζ transmembrane | LCYLLDGILFIYGVILTALFL |
| | domain | |
| 77 | CD3ζ endodomain | RVKFSRSADAPAYQQGQNQLYNELNLGRREEYDVL |
| | | DKRRGRDPEMGGKPQRRKNPQEGLYNELQKDKMA |
| | | EAYSEIGMKGERRRGKGHDGLYQGLSTATKDTYDA |
| | | LHMQALPPR |
| 78 | CD8α_EC (with | MALPVTALLLPLALLLHAARPSQFRVSPLDRTWNLG |
| | signal peptide) | ETVELKCQVLLSNPTSGCSWLFQPRGAAASPTFLLYL |
| | | SQNKPKAAEGLDTQRFSGKRLGDTFVLTLSDFRREN |
| | | EGYYFCSALSNSIMYFSHFVPVFLPA |
| 79 | CD8α_EC fragment | SQFRVSPLDRTWNLGETVELKCQVLLSNPTSGCSWL |
| | (without signal | FQPRGAAASPTFLLYLSQNKPKAAEGLDTQRFSGKR |
| | peptide) | LGDTFVLTLSDFRRENEGYYFCSALSNSIMYFSHFVP |
| | | VFLPA |
| 80 | CD28_TM | FWVLVVVGGVLACYSLLVTVAFIIFWV |
| 81 | CD28 IC partial | RSKRSRGGHSDAMNMTARRAGPTRKHYQAYAAPR |
| | signaling mutant with | DFAAYRS |
| | GG mutation | |
| 82 | CD8 co-receptor β | MRPRLWLLLAAQLTVLHGNSVLQQTPAYIKVQTNK |
| | chain, isoform 1, | MVMLSCEAKISLSNMRIYWLRQRQAPSSDSHHEFLA |
| | extracellular domain | LWDSAKGTIHGEEVEQEKIAVFRDASRFILNLTSVKP |
| | (with signal peptide) | EDSGIYFCMIVGSPELTFGKGTQLSVVDFLPTTAQPT |
| | | KKSTLKKRVCRLPRPETQKGPLCSP |
| 83 | CD8 co-receptor β- | HLCCRRRSKRSRGGHSDYMNMTPRRPGPTRKHYQP |
| | IC-CD28-IC fusion | YAPPRDFAAYRS |
| | with GG mutation | |
| 84 | CD8 co-receptor β- | HLCCRRRSKRSRLLHSDYMNMTPRRPGPTRKHYQPY |
| | IC-CD28-IC fusion | APPRDFAAYRS |
| 85 | CD8 co-receptor β- | HLCCRRKRGRKKLLYIFKQPFMRPVQTTQEEDGCSC |
| | IC-41BB-IC fusion | RFPEEEEGGCEL |
| 86 | CD8 co-receptor β- | HLCCRRCWLTKKKYSSSVHDPNGEYMFMRAVNTAK |
| | IC-ICOS-IC fusion | KSRLTDVTL |
| 87 | CD8 co-receptor β- | HLCCRRALYLLRRDQRLPPDAHKPPGGGSFRTPIQEE |
| | IC-OX40-IC fusion | QADAHSTLAKI |
| 88 | CD8 co-receptor β- | HLCCRRQLGLHIWQLRSQCMWPRETQLLLEVPPSTE |
| | IC-GITR-IC fusion | DARSCQFPEEERGERSAEEKGRLGDLWV |
| 89 | CD8 co-receptor β- | HLCCRRRRARLRFMKQFYKPLQDNLVIALHSYEPSH |
| | IC-Lck-IC fusion | DGDLGFEKGEQLRILEQSGEWWKAQSLTTGQEGFIPF |
| | | NFVAKANSLEPEPWFFKNLSRKDAERQLLAPGNTHG |
| | | SFLIRESESTAGSFSLSVRDFDQNQGEVVKHYKIRNL |
| | | DNGGFYISPRITFPGLHELVRHYTNASDGLCTRLSRPC |
| | | QTQKPQKPWWEDEWEVPRETLKLVERLGAGQFGEV |
| | | WMGYYNGHTKVAVKSLKQGSMSPDAFLAEANLMK |
| | | QLQHQRLVRLYAVVTQEPIYIITEYMENGSLVDFLKT |
| | | PSGIKLTINKLLDMAAQIAEGMAFIEERNYIHRDLRA |
| | | ANILVSDTLSCKIADFGLARLIEDNEYTAREGAKFPIK |
| | | WTAPEAINYGTFTIKSDVWSFGILLTEIVTHGRIPYPG |
| | | MTNPEVIQNLERGYRMVRPDNCPEELYQLMRLCWK |
| | | ERPEDRPTFDYLRSVLEDFFTATEGQYQPQP |
| 90 | CD8 co-receptor-α-β- | SQFRVSPLDRTWNLGETVELKCQVLLSNPTSGCSWL |
| | CD28 chimeric stalk | FQPRGAAASPTFLLYLSQNKPKAAEGLDTQRFSGKR |
| | (without signal | LGDTFVLTLSDFRRENEGYYFCSALSNSIMYFSHFVP |
| | peptide) | VFLPADFLPTTAQPTKKSTLKKRVCRLPRPETQKGPL |
| | | CCPSPLFPGPSKP |
| 91 | CD8 co-receptor-α-β- | MALPVTALLLPLALLLHAARPSQFRVSPLDRTWNLG |
| | CD28 chimeric stalk | ETVELKCQVLLSNPTSGCSWLFQPRGAAASPTFLLYL |
| | (with signal peptide) | SQNKPKAAEGLDTQRFSGKRLGDTFVLTLSDFRREN |
| | | EGYYFCSALSNSIMYFSHFVPVFLPADFLPTTAQPTK |
| | | KSTLKKRVCRLPRPETQKGPLCCPSPLFPGPSKP |
| 92 | CD8 co-receptor-α-β | SQFRVSPLDRTWNLGETVELKCQVLLSNPTSGCSWL |
| | chimeric stalk | FQPRGAAASPTFLLYLSQNKPKAAEGLDTQRFSGKR |
| | (without signal | LGDTFVLTLSDFRRENEGYYFCSALSNSIMYFSHFVP |
| | peptide) | VFLPADFLPTTAQPTKKSTLKKRVCRLPRPETQKGPL |
| | | C |
| 93 | CD8 co-receptor-α-β | MALPVTALLLPLALLLHAARPSQFRVSPLDRTWNLG |
| | chimeric stalk (with | ETVELKCQVLLSNPTSGCSWLFQPRGAAASPTFLLYL |
| | signal peptide) | SQNKPKAAEGLDTQRFSGKRLGDTFVLTLSDFRREN |
| | | EGYYFCSALSNSIMYFSHFVPVFLPADFLPTTAQPTK |
| | | KSTLKKRVCRLPRPETQKGPLC |
| 94 | CD8 co-receptor-α-β | SQFRVSPLDRTWNLGETVELKCQVLLSNPTSGCSWL |
| | chimeric stalk | FQPRGAAASPTFLLYLSQNKPKAAEGLDTQRFSGKR |
| | (without signal | LGDTFVLTLSDFRRENEGYYFCSALSNSIMYFSHFVP |
| | peptide) | VFLPADFLPTTAQPTKKSTLKKRVCRLPRPETQKGPL |
| | | CD |
| 95 | CD8 co-receptor-α-β | MALPVTALLLPLALLLHAARPSQFRVSPLDRTWNLG |
| | chimeric stalk (with | ETVELKCQVLLSNPTSGCSWLFQPRGAAASPTFLLYL |
| | signal peptide) | SQNKPKAAEGLDTQRFSGKRLGDTFVLTLSDFRREN |
| | | EGYYFCSALSNSIMYFSHFVPVFLPADFLPTTAQPTK |
| | | KSTLKKRVCRLPRPETQKGPLCD |
| 96 | hNKG2D_CD28 | MLRLLLALNLFPSIQVTGIPLTESYCGPCPKNWICYK |
| | | NNCYQFFDESKNWYESQASCMSQNASLLKVYSKED |
| | | QDLLKLVKSYHWMGLVHIPTNGSWQWEDGSILSPN |
| | | LLTIIEMQKGDCALYASSFKGYIENCSTPNTYICMQR |
| | | TVKGKHLCPSPLFPGPSKP |
| 97 | CD28_IC (GG | RSKRSRGGHSDYMNMTPRRPGPTRKHYQPYAPPRDF |
| | mutation)_CD8α_IC | AAYRSLYCNHRNRRRVCKCPRPVVKSGDKPSLSARY |
| | | V |
| 98 | Fas_EC (without | QVTDINSKGLELRKTVTTVETQNLEGLHHDGQFCHK |
| | signal peptide) | PCPPGERKARDCTVNGDEPDCVPCQEGKEYTDKAHF |
| | | SSKCRRCRLCDEGHGLEVEINCTRTQNTKCRCKPNFF |
| | | CNSTVCEHCDPCTKCEHGIIKECTLTSNTKCKEEGSR |
| | | SN |
| 99 | Fas_EC (with signal | MLGIWTLLPLVLTSVARLSSKSVNAQVTDINSKGLEL |
| | peptide) | RKTVTTVETQNLEGLHHDGQFCHKPCPPGERKARDC |
| | | TVNGDEPDCVPCQEGKEYTDKAHFSSKCRRCRLCDE |
| | | GHGLEVEINCTRTQNTKCRCKPNFFCNSTVCEHCDPC |
| | | TKCEHGIIKECTLTSNTKCKEEGSRSN |
| 100 | Fas_TM | LGWLCLLLLPIPLIVWV |
| 101 | Fas_IC truncated | K |
| 102 | Lck_IC | PLQDNLVIALHSYEPSHDGDLGFEKGEQLRILEQSGE |
| | | WWKAQSLTTGQEGFIPFNFVAKANSLEPEPWFFKNL |
| | | SRKDAERQLLAPGNTHGSFLIRESESTAGSFSLSVRDF |
| | | DQNQGEVVKHYKIRNLDNGGFYISPRITFPGLHELVR |
| | | HYTNASDGLCTRLSRPCQTQKPQKPWWEDEWEVPR |
| | | ETLKLVERLGAGQFGEVWMGYYNGHTKVAVKSLK |
| | | QGSMSPDAFLAEANLMKQLQHQRLVRLYAVVTQEPI |
| | | YIITEYMENGSLVDFLKTPSGIKLTINKLLDMAAQIAE |
| | | GMAFIEERNYIHRDLRAANILVSDTLSCKIADFGLAR |
| | | LIEDNEYTAREGAKFPIKWTAPEAINYGTFTIKSDVW |
| | | SFGILLTEIVTHGRIPYPGMTNPEVIQNLERGYRMVRP |
| | | DNCPEELYQLMRLCWKERPEDRPTFDYLRSVLEDFF |
| | | TATEGQYQPQP |
| 103 | G-S-T | GSGGGTGGGSGGSGGGTGGGSGMASSSGSSPRPAPD |
| | linker_TRAFI_IC | ENEFPFGCPPTVCQDPKEPRALCCAGCLSENPRNGED |
| | | QICPKCRGEDLQSISPGSRLRTQEKAHPEVAEAGIGCP |
| | | FAGVGCSFKGSPQSVQEHEVTSQTSHLNLLLGFMKQ |
| | | WKARLGCGLESGPMALEQNLSDLQLQAAVEVAGDL |
| | | EVDCYRAPCSESQEELALQHFMKEKLLAELEGKLRV |
| | | FENIVAVLNKEVEASHLALATSIHQSQLDRERILSLEQ |
| | | RVVELQQTLAQKDQALGKLEQSLRLMEEASFDGTFL |
| | | WKITNVTRRCHESACGRTVSLFSPAFYTAKYGYKLC |
| | | LRLYLNGDGTGKRTHLSLFIVIMRGEYDALLPWPFR |
| | | NKVTFMLLDQNNREHAIDAFRPDLSSASFQRPQSETN |
| | | VASGCPLFFPLSKLQSPKHAYVKDDTMFLKCIVETST |
| 104 | PD-1_EC_CD28_EC | FLDSPDRPWNPPTFSPALLVVTEGDNATFTCSFSNTSE |
| | fusion (without signal | SFVLNWYRMSPSNQTDKLAAFPEDRSQPGQDCRFRV |
| | peptide) | TQLPNGRDFHMSVVRARRNDSGTYLCGAISLAPKAQ |
| | | IKESLRAELRVTERRAEVPTAHPSPCPSPLFPGPSKP |
| 105 | PD-1_EC_CD28_EC | MQIPQAPWPVVWAVLQLGWRPGWFLDSPDRPWNPP |
| | fusion (with signal | TFSPALLVVTEGDNATFTCSFSNTSESFVLNWYRMSP |
| | peptide) | SNQTDKLAAFPEDRSQPGQDCRFRVTQLPNGRDFHM |
| | | SVVRARRNDSGTYLCGAISLAPKAQIKESLRAELRVT |
| | | ERRAEVPTAHPSPCPSPLFPGPSKP |
| 106 | CCR4 | MNPTDIADTTLDESIYSNYYLYESIPKPCTKEGIKAFG |
| | | ELFLPPLYSLVFVFGLLGNSVVVLVLFKYKRLRSMTD |
| | | VYLLNLAISDLLFVFSLPFWGYYAADQWVFGLGLCK |
| | | MISWMYLVGFYSGIFFVMLMSIDRYLAIVHAVFSLR |
| | | ARTLTYGVITSLATWSVAVFASLPGFLFSTCYTERNH |
| | | TYCKTKYSLNSTTWKVLSSLEINILGLVIPLGIMLFCY |
| | | SMIIRTLQHCKNEKKNKAVKMIFAVVVLFLGFWTPY |
| | | NIVLFLETLVELEVLQDCTFERYLDYAIQATETLAFV |
| | | HCCLNPIIYFFLGEKFRKYILQLFKTCRGLFVLCQYCG |
| | | LLQIYSADTPSSSYTQSTMDHDLHDAL |
| 107 | CCR2B | MLSTSRSRFIRNTNESGEEVTTFFDYDYGAPCHKFDV |
| | | KQIGAQLLPPLYSLVFIFGFVGNMLVVLILINCKKLKC |
| | | LTDIYLLNLAISDLLFLITLPLWAHSAANEWVFGNAM |
| | | CKLFTGLYHIGYFGGIFFIILLTIDRYLAIVHAVFALKA |
| | | RTVTFGVVTSVITWLVAVFASVPGIIFTKCQKEDSVY |
| | | VCGPYFPRGWNNFHTIMRNILGLVLPLLIMVICYSGIL |
| | | KTLLRCRNEKKRHRAVRVIFTIMIVYFLFWTPYNIVIL |
| | | LNTFQEFFGLSNCESTSQLDQATQVTETLGMTHCCIN |
| | | PIIYAFVGEKFRRYLSVFFRKHITKRFCKQCPVFYRET |
| | | VDGVTSTNTPSTGEQEVSAGL |
| 108 | CD28_IC (GG | RSKRSRGGHSDAMNMTARRAGPTRKHFQAFAAPRD |
| | mutation, full | FAAFRS |
| | signaling mutation) | |
| 109 | CD8 co-receptor | ATGGCTCTGCCTGTGACAGCTCTGCTGCTGCCTCTG |
| | α_EC (nt_1) | GCTCTGCTTCTGCATGCCGCTAGACCCAGCCAGTT |
| | | CAGAGTGTCCCCTCTGGACAGAACCTGGAACCTGG |
| | | GCGAGACAGTGGAACTGAAGTGCCAGGTGCTGCT |
| | | GAGCAATCCTACCAGCGGCTGCAGCTGGCTGTTTC |
| | | AGCCTAGAGGTGCTGCCGCCTCTCCTACCTTTCTGC |
| | | TGTACCTGAGCCAGAACAAGCCCAAGGCCGCCGA |
| | | AGGACTGGACACCCAGAGATTCAGCGGCAAGAGA |
| | | CTGGGCGACACCTTCGTGCTGACCCTGAGCGACTT |
| | | CAGAAGAGAGAACGAGGGCTACTACTTCTGCAGC |
| | | GCCCTGAGCAACAGCATCATGTACTTCAGCCACTT |
| | | CGTGCCCGTGTTTCTGCCCGCCAAGCCTACAACAA |
| | | CCCCTGCTCCTAGACCTCCTACACCAGCTCCTACA |
| | | ATCGCCAGCCAGCCTCTGTCTCTGAGGCCAGAAGC |
| | | TTGTAGACCTGCTGCTGGCGGAGCCGTGCATACAA |
| | | GAGGACTGGATTTCGCCTGCGAC |
| 110 | CD8 co-receptor | ATGGCATTGCCTGTTACAGCTCTGCTGCTGCCCCTG |
| | α_EC (nt_2) | GCTCTGCTTCTGCATGCTGCTAGACCCAGCCAGTT |
| | | CAGAGTGTCCCCTCTGGACAGAACCTGGAACCTGG |
| | | GCGAGACAGTGGAACTGAAGTGCCAGGTGCTGCT |
| | | GAGCAATCCTACCAGCGGCTGCAGCTGGCTGTTTC |
| | | AGCCTAGAGGTGCTGCCGCCTCTCCTACCTTTCTGC |
| | | TGTACCTGAGCCAGAACAAGCCCAAGGCCGCCGA |
| | | AGGACTGGACACCCAGAGATTCAGCGGCAAGAGA |
| | | CTGGGCGACACCTTCGTGCTGACCCTGAGCGACTT |
| | | CAGAAGAGAGAACGAGGGCTACTACTTCTGCAGC |
| | | GCCCTGAGCAACAGCATCATGTACTTCAGCCACTT |
| | | CGTGCCCGTGTTTCTGCCCGCCAAGCCTACAACAA |
| | | CCCCTGCTCCTAGACCTCCTACACCAGCTCCTACA |
| | | ATCGCCAGCCAGCCTCTGTCTCTGAGGCCAGAAGC |
| | | TTGTAGACCTGCTGCTGGCGGAGCCGTGCATACAA |
| | | GAGGACTGGATTTCGCCTGCGAC |
| 111 | CD8 co-receptor | atggctcttccggtaactgcacttttactgccacttgctctgctgttacacgctgctaga |
| | α_EC (nt_3) | ccatctcagttccgtgtttctccactggatagaacttggaatctgggcgagactgttga |
| | | gctgaaatgccaggttctgctgtctaacccgacttctggttgttcttggctgtttcaacc |
| | | aagaggtgctgctgcttctccgacttttctgctgtacctgtctcagaacaaaccgaaa |
| | | gctgctgaaggtctggatactcagcgtttctccggtaaacgtctgggtgacacctttgt |
| | | tctgactctgtctgatttccgtcgcgaaaacgaaggctactacttctgctctgctctgtc |
| | | caactccatcatgtacttctcccactttgtgccggtttttctgccggctaaaccgactac |
| | | tactccagctccaagaccaccaactccagctccaactattgcttctcaaccactgtctt |
| | | tacgtccagaagcttgtcgtccagctgctggtggtgctgttcatactcgtggtttagatt |
| | | tcgcttgtgac |
| 112 | CD8 co-receptor | ATGGCTCTTCCTGTAACCGCACTTCTGCTTCCTCTT |
| | α_EC (nt_4) | GCTCTGCTGCTTCATGCTGCTAGACCTAGCCAGTTC |
| | | AGAGTGTCTCCACTGGATAGAACCTGGAATCTGGG |
| | | CGAAACAGTGGAGCTGAAGTGTCAGGTGCTGCTG |
| | | AGCAATCCTACATCTGGCTGTTCTTGGCTGTTCCAG |
| | | CCTAGAGGAGCTGCTGCTTCTCCTACCTTTCTGCTG |
| | | TATCTGAGCCAGAATAAGCCTAAAGCCGCCGAAG |
| | | GACTGGATACCCAGAGGTTTAGCGGCAAGAGATT |
| | | GGGCGATACCTTTGTTCTGACACTGAGCGACTTCC |
| | | GGAGAGAGAATGAGGGCTACTACTTCTGTTCTGCC |
| | | CTGAGCAACAGCATCATGTACTTCAGCCACTTCGT |
| | | GCCCGTGTTTCTGCCTGCC |
| 113 | CD8b including 6 | MRPRLWLLLAAQLTVLHGNSVLQQTPAYIKVQTNK |
| | AA of IC region with | MVMLSCEAKISLSNMRIYWLRQRQAPSSDSHHEFLA |
| | CD28 intracellular | LWDSAKGTIHGEEVEQEKIAVFRDASRFILNLTSVKP |
| | region (GG | EDSGIYFCMIVGSPELTFGKGTQLSVVDFLPTTAQPT |
| | mutation), with signal | KKSTLKKRVCRLPRPETQKGPLCSPITLGLLVAGVLV |
| | peptide | LLVSLGVAIHLCCRRRSKRSRGGHSDYMNMTPRRPG |
| | | PTRKHYQPYAPPRDFAAYRS |
| 114 | CD8a chain, without | SQFRVSPLDRTWNLGETVELKCQVLLSNPTSGCSWL |
| | signal peptide | FQPRGAAASPTFLLYLSQNKPKAAEGLDTQRFSGKR |
| | | LGDTFVLTLSDFRRENEGYYFCSALSNSIMYFSHFVP |
| | | VFLPAKPTTTPAPRPPTPAPTIASQPLSLRPEACRPAA |
| | | GGAVHTRGLDFACDIYIWAPLAGTCGVLLLSLVITLY |
| | | CNHRNRRRVCKCPRPVVKSGDKPSLSARYV |
| 115 | CD8b maintaining 6 | LQQTPAYIKVQTNKMVMLSCEAKISLSNMRIYWLRQ |
| | AA of IC region with | RQAPSSDSHHEFLALWDSAKGTIHGEEVEQEKIAVFR |
| | CD28 intracellular | DASRFILNLTSVKPEDSGIYFCMIVGSPELTFGKGTQL |
| | region (GG | SVVDFLPTTAQPTKKSTLKKRVCRLPRPETQKGPLCS |
| | mutation), without | PITLGLLVAGVLVLLVSLGVAIHLCCRRRSKRSRGGH |
| | signal peptide | SDYMNMTPRRPGPTRKHYQPYAPPRDFAAYRS |
| 116 | CD8 beta with CD28 | MRPRLWLLLAAQLTVLHGNSVLQQTPAYIKVQTNK |
| | intracellular region | MVMLSCEAKISLSNMRIYWLRQRQAPSSDSHHEFLA |
| | (GG mutation), with | LWDSAKGTIHGEEVEQEKIAVFRDASRFILNLTSVKP |
| | signal peptide | EDSGIYFCMIVGSPELTFGKGTQLSVVDFLPTTAQPT |
| | | KKSTLKKRVCRLPRPETQKGPLCSPITLGLLVAGVLV |
| | | LLVSLGVAIRSKRSRGGHSDYMNMTPRRPGPTRKHY |
| | | QPYAPPRDFAAYRS |
| 117 | CD8 beta with CD28 | LQQTPAYIKVQTNKMVMLSCEAKISLSNMRIYWLRQ |
| | intracellular region | RQAPSSDSHHEFLALWDSAKGTIHGEEVEQEKIAVFR |
| | (GG mutation), | DASRFILNLTSVKPEDSGIYFCMIVGSPELTFGKGTQL |
| | without signal | SVVDFLPTTAQPTKKSTLKKRVCRLPRPETQKGPLCS |
| | peptide | PITLGLLVAGVLVLLVSLGVAIRSKRSRGGHSDYMN |
| | | MTPRRPGPTRKHYQPYAPPRDFAAYRS |
| 118 | CD8a with CD28 | MALPVTALLLPLALLLHAARPSQFRVSPLDRTWNLG |
| | intracellular region | ETVELKCQVLLSNPTSGCSWLFQPRGAAASPTFLLYL |
| | (GG mutation), with | SQNKPKAAEGLDTQRFSGKRLGDTFVLTLSDFRREN |
| | signal peptide | EGYYFCSALSNSIMYFSHFVPVFLPAKPTTTPAPRPPT |
| | | PAPTIASQPLSLRPEACRPAAGGAVHTRGLDFACDIYI |
| | | WAPLAGTCGVLLLSLVITRSKRSRGGHSDYMNMTPR |
| | | RPGPTRKHYQPYAPPRDFAAYRS |
| 119 | CD8 beta isoform 1, | LQQTPAYIKVQTNKMVMLSCEAKISLSNMRIYWLRQ |
| | without signal | RQAPSSDSHHEFLALWDSAKGTIHGEEVEQEKIAVFR |
| | peptide | DASRFILNLTSVKPEDSGIYFCMIVGSPELTFGKGTQL |
| | | SVVDFLPTTAQPTKKSTLKKRVCRLPRPETQKGPLCS |
| | | PITLGLLVAGVLVLLVSLGVAIHLCCRRRRARLRFMK |
| | | QFYK |
| 120 | CD8b with CD28 | MRPRLWLLLAAQLTVLHGNSVLQQTPAYIKVQTNK |
| | intracellular region | MVMLSCEAKISLSNMRIYWLRQRQAPSSDSHHEFLA |
| | (GG mutation + | LWDSAKGTIHGEEVEQEKIAVFRDASRFILNLTSVKP |
| | partial signaling | EDSGIYFCMIVGSPELTFGKGTQLSVVDFLPTTAQPT |
| | mutation), with signal | KKSTLKKRVCRLPRPETQKGPLCSPITLGLLVAGVLV |
| | peptide | LLVSLGVAIHLCCRRRSKRSRGGHSDAMNMTARRA |
| | | GPTRKHYQAYAAPRDFAAYRS |
| 121 | CD8b with CD28 | LQQTPAYIKVQTNKMVMLSCEAKISLSNMRIYWLRQ |
| | intracellular region | RQAPSSDSHHEFLALWDSAKGTIHGEEVEQEKIAVFR |
| | (GG mutation + | DASRFILNLTSVKPEDSGIYFCMIVGSPELTFGKGTQL |
| | partial signaling | SVVDFLPTTAQPTKKSTLKKRVCRLPRPETQKGPLCS |
| | mutation), without | PITLGLLVAGVLVLLVSLGVAIHLCCRRRSKRSRGGH |
| | signal peptide | SDAMNMTARRAGPTRKHYQAYAAPRDFAAYRS |
| 122 | CD8a with CD28 | MALPVTALLLPLALLLHAARPSQFRVSPLDRTWNLG |
| | intracellular region | ETVELKCQVLLSNPTSGCSWLFQPRGAAASPTFLLYL |
| | (GG mutation + | SQNKPKAAEGLDTQRFSGKRLGDTFVLTLSDFRREN |
| | partial signaling | EGYYFCSALSNSIMYFSHFVPVFLPAKPTTTPAPRPPT |
| | mutation), with signal | PAPTIASQPLSLRPEACRPAAGGAVHTRGLDFACDIYI |
| | peptide | WAPLAGTCGVLLLSLVITRSKRSRGGHSDAMNMTA |
| | | RRAGPTRKHYQAYAAPRDFAAYRS |
| 123 | CD8b with CD28 | MRPRLWLLLAAQLTVLHGNSVLQQTPAYIKVQTNK |
| | intracellular region | MVMLSCEAKISLSNMRIYWLRQRQAPSSDSHHEFLA |
| | (GG mutation + | LWDSAKGTIHGEEVEQEKIA VFRDASRFILNLTSVKP |
| | partial signaling | EDSGIYFCMIVGSPELTFGKGTQLSVVDFLPTTAQPT |
| | mutation), with signal | KKSTLKKRVCRLPRPETQKGPLCSPITLGLLVAGVLV |
| | peptide | LLVSLGVAIRSKRSRGGHSDAMNMTARRAGPTRKH |
| | | YQAYAAPRDFAAYRS |
| 124 | CD8a with CD28 | SQFRVSPLDRTWNLGETVELKCQVLLSNPTSGCSWL |
| | intracellular region | FQPRGAAASPTFLLYLSQNKPKAAEGLDTQRFSGKR |
| | (GG mutation + | LGDTFVLTLSDFRRENEGYYFCSALSNSIMYFSHFVP |
| | partial signaling | VFLPAKPTTTPAPRPPTPAPTIASQPLSLRPEACRPAA |
| | mutation), without | GGAVHTRGLDFACDIYIWAPLAGTCGVLLLSLVITRS |
| | signal peptide | KRSRGGHSDAMNMTARRAGPTRKHYQAYAAPRDF |
| | | AAYRS |
| 125 | CD8b with CD28 | LQQTPAYIKVQTNKMVMLSCEAKISLSNMRIYWLRQ |
| | intracellular region | RQAPSSDSHHEFLALWDSAKGTIHGEEVEQEKIAVFR |
| | (GG mutation + | DASRFILNLTSVKPEDSGIYFCMIVGSPELTFGKGTQL |
| | partial signaling | SVVDFLPTTAQPTKKSTLKKRVCRLPRPETQKGPLCS |
| | mutation), without | PITLGLLVAGVLVLLVSLGVAIRSKRSRGGHSDAMN |
| | signal peptide | MTARRAGPTRKHYQAYAAPRDFAAYRS |
| 126 | CD8b maintaining 6 | MRPRLWLLLAAQLTVLHGNSVLQQTPAYIKVQTNK |
| | AA of IC region with | MVMLSCEAKISLSNMRIYWLRQRQAPSSDSHHEFLA |
| | CD28 intracellular | LWDSAKGTIHGEEVEQEKIAVFRDASRFILNLTSVKP |
| | region (wt LL), with | EDSGIYFCMIVGSPELTFGKGTQLSVVDFLPTTAQPT |
| | signal peptide | KKSTLKKRVCRLPRPETQKGPLCSPITLGLLVAGVLV |
| | | LLVSLGVAIHLCCRRRSKRSRLLHSDYMNMTPRRPG |
| | | PTRKHYQPYAPPRDFAAYRS |
| 127 | CD8b maintaining 6 | LQQTPAYIKVQTNKMVMLSCEAKISLSNMRIYWLRQ |
| | AA of IC region with | RQAPSSDSHHEFLALWDSAKGTIHGEEVEQEKIAVFR |
| | CD28 intracellular | DASRFILNLTSVKPEDSGIYFCMIVGSPELTFGKGTQL |
| | region (wt LL), | SVVDFLPTTAQPTKKSTLKKRVCRLPRPETQKGPLCS |
| | without signal | PITLGLLVAGVLVLLVSLGVAIHLCCRRRSKRSRLLH |
| | peptide | SDYMNMTPRRPGPTRKHYQPYAPPRDFAAYRS |
| 128 | CD8b with 41BB | MRPRLWLLLAAQLTVLHGNSVLQQTPAYIKVQTNK |
| | intracellular region, | MVMLSCEAKISLSNMRIYWLRQRQAPSSDSHHEFLA |
| | with signal peptide | LWDSAKGTIHGEEVEQEKIAVFRDASRFILNLTSVKP |
| | | EDSGIYFCMIVGSPELTFGKGTQLSVVDFLPTTAQPT |
| | | KKSTLKKRVCRLPRPETQKGPLCSPITLGLLVAGVLV |
| | | LLVSLGVAIHLCCRRKRGRKKLLYIFKQPFMRPVQTT |
| | | QEEDGCSCRFPEEEEGGCEL |
| 129 | CD8b with 41BB | LQQTPAYIKVQTNKMVMLSCEAKISLSNMRIYWLRQ |
| | intracellular region, | RQAPSSDSHHEFLALWDSAKGTIHGEEVEQEKIAVFR |
| | with signal peptide | DASRFILNLTSVKPEDSGIYFCMIVGSPELTFGKGTQL |
| | | SVVDFLPTTAQPTKKSTLKKRVCRLPRPETQKGPLCS |
| | | PITLGLLVAGVLVLLVSLGVAIHLCCRRKRGRKKLLY |
| | | IFKQPFMRPVQTTQEEDGCSCRFPEEEEGGCEL |
| 130 | CD8b with ICOS | MRPRLWLLLAAQLTVLHGNSVLQQTPAYIKVQTNK |
| | intracellular region, | MVMLSCEAKISLSNMRIYWLRQRQAPSSDSHHEFLA |
| | with signal peptide | LWDSAKGTIHGEEVEQEKIAVFRDASRFILNLTSVKP |
| | | EDSGIYFCMIVGSPELTFGKGTQLSVVDFLPTTAQPT |
| | | KKSTLKKRVCRLPRPETQKGPLCSPITLGLLVAGVLV |
| | | LLVSLGVAIHLCCRRCWLTKKKYSSSVHDPNGEYMF |
| | | MRAVNTAKKSRLTDVTL |
| 131 | CD8b with ICOS | LQQTPAYIKVQTNKMVMLSCEAKISLSNMRIYWLRQ |
| | intracellular region, | RQAPSSDSHHEFLALWDSAKGTIHGEEVEQEKIAVFR |
| | without signal | DASRFILNLTSVKPEDSGIYFCMIVGSPELTFGKGTQL |
| | peptide | SVVDFLPTTAQPTKKSTLKKRVCRLPRPETQKGPLCS |
| | | PITLGLLVAGVLVLLVSLGVAIHLCCRRCWLTKKKY |
| | | SSSVHDPNGEYMFMRAVNTAKKSRLTDVTL |
| 132 | CD8b with OX40 | MRPRLWLLLAAQLTVLHGNSVLQQTPAYIKVQTNK |
| | intracellular region, | MVMLSCEAKISLSNMRIYWLRQRQAPSSDSHHEFLA |
| | with signal peptide | LWDSAKGTIHGEEVEQEKIAVFRDASRFILNLTSVKP |
| | | EDSGIYFCMIVGSPELTFGKGTQLSVVDFLPTTAQPT |
| | | KKSTLKKRVCRLPRPETQKGPLCSPITLGLLVAGVLV |
| | | LLVSLGVAIHLCCRRALYLLRRDQRLPPDAHKPPGG |
| | | GSFRTPIQEEQADAHSTLAKI |
| 133 | CD8b with OX40 | LQQTPAYIKVQTNKMVMLSCEAKISLSNMRIYWLRQ |
| | intracellular region, | RQAPSSDSHHEFLALWDSAKGTIHGEEVEQEKIAVFR |
| | without signal | DASRFILNLTSVKPEDSGIYFCMIVGSPELTFGKGTQL |
| | peptide | SVVDFLPTTAQPTKKSTLKKRVCRLPRPETQKGPLCS |
| | | PITLGLLVAGVLVLLVSLGVAIHLCCRRALYLLRRDQ |
| | | RLPPDAHKPPGGGSFRTPIQEEQADAHSTLAKI |
| 134 | CD8b with GITR | MRPRLWLLLAAQLTVLHGNSVLQQTPAYIKVQTNK |
| | intracellular region, | MVMLSCEAKISLSNMRIYWLRQRQAPSSDSHHEFLA |
| | with signal peptide | LWDSAKGTIHGEEVEQEKIAVFRDASRFILNLTSVKP |
| | | EDSGIYFCMIVGSPELTFGKGTQLSVVDFLPTTAQPT |
| | | KKSTLKKRVCRLPRPETQKGPLCSPITLGLLVAGVLV |
| | | LLVSLGVAIHLCCRRQLGLHIWQLRSQCMWPRETQL |
| | | LLEVPPSTEDARSCQFPEEERGERSAEEKGRLGDLWV |
| 135 | CD8b with GITR | LQQTPAYIKVQTNKMVMLSCEAKISLSNMRIYWLRQ |
| | intracellular region, | RQAPSSDSHHEFLALWDSAKGTIHGEEVEQEKIAVFR |
| | without signal | DASRFILNLTSVKPEDSGIYFCMIVGSPELTFGKGTQL |
| | peptide | SVVDFLPTTAQPTKKSTLKKRVCRLPRPETQKGPLCS |
| | | PITLGLLVAGVLVLLVSLGVAIHLCCRRQLGLHIWQL |
| | | RSQCMWPRETQLLLEVPPSTEDARSCQFPEEERGERS |
| | | AEEKGRLGDLWV |
| 136 | CD8a ex. cell-CD28 | MALPVTALLLPLALLLHAARPSQFRVSPLDRTWNLG |
| | transmembrane with | ETVELKCQVLLSNPTSGCSWLFQPRGAAASPTFLLYL |
| | CD28 intracellular | SQNKPKAAEGLDTQRFSGKRLGDTFVLTLSDFRREN |
| | region (GG | EGYYFCSALSNSIMYFSHFVPVFLPAFWVLVVVGGV |
| | mutation), with signal | LACYSLLVTVAFIIFWVRSKRSRGGHSDYMNMTPRR |
| | peptide | PGPTRKHYQPYAPPRDFAAYRS |
| 137 | CD8b ex/tm/ic with | MRPRLWLLLAAQLTVLHGNSVLQQTPAYIKVQTNK |
| | 47-496 of Lck | MVMLSCEAKISLSNMRIYWLRQRQAPSSDSHHEFLA |
| | intracellular region, | LWDSAKGTIHGEEVEQEKIAVFRDASRFILNLTSVKP |
| | with signal peptide | EDSGIYFCMIVGSPELTFGKGTQLSVVDFLPTTAQPT |
| | | KKSTLKKRVCRLPRPETQKGPLCSPITLGLLVAGVLV |
| | | LLVSLGVAIHLCCRRRRARLRFMKQFYKPLQDNLVI |
| | | ALHSYEPSHDGDLGFEKGEQLRILEQSGEWWKAQSL |
| | | TTGQEGFIPFNFVAKANSLEPEPWFFKNLSRKDAERQ |
| | | LLAPGNTHGSFLIRESESTAGSFSLSVRDFDQNQGEV |
| | | VKHYKIRNLDNGGFYISPRITFPGLHELVRHYTNASD |
| | | GLCTRLSRPCQTQKPQKPWWEDEWEVPRETLKLVE |
| | | RLGAGQFGEVWMGYYNGHTKVAVKSLKQGSMSPD |
| | | AFLAEANLMKQLQHQRLVRLYAVVTQEPIYIITEYM |
| | | ENGSLVDFLKTPSGIKLTINKLLDMAAQIAEGMAFIE |
| | | ERNYIHRDLRAANILVSDTLSCKIADFGLARLIEDNEY |
| | | TAREGAKFPIKWTAPEAINYGTFTIKSDVWSFGILLTE |
| | | IVTHGRIPYPGMTNPEVIQNLERGYRMVRPDNCPEEL |
| | | YQLMRLCWKERPEDRPTFDYLRSVLEDFFTATEGQY |
| | | QPQP |
| 138 | CD8a ex. cell-CD28 | SQFRVSPLDRTWNLGETVELKCQVLLSNPTSGCSWL |
| | transmembrane with | FQPRGAAASPTFLLYLSQNKPKAAEGLDTQRFSGKR |
| | CD28 intracellular | LGDTFVLTLSDFRRENEGYYFCSALSNSIMYFSHFVP |
| | region (GG | VFLPAFWVLVVVGGVLACYSLLVTVAFIIFWVRSKR |
| | mutation), without | SRGGHSDYMNMTPRRPGPTRKHYQPYAPPRDFAAY |
| | signal peptide | RS |
| 139 | CD8b ex/tm/ic with | LQQTPAYIKVQTNKMVMLSCEAKISLSNMRIYWLRQ |
| | 47-496 of Lck | RQAPSSDSHHEFLALWDSAKGTIHGEEVEQEKIAVFR |
| | intracellular region, | DASRFILNLTSVKPEDSGIYFCMIVGSPELTFGKGTQL |
| | without signal | SVVDFLPTTAQPTKKSTLKKRVCRLPRPETQKGPLCS |
| | peptide | PITLGLLVAGVLVLLVSLGVAIHLCCRRRRARLRFMK |
| | | QFYKPLQDNLVIALHSYEPSHDGDLGFEKGEQLRILE |
| | | QSGEWWKAQSLTTGQEGFIPFNFVAKANSLEPEPWF |
| | | FKNLSRKDAERQLLAPGNTHGSFLIRESESTAGSFSLS |
| | | VRDFDQNQGEVVKHYKIRNLDNGGFYISPRITFPGLH |
| | | ELVRHYTNASDGLCTRLSRPCQTQKPQKPWWEDEW |
| | | EVPRETLKLVERLGAGQFGEVWMGYYNGHTKVAV |
| | | KSLKQGSMSPDAFLAEANLMKQLQHQRLVRLYAVV |
| | | TQEPIYIITEYMENGSLVDFLKTPSGIKLTINKLLDMA |
| | | AQIAEGMAFIEERNYIHRDLRAANILVSDTLSCKIADF |
| | | GLARLIEDNEYTAREGAKFPIKWTAPEAINYGTFTIKS |
| | | DVWSFGILLTEIVTHGRIPYPGMTNPEVIQNLERGYR |
| | | MVRPDNCPEELYQLMRLCWKERPEDRPTFDYLRSVL |
| | | EDFFTATEGQYQPQP |
| 140 | CD8a/CD8b | MALPVTALLLPLALLLHAARPSQFRVSPLDRTWNLG |
| | stalk/CD28 up to | ETVELKCQVLLSNPTSGCSWLFQPRGAAASPTFLLYL |
| | cysteine in | SQNKPKAAEGLDTQRFSGKRLGDTFVLTLSDFRREN |
| | extracellular region + | EGYYFCSALSNSIMYFSHFVPVFLPADFLPTTAQPTK |
| | CD28 transmembrane | KSTLKKRVCRLPRPETQKGPLCCPSPLFPGPSKPFWV |
| | with CD28 | LVVVGGVLACYSLLVTVAFIIFWVRSKRSRGGHSDY |
| | intracellular region | MNMTPRRPGPTRKHYQPYAPPRDFAAYRS |
| | (GG mutation), with | |
| | signal peptide | |
| 141 | CD8a/CD8b | SQFRVSPLDRTWNLGETVELKCQVLLSNPTSGCSWL |
| | stalk/CD28 up to | FQPRGAAASPTFLLYLSQNKPKAAEGLDTQRFSGKR |
| | cysteine in | LGDTFVLTLSDFRRENEGYYFCSALSNSIMYFSHFVP |
| | extracellular region + | VFLPADFLPTTAQPTKKSTLKKRVCRLPRPETQKGPL |
| | CD28 transmembrane | CCPSPLFPGPSKPFWVLVVVGGVLACYSLLVTVAFIIF |
| | with CD28 | WVRSKRSRGGHSDYMNMTPRRPGPTRKHYQPYAPP |
| | intracellular region | RDFAAYRS |
| | (GG mutation), | |
| | without signal | |
| | peptide | |
| 142 | CD8a/CD8b stalk + | MALPVTALLLPLALLLHAARPSQFRVSPLDRTWNLG |
| | CD28 transmembrane | ETVELKCQVLLSNPTSGCSWLFQPRGAAASPTFLLYL |
| | with CD28 | SQNKPKAAEGLDTQRFSGKRLGDTFVLTLSDFRREN |
| | intracellular region | EGYYFCSALSNSIMYFSHFVPVFLPADFLPTTAQPTK |
| | (GG mutation), with | KSTLKKRVCRLPRPETQKGPLCFWVLVVVGGVLAC |
| | signal peptide | YSLLVTVAFIIFWVRSKRSRGGHSDYMNMTPRRPGP |
| | | TRKHYQPYAPPRDFAAYRS |
| 143 | CD8a/CD8b stalk + | SQFRVSPLDRTWNLGETVELKCQVLLSNPTSGCSWL |
| | CD28 transmembrane | FQPRGAAASPTFLLYLSQNKPKAAEGLDTQRFSGKR |
| | with CD28 | LGDTFVLTLSDFRRENEGYYFCSALSNSIMYFSHFVP |
| | intracellular region | VFLPADFLPTTAQPTKKSTLKKRVCRLPRPETQKGPL |
| | (GG mutation), | CFWVLVVVGGVLACYSLLVTVAFIIFWVRSKRSRGG |
| | without signal | HSDYMNMTPRRPGPTRKHYQPYAPPRDFAAYRS |
| | peptide | |
| 144 | CD8a/CD8b stalk + | MALPVTALLLPLALLLHAARPSQFRVSPLDRTWNLG |
| | CD28 transmembrane | ETVELKCQVLLSNPTSGCSWLFQPRGAAASPTFLLYL |
| | with CD28 | SQNKPKAAEGLDTQRFSGKRLGDTFVLTLSDFRREN |
| | intracellular region | EGYYFCSALSNSIMYFSHFVPVFLPADFLPTTAQPTK |
| | (GG mutation) fused | KSTLKKRVCRLPRPETQKGPLCFWVLVVVGGVLAC |
| | in front of CD8a | YSLLVTVAFIIFWVRSKRSRGGHSDYMNMTPRRPGP |
| | intracellular region, | TRKHYQPYAPPRDFAAYRSLYCNHRNRRRVCKCPRP |
| | with signal peptide | VVKSGDKPSLSARYV |
| 145 | CD8a/CD8b stalk + | SQFRVSPLDRTWNLGETVELKCQVLLSNPTSGCSWL |
| | CD28 transmembrane | FQPRGAAASPTFLLYLSQNKPKAAEGLDTQRFSGKR |
| | with CD28 | LGDTFVLTLSDFRRENEGYYFCSALSNSIMYFSHFVP |
| | intracellular region | VFLPADFLPTTAQPTKKSTLKKRVCRLPRPETQKGPL |
| | (GG mutation) fused | CFWVLVVVGGVLACYSLLVTVAFIIFWVRSKRSRGG |
| | in front of CD8a | HSDYMNMTPRRPGPTRKHYQPYAPPRDFAAYRSLY |
| | intracellular region, | CNHRNRRRVCKCPRPVVKSGDKPSLSARYV |
| | without signal | |
| | peptide | |
| 146 | CD8a/CD8b stalk + | MALPVTALLLPLALLLHAARPSQFRVSPLDRTWNLG |
| | CD8a transmembrane | ETVELKCQVLLSNPTSGCSWLFQPRGAAASPTFLLYL |
| | with CD8a | SQNKPKAAEGLDTQRFSGKRLGDTFVLTLSDFRREN |
| | intracellular region, | EGYYFCSALSNSIMYFSHFVPVFLPADFLPTTAQPTK |
| | with signal peptide | KSTLKKRVCRLPRPETQKGPLCDIYIWAPLAGTCGVL |
| | | LLSLVITLYCNHRNRRRVCKCPRPVVKSGDKPSLSAR |
| | | YV |
| 147 | CD8a/CD8b stalk + | SQFRVSPLDRTWNLGETVELKCQVLLSNPTSGCSWL |
| | CD8a transmembrane | FQPRGAAASPTFLLYLSQNKPKAAEGLDTQRFSGKR |
| | with CD8a | LGDTFVLTLSDFRRENEGYYFCSALSNSIMYFSHFVP |
| | intracellular region, | VFLPADFLPTTAQPTKKSTLKKRVCRLPRPETQKGPL |
| | without signal | CDIYIWAPLAGTCGVLLLSLVITLYCNHRNRRRVCKC |
| | peptide | PRPVVKSGDKPSLSARYV |
| 148 | NKG2D extracellular | MLRLLLALNLFPSIQVTGIPLTESYCGPCPKNWICYK |
| | and transmembrane | NNCYQFFDESKNWYESQASCMSQNASLLKVYSKED |
| | sequence with CD28 | QDLLKLVKSYHWMGLVHIPTNGSWQWEDGSILSPN |
| | intracellular region | LLTIIEMQKGDCALYASSFKGYIENCSTPNTYICMQR |
| | (wt), with signal | TVKGKHLCPSPLFPGPSKPFWVLVVVGGVLACYSLL |
| | peptide | VTVAFIIFWVRSKRSRLLHSDYMNMTPRRPGPTRKH |
| | | YQPYAPPRDFAAYRS |
| 149 | NKG2D extracellular | IPLTESYCGPCPKNWICYKNNCYQFFDESKNWYESQ |
| | and transmembrane | ASCMSQNASLLKVYSKEDQDLLKLVKSYHWMGLV |
| | sequence with CD28 | HIPTNGSWQWEDGSILSPNLLTIIEMQKGDCALYASS |
| | intracellular region | FKGYIENCSTPNTYICMQRTVKGKHLCPSPLFPGPSKP |
| | (wt), without signal | FWVLVVVGGVLACYSLLVTVAFIIFWVRSKRSRLLH |
| | peptide | SDYMNMTPRRPGPTRKHYQPYAPPRDFAAYRS |
| 150 | CD8a with CD28 | MALPVTALLLPLALLLHAARPSQFRVSPLDRTWNLG |
| | intracellular region | ETVELKCQVLLSNPTSGCSWLFQPRGAAASPTFLLYL |
| | (GG mutation + full | SQNKPKAAEGLDTQRFSGKRLGDTFVLTLSDFRREN |
| | signaling mutation), | EGYYFCSALSNSIMYFSHFVPVFLPAKPTTTPAPRPPT |
| | with signal peptide | PAPTIASQPLSLRPEACRPAAGGAVHTRGLDFACDIYI |
| | | WAPLAGTCGVLLLSLVITRSKRSRGGHSDAMNMTA |
| | | RRAGPTRKHFQAFAAPRDFAAFRS |
| 151 | CD8b with CD28 | MRPRLWLLLAAQLTVLHGNSVLQQTPAYIKVQTNK |
| | intracellular region | MVMLSCEAKISLSNMRIYWLRQRQAPSSDSHHEFLA |
| | (GG mutation + full | LWDSAKGTIHGEEVEQEKIAVFRDASRFILNLTSVKP |
| | signaling mutation), | EDSGIYFCMIVGSPELTFGKGTQLSVVDFLPTTAQPT |
| | with signal peptide | KKSTLKKRVCRLPRPETQKGPLCSPITLGLLVAGVLV |
| | | LLVSLGVAIRSKRSRGGHSDAMNMTARRAGPTRKHF |
| | | QAFAAPRDFAAFRS |
| 152 | CD8a with CD28 | SQFRVSPLDRTWNLGETVELKCQVLLSNPTSGCSWL |
| | intracellular region | FQPRGAAASPTFLLYLSQNKPKAAEGLDTQRFSGKR |
| | (GG mutation + full | LGDTFVLTLSDFRRENEGYYFCSALSNSIMYFSHFVP |
| | signaling mutation), | VFLPAKPTTTPAPRPPTPAPTIASQPLSLRPEACRPAA |
| | without signal | GGAVHTRGLDFACDIYIWAPLAGTCGVLLLSLVITRS |
| | peptide | KRSRGGHSDAMNMTARRAGPTRKHFQAFAAPRDFA |
| | | AFRS |
| 153 | CD8b with CD28 | LQQTPAYIKVQTNKMVMLSCEAKISLSNMRIYWLRQ |
| | intracellular region | RQAPSSDSHHEFLALWDSAKGTIHGEEVEQEKIAVFR |
| | (GG mutation + full | DASRFILNLTSVKPEDSGIYFCMIVGSPELTFGKGTQL |
| | signaling mutation), | SVVDFLPTTAQPTKKSTLKKRVCRLPRPETQKGPLCS |
| | without signal | PITLGLLVAGVLVLLVSLGVAIRSKRSRGGHSDAMN |
| | peptide | MTARRAGPTRKHFQAFAAPRDFAAFRS |
| 154 | Fas extracellular + | MLGIWTLLPLVLTSVARLSSKSVNAQVTDINSKGLEL |
| | Fas Tm truncated no | RKTVTTVETQNLEGLHHDGQFCHKPCPPGERKARDC |
| | signaling domain, | TVNGDEPDCVPCQEGKEYTDKAHFSSKCRRCRLCDE |
| | with signal peptide | GHGLEVEINCTRTQNTKCRCKPNFFCNSTVCEHCDPC |
| | | TKCEHGIIKECTLTSNTKCKEEGSRSNLGWLCLLLLPI |
| | | PLIVWVK |
| 155 | Fas extracellular + | QVTDINSKGLELRKTVTTVETQNLEGLHHDGQFCHK |
| | Fas Tm truncated no | PCPPGERKARDCTVNGDEPDCVPCQEGKEYTDKAHF |
| | signaling domain, | SSKCRRCRLCDEGHGLEVEINCTRTQNTKCRCKPNFF |
| | without signal | CNSTVCEHCDPCTKCEHGIIKECTLTSNTKCKEEGSR |
| | peptide | SNLGWLCLLLLPIPLIVWVK |
| 156 | Fas extracellular + | MLGIWTLLPLVLTSVARLSSKSVNAQVTDINSKGLEL |
| | FasTM + lck | RKTVTTVETQNLEGLHHDGQFCHKPCPPGERKARDC |
| | intracellular (47-496), | TVNGDEPDCVPCQEGKEYTDKAHFSSKCRRCRLCDE |
| | with signal peptide | GHGLEVEINCTRTQNTKCRCKPNFFCNSTVCEHCDPC |
| | | TKCEHGIIKECTLTSNTKCKEEGSRSNLGWLCLLLLPI |
| | | PLIVWVPLQDNLVIALHSYEPSHDGDLGFEKGEQLRI |
| | | LEQSGEWWKAQSLTTGQEGFIPFNFVAKANSLEPEP |
| | | WFFKNLSRKDAERQLLAPGNTHGSFLIRESESTAGSF |
| | | SLSVRDFDQNQGEVVKHYKIRNLDNGGFYISPRITFP |
| | | GLHELVRHYTNASDGLCTRLSRPCQTQKPQKPWWE |
| | | DEWEVPRETLKLVERLGAGQFGEVWMGYYNGHTK |
| | | VAVKSLKQGSMSPDAFLAEANLMKQLQHQRLVRLY |
| | | AVVTQEPIYIITEYMENGSLVDFLKTPSGIKLTINKLL |
| | | DMAAQIAEGMAFIEERNYIHRDLRAANILVSDTLSCK |
| | | IADFGLARLIEDNEYTAREGAKFPIKWTAPEAINYGT |
| | | FTIKSDVWSFGILLTEIVTHGRIPYPGMTNPEVIQNLE |
| | | RGYRMVRPDNCPEELYQLMRLCWKERPEDRPTFDY |
| | | LRSVLEDFFTATEGQYQPQP |
| 157 | Fas extracellular + | QVTDINSKGLELRKTVTTVETQNLEGLHHDGQFCHK |
| | FasTM + lck | PCPPGERKARDCTVNGDEPDCVPCQEGKEYTDKAHF |
| | intracellular (47-496), | SSKCRRCRLCDEGHGLEVEINCTRTQNTKCRCKPNFF |
| | without signal | CNSTVCEHCDPCTKCEHGIIKECTLTSNTKCKEEGSR |
| | peptide | SNLGWLCLLLLPIPLIVWVPLQDNLVIALHSYEPSHD |
| | | GDLGFEKGEQLRILEQSGEWWKAQSLTTGQEGFIPFN |
| | | FVAKANSLEPEPWFFKNLSRKDAERQLLAPGNTHGS |
| | | FLIRESESTAGSFSLSVRDFDQNQGEVVKHYKIRNLD |
| | | NGGFYISPRITFPGLHELVRHYTNASDGLCTRLSRPCQ |
| | | TQKPQKPWWEDEWEVPRETLKLVERLGAGQFGEV |
| | | WMGYYNGHTKVAVKSLKQGSMSPDAFLAEANLMK |
| | | QLQHQRLVRLYAVVTQEPIYIITEYMENGSLVDFLKT |
| | | PSGIKLTINKLLDMAAQIAEGMAFIEERNYIHRDLRA |
| | | ANILVSDTLSCKIADFGLARLIEDNEYTAREGAKFPIK |
| | | WTAPEAINYGTFTIKSDVWSFGILLTEIVTHGRIPYPG |
| | | MTNPEVIQNLERGYRMVRPDNCPEELYQLMRLCWK |
| | | ERPEDRPTFDYLRSVLEDFFTATEGQYQPQP |
| 158 | Fas extracellular/ | MLGIWTLLPLVLTSVARLSSKSVNAQVTDINSKGLEL |
| | TM + CD8a intracellular, | RKTVTTVETQNLEGLHHDGQFCHKPCPPGERKARDC |
| | with signal peptide | TVNGDEPDCVPCQEGKEYTDKAHFSSKCRRCRLCDE |
| | | GHGLEVEINCTRTQNTKCRCKPNFFCNSTVCEHCDPC |
| | | TKCEHGIIKECTLTSNTKCKEEGSRSNLGWLCLLLLPI |
| | | PLIVWVLYCNHRNRRRVCKCPRPVVKSGDKPSLSAR |
| | | YV |
| 159 | Fas extracellular/ | QVTDINSKGLELRKTVTTVETQNLEGLHHDGQFCHK |
| | TM + CD8a intracellular, | PCPPGERKARDCTVNGDEPDCVPCQEGKEYTDKAHF |
| | without signal | SSKCRRCRLCDEGHGLEVEINCTRTQNTKCRCKPNFF |
| | peptide | CNSTVCEHCDPCTKCEHGIIKECTLTSNTKCKEEGSR |
| | | SNLGWLCLLLLPIPLIVWVLYCNHRNRRRVCKCPRP |
| | | VVKSGDKPSLSARYV |
| 160 | Fas extracellular/ | MLGIWTLLPLVLTSVARLSSKSVNAQVTDINSKGLEL |
| | TM + linker (G/S/T 22 | RKTVTTVETQNLEGLHHDGQFCHKPCPPGERKARDC |
| | amino acid linker) + | TVNGDEPDCVPCQEGKEYTDKAHFSSKCRRCRLCDE |
| | TRAFI intracellular, | GHGLEVEINCTRTQNTKCRCKPNFFCNSTVCEHCDPC |
| | with signal peptide | TKCEHGIIKECTLTSNTKCKEEGSRSNLGWLCLLLLPI |
| | | PLIVWVGSGGGTGGGSGGSGGGTGGGSGMASSSGSS |
| | | PRPAPDENEFPFGCPPTVCQDPKEPRALCCAGCLSEN |
| | | PRNGEDQICPKCRGEDLQSISPGSRLRTQEKAHPEVA |
| | | EAGIGCPFAGVGCSFKGSPQSVQEHEVTSQTSHLNLL |
| | | LGFMKQWKARLGCGLESGPMALEQNLSDLQLQAAV |
| | | EVAGDLEVDCYRAPCSESQEELALQHFMKEKLLAEL |
| | | EGKLRVFENIVAVLNKEVEASHLALATSIHQSQLDRE |
| | | RILSLEQRVVELQQTLAQKDQALGKLEQSLRLMEEA |
| | | SFDGTFLWKITNVTRRCHESACGRTVSLFSPAFYTAK |
| | | YGYKLCLRLYLNGDGTGKRTHLSLFIVIMRGEYDAL |
| | | LPWPFRNKVTFMLLDQNNREHAIDAFRPDLSSASFQ |
| | | RPQSETNVASGCPLFFPLSKLQSPKHAYVKDDTMFL |
| | | KCIVETST |
| 161 | Fas extracellular/ | QVTDINSKGLELRKTVTTVETQNLEGLHHDGQFCHK |
| | TM + linker (G/S/T 22 | PCPPGERKARDCTVNGDEPDCVPCQEGKEYTDKAHF |
| | amino acid linker) + | SSKCRRCRLCDEGHGLEVEINCTRTQNTKCRCKPNFF |
| | TRAFI intracellular, | CNSTVCEHCDPCTKCEHGIIKECTLTSNTKCKEEGSR |
| | without signal | SNLGWLCLLLLPIPLIVWVGSGGGTGGGSGGSGGGT |
| | peptide | GGGSGMASSSGSSPRPAPDENEFPFGCPPTVCQDPKE |
| | | PRALCCAGCLSENPRNGEDQICPKCRGEDLQSISPGS |
| | | RLRTQEKAHPEVAEAGIGCPFAGVGCSFKGSPQSVQE |
| | | HEVTSQTSHLNLLLGFMKQWKARLGCGLESGPMAL |
| | | EQNLSDLQLQAAVEVAGDLEVDCYRAPCSESQEELA |
| | | LQHFMKEKLLAELEGKLRVFENIVAVLNKEVEASHL |
| | | ALATSIHQSQLDRERILSLEQRVVELQQTLAQKDQAL |
| | | GKLEQSLRLMEEASFDGTFLWKITNVTRRCHESACG |
| | | RTVSLFSPAFYTAKYGYKLCLRLYLNGDGTGKRTHL |
| | | SLFIVIMRGEYDALLPWPFRNKVTFMLLDQNNREHAI |
| | | DAFRPDLSSASFQRPQSETNVASGCPLFFPLSKLQSPK |
| | | HAYVKDDTMFLKCIVETST |
| 162 | PD-1 extracellular + | MQIPQAPWPVVWAVLQLGWRPGWFLDSPDRPWNPP |
| | CD28 extra to | TFSPALLVVTEGDNATFTCSFSNTSESFVLNWYRMSP |
| | cysteine +CD28TM + | SNQTDKLAAFPEDRSQPGQDCRFRVTQLPNGRDFHM |
| | CD28 intracellular | SVVRARRNDSGTYLCGAISLAPKAQIKESLRAELRVT |
| | region (GG | ERRAEVPTAHPSPCPSPLFPGPSKPFWVLVVVGGVLA |
| | mutation), with signal | CYSLLVTVAFIIFWVRSKRSRGGHSDYMNMTPRRPG |
| | peptide | PTRKHYQPYAPPRDFAAYRS |
| 163 | PD-1 extracellular + | FLDSPDRPWNPPTFSPALLVVTEGDNATFTCSFSNTSE |
| | CD28 extracellular to | SFVLNWYRMSPSNQTDKLAAFPEDRSQPGQDCRFRV |
| | cysteine +CD28TM + | TQLPNGRDFHMSVVRARRNDSGTYLCGAISLAPKAQ |
| | CD28 intracellular | IKESLRAELRVTERRAEVPTAHPSPCPSPLFPGPSKPF |
| | region (GG | WVLVVVGGVLACYSLLVTVAFIIFWVRSKRSRGGHS |
| | mutation), without | DYMNMTPRRPGPTRKHYQPYAPPRDFAAYRS |
| | signal peptide | |
| 164 | CD3z-CD28IC- | MKWKALFTAAILQAQLPITEAQSFGLLDPKLCYLLD |
| | CD3zIC, with signal | GILFIYGVILTALFLRSKRSRGGHSDYMNMTPRRPGP |
| | peptide | TRKHYQPYAPPRDFAAYRSRVKFSRSADAPAYQQGQ |
| | | NQLYNELNLGRREEYDVLDKRRGRDPEMGGKPRRK |
| | | NPQEGLYNELQKDKMAEAYSEIGMKGERRRGKGHD |
| | | GLYQGLSTATKDTYDALHMQALPPR |
| 165 | CD3z-CD28IC- | QSFGLLDPKLCYLLDGILFIYGVILTALFLRSKRSRGG |
| | CD3zIC, without | HSDYMNMTPRRPGPTRKHYQPYAPPRDFAAYRSRV |
| | signal peptide | KFSRSADAPAYQQGQNQLYNELNLGRREEYDVLDK |
| | | RRGRDPEMGGKPRRKNPQEGLYNELQKDKMAEAYS |
| | | EIGMKGERRRGKGHDGLYQGLSTATKDTYDALHMQ |
| | | ALPPR |
| 166 | CD3z-41BBIC- | MKWKALFTAAILQAQLPITEAQSFGLLDPKLCYLLD |
| | CD3zIC, with signal | GILFIYGVILTALFLRGRKKLLYIFKQPFMRPVQTTQE |
| | peptide | EDGCSCRFPEEEEGGCELRVKFSRSADAPAYQQGQN |
| | | QLYNELNLGRREEYDVLDKRRGRDPEMGGKPRRKN |
| | | PQEGLYNELQKDKMAEAYSEIGMKGERRRGKGHDG |
| | | LYQGLSTATKDTYDALHMQALPPR |
| 167 | CD3z-41BBIC- | QSFGLLDPKLCYLLDGILFIYGVILTALFLRGRKKLLY |
| | CD3zIC, with signal | IFKQPFMRPVQTTQEEDGCSCRFPEEEEGGCELRVKF |
| | peptide | SRSADAPAYQQGQNQLYNELNLGRREEYDVLDKRR |
| | | GRDPEMGGKPRRKNPQEGLYNELQKDKMAEAYSEI |
| | | GMKGERRRGKGHDGLYQGLSTATKDTYDALHMQA |
| | | LPPR |
| 168 | CD8α signal peptide | MALPVTALLLPLALLLHAARP |
| 169 | CD28 signal peptide | MLRLLLALNLFPSIQVTG |
| 170 | Fas signal peptide | MLGIWTLLPLVLTSVARLSSKSVNA |
| 171 | PD-1 signal peptide | MQIPQAPWPVVWAVLQLGWRPGW |
| 172 | CD3 zeta signal | MKWKALFTAAILQAQLPITEA |
| | peptide | |
| 173 | CD8α partial | SQFRVSPLDRTWNLGETVELKCQVLLSNPTSGCSWL |
| | extracellular domain | FQPRGAAASPTFLLYLSQNKPKAAEGLDTQRFSGKR |
| | | LGDTFVLTLSDFRRENEGYYFCSALSNSIMYFSHFVP |
| | | VFLPA |
| 174 | NKG2D extracellular | IPLTESYCGPCPKNWICYKNNCYQFFDESKNWYESQ |
| | sequence | ASCMSQNASLLKVYSKEDQDLLKLVKSYHWMGLV |
| | | HIPTNGSWQWEDGSILSPNLLTIIEMQKGDCALYASS |
| | | FKGYIENCSTPNTYICMQRTVKGKHLCPSPLFPGPSKP |
| 175 | Lck intracellullar | PLQDNLVIALHSYEPSHDGDLGFEKGEQLRILEQ |
| | sequence (47-496) | SGEWWKAQSLTTGQEGFIPFNFVAKANSLEPEP |
| | | WFFKNLSRKDAERQLLAPGNTHGSFLIRESESTA |
| | | GSFSLSVRDFDQNQGEVVKHYKIRNLDNGGFYI |
| | | SPRITFPGLHELVRHYTNASDGLCTRLSRPCQTQ |
| | | KPQKPWWEDEWEVPRETLKLVERLGAGQFGEV |
| | | WMGYYNGHTKVAVKSLKQGSMSPDAFLAEAN |
| | | LMKQLQHQRLVRLYAVVTQEPIYIITEYMENGS |
| | | LVDFLKTPSGIKLTINKLLDMAAQIAEGMAFIEE |
| | | RNYIHRDLRAANILVSDTLSCKIADFGLARLIED |
| | | NEYTAREGAKFPIKWTAPEAINYGTFTIKSDVWS |
| | | FGILLTEIVTHGRIPYPGMTNPEVIQNLERGYRM |
| | | VRPDNCPEELYQLMRLCWKERPEDRPTFDYLRS |
| | | VLEDFFTATEGQYQPQP |
| 176 | Linker (G/S/T 22 | GSGGGTGGGSGGSGGGTGGGSGMASSSGSSPRPAPD |
| | amino acid linker)+ | ENEFPFGCPPTVCQDPKEPRALCCAGCLSENPRNGED |
| | TRAFI intracellullar | QICPKCRGEDLQSISPGSRLRTQEKAHPEVAEAGIGCP |
| | sequence | FAGVGCSFKGSPQSVQEHEVTSQTSHLNLLLGFMKQ |
| | | WKARLGCGLESGPMALEQNLSDLQLQAAVEVAGDL |
| | | EVDCYRAPCSESQEELALQHFMKEKLLAELEGKLRV |
| | | FENIVAVLNKEVEASHLALATSIHQSQLDRERILSLEQ |
| | | RVVELQQTLAQKDQALGKLEQSLRLMEEASFDGTFL |
| | | WKITNVTRRCHESACGRTVSLFSPAFYTAKYGYKLC |
| | | LRLYLNGDGTGKRTHLSLFIVIMRGEYDALLPWPFR |
| | | NKVTFMLLDQNNREHAIDAFRPDLSSASFQRPQSETN |
| | | VASGCPLFFPLSKLQSPKHAYVKDDTMFLKCIVETST |
| 177 | CD28 intracellular | RSKRSRGGHSDYMNMTPRRPGPTRKHYQPYAPPRDF |
| | sequence fused to | AAYRSRVKFSRSADAPAYQQGQNQLYNELNLGRRE |
| | CD3ζ intracellular | EYDVLDKRRGRDPEMGGKPRRKNPQEGLYNELQKD |
| | sequence | KMAEAYSEIGMKGERRRGKGHDGLYQGLSTATKDT |
| | | YDALHMQALPPR |
| 178 | 4-1BB intracellular | RGRKKLLYIFKQPFMRPVQTTQEEDGCSCRFPEEEEG |
| | sequence fused to | GCELRVKFSRSADAPAYQQGQNQLYNELNLGRREE |
| | CD3ζ intracellular | YDVLDKRRGRDPEMGGKPRRKNPQEGLYNELQKDK |
| | sequence | MAEAYSEIGMKGERRRGKGHDGLYQGLSTATKDTY |
| | | DALHMQALPPR |
| 179 | CD8β signal peptide | MRPRLWLLLAAQLTVLHGNSV |
| 180 | CD28 intracellular | HLCCRRRSKRSRGGHSDAMNMTARRAGPTRKH |
| | sequence with GG | YQAYAAPRDFAAYRS |
| | mutation and partial | |
| | signalling mutation | |
| 181 | TRAF1 amino acid | MASSSGSSPRPAPDENEFPFGCPPTVCQDPKEPRALC |
| | sequence | CAGCLSENPRNGEDQICPKCRGEDLQSISPGSRLRTQ |
| | | EKAHPEVAEAGIGCPFAGVGCSFKGSPQSVQEHEVTS |
| | | QTSHLNLLLGFMKQWKARLGCGLESGPMALEQNLS |
| | | DLQLQAAVEVAGDLEVDCYRAPCSESQEELALQHF |
| | | MKEKLLAELEGKLRVFENIVAVLNKEVEASHLALAT |
| | | SIHQSQLDRERILSLEQRVVELQQTLAQKDQALGKLE |
| | | QSLRLMEEASFDGTFLWKITNVTRRCHESACGRTVS |
| | | LFSPAFYTAKYGYKLCLRLYLNGDGTGKRTHLSLFIV |
| | | IMRGEYDALLPWPFRNKVTFMLLDQNNREHAIDAFR |
| | | PDLSSASFQRPQSETNVASGCPLFFPLSKLQSPKHAY |
| | | VKDDTMFLKCIVETST |
| 182 | Linker | GSGGGTGGGSGGSGGGTGGGSG |
| 183 | Whitlow linker | GSTSGSGKPGSGEGSTKG |
| 184 | Linker | GGGGSGGGGSGGGGS |
| 185 | CD28 intracellular | DAMNMTARRAGPTRKHYQAYAAPRDFAAYRS |
| | sequence with partial | |
| | signaling mutation | |
| 186 | CD28 intracellular | DAMNMTARRAGPTRKHFQAFAAPRDFAAFRS |
| | sequence with partial | |
| | signaling mutation | |
| 187 | OX40 intacellular | GGSFRPI |
| | motif | |
| 188 | Lck amino acid | MGCGCSSHPEDDWMENIDVCENCHYPIVPLDGKGTL |
| | sequence | LIRNGSEVRDPLVTYEGSNPPASPLQDNLVIALHSYEP |
| | | SHDGDLGFEKGEQLRILEQSGEWWKAQSLTTGQEGF |
| | | IPFNFVAKANSLEPEPWFFKNLSRKDAERQLLAPGNT |
| | | HGSFLIRESESTAGSFSLSVRDFDQNQGEVVKHYKIR |
| | | NLDNGGFYISPRITFPGLHELVRHYTNASDGLCTRLS |
| | | RPCQTQKPQKPWWEDEWEVPRETLKLVERLGAGQF |
| | | GEVWMGYYNGHTKVAVKSLKQGSMSPDAFLAEAN |
| | | LMKQLQHQRLVRLYAVVTQEPIYIITEYMENGSLVD |
| | | FLKTPSGIKLTINKLLDMAAQIAEGMAFIEERNYIHRD |
| | | LRAANILVSDTLSCKIADFGLARLIEDNEYTAREGAK |
| | | FPIKWTAPEAINYGTFTIKSDVWSFGILLTEIVTHGRIP |
| | | YPGMTNPEVIQNLERGYRMVRPDNCPEELYQLMRLC |
| | | WKERPEDRPTFDYLRSVLEDFFTATEGQYQPQP |
| 189 | GITR amino acid | MAQHGAMGAFRALCGLALLCALSLGQRPTGGPGCG |
| | sequence | PGRLLLGTGTDARCCRVHTTRCCRDYPGEECCSEWD |
| | | CMCVQPEFHCGDPCCTTCRHHPCPPGQGVQSQGKFS |
| | | FGFQCIDCASGTFSGGHEGHCKPWTDCTQFGFLTVFP |
| | | GNKTHNAVCVPGSPPAEPLGWLTVVLLAVAACVLL |
| | | LTSAQLGLHIWQLRSQCMWPRETQLLLEVPPSTEDA |
| | | RSCQFPEEERGERSAEEKGRLGDLWV |
| 190 | GITR intracellular | QLGLHIWQLRSQCMWPRETQLLLEVPPSTEDARSCQ |
| | amino acid sequence | FPEEERGERSAEEKGRLGDLWV |
| 191 | Fas intracellular | KRKEVQKTCRKHRKENQGSHESPTLNPETVAIN |
| | amino acid sequence | LSDVDLSKYITTIAGVMTLSQVKGFVRKNGVNE |
| | | AKIDEIKNDNVQDTAEQKVQLLRNWHQLHGKK |
| | | EAYDTLIKDLKKANLCTLAEKIQTIILKDITSDSE |
| | | NSNFRNEIQSLV (SEQ ID NO.: |

### EXAMPLES

### Example 1

### Generation of Chimeric Co-Receptor Proteins

Several cancers express MHC-I-restricted antigens. Adoptive T cell therapies have been developed using MHC-I-restricted TCRs that specifically recognize cancer antigens (see, e.g., PCT Publication Nos. WO 2016/022400; WO 2018/170338; WO 2018/090057; WO 2017/112944; WO 2017/193104; WO 2018/058002; and WO 2013/071154). However, it can be advantageous for cell therapy products to comprise both CD8⁺ T cells (which typically naturally express MHC-I-restricted TCR) and CD4+ T cells (which typically naturally express MHC-II-restricted TCR) (see, e.g., Sommermeyer et al., Leukemia 30 (2): 1888 (2016)). When targeting a MHC-I-expressing cancer with a CD4⁺: CD8⁺ T cell composition, a CD4⁺ T cell population engineered to express an appropriate MHC-I-restricted TCR may be used.

In the present disclosure, it was determined that T cells expressing such “cross-MHC-restricted” TCRs perform favorably when also expressing a cognate co-receptor. Further, the addition of exogenous costimulatory proteins improved CD4⁺ T cell responses to MHC-I-antigen in the context of an exogenous MHC-I TCR. To reduce the risk that introduced co-stimulatory domains could undesirably provide co-stimulation to endogenous MHC-II-TCR signaling, single-chain fusion proteins were generated that combined the MHC-I-binding of a CD8 co-receptor complex with co-stimulatory signaling, thereby effectively tethering costimulatory activity to MHC-I-specific response.

Certain fusion proteins are shown schematically in FIGS. 1 and 8; these and additional constructs are summarized in FIGS. 9A-12C and Tables 1-3. In some fusions, costimulatory domains from CD28, 4-1BB, ICOS, and OX40 were fused to CD8β (M1 isotype) and/or CD8α co-receptor chains. In some of the CD8β-containing fusions, a six-amino-acid sequence from the CD8β transmembrane domain (“HLCCRR”; SEQ ID NO.:10) was included adjacent to the costimulatory domain. In some of the CD28 costimulatory domain-containing constructs, a native dileucine sequence motif (“LL”) was mutated to diglycine (“GG”) in order to improve expression. Tested constructs included Constructs A, B, E, F, G, H, I, J, K, O, Q, S, T, W, and Y (See e.g. Table 1). Constructs comprising costimulatory domains from CD28 or 4-1BB were among those selected for further testing.

### Example 2

### Functional Characterization of Chimeric Co-Receptor Fusions in CD4⁺ T Cells Transduced with MHC-I-Restricted TCR

### T Cell Proliferation in Response to Antigen

Primary human CD4⁺ T cells from healthy donors were transduced with lentivirus containing the co-receptor constructs and separately transduced with lentivirus containing a MHC-I-restricted TCR construct described in PCT Publication No. WO 2018/170338. Transduced cells were sorted at Day 7 following transduction and expanded. Cells were stained with anti-CD8 antibody and flow cytometry was performed. As shown in FIG. 2, chimeric CD8 constructs were expressed at similar levels to wild-type CD8 co-receptors by the transduced CD4⁺ T cells.

To investigate the ability of the chimeric co-receptor-expressing cells to respond to antigen, CD4⁺ T cells were transduced to express MHC-I-restricted TCR either without CD8 or with a CD8 chimeric fusion construct. Cells were sorted at Day 7 post-transduction and expanded using a rapid expansion protocol (REP), then stimulated at Day 9 post-REP with antigen-expressing MEL-275 cells at various effector:target ratios. As shown in FIG. 3, inclusion of a chimeric CD8 co-receptor construct improved proliferation of transduced CD4⁺ T cells in response to antigen (cells encoding a fusion with a CD28 co-stimulatory domain having proliferation more than cells encoding a fusion with a 4-1BB co-stimulatory domain) over CD4⁺ T cells expressing MHC-I-TCR in the absence of a CD8 co-receptor. Moreover, proliferation of the fusion-expressing cells was somewhat more consistently tied to E:T ratio.

### Cytokine Production

CD4⁺ T cells were transduced to express MHC-I-restricted TCR either without CD8 or with a CD8 chimeric fusion construct. Cells were sorted at Day 7 post-transduction and expanded using a rapid expansion protocol (REP), and then stimulated at Day 9 post-REP with peptide antigen, or not. Secretion of IFN-γ and TNF-α was measured by flow cytometry. As shown in FIGS. 4A and 4B, the fusion-expressing cells produced more cytokines than cells expressing MHC-I-restricted TCR (alone or with a (heterologous) wild-type CD8 co-receptor). Cells encoding a fusion comprising a CD28 co-stimulatory domain performed best in this assay.

### Example 3

### Design and Testing of CD3z-Based Fusion Proteins

Fusion proteins were designed that included (amino-terminal to carboxy-terminal direction): a CD3ζ extracellular domain; a CD3ζ transmembrane domain; a costimulatory domain from CD28 or 4-1BB; and a CD3ζ intracellular signaling (effector) domain. See Tables 1-3, Constructs Z and AA. Primary T cells from healthy donors were transduced with lentivirus encoding the fusion construct only, the fusion construct and an antigen-specific TCR, or the TCR only. Expression data is shown in FIGS. 17A-17C. Transduced T cells were assessed for killing activity against WT-1-expressing cancer cells using IncuCyte® assays. Data are shown in FIG. 5 (T cells from Donor 18575; “10” denotes the TCR) and 6 (T cells from Donor 18648; “37” denotes the TCR).

### Example 4

### Functional Characterization of Chimeric Co-Receptor Fusions in CD4⁺ and CD8⁺ T Cells Transduced with MHC-I-Restricted TCR

### T Cell Cytokine Production in Response to Antigen

Primary human CD4⁺ and CD8⁺ T cells from healthy donors were selected using CD4 and CD8 selection kits, and separately transduced with lentivirus containing a co-receptor construct and lentivirus containing a HLA-A2 restricted MAGE-A1-278 specific TCR construct. Five to seven days post-transduction, cells were sorted by MAGE-A1-278 tetramer and expanded for seven to nine days prior to downstream analysis.

Expanded CD4⁺ and CD8⁺ T cells were stimulated with MAGE-A1 and HLA-A2 positive tumor cell lines ME275 and H1299 at an E:T ratio of 5:1 overnight at 37° C., in the presence of Golgistop and Golgiplug (BD biosciences). After stimulation, cells were fixed, permeabilized, and stained for intracellular IFN-γ and TNFα. Intracellular cytokine levels were measured by flow cytometry. Data are shown in FIGS. 15A-15D. Certain of the tested constructs showed increased production of TNFα (and in for some constructs, also IFNγ) by cells (including CD4+ T cells) against H1299 and ME275 target cells.

### Tumor Cell Killing and Control

Primary human CD4⁺ and CD8⁺ T cells from healthy donors were selected using CD4 and CD8 selection kits, and separately transduced with lentivirus containing a co-receptor construct and a lentivirus containing a HLA-A2 restricted MAGE-A1-278 specific TCR construct. Five to seven days post-transduction, cells were sorted by MAGE-A1-278 tetramer and expanded for seven to nine days prior to downstream analysis.

Expanded CD4⁺ and CD8⁺ T cells were co-cultured with mCherry-positive ME275 cells at an E:T ratio of 5:1. Tumor cell killing and control were monitored by an IncuCyte® instrument, which took images every 2 hours over an eleven-day span. Additional ME275 tumor cells were added on day 4 in order to stress the T cells. As shown in FIG. 16A, CD8+ T cells transduced with CD8α/CD8β-CD28 or CD8α/CD8β-41BB fusions performed better than CD8+ T cells transduced with the TCR alone. As shown in FIG. 16B, CD4⁺ T cells transduced with CD8α/CD8β-CD28 or CD8α/CD8β-41BB fusions performed noticeably better than CD4⁺ T cells transduced with either the wild-type CD8α/CD8β co-receptors or the TCR alone. It was observed that CD8β fusions comprising the CD8β intracellular amino acid sequence of SEQ ID NO.:10 demonstrated improved function over those that did not.

### Example 5

### Functional Characterization of Chimeric Co-Receptor Fusions in CD4⁺ and CD8⁺ Jurkat Cells Transduced with MHC-I-Restricted TCR

### Jurkat Reporter Sensitivity in Response to Peptide Antigen

CD4⁺ and CD8⁺ Jurkat reporter cells were separately transduced with lentivirus containing a co-receptor construct and lentivirus containing an HLA-A2-restricted MAGE-A1-278 specific TCR construct. Comparators were reporter cells transduced only with lentivirus encoding the TCR, with lentivirus encoding the TCR and wild-type CD8αβ, and with lentivirus encoding an irrelevant TCR with wild-type CD8αβ. The Jurkat reporter cells had MHC class I molecules knocked out, endogenous TCR α and β chains knocked out, and a reporter (neogreen) knocked in downstream of the Nur77 gene. Nur77 is upregulated after TCR activation.

The transduced Jurkat reporter cells were co-cultured with peptide-loaded T2 cells at an E:T ratio of 5:1. Multiple populations of T2 cells were peptide-loaded in the presence of various concentrations of peptide, as shown on the x-axis (left to right) in

FIGS. 14A and 14B. In this experiment, some, but not all, of the tested constructs provided a higher percentage of reporter-positive transduced cells, as compared to Jurkat cells that contained the TCR construct (alone or with CD8αβ), including at lower concentrations of peptide (ug/mL). For CD8αβ-containing constructs, modification of the CD8α chain produced less robust results as compared to modification of the CD8β chain.

### Example 6

### Material and Methods

### Lentivirus Production:

Costimulatory molecule genes along with a HLA-A2 restricted MAGE-A1-278 specific TCR were cloned in the lentivirus backbone pRRLSIN. Transfer plasmids encoding the costimulatory molecules along with helper and envelope plasmids were co-transfected into HEK293 cells using the effectene transfection reagent from Qiagen according to the manufacturer's instruction. Cell culture supernatant containing the lentivirus was collected 48 and 72 hours post transfection. Viral supernatant was concentrated using the Lenti-X concentrator from Takara per manufacturer's protocol.

### T Cell Transduction:

CD4 and CD8 T cells from healthy donors were selected using the stemcell CD4 or CD8 T cell selection kits and activated with TransAct (Miltenyi) according to manufacturer's protocols. After 48 hours, concentrated virus was added to activated T cells in the presence of 10 ug/ml of protamine sulfate. Five to seven days post transduction, cells were sorted by MAGE-A1-278 tetramer and expanded for seven to nine days prior to downstream analysis.

### T Cell Functionality Analysis:

Transduced, sorted and expanded T cells were stimulated with tumor cell lines ME275 and H1299, which are positive for MAGE-A1 and HLA-A2, at E:T ratio at 5:1 overnight at 37° C. in the presence of Golgistop and Golgiplug (BD biosciences). After stimulation, cells were fixed, permeabilized and stained for intracellular IFNγ, TNFα and IL2. Stained cells were then analyzed by flow cytometry.

### Tumor Cell Killing and Inhibition:

Tumor cell line ME275 was transduced with mCherry and sorted on mCherry positive cells. Transduced, sorted and expanded T cells were cocultured with mCherry+ tumor cells at 5:1 E:T ratio in 96 well plates. Images were taken in the incucyte S3 every 2 hours over the span of 11 days. More tumor cells were added to the tested wells on day 4 to future stress the T cells. The intensity of mCherry was measured and analyzed by the Incucyte software. The intensity of the first time point of each well was normalized to 1. Same experimental conditions were measured in duplicates.

### Jurkat Reporter Stimulation:

Jurkat reporter cell with MHC class I molecules knocked out, endogenous TCR alpha and beta chains knocked out and neogreen knocked in downstream of Nur77 was generated in the lab. Jurkat reporter cells were transduced with lentivirus encoding the costimulatory molecules and cocultured with peptide loaded T2 cells at E:T ratio of 5:1. After an overnight stimulation, cells were analyzed by flow cytometry. Transduced Jurkat cells are CD3 positive, and stimulated cells are reporter positive.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, including U.S. provisional patent application Ser. No. 63/125,347, filed Dec. 14, 2020, are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1.-162. (canceled)

163. A polypeptide comprising:
an extracellular component comprising an extracellular domain from a CD8 β-chain (CD8β), or a functional portion or variant thereof that is capable of binding to a MHC Class I molecule;
a transmembrane domain; and
an intracellular component comprising (1) a CD8β intracellular region amino acid sequence that comprises or consists of the amino acid sequence set forth in SEQ ID NO.:10 or SEQ ID NO.:9, and (2) a CD28 costimulatory domain, or a functional portion or variant thereof.

164. The polypeptide of claim 163, wherein the CD8β intracellular region amino acid sequence comprises or consists of the amino acid sequence set forth in SEQ ID NO.:10.

165. The polypeptide of claim 163, wherein the CD8β intracellular region amino acid sequence comprises or consists of the amino acid sequence set forth in SEQ ID NO.:9.

166. The polypeptide of claim 163, wherein the transmembrane domain is a CD8β transmembrane domain.

167. The polypeptide of claim 163, wherein the CD28 costimulatory domain or a functional portion or variant thereof comprises or consists of an amino acid sequence having at least 80% identity to the amino acid sequence shown in SEQ ID NO.:19, provided that one or both of the leucine residues corresponding to positions 7 and 8 of SEQ ID NO.:19 is substituted for a different amino acid.

168. The polypeptide of claim 167, wherein one or both of the leucine residues corresponding to positions 7 and 8 of SEQ ID NO.:19 is independently substituted with a glycine residue.

169. The polypeptide of claim 163, wherein:
the extracellular domain from a CD8 β-chain (CD8β), or a functional portion or variant thereof that is capable of binding to a MHC Class I molecule comprises or consists of an amino acid sequence having at least 80% identity to the amino acid sequence set forth in SEQ ID NO.:7, SEQ ID NO.:68, or SEQ ID NO.:82; and/or
the transmembrane domain comprises or consists of an amino acid sequence having at least 80% identity to the amino acid sequence set forth in SEQ ID NO.:8; and/or
the intracellular component comprises or consists of an amino acid sequence having at least 80% identity to the amino acid sequence shown in any one of SEQ ID NOs.: 19, 20, 81, 83, 84, 108, 180, 185, and 186.

170. The polypeptide of claim 163, wherein the extracellular component comprises an extracellular domain from a human CD8β M1 isoform, or a functional portion or variant thereof that is capable of binding to a MHC Class I molecule, and the transmembrane domain is a CD8β M1 isoform transmembrane domain.

171. The polypeptide of claim 163, comprising or consisting of the amino acid sequence set forth in any one of SEQ ID NOs.: 36, 37, 113, 115, 120, 121, 126, and 127.

172. A polynucleotide comprising a polynucleotide encoding the polypeptide of claim 163.

173. The polynucleotide of claim 172, wherein the polypeptide is a first polypeptide and the polynucleotide further comprises a polynucleotide encoding a second polypeptide, wherein the second polypeptide comprises:
(i) a CD8 co-receptor α-chain (CD8α), or a functional portion or variant thereof; or
(ii) an extracellular domain from a CD8α, or a functional portion or variant thereof.

174. The polynucleotide of claim 173, wherein the polynucleotide further comprises:
a polynucleotide encoding a self-cleaving peptide and/or a furin cleavage site, wherein the self-cleaving peptide and/or furin cleavage site is disposed between the first polypeptide and the second polypeptide; and/or
a polynucleotide comprising an IRES, wherein the IRES is disposed between the polynucleotide encoding the first polypeptide and the polynucleotide encoding the second polypeptide.

175. The polynucleotide of claim 173, wherein the second polypeptide comprises an amino acid sequence having at least 80% identity to, or comprising or consisting of, the amino acid sequence set forth in any one of SEQ ID NOs.: 1-5, 39, 67, 78, 79, 90-95, 118, 122, 124, 136, 138, 140-147, 150, 152, and 173.

176. The polynucleotide of claim 172, encoding any one or more of Constructs A, E, and G as set forth in Table 1.

177. The polynucleotide of claim 173, wherein the first polypeptide and the second polypeptide comprise or consist of amino acid sequences having at least 90% identity to, or comprising or consisting of, the amino acid sequences set forth in SEQ ID NOs.:
(i) 1 and 113, respectively;
(ii) 114 and 115, respectively;
(iii) 1 and 120, respectively;
(iv) 114 and 121, respectively;
(v) 1 and 126, respectively; or
(vi) 114 and 127, respectively.

178. The polynucleotide of claim 173, further comprising a polynucleotide encoding a binding protein that specifically binds to an antigen or to an antigen: MHC complex.

179. The polynucleotide of claim 178, wherein the binding protein comprises a TCR or an antigen-binding fragment thereof.

180. The polynucleotide of claim 178, wherein the binding protein comprises a binding domain from a MHC-I-restricted TCR, or a functional variant or portion thereof.

181. A vector comprising the polynucleotide of claim 172.

182. A host cell comprising the polynucleotide of claim 172.

183. The host cell of claim 182, wherein the host cell comprises a T cell.

184. The host cell of claim 182, wherein the host cell comprises a CD4+ T cell.

185. A composition comprising the host cell of claim 182 and a pharmaceutically acceptable carrier, excipient, or diluent.

186. A method of treating a disease or condition in a subject, the method comprising administering to the subject an effective amount of the host cell of claim 182.

187. A method comprising introducing the polynucleotide of claim 172 into a host cell.

188. A polypeptide comprising:
(1)(i) an extracellular component from a CD3; (ii) a transmembrane domain that is optionally from CD3ζ; and (iii) an intracellular component comprising (iii)(a) a CD28 costimulatory domain and (iii)(b) a CD3 intracellular signaling domain, wherein, optionally, the extracellular component does not further comprise a target-binding domain (e.g. an antigen-binding domain, such as from an antibody or antigen-binding fragment thereof, a T cell receptor, or a receptor ectodomain); or
(2)(i) an extracellular component from a CD3ζ; (ii) a transmembrane domain that is optionally from CD3ζ; and (iii) an intracellular component comprising (iii)(a) a 4-1BB costimulatory domain and (iii)(b) a CD3ζ costimulatory domain, wherein, optionally, the extracellular component does not further comprise a target-binding domain (e.g. an antigen-binding domain, such as from an antibody or antigen-binding fragment thereof, a T cell receptor, or a receptor ectodomain).

189. A polynucleotide encoding the polypeptide of claim 188.

190. A host cell comprising the polynucleotide of claim 189.

191. A composition comprising the host cell of claim 190 and a pharmaceutically acceptable carrier, excipient, or diluent.

192. A method of treating a disease or condition in a subject, the method comprising administering to the subject an effective amount of the host cell of claim 190.

193. A method comprising introducing the polynucleotide of claim 189 into a host cell.

194. A polypeptide selected from any one of (1)-(24):
(1) a polypeptide comprising, consisting essentially of, or consisting of: (i) an extracellular component comprising an extracellular domain from a CD8 β-chain (CD8β), or a functional portion or variant thereof that is capable of binding to a MHC Class I molecule, (ii) a transmembrane domain from a CD8β, and (a)(iii) an intracellular component comprising (iii)(1) a CD8β intracellular region amino acid sequence that comprises or consists of the amino acid sequence set forth in SEQ ID NO.:10 or SEQ ID NO.:9, and (iii)(2) a costimulatory domain or a functional portion or variant thereof, wherein, optionally, the costimulatory domain or a functional portion or variant thereof is from one or more of CD28 (optionally comprising a LL→GG mutation, a partial signaling mutation, and/or a full signaling mutation), 4-1BB (CD137), OX40 (CD134), ICOS (CD278), GITR, CD27, CD2, CD5, ICAM-1 (CD54), LFA-1 (CD11a/CD18), GITR, CD30, CD40, BAFF-R, HVEM, LIGHT, NKG2C, SLAMF7, NKp80, CD160, B7-H3, a ligand that specifically binds with CD83, CD94, or DAP12;
(2) a polypeptide comprising, consisting essentially of, or consisting of: (i) an extracellular component comprising an extracellular domain from a CD8 co-receptor β-chain or a functional portion or variant thereof, or from a CD8 co-receptor α-chain or a functional portion or variant thereof, that is capable of binding to a MHC class I molecule; (ii) a transmembrane domain, provided that the transmembrane domain is not a transmembrane domain from a CD8 co-receptor α-chain when the extracellular component comprises a full length extracellular domain from the CD8 co-receptor α-chain; and (ii) an intracellular component comprising a co-stimulatory domain or a functional portion or variant thereof, wherein, optionally, the co-stimulatory domain comprises a co-stimulatory domain from one or more of CD28, 4-1BB (CD137), OX40 (CD134), ICOS (CD278), CD27, CD2, CD5, ICAM-1 (CD54), LFA-1 (CD11a/CD18), GITR, CD30, CD40, BAFF-R, HVEM, LIGHT, NKG2C, SLAMF7, NKp80, CD160, B7-H3, a ligand that specifically binds with CD83, CD94, DAP12, and/or comprises a functional variant of a co-stimulatory domain thereof;
(3) a polypeptide comprising, consisting essentially of, or consisting of: (i) an extracellular component comprising an extracellular domain from a CD8 co-receptor β-chain or a functional portion or variant thereof, or from a CD8 co receptor α-chain or a functional portion or variant thereof, that is capable of binding to a MHC class I molecule; (ii) a transmembrane domain; and (iii) an intracellular component comprising a co stimulatory domain from one, two, or three of: (a) a variant sequence of CD28 comprising or consisting of an amino acid sequence having at least 80% identity to the amino acid sequence shown in SEQ ID NO: 19 or 20, provided that: (1) no Tyr residue corresponding to position 12, 27, 30, or 39 of SEQ ID NO: 19 is substituted with Phe when the extracellular component comprises a full length extracellular domain from a CD8 co receptor α chain and the transmembrane domain comprises a transmembrane domain from the CD8 co receptor α chain; and/or (2) one or both of the leucine residues corresponding to positions 7 and 8 of SEQ ID NO: 19 is substituted for a different amino acid, wherein the different amino acid optionally comprises glycine; (b) CD27, or a functional portion or variant thereof; (c) 4-1BB, or a functional portion or variant thereof; (d) ICOS, or a functional portion or variant thereof; (e) OX40, or a functional portion or variant thereof; (f) CD30, or a functional portion or variant thereof; (g) LFA-1, or a functional portion or variant thereof; (h) CD2, or a functional portion or variant thereof; (i) CD7, or a functional portion or variant thereof; (j) LIGHT, or a functional portion or variant thereof; (k) NKG2C, or a functional portion or variant thereof; (l) B7-H3, or a functional portion or variant thereof; (m) GITR, or a functional portion or variant thereof; (n) BAFF-R, or a functional portion or variant thereof; (o) CD5, or a functional portion or variant thereof; (p) HVEM, or a functional portion or variant thereof; (q) CD160, or a functional portion or variant thereof; (r) LFA-1, or a functional portion or variant thereof; (s) SLAMF7, or a functional portion or variant thereof; (t) NKp80, or a functional portion or variant thereof; (u) ICAM-1, or a functional portion or variant thereof; (v) CD94, or a functional portion or variant thereof; (w) DAP12, or a functional portion or variant thereof; or (x) a ligand that specifically binds with CD83;
(4) a polypeptide comprising, consisting essentially of, or consisting of: (i) an extracellular component from a CD8β; (ii) a transmembrane domain that is optionally from a CD8β; and (iii) an intracellular component comprising a CD28 costimulatory domain and an optional a LL→GG mutation, wherein the polypeptide is capable of binding to a MHC Class I molecule;
(5) a polypeptide comprising, consisting essentially of, or consisting of: (i) an extracellular component from a CD8α; (ii) a transmembrane domain that is optionally from a CD8α; and (iii) an intracellular component comprising a CD28 costimulatory domain and an optional LL→GG mutation, wherein the polypeptide is capable of binding to a MHC Class I molecule;
(6) a polypeptide comprising, consisting essentially of, or consisting of: (i) an extracellular component from a CD8β; (ii) a transmembrane domain that is optionally from a CD8β; and (iii) an intracellular component comprising (1) a CD28 costimulatory domain comprising the amino acid sequence DAMNMTARRAGPTRKHYQAYAAPRDFAAYRS (SEQ ID NO.185) and (2) an optional LL→GG mutation, wherein the polypeptide is capable of binding to a MHC Class I molecule;
(7) a polypeptide comprising, consisting essentially of, or consisting of: (i) an extracellular component from a CD8α; (ii) a transmembrane domain that is optionally from a CD8α; and (iii) an intracellular component comprising (1) a CD28 costimulatory domain comprising the amino acid sequence DAMNMTARRAGPTRKHYQAYAAPRDFAAYRS (SEQ ID NO.185) and (2) an optional LL→GG mutation, wherein the polypeptide is capable of binding to a MHC Class I molecule;
(8) a polypeptide comprising, consisting essentially of, or consisting of: (i) an extracellular component from a CD8β; (ii) a transmembrane domain that is optionally from a CD8β; and (iii) an intracellular component comprising a costimulatory domain from (iii)(1) a 4-1BB, (iii)(2) an ICOS, (iii)(3), an OX40, or (iii)(4) a GITR, wherein the polypeptide is capable of binding to a MHC Class I molecule;
(9) a polypeptide comprising, consisting essentially of, or consisting of: (i) an extracellular component from a CD8α; (ii) a transmembrane domain from a CD28; and (iii) an intracellular component comprising a CD28 costimulatory domain and, optionally, a LL→GG mutation, wherein the polypeptide is capable of binding to a MHC Class I molecule;
(10) a polypeptide comprising, consisting essentially of, or consisting of: (i) an extracellular component from a CD8β; (ii) a transmembrane domain from a CD8β; and (iii) an intracellular component comprising (iii)(1) a CD8β intracellular region amino acid sequence (optionally comprising or consisting of SEQ ID NO.:9 or 10) and (iii)(2) a signaling domain from Lck, wherein the fusion protein is capable of binding to a MHC Class I molecule;
(11) a polypeptide comprising, consisting essentially of, or consisting of: (i) an extracellular component from a CD8α; (ii) a transmembrane domain that is optionally from a CD8 α; and (iii) an intracellular component comprising a CD28 costimulatory domain comprising the amino acid sequence DAMNMTARRAGPTRKHFQAFAAPRDFAAFRS (SEQ ID NO.:186), and optionally further comprising a LL→GG mutation, wherein the polypeptide is capable of binding to a MHC Class I molecule;
(12) a polypeptide comprising, consisting essentially of, or consisting of: (i) an extracellular component from a CD8β; (ii) a transmembrane domain that is optionally from a CD8β; and (iii) an intracellular component comprising a CD28 costimulatory domain comprising the amino acid sequence DAMNMTARRAGPTRKHFQAFAAPRDFAAFRS (SEQ ID NO.:186), and optionally further comprising a LL→GG mutation, wherein the polypeptide is capable of binding to a MHC Class I molecule;
(13) a polypeptide comprising, consisting essentially of, or consisting of: (i) an extracellular component comprising (i)(1) a CD8α extracellular region amino acid sequence (e.g. comprising or consisting of a CD8α Ig V-like domain), (i)(2) a CD8β stalk region amino acid sequence, and (i)(3) a CD28 extracellular region amino acid sequence; (ii) a transmembrane domain from CD28; and (iii) an intracellular component comprising a CD28 costimulatory domain and an optional LL→GG mutation, wherein the polypeptide is capable of binding to a MHC Class I molecule;
(14) a polypeptide comprising, consisting essentially of, or consisting of: (i) an extracellular component comprising (i)(1) a CD8α extracellular region amino acid sequence (e.g. comprising or consisting of a CD8α Ig V-like domain), (i)(2) a CD8β stalk region amino acid sequence, and (i)(3) a CD28 extracellular region amino acid sequence; (ii) a transmembrane domain from CD28; and (iii) an intracellular component comprising (iii)(1) a CD28 costimulatory domain and an optional LL→GG mutation and (iii)(2) a CD8α intracellular region amino acid sequence, wherein the polypeptide is capable of binding to a MHC Class I molecule;
(15) a polypeptide comprising, consisting essentially of, or consisting of: (i) an extracellular component from a NKG2D; (ii) a transmembrane domain from a NKG2D; and (iii) an intracellular component comprising a CD28 costimulatory domain and an optional LL→GG mutation, wherein the polypeptide is capable of binding to a NKG2D ligand;
(16) a polypeptide comprising, consisting essentially of, or consisting of: a Fas extracellular component and a transmembrane domain that is optionally from Fas, wherein the polypeptide does not comprise a functional Fas intracellular signaling domain, wherein the polypeptide is capable of binding to a FasL, and wherein the polypeptide optionally comprises a truncated Fas protein that does not comprise a full-length Fas intracellular region;
(17) a polypeptide comprising, consisting essentially of, or consisting of: (i) an extracellular component from a Fas; (ii) a transmembrane domain that is optionally from Fas; and (iii) an intracellular component comprising a Lck signaling domain, wherein the polypeptide is capable of binding to a FasL;
(18) a polypeptide comprising, consisting essentially of, or consisting of: (i) an extracellular component from a Fas; (ii) a transmembrane domain that is optionally from Fas;
and (iii) an intracellular component comprising a CD8α intracellular amino acid sequence, wherein the polypeptide is capable of binding to a FasL and, optionally, associating with a Lck;
(19) a polypeptide comprising, consisting essentially of, or consisting of: (i) an extracellular component from a Fas; (ii) a transmembrane domain that is optionally from a Fas; (iii) an intracellular component comprising a TRAF1 intracellular signaling domain, and, optionally, (iv) a linker amino acid sequence disposed between and connecting the transmembrane domain and the TRAF1 intracellular signaling domain, wherein the polypeptide is capable of binding to a FasL;
(20) a polypeptide comprising, consisting essentially of, or consisting of: (i) an extracellular component comprising an amino acid sequence having at least 90%, at least 91%, at least 92%, at least 93%, at least 94%, at least 95%, at least 96%, at least 97%, at least 98%, or at least 99% identity to, or comprising or consisting of, the amino acid sequence set forth in any one of SEQ ID NOs.: 2, 173, 90, 92, and 7; (ii) a transmembrane domain comprising an amino acid sequence having at least 90%, at least 91%, at least 92%, at least 93%, at least 94%, at least 95%, at least 96%, at least 97%, at least 98%, or at least 99% identity to, or comprising or consisting of, the amino acid sequence set forth in any one of SEQ ID NOs.: 3, 8, and 80; and (iii) an intracellular component comprising an amino acid sequence having at least 90%, at least 91%, at least 92%, at least 93%, at least 94%, at least 95%, at least 96%, at least 97%, at least 98%, or at least 99% identity to, or comprising or consisting of, the amino acid sequence set forth in any one of SEQ ID NOs.: 4, 83, 20, 9, 180, 81, 84, 85, 86, 87, 88, 89, 97, and 108;
(21) a polypeptide comprising, consisting essentially of, or consisting of, an extracellular component, a transmembrane domain, and an intracellular component according to Polypeptide 1 of any one of Constructs A-AA in Table 2;
(22) a polypeptide comprising, consisting essentially of, or consisting of, an extracellular component, a transmembrane domain, and an intracellular component according to Polypeptide 2 of any one of Constructs A-AA in Table 2;
(23) a polypeptide of Type A1, of Type A2, of Type B, of Type C1, of Type C2, of Type D1, of Type D2, of Type E, of Type F1, of Type F2, of Type G, of Type H, of Type I, of Type J, of Type K, of Type L1, of Type L2, of Type M, of Type N, of Type O, of Type P, of Type Q, of Type R, of Type S, of Type T1, of Type T2, of Type U, of Type V, of Type W, of Type X, of Type Y, of Type Z, or of Type AA, in accordance with Table 5;
(24) a polypeptide comprising two amino acid sequences, wherein each of the two amino acid sequences is independently selected from the amino acid sequence of the polypeptide of any one of (1)-(23).

195. A polynucleotide encoding the polypeptide of claim 194.

196. A polynucleotide selected from any one of (1)-(16):
(1) a polynucleotide comprising (a) a nucleotide sequence encoding a CD8α polypeptide and (b) a nucleotide sequence encoding a CCR4, wherein the CD8α polypeptide is optionally an engineered polypeptide comprising (i) a portion of a CD8β extracellular component, such as a CD8β stalk region amino acid sequence, (ii) a portion of a CD28 extracellular component; (iii) a CD28 transmembrane domain; (iv) an intracellular component comprising a CD28 costimulatory domain and, further optionally, a LL→GG mutation, wherein, still further optionally, the CD28 costimulatory domain comprises a partial signaling mutant or a full signaling mutant; and/or (v) a intracellular component comprising a costimulatory domain from 4-1BB, ICOS, OX40, GITR, TRAF1, or Lck;
(2) a polynucleotide comprising (a) a nucleotide sequence encoding a CD8α and (b) a nucleotide sequence encoding a CCR2b, wherein the CD8α polypeptide is optionally an engineered polypeptide comprising (i) a portion of a CD8β extracellular component, such as a CD8β stalk region amino acid sequence, (ii) a portion of a CD28 extracellular component; (iii) a CD28 transmembrane domain; (iv) an intracellular component comprising a CD28 costimulatory domain and, further optionally, a LL→GG mutation, wherein, still further optionally, the CD28 costimulatory domain comprises a partial signaling mutant or a full signaling mutant; and/or (v) a intracellular component comprising a costimulatory domain from 4-1BB, ICOS, OX40, GITR, TRAF1, or Lck;
(3) a polynucleotide comprising (a) a nucleotide sequence encoding a polypeptide comprising: (i) an extracellular component from a CD8β; (ii) a transmembrane domain that is optionally from a CD8β; and (iii) an intracellular component comprising (iii)(1) a CD8β intracellular region amino acid sequence comprising or consisting of SEQ ID NO.:9 or SEQ ID NO.:10 and (iii)(2) a wild-type CD28 costimulatory domain, wherein the polypeptide is capable of binding to a MHC Class I molecule; and (b) a nucleotide sequence encoding a CD8α polypeptide, wherein the CD8α polypeptide is optionally an engineered polypeptide comprising (i) a portion of a CD8β extracellular component, such as a CD8β stalk region amino acid sequence, (ii) a portion of a CD28 extracellular component; (iii) a CD28 transmembrane domain; (iv) an intracellular component comprising a CD28 costimulatory domain and, further optionally, a LL→GG mutation, wherein, still further optionally, the CD28 costimulatory domain comprises a partial signaling mutant or a full signaling mutant; and/or (v) a intracellular component comprising a costimulatory domain from 4-1BB, ICOS, OX40, GITR, TRAF1, or Lck;
(4) a polynucleotide comprising (a) a nucleotide sequence encoding a polypeptide comprising: (i) an extracellular component from a CD8β; (ii) a transmembrane domain that is optionally from a CD8β; and (iii) an intracellular component comprising a costimulatory domain from (iii)(1) a 4-1BB, (iii)(2) an ICOS, (iii)(3), an OX40, or (iii)(4) a GITR, wherein the polypeptide is capable of binding to a MHC Class I molecule; and (b) a nucleotide sequence encoding CD8α polypeptide, wherein the CD8α polypeptide is optionally an engineered polypeptide comprising (i) a portion of a CD8β extracellular component, such as a CD8β stalk region amino acid sequence, (ii) a portion of a CD28 extracellular component; (iii) a CD28 transmembrane domain; (iv) an intracellular component comprising a CD28 costimulatory domain and, further optionally, a LL→GG mutation, wherein, still further optionally, the CD28 costimulatory domain comprises a partial signaling mutant or a full signaling mutant; and/or (v) a intracellular component comprising a costimulatory domain from 4-1BB, ICOS, OX40, GITR, TRAF1, or Lck;
(5) a polynucleotide comprising a nucleotide sequence encoding a polypeptide, wherein the polypeptide has at least 90%, at least 91%, at least 92%, at least 93%, at least 94%, at least 95%, at least 96%, at least 97%, at least 98%, or at least 99% identity to, or comprising or consisting of, the amino acid sequence set forth in any one of SEQ ID NOs.: 36-42, 83-97, and 103-105;
(6) a polynucleotide encoding two or more polypeptides, each of the two or more polypeptides independently having at least 90%, at least 91%, at least 92%, at least 93%, at least 94%, at least 95%, at least 96%, at least 97%, at least 98%, or at least 99% identity to, or comprising or consisting of, an amino acid sequence set forth in any one of SEQ ID NOs.: 36-42, 83-97, and 103-105;
(7) a polynucleotide comprising a nucleic acid sequence encoding a polypeptide having at least 90%, at least 91%, at least 92%, at least 93%, at least 94%, at least 95%, at least 96%, at least 97%, at least 98%, or at least 99% identity to, or comprising or consisting of, the amino acid sequence set forth in any one of SEQ ID NOs.: 113, 115-118, and 120-167;
(8) a polynucleotide encoding a first polypeptide and a second polypeptide, wherein the first polypeptide and the second polypeptide have at least 90%, at least 91%, at least 92%, at least 93%, at least 94%, at least 95%, at least 96%, at least 97%, at least 98%, or at least 99% identity to, or comprise or consisting of, the amino acid sequences set forth in SEQ ID NOs.: (a) 1 and 113, respectively; (b) 1 and 115, respectively; (c) 1 and 116, respectively; (d) 1 and 117, respectively; ® 1 and 119, respectively; (f) 1 and 120, respectively; (g) 1 and 121, respectively; (h) 1 and 123, respectively; (i) 1 and any one of 125-135, respectively; (j) 1 and 137, respectively; (k) 1 and 139 respectively; (l) 114 and 113, respectively; (m) 114 and 115, respectively; (n) 114 and 116, respectively; (o) 114 and 117, respectively; (p) 114 and 119, respectively; (q) 114 and 120, respectively; ® 114 and 121, respectively; (s) 114 and 123, respectively; (t) 114 and any one of 125-135, respectively; (u) 114 and 137, respectively; (v) 114 and 139 respectively; (w) 1 or 114 and 6, respectively; (x) 118 and 113, respectively; (y) 118 and 115, respectively; (z) 118 and 116, respectively; (aa) 118 and 117, respectively; (bb) 118 and 119, respectively; (cc) 118 and 120, respectively; (dd) 118 and 121, respectively; (ee) 118 and 123, respectively; (ff) 118 and any one of 125-135, respectively; (gg) 118 and 137, respectively; (hh) 118 and 139 respectively; (ii) 118 and 6, respectively; (jj) 39 and 113, respectively; (kk) 39 and 115, respectively; (ll) 39 and 116, respectively; (mm) 39 and 117, respectively; (nn) 39 and 119, respectively; (oo) 39 and 120, respectively; (pp) 39 and 121, respectively; (qq) 39 and 123, respectively; (rr) 39 and any one of 125-135, respectively; (ss) 39 and 137, respectively; (tt) 39 and 139 respectively; (uu) 39 and 6 respectively; (vv) 122 and 113, respectively; (ww) 122 and 115, respectively; (xx) 122 and 116, respectively; (yy) 122 and 117, respectively; (zz) 122 and 119, respectively; (aaa) 122 and 120, respectively; (bbb) 122 and 121, respectively; (ccc) 122 and 123, respectively; (ddd) 122 and any one of 125-135, respectively; (eee) 122 and 137, respectively; (fff) 122 and 139 respectively; (ggg) 122 and 6, respectively; (hhh) 124 and 113, respectively; (xv) 124 and 115, respectively; (xvi) 124 and 116, respectively; (xvii) 124 and 117, respectively; (xviii) 124 and 119, respectively; (iii) 124 and 120, respectively; (jjj) 124 and 121, respectively; (kkk) 124 and 123, respectively; (lll) 124 and any one of 125-135, respectively; (mmm) 124 and 137, respectively; (nnn) 124 and 139 respectively; (ooo) 124 and 6, respectively; (ppp) 136 and 113, respectively; (qqq) 136 and 115, respectively; (rrr) 136 and 116, respectively; (sss) 136 and 117, respectively; (ttt) 136 and 119, respectively; (uuu) 136 and 120, respectively; (vvv) 136 and 121, respectively; (www) 136 and 123, respectively; (xxx) 136 and any one of 125-135, respectively; (yyy) 136 and 137, respectively; (zzz) 136 and 139 respectively; (aaaa) 136 and 6 respectively; (bbbb) 138 and 113, respectively; (cccc) 138 and 115, respectively; (dddd) 138 and 116, respectively; (eeee) 138 and 117, respectively; (ffff) 138 and 119, respectively; (gggg) 138 and 120, respectively; (hhhh) 138 and 121, respectively; (iiii) 138 and 123, respectively; (jjjj) 138 and any one of 125-135, respectively; (kkkk) 138 and 137, respectively; (llll) 138 and 139 respectively; (mmmm) 138 and 6, respectively; (nnnn) 1 or 114 and 106, respectively; (oooo) 1 or 114 and 107, respectively; (pppp) 150 and 151, respectively; (qqqq) 150 and 153, respectively; (rrrr) 152 and 151, respectively; or (ssss) 152 and 153, respectively;
(9) a polynucleotide encoding a first polypeptide and a second polypeptide, wherein the first polypeptide and the second polypeptide have at least 90%, at least 91%, at least 92%, at least 93%, at least 94%, at least 95%, at least 96%, at least 97%, at least 98%, or at least 99% identity to, or comprise or consisting of, the amino acid sequences set forth in SEQ ID NOs.: (a) 1 and 113, respectively; (b) 1 and 116, respectively; (c) 1 and 120, respectively; (d) 1 and 126, respectively; (e) 1 and 128, respectively; (f) 1 and 130, respectively; (g) 1 and 132, respectively; (h) 1 and 134, respectively; (i) 114 and 115, respectively; (j) 114 and 117, respectively; (k) 114 and 121, respectively; (l) 114 and 127, respectively; (m) 114 and 129, respectively; (n) 114 and 131, respectively; (o) 114 and 133, respectively; (p) 114 and 135, respectively; (q) 118 and 116, respectively; (r) 118 and 6, respectively; (s) 39 and 117, respectively; (t) 39 and 119, respectively; (u) 122 and 123, respectively; (v) 124 and 125, respectively; (w) 136 and 137, respectively; (x) 138 and 139 respectively; (y) 1 or 114 and 106, respectively; (z) 1 or 114 and 107, respectively; (aa) 150 and 151, respectively; or (bb) 152 and 153, respectively;
(10) a polynucleotide encoding a polypeptide that comprises: (i) an extracellular component comprising an amino acid sequence having at least 90%, at least 91%, at least 92%, at least 93%, at least 94%, at least 95%, at least 96%, at least 97%, at least 98%, or at least 99% identity to, or comprising or consisting of, the amino acid sequence set forth in any one of SEQ ID NOs.: 2, 173, 90, 92, and 7; (ii) a transmembrane domain comprising an amino acid sequence having at least 90%, at least 91%, at least 92%, at least 93%, at least 94%, at least 95%, at least 96%, at least 97%, at least 98%, or at least 99% identity to, or comprising or consisting of, the amino acid sequence set forth in any one of SEQ ID NOs.: 3, 8, and 80; and (iii) an intracellular component comprising an amino acid sequence having at least 90%, at least 91%, at least 92%, at least 93%, at least 94%, at least 95%, at least 96%, at least 97%, at least 98%, or at least 99% identity to, or comprising or consisting of, the amino acid sequence set forth in any one of SEQ ID NOs.: 4, 83, 20, 9, 180, 81, 84, 85, 86, 87, 88, 89, 97, and 108;
(11) a polynucleotide encoding a polypeptide comprising an extracellular component, a transmembrane domain, and an intracellular component according to Polypeptide 1 of any one of Constructs A-AA in Table 2;
(12) a polynucleotide encoding a polypeptide comprising an extracellular component, a transmembrane domain, and an intracellular component according to Polypeptide 2 of any one of Constructs A-AA in Table 2;
(13) a polynucleotide encoding: (i) a polypeptide that comprises an extracellular component, a transmembrane domain, and an intracellular component according to Polypeptide 1 of any one of Constructs A-AA in Table 2; and (ii) a polypeptide that comprises an extracellular component, a transmembrane domain, and an intracellular component according to Polypeptide 2 of any one of Constructs A-AA in Table 2;
(14) a polynucleotide encoding a polypeptide of Type A1, of Type A2, of Type B, of Type C1, of Type C2, of Type D1, of Type D2, of Type E, of Type F1, of Type F2, of Type G, of Type H, of Type I, of Type J, of Type K, of Type L1, of Type L2, of Type M, of Type N, of Type O, of Type P, of Type Q, of Type R, of Type S, of Type T1, of Type T2, of Type U, of Type V, of Type W, of Type X, of Type Y, of Type Z, or of Type AA, in accordance with Table 5;
(15) a polynucleotide encoding any two or more polypeptides selected from Types A1-AA in Table 5;
(16) a polynucleotide comprising two nucleotide sequences, wherein each of the two nucleotide sequences is independently selected from the nucleotide sequence of the polynucleotide of any one of (1)-(15).

197. A vector comprising the polynucleotide of claim 195.

198. A vector comprising the polynucleotide of claim 196.

199. A host cell comprising the polynucleotide of claim 195.

200. A host cell comprising the polynucleotide of claim 196.

201. A host cell selected from (1) and (2):
(1) a host cell expressing and/or encoding any two or more polypeptides selected from Types A1-AA in Table 5, wherein, optionally, the host cell comprises an immune cell, wherein, further optionally, the immune cell comprises a T cell, such as a CD4+ T cell, a CD8+ T cell, a CD4− CD8− double negative T cell, a γδ T cell, a naïve T cell, a central memory T cell, a stem cell memory T cell, an effector memory T cell, or any combination thereof;
(2) a host cell expressing a first polypeptide and a second polypeptide, wherein the first polypeptide and the second polypeptide have at least 90%, at least 91%, at least 92%, at least 93%, at least 94%, at least 95%, at least 96%, at least 97%, at least 98%, or at least 99% identity to, or comprise or consist of, the amino acid sequences set forth in SEQ ID NOs.: (a) 1 and 113, respectively; (b) 1 and 115, respectively; (c) 1 and 116, respectively; (d) 1 and 117, respectively; (e) 1 and 119, respectively; (f) 1 and 120, respectively; (g) 1 and 121, respectively; (h) 1 and 123, respectively; (i) 1 and any one of 125-135, respectively; (j) 1 and 137, respectively; (k) 1 and 139 respectively; (l) 114 and 113, respectively; (m) 114 and 115, respectively; (n) 114 and 116, respectively; (o) 114 and 117, respectively; (p) 114 and 119, respectively; (q) 114 and 120, respectively; (r) 114 and 121, respectively; (s) 114 and 123, respectively; (t) 114 and any one of 125-135, respectively; (u) 114 and 137, respectively; (v) 114 and 139 respectively; (w) 1 or 114 and 6, respectively; (x) 118 and 113, respectively; (y) 118 and 115, respectively; (z) 118 and 116, respectively; (aa) 118 and 117, respectively; (bb) 118 and 119, respectively; (cc) 118 and 120, respectively; (dd) 118 and 121, respectively; (ee) 118 and 123, respectively; (ff) 118 and any one of 125-135, respectively; (gg) 118 and 137, respectively; (hh) 118 and 139 respectively; (ii) 118 and 6, respectively; (jj) 39 and 113, respectively; (kk) 39 and 115, respectively; (ll) 39 and 116, respectively; (mm) 39 and 117, respectively; (nn) 39 and 119, respectively; (oo) 39 and 120, respectively; (pp) 39 and 121, respectively; (qq) 39 and 123, respectively; (rr) 39 and any one of 125-135, respectively; (ss) 39 and 137, respectively; (tt) 39 and 139 respectively; (uu) 39 and 6 respectively; (vv) 122 and 113, respectively; (ww) 122 and 115, respectively; (xx) 122 and 116, respectively; (yy) 122 and 117, respectively; (zz) 122 and 119, respectively; (aaa) 122 and 120, respectively; (bbb) 122 and 121, respectively; (ccc) 122 and 123, respectively; (ddd) 122 and any one of 125-135, respectively; (eee) 122 and 137, respectively; (fff) 122 and 139 respectively; (ggg) 122 and 6, respectively; (hhh) 124 and 113, respectively; (xv) 124 and 115, respectively; (xvi) 124 and 116, respectively; (xvii) 124 and 117, respectively; (xviii) 124 and 119, respectively; (iii) 124 and 120, respectively; (jjj) 124 and 121, respectively; (kkk) 124 and 123, respectively; (lll) 124 and any one of 125-135, respectively; (mmm) 124 and 137, respectively; (nnn) 124 and 139 respectively; (ooo) 124 and 6, respectively; (ppp) 136 and 113, respectively; (qqq) 136 and 115, respectively; (rrr) 136 and 116, respectively; (sss) 136 and 117, respectively; (ttt) 136 and 119, respectively; (uuu) 136 and 120, respectively; (vvv) 136 and 121, respectively; (www) 136 and 123, respectively; (xxx) 136 and any one of 125-135, respectively; (yyy) 136 and 137, respectively; (zzz) 136 and 139 respectively; (aaaa) 136 and 6 respectively; (bbbb) 138 and 113, respectively; (cccc) 138 and 115, respectively; (dddd) 138 and 116, respectively; (eeee) 138 and 117, respectively; (ffff) 138 and 119, respectively; (gggg) 138 and 120, respectively; (hhhh) 138 and 121, respectively; (iiii) 138 and 123, respectively; (jjjj) 138 and any one of 125-135, respectively; (kkkk) 138 and 137, respectively; (llll) 138 and 139 respectively; (mmmm) 138 and 6, respectively; (nnnn) 1 or 114 and 106, respectively; (oooo) 1 or 114 and 107, respectively; (pppp) 150 and 151, respectively; (qqqq) 150 and 153, respectively; (rrrr) 152 and 151, respectively; or (ssss) 152 and 153, respectively.

202. A method of treating a disease or condition in a subject, the method comprising administering to the subject an effective amount of the host cell of claim 199.

203. A method of treating a disease or condition in a subject, the method comprising administering to the subject an effective amount of the host cell of claim 200.

204. A method of treating a disease or condition in a subject, the method comprising administering to the subject an effective amount of the host cell of claim 201.


SEQUENCE LISTING (197 sequences)

SEQ ID NO: 1 - protein, 235 aa, Homo sapiens
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 2 - protein, 161 aa, Artificial Sequence
  Synthetic sequence
SQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACD

SEQ ID NO: 3 - protein, 21 aa, Artificial Sequence
  Synthetic sequence
IYIWAPLAGTCGVLLLSLVIT

SEQ ID NO: 4 - protein, 32 aa, Artificial Sequence
  Synthetic sequence
LYCNHRNRRRVCKCPRPVVKSGDKPSLSARYV

SEQ ID NO: 5 - protein, 182 aa, Artificial Sequence
  Synthetic sequence
SQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVIT

SEQ ID NO: 6 - protein, 210 aa, Homo sapiens
MRPRLWLLLAAQLTVLHGNSVLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRRRARLRFMKQFYK

SEQ ID NO: 7 - protein, 149 aa, Artificial Sequence
  Synthetic sequence
LQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSP

SEQ ID NO: 8 - protein, 21 aa, Artificial Sequence
  Synthetic sequence
ITLGLLVAGVLVLLVSLGVAI

SEQ ID NO: 9 - protein, 19 aa, Artificial Sequence
  Synthetic sequence
HLCCRRRRARLRFMKQFYK

SEQ ID NO: 10 - protein, 6 aa, Artificial Sequence
  Synthetic sequence
HLCCRR

SEQ ID NO: 11 - protein, 221 aa, Homo sapiens
MRPRLWLLLAAQLTVLHGNSVLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRRRARLRFMKQLRLHPLEKCSRMDY

SEQ ID NO: 12 - protein, 213 aa, Homo sapiens
MRPRLWLLLAAQLTVLHGNSVLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGRRRRARLRFMKQPQGEGISGTFVPQCLHGYYSNTTTSQKLLNPWILKT

SEQ ID NO: 13 - protein, 246 aa, Homo sapiens
MRPRLWLLLAAQLTVLHGNSVLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRRRARLRFMKQKFNIVCLKISGFTTCCCFQILQISREYGFGVLLQKDIGQ

SEQ ID NO: 14 - protein, 243 aa, Homo sapiens
MRPRLWLLLAAQLTVLHGNSVLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRRRARLRFMKQPQGEGISGTFVPQCLHGYYSNTTTSQKLLNPWILKT

SEQ ID NO: 15 - protein, 180 aa, Homo sapiens
MRPRLWLLLAAQLTVLHGNSVLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGRRRRARLRFMKQFYK

SEQ ID NO: 16 - protein, 206 aa, Homo sapiens
MRPRLWLLLAAQLTVLHGNSVLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKDFTNKQRIGFWCPATKRHRSVMSTMWKNERRDTFNPGEFNGC

SEQ ID NO: 17 - protein, 198 aa, Homo sapiens
MRPRLWLLLAAQLTVLHGNSVLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGLKGKVYQEPLSPNACMDTTAILQPHRSCLTHGS

SEQ ID NO: 18 - protein, 220 aa, Homo sapiens
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 19 - protein, 41 aa, Artificial Sequence
  Synthetic sequence
RSKRSRLLHSDYMNMTPRRPGPTRKHYQPYAPPRDFAAYRS

SEQ ID NO: 20 - protein, 41 aa, Artificial Sequence
  Synthetic sequence
RSKRSRGGHSDYMNMTPRRPGPTRKHYQPYAPPRDFAAYRS

SEQ ID NO: 21 - protein, 255 aa, Homo sapiens
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 22 - protein, 42 aa, Artificial Sequence
  Synthetic sequence
KRGRKKLLYIFKQPFMRPVQTTQEEDGCSCRFPEEEEGGCEL

SEQ ID NO: 23 - protein, 258 aa, homo sapiens
MCVGARRLGRGPCAALLLLGLGLSTVTGLHCVGDTYPSNDRCCHECRPGNGMVSRCSRSQNTVCRPCGPGFYNDVVSSKPKPCTWCNLRSGSERKQLCTATQDTVCRCRAGTQPLDSYKPGVDCAPCPPGHFSPGDNQACKPWTNCTLAGKHTLQPASNSDAICEDRDPPATQPQETQGPPARPITVQPTEAWPRTSQGPSTRPVEVPGGRAVAAILGLGLVLGLLGPLAILLALYLLRRDQRLPPDAHKPPGGGSFR

SEQ ID NO: 24 - protein, 42 aa, Artificial Sequence
  Synthetic sequence
ALYLLRRDQRLPPDAHKPPGGGSFRTPIQEEQADAHSTLAKI

SEQ ID NO: 25 - protein, 199 aa, Homo sapiens
MKSGLWYFFLFCLRIKVLTGEINGSANYEMFIFHNGGVQILCKYPDIVQQFKMQLLKGGQILCDLTKTKGSGNTVSIKSLKFCHSQLSNNSVSFFLYNLDHSHANYYFCNLSIFDPPPFKVTLTGGYLHIYESQLCCQLKFWLPIGCAAFVVVCILGCILICWLTKKKYSSSVHDPNGEYMFMRAVNTAKKSRLTDVTL

SEQ ID NO: 26 - protein, 38 aa, Artificial Sequence
  Synthetic sequence
CWLTKKKYSSSVHDPNGEYMFMRAVNTAKKSRLTDVTL

SEQ ID NO: 27 - DNA, 705 bp, Artificial Sequence
  Synthetic sequence
atggctctgcctgtgacagctctgctgctgcctctggctctgcttctgcatgccgctagacccagccagttcagagtgtcccctctggacagaacctggaacctgggcgagacagtggaactgaagtgccaggtgctgctgagcaatcctaccagcggctgcagctggctgtttcagcctagaggtgctgccgcctctcctacctttctgctgtacctgagccagaacaagcccaaggccgccgaaggactggacacccagagattcagcggcaagagactgggcgacaccttcgtgctgaccctgagcgacttcagaagagagaacgagggctactacttctgcagcgccctgagcaacagcatcatgtacttcagccacttcgtgcccgtgtttctgcccgccaagcctacaacaacccctgctcctagacctcctacaccagctcctacaatcgccagccagcctctgtctctgaggccagaagcttgtagacctgctgctggcggagccgtgcatacaagaggactggatttcgcctgcgacatctacatctgggcccctctggctggaacatgtggcgtgctgctgctgtccctggtcatcaccctgtactgcaaccaccggaacaggcggagagtgtgcaagtgccctagacctgtggtcaagagcggcgacaagcctagcctgagcgccagatatgtt

SEQ ID NO: 28 - DNA, 633 bp, Artificial Sequence
  Synthetic sequence
atgaggcctagactgtggctgctgctggctgctcagctgacagtgctgcacggcaattctgtcctgcagcagacccctgcctacatcaaggtgcagaccaacaagatggtcatgctgagctgcgaggccaagatcagcctgagcaacatgcggatctactggctgcggcagagacaggcccctagctctgatagccaccacgagtttctggccctgtgggattctgccaagggcaccattcacggcgaggaagtggaacaagagaagatcgccgtgttccgggacgccagcagattcatcctgaacctgaccagcgtgaagcccgaggacagcggcatctatttctgcatgatcgtgggcagccccgagctgacatttggcaagggaacacagctgagcgtggtggacttcctgcctactacagcccagcctaccaagaagtctaccctgaagaaaagagtgtgcagactgcccagacctgagacacagaaaggccctctgtgcagccctatcacactgggattgctggtggctggcgtgctggtgctgctggtttctctgggagttgccatccacctgtgctgtagacggcggagagcccggctgcggttcatgaagcagttctacaagtga

SEQ ID NO: 29 - DNA, 573 bp, Artificial Sequence
  Synthetic sequence
atgaggcctagactgtggctgctgctggctgctcagctgacagtgctgcacggcaattctgtcctgcagcagacccctgcctacatcaaggtgcagaccaacaagatggtcatgctgagctgcgaggccaagatcagcctgagcaacatgcggatctactggctgcggcagagacaggcccctagctctgatagccaccacgagtttctggccctgtgggattctgccaagggcaccattcacggcgaggaagtggaacaagagaagatcgccgtgttccgggacgccagcagattcatcctgaacctgaccagcgtgaagcccgaggacagcggcatctatttctgcatgatcgtgggcagccccgagctgacatttggcaagggaacacagctgagcgtggtggacttcctgcctactacagcccagcctaccaagaagtctaccctgaagaaaagagtgtgcagactgcccagacctgagacacagaaaggccctctgtgcagccctatcacactgggattgctggtggctggcgtgctggtgctgctggtttctctgggagttgccatc

SEQ ID NO: 30 - DNA, 591 bp, Artificial Sequence
  Synthetic sequence
atgaggcctagactgtggctgctgctggctgctcagctgacagtgctgcacggcaattctgtcctgcagcagacccctgcctacatcaaggtgcagaccaacaagatggtcatgctgagctgcgaggccaagatcagcctgagcaacatgcggatctactggctgcggcagagacaggcccctagctctgatagccaccacgagtttctggccctgtgggattctgccaagggcaccattcacggcgaggaagtggaacaagagaagatcgccgtgttccgggacgccagcagattcatcctgaacctgaccagcgtgaagcccgaggacagcggcatctatttctgcatgatcgtgggcagccccgagctgacatttggcaagggaacacagctgagcgtggtggacttcctgcctactacagcccagcctaccaagaagtctaccctgaagaaaagagtgtgcagactgcccagacctgagacacagaaaggccctctgtgcagccctatcacactgggattgctggtggctggcgtgctggtgctgctggtttctctgggagttgccatccacctgtgctgcagaaga

SEQ ID NO: 31 - DNA, 126 bp, Artificial Sequence
  Synthetic sequence
cggagcaagagaagcagactgctgcacagcgactacatgaacatgacccctagacggcccggacctaccagaaagcactaccagccttacgctcctcctagagacttcgccgcctacagatcttga

SEQ ID NO: 32 - DNA, 126 bp, Artificial Sequence
  Synthetic sequence
cgcagcaagcggagcagaggcggccacagcgactacatgaacatgacccctagacggcctggccccaccagaaagcactaccagccctacgcccctccccgggactttgccgcctacagaagctga

SEQ ID NO: 33 - DNA, 129 bp, Artificial Sequence
  Synthetic sequence
aagcggggcagaaagaagctgctgtacatcttcaagcagcccttcatgcggcccgtgcagaccacacaagaggaagatggctgctcctgcagattccccgaggaagaagaaggcggctgcgaactttga

SEQ ID NO: 34 - DNA, 117 bp, Artificial Sequence
  Synthetic sequence
tgctggctgaccaagaaaaagtacagcagcagcgtgcacgaccccaacggcgagtacatgttcatgagagccgtgaacaccgccaagaagtccagactgaccgacgtgacactgtga

SEQ ID NO: 35 - DNA, 129 bp, Artificial Sequence
  Synthetic sequence
gccctgtatctgctgagaagggaccagagactgcctcctgacgctcacaaacctccaggcggcggaagcttcagaacccctatccaagaggaacaggctgacgcccacagcaccctggccaaaatttga

SEQ ID NO: 36 - protein, 216 aa, Artificial Sequence
  Synthetic sequence
QQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRRSKRSRLLHSDYMNMTPRRPGPTRKHYQPYAPPRDFAAYRS

SEQ ID NO: 37 - protein, 216 aa, Artificial Sequence
  Synthetic sequence
QQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRRSKRSRGGHSDYMNMTPRRPGPTRKHYQPYAPPRDFAAYRS

SEQ ID NO: 38 - protein, 210 aa, Artificial Sequence
  Synthetic sequence
QQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIRSKRSRGGHSDYMNMTPRRPGPTRKHYQPYAPPRDFAAYRS

SEQ ID NO: 39 - protein, 223 aa, Artificial Sequence
  Synthetic sequence
SQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITRSKRSRGGHSDYMNMTPRRPGPTRKHYQPYAPPRDFAAYRS

SEQ ID NO: 40 - protein, 217 aa, Artificial Sequence
  Synthetic sequence
QQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRKRGRKKLLYIFKQPFMRPVQTTQEEDGCSCRFPEEEEGGCEL

SEQ ID NO: 41 - protein, 213 aa, Artificial Sequence
  Synthetic sequence
QQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRCWLTKKKYSSSVHDPNGEYMFMRAVNTAKKSRLTDVTL

SEQ ID NO: 42 - protein, 217 aa, Artificial Sequence
  Synthetic sequence
QQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRALYLLRRDQRLPPDAHKPPGGGSFRTPIQEEQADAHSTLAKI

SEQ ID NO: 43 - DNA, 717 bp, Artificial Sequence
  Synthetic sequence
atgaggcctagactgtggctgctgctggctgctcagctgacagtgctgcacggcaattctgtcctgcagcagacccctgcctacatcaaggtgcagaccaacaagatggtcatgctgagctgcgaggccaagatcagcctgagcaacatgcggatctactggctgcggcagagacaggcccctagctctgatagccaccacgagtttctggccctgtgggattctgccaagggcaccattcacggcgaggaagtggaacaagagaagatcgccgtgttccgggacgccagcagattcatcctgaacctgaccagcgtgaagcccgaggacagcggcatctatttctgcatgatcgtgggcagccccgagctgacatttggcaagggaacacagctgagcgtggtggacttcctgcctactacagcccagcctaccaagaagtctaccctgaagaaaagagtgtgcagactgcccagacctgagacacagaaaggccctctgtgcagccctatcacactgggattgctggtggctggcgtgctggtgctgctggtttctctgggagttgccatccacctgtgctgcagaagacggagcaagagaagcagactgctgcacagcgactacatgaacatgacccctagacggcccggacctaccagaaagcactaccagccttacgctcctcctagagacttcgccgcctacagatcttga

SEQ ID NO: 44 - DNA, 699 bp, Artificial Sequence
  Synthetic sequence
atgaggcctagactgtggctgctgctggctgctcagctgacagtgctgcacggcaattctgtcctgcagcagacccctgcctacatcaaggtgcagaccaacaagatggtcatgctgagctgcgaggccaagatcagcctgagcaacatgcggatctactggctgcggcagagacaggcccctagctctgatagccaccacgagtttctggccctgtgggattctgccaagggcaccattcacggcgaggaagtggaacaagagaagatcgccgtgttccgggacgccagcagattcatcctgaacctgaccagcgtgaagcccgaggacagcggcatctatttctgcatgatcgtgggcagccccgagctgacatttggcaagggaacacagctgagcgtggtggacttcctgcctactacagcccagcctaccaagaagtctaccctgaagaaaagagtgtgcagactgcccagacctgagacacagaaaggccctctgtgcagccctatcacactgggattgctggtggctggcgtgctggtgctgctggtttctctgggagttgccatccgcagcaagcggagcagaggcggccacagcgactacatgaacatgacccctagacggcctggccccaccagaaagcactaccagccctacgcccctccccgggactttgccgcctacagaagctga

SEQ ID NO: 45 - DNA, 699 bp, Artificial Sequence; identical to SEQ ID NO: 44
  Synthetic sequence

SEQ ID NO: 46 - DNA, 733 bp, Artificial Sequence
  Synthetic sequence
atggctctgcctgtgacagctctgctgctgcctctggctctgcttctgcatgccgctagacccagccagttcagagtgtcccctctggacagaacctggaacctgggcgagacagtggaactgaagtgccaggtgctgctgagcaatcctaccagcggctgcagctggctgtttcagcctagaggtgctgccgcctctcctacctttctgctgtacctgagccagaacaagcccaaggccgccgaaggactggacacccagagattcagcggcaagagactgggcgacaccttcgtgctgaccctgagcgacttcagaagagagaacgagggctactacttctgcagcgccctgagcaacagcatcatgtacttcagccacttcgtgcccgtgtttctgcccgccaagcctacaacaacccctgctcctagacctcctacaccagctcctacaatcgccagccagcctctgtctctgaggccagaagcttgtagacctgctgctggcggagccgtgcatacaagaggactggatttcgcctgcgacatctacatctgggcccctctggctggaacatgtggcgtgctgctgctgtccctggtcatcacccgcagcaagcggagcagaggcggccacagcgactacatgaacatgacccctagacggcctggccccaccagaaagcactaccagccctacgcccctccccgggactttgccgcctacagaagcg

SEQ ID NO: 47 - DNA, 720 bp, Artificial Sequence
  Synthetic sequence
atgaggcctagactgtggctgctgctggctgctcagctgacagtgctgcacggcaattctgtcctgcagcagacccctgcctacatcaaggtgcagaccaacaagatggtcatgctgagctgcgaggccaagatcagcctgagcaacatgcggatctactggctgcggcagagacaggcccctagctctgatagccaccacgagtttctggccctgtgggattctgccaagggcaccattcacggcgaggaagtggaacaagagaagatcgccgtgttccgggacgccagcagattcatcctgaacctgaccagcgtgaagcccgaggacagcggcatctatttctgcatgatcgtgggcagccccgagctgacatttggcaagggaacacagctgagcgtggtggacttcctgcctactacagcccagcctaccaagaagtctaccctgaagaaaagagtgtgcagactgcccagacctgagacacagaaaggccctctgtgcagccctatcacactgggattgctggtggctggcgtgctggtgctgctggtttctctgggagttgccatccacctgtgctgcagaagaaagcggggcagaaagaagctgctgtacatcttcaagcagcccttcatgcggcccgtgcagaccacacaagaggaagatggctgctcctgcagattccccgaggaagaagaaggcggctgcgaactttga

SEQ ID NO: 48 - DNA, 708 bp, Artificial Sequence
  Synthetic sequence
atgaggcctagactgtggctgctgctggctgctcagctgacagtgctgcacggcaattctgtcctgcagcagacccctgcctacatcaaggtgcagaccaacaagatggtcatgctgagctgcgaggccaagatcagcctgagcaacatgcggatctactggctgcggcagagacaggcccctagctctgatagccaccacgagtttctggccctgtgggattctgccaagggcaccattcacggcgaggaagtggaacaagagaagatcgccgtgttccgggacgccagcagattcatcctgaacctgaccagcgtgaagcccgaggacagcggcatctatttctgcatgatcgtgggcagccccgagctgacatttggcaagggaacacagctgagcgtggtggacttcctgcctactacagcccagcctaccaagaagtctaccctgaagaaaagagtgtgcagactgcccagacctgagacacagaaaggccctctgtgcagccctatcacactgggattgctggtggctggcgtgctggtgctgctggtttctctgggagttgccatccacctgtgctgcagaagatgctggctgaccaagaaaaagtacagcagcagcgtgcacgaccccaacggcgagtacatgttcatgagagccgtgaacaccgccaagaagtccagactgaccgacgtgacactgtga

SEQ ID NO: 49 - DNA, 720 bp, Artificial Sequence
  Synthetic sequence
atgaggcctagactgtggctgctgctggctgctcagctgacagtgctgcacggcaattctgtcctgcagcagacccctgcctacatcaaggtgcagaccaacaagatggtcatgctgagctgcgaggccaagatcagcctgagcaacatgcggatctactggctgcggcagagacaggcccctagctctgatagccaccacgagtttctggccctgtgggattctgccaagggcaccattcacggcgaggaagtggaacaagagaagatcgccgtgttccgggacgccagcagattcatcctgaacctgaccagcgtgaagcccgaggacagcggcatctatttctgcatgatcgtgggcagccccgagctgacatttggcaagggaacacagctgagcgtggtggacttcctgcctactacagcccagcctaccaagaagtctaccctgaagaaaagagtgtgcagactgcccagacctgagacacagaaaggccctctgtgcagccctatcacactgggattgctggtggctggcgtgctggtgctgctggtttctctgggagttgccatccacctgtgctgcagaagagccctgtatctgctgagaagggaccagagactgcctcctgacgctcacaaacctccaggcggcggaagcttcagaacccctatccaagaggaacaggctgacgcccacagcaccctggccaaaatttga

SEQ ID NO: 50 - DNA, 66 bp, Artificial Sequence
  Synthetic sequence
ggttccggagccacgaacttctctctgttaaagcaagcaggagacgtggaagaaaaccccggtccc

SEQ ID NO: 51 - DNA, 66 bp, Artificial Sequence
  Synthetic sequence
ggaagcggagctactaacttcagcctgctgaagcaggctggagacgtggaggagaaccctggacct

SEQ ID NO: 52 - DNA, 63 bp, Artificial Sequence
  Synthetic sequence
ggaagcggagagggcagaggaagtctgctaacatgcggtgacgtcgaggagaatcctggacct

SEQ ID NO: 53 - DNA, 69 bp, Artificial Sequence
  Synthetic sequence
ggaagcggacagtgtactaattatgctctcttgaaattggctggagatgttgagagcaaccctggacct

SEQ ID NO: 54 - DNA, 75 bp, Artificial Sequence
  Synthetic sequence
ggaagcggagtgaaacagactttgaattttgaccttctcaagttggcgggagacgtggagtccaaccctggacct

SEQ ID NO: 55 - protein, 22 aa, Artificial Sequence
  Synthetic sequence
GSGATNFSLLKQAGDVEENPGP

SEQ ID NO: 56 - protein, 24 aa, Artificial Sequence
  Synthetic sequence
LEGGGEGRGSLLTCGDVEENPGPR

SEQ ID NO: 57 - protein, 20 aa, Artificial Sequence
  Synthetic sequence
QCTNYALLKLAGDVESNPGP

SEQ ID NO: 58 - protein, 25 aa, Artificial Sequence
  Synthetic sequence
GSGVKQTLNFDLLKLAGDVESNPGP

SEQ ID NO: 59 - DNA, 1488 bp, Artificial Sequence
  Synthetic sequence
atggctctgcctgtgacagctctgctgctgcctctggctctgcttctgcatgccgctagacccagccagttcagagtgtcccctctggacagaacctggaacctgggcgagacagtggaactgaagtgccaggtgctgctgagcaatcctaccagcggctgcagctggctgtttcagcctagaggtgctgccgcctctcctacctttctgctgtacctgagccagaacaagcccaaggccgccgaaggactggacacccagagattcagcggcaagagactgggcgacaccttcgtgctgaccctgagcgacttcagaagagagaacgagggctactacttctgcagcgccctgagcaacagcatcatgtacttcagccacttcgtgcccgtgtttctgcccgccaagcctacaacaacccctgctcctagacctcctacaccagctcctacaatcgccagccagcctctgtctctgaggccagaagcttgtagacctgctgctggcggagccgtgcatacaagaggactggatttcgcctgcgacatctacatctgggcccctctggctggaacatgtggcgtgctgctgctgtccctggtcatcaccctgtactgcaaccaccggaacaggcggagagtgtgcaagtgccctagacctgtggtcaagagcggcgacaagcctagcctgagcgccagatatgttggttccggagccacgaacttctctctgttaaagcaagcaggagacgtggaagaaaaccccggtcccatgaggcctagactgtggctgctgctggctgctcagctgacagtgctgcacggcaattctgtcctgcagcagacccctgcctacatcaaggtgcagaccaacaagatggtcatgctgagctgcgaggccaagatcagcctgagcaacatgcggatctactggctgcggcagagacaggcccctagctctgatagccaccacgagtttctggccctgtgggattctgccaagggcaccattcacggcgaggaagtggaacaagagaagatcgccgtgttccgggacgccagcagattcatcctgaacctgaccagcgtgaagcccgaggacagcggcatctatttctgcatgatcgtgggcagccccgagctgacatttggcaagggaacacagctgagcgtggtggacttcctgcctactacagcccagcctaccaagaagtctaccctgaagaaaagagtgtgcagactgcccagacctgagacacagaaaggccctctgtgcagccctatcacactgggattgctggtggctggcgtgctggtgctgctggtttctctgggagttgccatccacctgtgctgcagaagacggagcaagagaagcagactgctgcacagcgactacatgaacatgacccctagacggcccggacctaccagaaagcactaccagccttacgctcctcctagagacttcgccgcctacagatcttga

SEQ ID NO: 60 - DNA, 1488 bp, Artificial Sequence
  Synthetic sequence
atggctctgcctgtgacagctctgctgctgcctctggctctgcttctgcatgccgctagacccagccagttcagagtgtcccctctggacagaacctggaacctgggcgagacagtggaactgaagtgccaggtgctgctgagcaatcctaccagcggctgcagctggctgtttcagcctagaggtgctgccgcctctcctacctttctgctgtacctgagccagaacaagcccaaggccgccgaaggactggacacccagagattcagcggcaagagactgggcgacaccttcgtgctgaccctgagcgacttcagaagagagaacgagggctactacttctgcagcgccctgagcaacagcatcatgtacttcagccacttcgtgcccgtgtttctgcccgccaagcctacaacaacccctgctcctagacctcctacaccagctcctacaatcgccagccagcctctgtctctgaggccagaagcttgtagacctgctgctggcggagccgtgcatacaagaggactggatttcgcctgcgacatctacatctgggcccctctggctggaacatgtggcgtgctgctgctgtccctggtcatcaccctgtactgcaaccaccggaacaggcggagagtgtgcaagtgccctagacctgtggtcaagagcggcgacaagcctagcctgagcgccagatatgttggttccggagccacgaacttctctctgttaaagcaagcaggagacgtggaagaaaaccccggtcccatgaggcctagactgtggctgctgctggctgctcagctgacagtgctgcacggcaattctgtcctgcagcagacccctgcctacatcaaggtgcagaccaacaagatggtcatgctgagctgcgaggccaagatcagcctgagcaacatgcggatctactggctgcggcagagacaggcccctagctctgatagccaccacgagtttctggccctgtgggattctgccaagggcaccattcacggcgaggaagtggaacaagagaagatcgccgtgttccgggacgccagcagattcatcctgaacctgaccagcgtgaagcccgaggacagcggcatctatttctgcatgatcgtgggcagccccgagctgacatttggcaagggaacacagctgagcgtggtggacttcctgcctactacagcccagcctaccaagaagtctaccctgaagaaaagagtgtgcagactgcccagacctgagacacagaaaggccctctgtgcagccctatcacactgggattgctggtggctggcgtgctggtgctgctggtttctctgggagttgccatccacctgtgctgcagaagacgcagcaagcggagcagaggcggccacagcgactacatgaacatgacccctagacggcctggccccaccagaaagcactaccagccctacgcccctccccgggactttgccgcctacagaagctga

SEQ ID NO: 61 - DNA, 1470 bp, Artificial Sequence
  Synthetic sequence
atggctctgcctgtgacagctctgctgctgcctctggctctgcttctgcatgccgctagacccagccagttcagagtgtcccctctggacagaacctggaacctgggcgagacagtggaactgaagtgccaggtgctgctgagcaatcctaccagcggctgcagctggctgtttcagcctagaggtgctgccgcctctcctacctttctgctgtacctgagccagaacaagcccaaggccgccgaaggactggacacccagagattcagcggcaagagactgggcgacaccttcgtgctgaccctgagcgacttcagaagagagaacgagggctactacttctgcagcgccctgagcaacagcatcatgtacttcagccacttcgtgcccgtgtttctgcccgccaagcctacaacaacccctgctcctagacctcctacaccagctcctacaatcgccagccagcctctgtctctgaggccagaagcttgtagacctgctgctggcggagccgtgcatacaagaggactggatttcgcctgcgacatctacatctgggcccctctggctggaacatgtggcgtgctgctgctgtccctggtcatcaccctgtactgcaaccaccggaacaggcggagagtgtgcaagtgccctagacctgtggtcaagagcggcgacaagcctagcctgagcgccagatatgttggttccggagccacgaacttctctctgttaaagcaagcaggagacgtggaagaaaaccccggtcccatgaggcctagactgtggctgctgctggctgctcagctgacagtgctgcacggcaattctgtcctgcagcagacccctgcctacatcaaggtgcagaccaacaagatggtcatgctgagctgcgaggccaagatcagcctgagcaacatgcggatctactggctgcggcagagacaggcccctagctctgatagccaccacgagtttctggccctgtgggattctgccaagggcaccattcacggcgaggaagtggaacaagagaagatcgccgtgttccgggacgccagcagattcatcctgaacctgaccagcgtgaagcccgaggacagcggcatctatttctgcatgatcgtgggcagccccgagctgacatttggcaagggaacacagctgagcgtggtggacttcctgcctactacagcccagcctaccaagaagtctaccctgaagaaaagagtgtgcagactgcccagacctgagacacagaaaggccctctgtgcagccctatcacactgggattgctggtggctggcgtgctggtgctgctggtttctctgggagttgccatccgcagcaagcggagcagaggcggccacagcgactacatgaacatgacccctagacggcctggccccaccagaaagcactaccagccctacgcccctccccgggactttgccgcctacagaagctga

SEQ ID NO: 62 - DNA, 1497 bp, Artificial Sequence
  Synthetic sequence
atggctctgcctgtgacagctctgctgctgcctctggctctgcttctgcatgccgctagacccagccagttcagagtgtcccctctggacagaacctggaacctgggcgagacagtggaactgaagtgccaggtgctgctgagcaatcctaccagcggctgcagctggctgtttcagcctagaggtgctgccgcctctcctacctttctgctgtacctgagccagaacaagcccaaggccgccgaaggactggacacccagagattcagcggcaagagactgggcgacaccttcgtgctgaccctgagcgacttcagaagagagaacgagggctactacttctgcagcgccctgagcaacagcatcatgtacttcagccacttcgtgcccgtgtttctgcccgccaagcctacaacaacccctgctcctagacctcctacaccagctcctacaatcgccagccagcctctgtctctgaggccagaagcttgtagacctgctgctggcggagccgtgcatacaagaggactggatttcgcctgcgacatctacatctgggcccctctggctggaacatgtggcgtgctgctgctgtccctggtcatcacccgcagcaagcggagcagaggcggccacagcgactacatgaacatgacccctagacggcctggccccaccagaaagcactaccagccctacgcccctccccgggactttgccgcctacagaagcggttccggagccacgaacttctctctgttaaagcaagcaggagacgtggaagaaaaccccggtcccatgaggcctagactgtggctgctgctggctgctcagctgacagtgctgcacggcaattctgtcctgcagcagacccctgcctacatcaaggtgcagaccaacaagatggtcatgctgagctgcgaggccaagatcagcctgagcaacatgcggatctactggctgcggcagagacaggcccctagctctgatagccaccacgagtttctggccctgtgggattctgccaagggcaccattcacggcgaggaagtggaacaagagaagatcgccgtgttccgggacgccagcagattcatcctgaacctgaccagcgtgaagcccgaggacagcggcatctatttctgcatgatcgtgggcagccccgagctgacatttggcaagggaacacagctgagcgtggtggacttcctgcctactacagcccagcctaccaagaagtctaccctgaagaaaagagtgtgcagactgcccagacctgagacacagaaaggccctctgtgcagccctatcacactgggattgctggtggctggcgtgctggtgctgctggtttctctgggagttgccatccgcagcaagcggagcagaggcggccacagcgactacatgaacatgacccctagacggcctggccccaccagaaagcactaccagccctacgcccctccccgggactttgccgcctacagaagctga

SEQ ID NO: 63 - DNA, 1431 bp, Artificial Sequence
  Synthetic sequence
atggctctgcctgtgacagctctgctgctgcctctggctctgcttctgcatgccgctagacccagccagttcagagtgtcccctctggacagaacctggaacctgggcgagacagtggaactgaagtgccaggtgctgctgagcaatcctaccagcggctgcagctggctgtttcagcctagaggtgctgccgcctctcctacctttctgctgtacctgagccagaacaagcccaaggccgccgaaggactggacacccagagattcagcggcaagagactgggcgacaccttcgtgctgaccctgagcgacttcagaagagagaacgagggctactacttctgcagcgccctgagcaacagcatcatgtacttcagccacttcgtgcccgtgtttctgcccgccaagcctacaacaacccctgctcctagacctcctacaccagctcctacaatcgccagccagcctctgtctctgaggccagaagcttgtagacctgctgctggcggagccgtgcatacaagaggactggatttcgcctgcgacatctacatctgggcccctctggctggaacatgtggcgtgctgctgctgtccctggtcatcacccgcagcaagcggagcagaggcggccacagcgactacatgaacatgacccctagacggcctggccccaccagaaagcactaccagccctacgcccctccccgggactttgccgcctacagaagcggttccggagccacgaacttctctctgttaaagcaagcaggagacgtggaagaaaaccccggtcccatgaggcctagactgtggctgctgctggctgctcagctgacagtgctgcacggcaattctgtcctgcagcagacccctgcctacatcaaggtgcagaccaacaagatggtcatgctgagctgcgaggccaagatcagcctgagcaacatgcggatctactggctgcggcagagacaggcccctagctctgatagccaccacgagtttctggccctgtgggattctgccaagggcaccattcacggcgaggaagtggaacaagagaagatcgccgtgttccgggacgccagcagattcatcctgaacctgaccagcgtgaagcccgaggacagcggcatctatttctgcatgatcgtgggcagccccgagctgacatttggcaagggaacacagctgagcgtggtggacttcctgcctactacagcccagcctaccaagaagtctaccctgaagaaaagagtgtgcagactgcccagacctgagacacagaaaggccctctgtgcagccctatcacactgggattgctggtggctggcgtgctggtgctgctggtttctctgggagttgccatccacctgtgctgtagacggcggagagcccggctgcggttcatgaagcagttctacaagtga

SEQ ID NO: 64 - DNA, 1491 bp, Artificial Sequence
  Synthetic sequence
atggctctgcctgtgacagctctgctgctgcctctggctctgcttctgcatgccgctagacccagccagttcagagtgtcccctctggacagaacctggaacctgggcgagacagtggaactgaagtgccaggtgctgctgagcaatcctaccagcggctgcagctggctgtttcagcctagaggtgctgccgcctctcctacctttctgctgtacctgagccagaacaagcccaaggccgccgaaggactggacacccagagattcagcggcaagagactgggcgacaccttcgtgctgaccctgagcgacttcagaagagagaacgagggctactacttctgcagcgccctgagcaacagcatcatgtacttcagccacttcgtgcccgtgtttctgcccgccaagcctacaacaacccctgctcctagacctcctacaccagctcctacaatcgccagccagcctctgtctctgaggccagaagcttgtagacctgctgctggcggagccgtgcatacaagaggactggatttcgcctgcgacatctacatctgggcccctctggctggaacatgtggcgtgctgctgctgtccctggtcatcaccctgtactgcaaccaccggaacaggcggagagtgtgcaagtgccctagacctgtggtcaagagcggcgacaagcctagcctgagcgccagatatgttggttccggagccacgaacttctctctgttaaagcaagcaggagacgtggaagaaaaccccggtcccatgaggcctagactgtggctgctgctggctgctcagctgacagtgctgcacggcaattctgtcctgcagcagacccctgcctacatcaaggtgcagaccaacaagatggtcatgctgagctgcgaggccaagatcagcctgagcaacatgcggatctactggctgcggcagagacaggcccctagctctgatagccaccacgagtttctggccctgtgggattctgccaagggcaccattcacggcgaggaagtggaacaagagaagatcgccgtgttccgggacgccagcagattcatcctgaacctgaccagcgtgaagcccgaggacagcggcatctatttctgcatgatcgtgggcagccccgagctgacatttggcaagggaacacagctgagcgtggtggacttcctgcctactacagcccagcctaccaagaagtctaccctgaagaaaagagtgtgcagactgcccagacctgagacacagaaaggccctctgtgcagccctatcacactgggattgctggtggctggcgtgctggtgctgctggtttctctgggagttgccatccacctgtgctgcagaagaaagcggggcagaaagaagctgctgtacatcttcaagcagcccttcatgcggcccgtgcagaccacacaagaggaagatggctgctcctgcagattccccgaggaagaagaaggcggctgcgaactttga

SEQ ID NO: 65 - DNA, 1479 bp, Artificial Sequence
  Synthetic sequence
atggctctgcctgtgacagctctgctgctgcctctggctctgcttctgcatgccgctagacccagccagttcagagtgtcccctctggacagaacctggaacctgggcgagacagtggaactgaagtgccaggtgctgctgagcaatcctaccagcggctgcagctggctgtttcagcctagaggtgctgccgcctctcctacctttctgctgtacctgagccagaacaagcccaaggccgccgaaggactggacacccagagattcagcggcaagagactgggcgacaccttcgtgctgaccctgagcgacttcagaagagagaacgagggctactacttctgcagcgccctgagcaacagcatcatgtacttcagccacttcgtgcccgtgtttctgcccgccaagcctacaacaacccctgctcctagacctcctacaccagctcctacaatcgccagccagcctctgtctctgaggccagaagcttgtagacctgctgctggcggagccgtgcatacaagaggactggatttcgcctgcgacatctacatctgggcccctctggctggaacatgtggcgtgctgctgctgtccctggtcatcaccctgtactgcaaccaccggaacaggcggagagtgtgcaagtgccctagacctgtggtcaagagcggcgacaagcctagcctgagcgccagatatgttggttccggagccacgaacttctctctgttaaagcaagcaggagacgtggaagaaaaccccggtcccatgaggcctagactgtggctgctgctggctgctcagctgacagtgctgcacggcaattctgtcctgcagcagacccctgcctacatcaaggtgcagaccaacaagatggtcatgctgagctgcgaggccaagatcagcctgagcaacatgcggatctactggctgcggcagagacaggcccctagctctgatagccaccacgagtttctggccctgtgggattctgccaagggcaccattcacggcgaggaagtggaacaagagaagatcgccgtgttccgggacgccagcagattcatcctgaacctgaccagcgtgaagcccgaggacagcggcatctatttctgcatgatcgtgggcagccccgagctgacatttggcaagggaacacagctgagcgtggtggacttcctgcctactacagcccagcctaccaagaagtctaccctgaagaaaagagtgtgcagactgcccagacctgagacacagaaaggccctctgtgcagccctatcacactgggattgctggtggctggcgtgctggtgctgctggtttctctgggagttgccatccacctgtgctgcagaagatgctggctgaccaagaaaaagtacagcagcagcgtgcacgaccccaacggcgagtacatgttcatgagagccgtgaacaccgccaagaagtccagactgaccgacgtgacactgtga

SEQ ID NO: 66 - DNA, 1491 bp, Artificial Sequence
  Synthetic sequence
atggctctgcctgtgacagctctgctgctgcctctggctctgcttctgcatgccgctagacccagccagttcagagtgtcccctctggacagaacctggaacctgggcgagacagtggaactgaagtgccaggtgctgctgagcaatcctaccagcggctgcagctggctgtttcagcctagaggtgctgccgcctctcctacctttctgctgtacctgagccagaacaagcccaaggccgccgaaggactggacacccagagattcagcggcaagagactgggcgacaccttcgtgctgaccctgagcgacttcagaagagagaacgagggctactacttctgcagcgccctgagcaacagcatcatgtacttcagccacttcgtgcccgtgtttctgcccgccaagcctacaacaacccctgctcctagacctcctacaccagctcctacaatcgccagccagcctctgtctctgaggccagaagcttgtagacctgctgctggcggagccgtgcatacaagaggactggatttcgcctgcgacatctacatctgggcccctctggctggaacatgtggcgtgctgctgctgtccctggtcatcaccctgtactgcaaccaccggaacaggcggagagtgtgcaagtgccctagacctgtggtcaagagcggcgacaagcctagcctgagcgccagatatgttggttccggagccacgaacttctctctgttaaagcaagcaggagacgtggaagaaaaccccggtcccatgaggcctagactgtggctgctgctggctgctcagctgacagtgctgcacggcaattctgtcctgcagcagacccctgcctacatcaaggtgcagaccaacaagatggtcatgctgagctgcgaggccaagatcagcctgagcaacatgcggatctactggctgcggcagagacaggcccctagctctgatagccaccacgagtttctggccctgtgggattctgccaagggcaccattcacggcgaggaagtggaacaagagaagatcgccgtgttccgggacgccagcagattcatcctgaacctgaccagcgtgaagcccgaggacagcggcatctatttctgcatgatcgtgggcagccccgagctgacatttggcaagggaacacagctgagcgtggtggacttcctgcctactacagcccagcctaccaagaagtctaccctgaagaaaagagtgtgcagactgcccagacctgagacacagaaaggccctctgtgcagccctatcacactgggattgctggtggctggcgtgctggtgctgctggtttctctgggagttgccatccacctgtgctgcagaagagccctgtatctgctgagaagggaccagagactgcctcctgacgctcacaaacctccaggcggcggaagcttcagaacccctatccaagaggaacaggctgacgcccacagcaccctggccaaaatttga

SEQ ID NO: 67 - protein, 114 aa, Artificial Sequence
  Synthetic sequence
SQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPA

SEQ ID NO: 68 - protein, 111 aa, Artificial Sequence
  Synthetic sequence
LQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQL

SEQ ID NO: 69 - protein, 104 aa, Artificial Sequence
  Synthetic sequence
DGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMD

SEQ ID NO: 70 - protein, 26 aa, Artificial Sequence
  Synthetic sequence
VMSVATIVIVDICITGGLLLLVYYWS

SEQ ID NO: 71 - protein, 84 aa, Artificial Sequence [sequence omitted]

SEQ ID NO: 72 - protein, 21 aa, Artificial Sequence
  Synthetic sequence
GIIVTDVIATLLLALGVFCFA

SEQ ID NO: 73 - protein, 94 aa, Artificial Sequence
  Synthetic sequence
QSIKGNHLVKVYDYQEDGSVLLTCDAEAKNITWFKDGKMIGFLTEDKKKWNLGSNAKDPRGMYQCKGSQNKSKPLQVYYRMCQNCIELNAATIS

SEQ ID NO: 74 - protein, 21 aa, Artificial Sequence
  Synthetic sequence
GFLFAEIVSIFVLAVGVYFIA

SEQ ID NO: 75 - protein, 9 aa, Artificial Sequence
  Synthetic sequence
QSFGLLDPK

SEQ ID NO: 76 - protein, 21 aa, Artificial Sequence
  Synthetic sequence
LCYLLDGILFIYGVILTALFL

SEQ ID NO: 77 - protein, 113 aa, Artificial Sequence
  Synthetic sequence
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 78 - protein, 135 aa, Artificial Sequence
  Synthetic sequence
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPA

SEQ ID NO: 79 - protein, 114 aa, Artificial Sequence; identical to SEQ ID NO: 67
  Synthetic sequence

SEQ ID NO: 80 - protein, 27 aa, Artificial Sequence
  Synthetic sequence
FWVLVVVGGVLACYSLLVTVAFIIFWV

SEQ ID NO: 81 - protein, 41 aa, Artificial Sequence
  Synthetic sequence
RSKRSRGGHSDAMNMTARRAGPTRKHYQAYAAPRDFAAYRS

SEQ ID NO: 82 - protein, 170 aa, Artificial Sequence
  Synthetic sequence
MRPRLWLLLAAQLTVLHGNSVLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSP

SEQ ID NO: 83 - protein, 47 aa, Artificial Sequence
  Synthetic sequence
HLCCRRRSKRSRGGHSDYMNMTPRRPGPTRKHYQPYAPPRDFAAYRS

SEQ ID NO: 84 - protein, 47 aa, Artificial Sequence
  Synthetic sequence
HLCCRRRSKRSRLLHSDYMNMTPRRPGPTRKHYQPYAPPRDFAAYRS

SEQ ID NO: 85 - protein, 48 aa, Artificial Sequence
  Synthetic sequence
HLCCRRKRGRKKLLYIFKQPFMRPVQTTQEEDGCSCRFPEEEEGGCEL

SEQ ID NO: 86 - protein, 44 aa, Artificial Sequence
  Synthetic sequence
HLCCRRCWLTKKKYSSSVHDPNGEYMFMRAVNTAKKSRLTDVTL

SEQ ID NO: 87 - protein, 48 aa, Artificial Sequence
  Synthetic sequence
HLCCRRALYLLRRDQRLPPDAHKPPGGGSFRTPIQEEQADAHSTLAKI

SEQ ID NO: 88 - protein, 64 aa, Artificial Sequence
  Synthetic sequence
HLCCRRQLGLHIWQLRSQCMWPRETQLLLEVPPSTEDARSCQFPEEERGERSAEEKGRLGDLWV

SEQ ID NO: 89 - protein, 469 aa, Artificial Sequence
  Synthetic sequence
HLCCRRRRARLRFMKQFYKPLQDNLVIALHSYEPSHDGDLGFEKGEQLRILEQSGEWWKAQSLTTGQEGFIPFNFVAKANSLEPEPWFFKNLSRKDAERQLLAPGNTHGSFLIRESESTAGSFSLSVRDFDQNQGEVVKHYKIRNLDNGGFYISPRITFPGLHELVRHYTNASDGLCTRLSRPCQTQKPQKPWWEDEWEVPRETLKLVERLGAGQFGEVWMGYYNGHTKVAVKSLKQGSMSPDAFLAEANLMKQLQHQRLVRLYAVVTQEPIYIITEYMENGSLVDFLKTPSGIKLTINKLLDMAAQIAEGMAFIEERNYIHRDLRAANILVSDTLSCKIADFGLARLIEDNEYTAREGAKFPIKWTAPEAINYGTFTIKSDVWSFGILLTEIVTHGRIPYPGMTNPEVIQNLERGYRMVRPDNCPEELYQLMRLCWKERPEDRPTFDYLRSVLEDFFTATEGQYQPQP

SEQ ID NO: 90 - protein, 159 aa, Artificial Sequence
  Synthetic sequence
SQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPADFLPTTAQPTKKSTLKKRVCRLPRPETQKGPLCCPSPLFPGPSKP

SEQ ID NO: 91 - protein, 180 aa, Artificial Sequence
  Synthetic sequence
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPADFLPTTAQPTKKSTLKKRVCRLPRPETQKGPLCCPSPLFPGPSKP

SEQ ID NO: 92 - protein, 147 aa, Artificial Sequence
  Synthetic sequence
SQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPADFLPTTAQPTKKSTLKKRVCRLPRPETQKGPLC

SEQ ID NO: 93 - protein, 168 aa, Artificial Sequence
  Synthetic sequence
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPADFLPTTAQPTKKSTLKKRVCRLPRPETQKGPLC

SEQ ID NO: 94 - protein, 148 aa, Artificial Sequence
  Synthetic sequence
SQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPADFLPTTAQPTKKSTLKKRVCRLPRPETQKGPLCD

SEQ ID NO: 95 - protein, 169 aa, Artificial Sequence
  Synthetic sequence
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPADFLPTTAQPTKKSTLKKRVCRLPRPETQKGPLCD

SEQ ID NO: 96 - protein, 163 aa, Artificial Sequence
  Synthetic sequence
MLRLLLALNLFPSIQVTGIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVKGKHLCPSPLFPGPSKP

SEQ ID NO: 97 - protein, 73 aa, Artificial Sequence
  Synthetic sequence
RSKRSRGGHSDYMNMTPRRPGPTRKHYQPYAPPRDFAAYRSLYCNHRNRRRVCKCPRPVVKSGDKPSLSARYV

SEQ ID NO: 98 - protein, 148 aa, Artificial Sequence
  Synthetic sequence
QVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSN

SEQ ID NO: 99 - protein, 173 aa, Artificial Sequence
  Synthetic sequence
MLGIWTLLPLVLTSVARLSSKSVNAQVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSN

SEQ ID NO: 100 - protein, 17 aa, Artificial Sequence
  Synthetic sequence
LGWLCLLLLPIPLIVWV

SEQ ID NO: 101 - protein, 1 aa, Artificial Sequence
  Synthetic sequence
K

SEQ ID NO: 102 - protein, 450 aa, Artificial Sequence
  Synthetic sequence
PLQDNLVIALHSYEPSHDGDLGFEKGEQLRILEQSGEWWKAQSLTTGQEGFIPFNFVAKANSLEPEPWFFKNLSRKDAERQLLAPGNTHGSFLIRESESTAGSFSLSVRDFDQNQGEVVKHYKIRNLDNGGFYISPRITFPGLHELVRHYTNASDGLCTRLSRPCQTQKPQKPWWEDEWEVPRETLKLVERLGAGQFGEVWMGYYNGHTKVAVKSLKQGSMSPDAFLAEANLMKQLQHQRLVRLYAVVTQEPIYIITEYMENGSLVDFLKTPSGIKLTINKLLDMAAQIAEGMAFIEERNYIHRDLRAANILVSDTLSCKIADFGLARLIEDNEYTAREGAKFPIKWTAPEAINYGTFTIKSDVWSFGILLTEIVTHGRIPYPGMTNPEVIQNLERGYRMVRPDNCPEELYQLMRLCWKERPEDRPTFDYLRSVLEDFFTATEGQYQPQP

SEQ ID NO: 103 - protein, 438 aa, Artificial Sequence
  Synthetic sequence
GSGGGTGGGSGGSGGGTGGGSGMASSSGSSPRPAPDENEFPFGCPPTVCQDPKEPRALCCAGCLSENPRNGEDQICPKCRGEDLQSISPGSRLRTQEKAHPEVAEAGIGCPFAGVGCSFKGSPQSVQEHEVTSQTSHLNLLLGFMKQWKARLGCGLESGPMALEQNLSDLQLQAAVEVAGDLEVDCYRAPCSESQEELALQHFMKEKLLAELEGKLRVFENIVAVLNKEVEASHLALATSIHQSQLDRERILSLEQRVVELQQTLAQKDQALGKLEQSLRLMEEASFDGTFLWKITNVTRRCHESACGRTVSLFSPAFYTAKYGYKLCLRLYLNGDGTGKRTHLSLFIVIMRGEYDALLPWPFRNKVTFMLLDQNNREHAIDAFRPDLSSASFQRPQSETNVASGCPLFFPLSKLQSPKHAYVKDDTMFLKCIVETST

SEQ ID NO: 104 - protein, 147 aa, Artificial Sequence
  Synthetic sequence
FLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPCPSPLFPGPSKP

SEQ ID NO: 105 - protein, 170 aa, Artificial Sequence
  Synthetic sequence
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPCPSPLFPGPSKP

SEQ ID NO: 106 - protein, 360 aa, Artificial Sequence
  Synthetic sequence
MNPTDIADTTLDESIYSNYYLYESIPKPCTKEGIKAFGELFLPPLYSLVFVFGLLGNSVVVLVLFKYKRLRSMTDVYLLNLAISDLLFVFSLPFWGYYAADQWVFGLGLCKMISWMYLVGFYSGIFFVMLMSIDRYLAIVHAVFSLRARTLTYGVITSLATWSVAVFASLPGFLFSTCYTERNHTYCKTKYSLNSTTWKVLSSLEINILGLVIPLGIMLFCYSMIIRTLQHCKNEKKNKAVKMIFAVVVLFLGFWTPYNIVLFLETLVELEVLQDCTFERYLDYAIQATETLAFVHCCLNPIIYFFLGEKFRKYILQLFKTCRGLFVLCQYCGLLQIYSADTPSSSYTQSTMDHDLHDAL

SEQ ID NO: 107 - protein, 360 aa, Artificial Sequence
  Synthetic sequence
MLSTSRSRFIRNTNESGEEVTTFFDYDYGAPCHKFDVKQIGAQLLPPLYSLVFIFGFVGNMLVVLILINCKKLKCLTDIYLLNLAISDLLFLITLPLWAHSAANEWVFGNAMCKLFTGLYHIGYFGGIFFIILLTIDRYLAIVHAVFALKARTVTFGVVTSVITWLVAVFASVPGIIFTKCQKEDSVYVCGPYFPRGWNNFHTIMRNILGLVLPLLIMVICYSGILKTLLRCRNEKKRHRAVRVIFTIMIVYFLFWTPYNIVILLNTFQEFFGLSNCESTSQLDQATQVTETLGMTHCCINPIIYAFVGEKFRRYLSVFFRKHITKRFCKQCPVFYRETVDGVTSTNTPSTGEQEVSAGL

SEQ ID NO: 108 - protein, 41 aa, Artificial Sequence
  Synthetic sequence
RSKRSRGGHSDAMNMTARRAGPTRKHFQAFAAPRDFAAFRS

SEQ ID NO: 109 - DNA, 546 bp, Artificial Sequence
  Synthetic sequence
atggctctgcctgtgacagctctgctgctgcctctggctctgcttctgcatgccgctagacccagccagttcagagtgtcccctctggacagaacctggaacctgggcgagacagtggaactgaagtgccaggtgctgctgagcaatcctaccagcggctgcagctggctgtttcagcctagaggtgctgccgcctctcctacctttctgctgtacctgagccagaacaagcccaaggccgccgaaggactggacacccagagattcagcggcaagagactgggcgacaccttcgtgctgaccctgagcgacttcagaagagagaacgagggctactacttctgcagcgccctgagcaacagcatcatgtacttcagccacttcgtgcccgtgtttctgcccgccaagcctacaacaacccctgctcctagacctcctacaccagctcctacaatcgccagccagcctctgtctctgaggccagaagcttgtagacctgctgctggcggagccgtgcatacaagaggactggatttcgcctgcgac

SEQ ID NO: 110 - DNA, 546 bp, Artificial Sequence
  Synthetic sequence
atggcattgcctgttacagctctgctgctgcccctggctctgcttctgcatgctgctagacccagccagttcagagtgtcccctctggacagaacctggaacctgggcgagacagtggaactgaagtgccaggtgctgctgagcaatcctaccagcggctgcagctggctgtttcagcctagaggtgctgccgcctctcctacctttctgctgtacctgagccagaacaagcccaaggccgccgaaggactggacacccagagattcagcggcaagagactgggcgacaccttcgtgctgaccctgagcgacttcagaagagagaacgagggctactacttctgcagcgccctgagcaacagcatcatgtacttcagccacttcgtgcccgtgtttctgcccgccaagcctacaacaacccctgctcctagacctcctacaccagctcctacaatcgccagccagcctctgtctctgaggccagaagcttgtagacctgctgctggcggagccgtgcatacaagaggactggatttcgcctgcgac

SEQ ID NO: 111 - DNA, 546 bp, Artificial Sequence
  Synthetic sequence
atggctcttccggtaactgcacttttactgccacttgctctgctgttacacgctgctagaccatctcagttccgtgtttctccactggatagaacttggaatctgggcgagactgttgagctgaaatgccaggttctgctgtctaacccgacttctggttgttcttggctgtttcaaccaagaggtgctgctgcttctccgacttttctgctgtacctgtctcagaacaaaccgaaagctgctgaaggtctggatactcagcgtttctccggtaaacgtctgggtgacacctttgttctgactctgtctgatttccgtcgcgaaaacgaaggctactacttctgctctgctctgtccaactccatcatgtacttctcccactttgtgccggtttttctgccggctaaaccgactactactccagctccaagaccaccaactccagctccaactattgcttctcaaccactgtctttacgtccagaagcttgtcgtccagctgctggtggtgctgttcatactcgtggtttagatttcgcttgtgac

SEQ ID NO: 112 - DNA, 405 bp, Artificial Sequence
  Synthetic sequence
atggctcttcctgtaaccgcacttctgcttcctcttgctctgctgcttcatgctgctagacctagccagttcagagtgtctccactggatagaacctggaatctgggcgaaacagtggagctgaagtgtcaggtgctgctgagcaatcctacatctggctgttcttggctgttccagcctagaggagctgctgcttctcctacctttctgctgtatctgagccagaataagcctaaagccgccgaaggactggatacccagaggtttagcggcaagagattgggcgatacctttgttctgacactgagcgacttccggagagagaatgagggctactacttctgttctgccctgagcaacagcatcatgtacttcagccacttcgtgcccgtgtttctgcctgcc

SEQ ID NO: 113 - protein, 238 aa, Artificial Sequence
  Synthetic sequence
MRPRLWLLLAAQLTVLHGNSVLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRRSKRSRGGHSDYMNMTPRRPGPTRKHYQPYAPPRDFAAYRS

SEQ ID NO: 114 - protein, 214 aa, Artificial Sequence
  Synthetic sequence
SQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 115 - protein, 217 aa, Artificial Sequence
  Synthetic sequence
LQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRRSKRSRGGHSDYMNMTPRRPGPTRKHYQPYAPPRDFAAYRS

SEQ ID NO: 116 - protein, 232 aa, Artificial Sequence
  Synthetic sequence
MRPRLWLLLAAQLTVLHGNSVLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIRSKRSRGGHSDYMNMTPRRPGPTRKHYQPYAPPRDFAAYRS

SEQ ID NO: 117 - protein, 211 aa, Artificial Sequence
  Synthetic sequence
LQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIRSKRSRGGHSDYMNMTPRRPGPTRKHYQPYAPPRDFAAYRS

SEQ ID NO: 118 - protein, 244 aa, Artificial Sequence
  Synthetic sequence
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITRSKRSRGGHSDYMNMTPRRPGPTRKHYQPYAPPRDFAAYRS

SEQ ID NO: 119 - protein, 189 aa, Artificial Sequence
  Synthetic sequence
LQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRRRARLRFMKQFYK

SEQ ID NO: 120 - protein, 238 aa, Artificial Sequence
  Synthetic sequence
MRPRLWLLLAAQLTVLHGNSVLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRRSKRSRGGHSDAMNMTARRAGPTRKHYQAYAAPRDFAAYRS

SEQ ID NO: 121 - protein, 217 aa, Artificial Sequence
  Synthetic sequence
LQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRRSKRSRGGHSDAMNMTARRAGPTRKHYQAYAAPRDFAAYRS

SEQ ID NO: 122 - protein, 244 aa, Artificial Sequence
  Synthetic sequence
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITRSKRSRGGHSDAMNMTARRAGPTRKHYQAYAAPRDFAAYRS

SEQ ID NO: 123 - protein, 232 aa, Artificial Sequence
  Synthetic sequence
MRPRLWLLLAAQLTVLHGNSVLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIRSKRSRGGHSDAMNMTARRAGPTRKHYQAYAAPRDFAAYRS

SEQ ID NO: 124 - protein, 223 aa, Artificial Sequence
  Synthetic sequence
SQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITRSKRSRGGHSDAMNMTARRAGPTRKHYQAYAAPRDFAAYRS

SEQ ID NO: 125 - protein, 211 aa, Artificial Sequence
  Synthetic sequence
LQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIRSKRSRGGHSDAMNMTARRAGPTRKHYQAYAAPRDFAAYRS

SEQ ID NO: 126 - protein, 238 aa, Artificial Sequence
  Synthetic sequence
MRPRLWLLLAAQLTVLHGNSVLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRRSKRSRLLHSDYMNMTPRRPGPTRKHYQPYAPPRDFAAYRS

SEQ ID NO: 127 - protein, 217 aa, Artificial Sequence
  Synthetic sequence
LQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRRSKRSRLLHSDYMNMTPRRPGPTRKHYQPYAPPRDFAAYRS

SEQ ID NO: 128 - protein, 239 aa, Artificial Sequence
  Synthetic sequence
MRPRLWLLLAAQLTVLHGNSVLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRKRGRKKLLYIFKQPFMRPVQTTQEEDGCSCRFPEEEEGGCEL

SEQ ID NO: 129 - protein, 218 aa, Artificial Sequence
  Synthetic sequence
LQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRKRGRKKLLYIFKQPFMRPVQTTQEEDGCSCRFPEEEEGGCEL

SEQ ID NO: 130 - protein, 235 aa, Artificial Sequence
  Synthetic sequence
MRPRLWLLLAAQLTVLHGNSVLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRCWLTKKKYSSSVHDPNGEYMFMRAVNTAKKSRLTDVTL

SEQ ID NO: 131 - protein, 214 aa, Artificial Sequence
  Synthetic sequence
LQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRCWLTKKKYSSSVHDPNGEYMFMRAVNTAKKSRLTDVTL

SEQ ID NO: 132 - protein, 239 aa, Artificial Sequence
  Synthetic sequence
MRPRLWLLLAAQLTVLHGNSVLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRALYLLRRDQRLPPDAHKPPGGGSFRTPIQEEQADAHSTLAKI

SEQ ID NO: 133 - protein, 218 aa, Artificial Sequence
  Synthetic sequence
LQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRALYLLRRDQRLPPDAHKPPGGGSFRTPIQEEQADAHSTLAKI

SEQ ID NO: 134 - protein, 255 aa, Artificial Sequence
  Synthetic sequence
MRPRLWLLLAAQLTVLHGNSVLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRQLGLHIWQLRSQCMWPRETQLLLEVPPSTEDARSCQFPEEERGERSAEEKGRLGDLWV

SEQ ID NO: 135 - protein, 234 aa, Artificial Sequence
  Synthetic sequence
LQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRQLGLHIWQLRSQCMWPRETQLLLEVPPSTEDARSCQFPEEERGERSAEEKGRLGDLWV

SEQ ID NO: 136 - protein, 203 aa, Artificial Sequence
  Synthetic sequence
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAFWVLVVVGGVLACYSLLVTVAFIIFWVRSKRSRGGHSDYMNMTPRRPGPTRKHYQPYAPPRDFAAYRS

SEQ ID NO: 137 - protein, 660 aa, Artificial Sequence
  Synthetic sequence
MRPRLWLLLAAQLTVLHGNSVLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRRRARLRFMKQFYKPLQDNLVIALHSYEPSHDGDLGFEKGEQLRILEQSGEWWKAQSLTTGQEGFIPFNFVAKANSLEPEPWFFKNLSRKDAERQLLAPGNTHGSFLIRESESTAGSFSLSVRDFDQNQGEVVKHYKIRNLDNGGFYISPRITFPGLHELVRHYTNASDGLCTRLSRPCQTQKPQKPWWEDEWEVPRETLKLVERLGAGQFGEVWMGYYNGHTKVAVKSLKQGSMSPDAFLAEANLMKQLQHQRLVRLYAVVTQEPIYIITEYMENGSLVDFLKTPSGIKLTINKLLDMAAQIAEGMAFIEERNYIHRDLRAANILVSDTLSCKIADFGLARLIEDNEYTAREGAKFPIKWTAPEAINYGTFTIKSDVWSFGILLTEIVTHGRIPYPGMTNPEVIQNLERGYRMVRPDNCPEELYQLMRLCWKERPEDRPTFDYLRSVLEDFFTATEGQYQPQP

SEQ ID NO: 138 - protein, 182 aa, Artificial Sequence
  Synthetic sequence
SQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAFWVLVVVGGVLACYSLLVTVAFIIFWVRSKRSRGGHSDYMNMTPRRPGPTRKHYQPYAPPRDFAAYRS

SEQ ID NO: 139 - protein, 639 aa, Artificial Sequence
  Synthetic sequence
LQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRRRARLRFMKQFYKPLQDNLVIALHSYEPSHDGDLGFEKGEQLRILEQSGEWWKAQSLTTGQEGFIPFNFVAKANSLEPEPWFFKNLSRKDAERQLLAPGNTHGSFLIRESESTAGSFSLSVRDFDQNQGEVVKHYKIRNLDNGGFYISPRITFPGLHELVRHYTNASDGLCTRLSRPCQTQKPQKPWWEDEWEVPRETLKLVERLGAGQFGEVWMGYYNGHTKVAVKSLKQGSMSPDAFLAEANLMKQLQHQRLVRLYAVVTQEPIYIITEYMENGSLVDFLKTPSGIKLTINKLLDMAAQIAEGMAFIEERNYIHRDLRAANILVSDTLSCKIADFGLARLIEDNEYTAREGAKFPIKWTAPEAINYGTFTIKSDVWSFGILLTEIVTHGRIPYPGMTNPEVIQNLERGYRMVRPDNCPEELYQLMRLCWKERPEDRPTFDYLRSVLEDFFTATEGQYQPQP

SEQ ID NO: 140 - protein, 248 aa, Artificial Sequence
  Synthetic sequence
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPADFLPTTAQPTKKSTLKKRVCRLPRPETQKGPLCCPSPLFPGPSKPFWVLVVVGGVLACYSLLVTVAFIIFWVRSKRSRGGHSDYMNMTPRRPGPTRKHYQPYAPPRDFAAYRS

SEQ ID NO: 141 - protein, 227 aa, Artificial Sequence
  Synthetic sequence
SQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPADFLPTTAQPTKKSTLKKRVCRLPRPETQKGPLCCPSPLFPGPSKPFWVLVVVGGVLACYSLLVTVAFIIFWVRSKRSRGGHSDYMNMTPRRPGPTRKHYQPYAPPRDFAAYRS

SEQ ID NO: 142 - protein, 236 aa, Artificial Sequence
  Synthetic sequence
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPADFLPTTAQPTKKSTLKKRVCRLPRPETQKGPLCFWVLVVVGGVLACYSLLVTVAFIIFWVRSKRSRGGHSDYMNMTPRRPGPTRKHYQPYAPPRDFAAYRS

SEQ ID NO: 143 - protein, 215 aa, Artificial Sequence
  Synthetic sequence
SQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPADFLPTTAQPTKKSTLKKRVCRLPRPETQKGPLCFWVLVVVGGVLACYSLLVTVAFIIFWVRSKRSRGGHSDYMNMTPRRPGPTRKHYQPYAPPRDFAAYRS

SEQ ID NO: 144 - protein, 268 aa, Artificial Sequence
  Synthetic sequence
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPADFLPTTAQPTKKSTLKKRVCRLPRPETQKGPLCFWVLVVVGGVLACYSLLVTVAFIIFWVRSKRSRGGHSDYMNMTPRRPGPTRKHYQPYAPPRDFAAYRSLYCNHRNRRRVCKCPRPVVKSGDKPSLSARYV

SEQ ID NO: 145 - protein, 247 aa, Artificial Sequence
  Synthetic sequence
SQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPADFLPTTAQPTKKSTLKKRVCRLPRPETQKGPLCFWVLVVVGGVLACYSLLVTVAFIIFWVRSKRSRGGHSDYMNMTPRRPGPTRKHYQPYAPPRDFAAYRSLYCNHRNRRRVCKCPRPVVKSGDKPSLSARYV

SEQ ID NO: 146 - protein, 222 aa, Artificial Sequence
  Synthetic sequence
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPADFLPTTAQPTKKSTLKKRVCRLPRPETQKGPLCDIYIWAPLAGTCGVLLLSLVITLYCNHRNRRRVCKCPRPVVKSGDKPSLSARYV

SEQ ID NO: 147 - protein, 201 aa, Artificial Sequence
  Synthetic sequence
SQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPADFLPTTAQPTKKSTLKKRVCRLPRPETQKGPLCDIYIWAPLAGTCGVLLLSLVITLYCNHRNRRRVCKCPRPVVKSGDKPSLSARYV

SEQ ID NO: 148 - protein, 231 aa, Artificial Sequence
  Synthetic sequence
MLRLLLALNLFPSIQVTGIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 149 - protein, 213 aa, Artificial Sequence
  Synthetic sequence
IPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 150 - protein, 244 aa, Artificial Sequence
  Synthetic sequence
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITRSKRSRGGHSDAMNMTARRAGPTRKHFQAFAAPRDFAAFRS

SEQ ID NO: 151 - protein, 232 aa, Artificial Sequence
  Synthetic sequence
MRPRLWLLLAAQLTVLHGNSVLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIRSKRSRGGHSDAMNMTARRAGPTRKHFQAFAAPRDFAAFRS

SEQ ID NO: 152 - protein, 223 aa, Artificial Sequence
  Synthetic sequence
SQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITRSKRSRGGHSDAMNMTARRAGPTRKHFQAFAAPRDFAAFRS

SEQ ID NO: 153 - protein, 211 aa, Artificial Sequence
  Synthetic sequence
LQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIRSKRSRGGHSDAMNMTARRAGPTRKHFQAFAAPRDFAAFRS

SEQ ID NO: 154 - protein, 191 aa, Artificial Sequence
  Synthetic sequence
MLGIWTLLPLVLTSVARLSSKSVNAQVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSNLGWLCLLLLPIPLIVWVK

SEQ ID NO: 155 - protein, 166 aa, Artificial Sequence
  Synthetic sequence
QVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSNLGWLCLLLLPIPLIVWVK

SEQ ID NO: 156 - protein, 640 aa, Artificial Sequence
  Synthetic sequence
MLGIWTLLPLVLTSVARLSSKSVNAQVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSNLGWLCLLLLPIPLIVWVPLQDNLVIALHSYEPSHDGDLGFEKGEQLRILEQSGEWWKAQSLTTGQEGFIPFNFVAKANSLEPEPWFFKNLSRKDAERQLLAPGNTHGSFLIRESESTAGSFSLSVRDFDQNQGEVVKHYKIRNLDNGGFYISPRITFPGLHELVRHYTNASDGLCTRLSRPCQTQKPQKPWWEDEWEVPRETLKLVERLGAGQFGEVWMGYYNGHTKVAVKSLKQGSMSPDAFLAEANLMKQLQHQRLVRLYAVVTQEPIYIITEYMENGSLVDFLKTPSGIKLTINKLLDMAAQIAEGMAFIEERNYIHRDLRAANILVSDTLSCKIADFGLARLIEDNEYTAREGAKFPIKWTAPEAINYGTFTIKSDVWSFGILLTEIVTHGRIPYPGMTNPEVIQNLERGYRMVRPDNCPEELYQLMRLCWKERPEDRPTFDYLRSVLEDFFTATEGQYQPQP

SEQ ID NO: 157 - protein, 615 aa, Artificial Sequence
  Synthetic sequence
QVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSNLGWLCLLLLPIPLIVWVPLQDNLVIALHSYEPSHDGDLGFEKGEQLRILEQSGEWWKAQSLTTGQEGFIPFNFVAKANSLEPEPWFFKNLSRKDAERQLLAPGNTHGSFLIRESESTAGSFSLSVRDFDQNQGEVVKHYKIRNLDNGGFYISPRITFPGLHELVRHYTNASDGLCTRLSRPCQTQKPQKPWWEDEWEVPRETLKLVERLGAGQFGEVWMGYYNGHTKVAVKSLKQGSMSPDAFLAEANLMKQLQHQRLVRLYAVVTQEPIYIITEYMENGSLVDFLKTPSGIKLTINKLLDMAAQIAEGMAFIEERNYIHRDLRAANILVSDTLSCKIADFGLARLIEDNEYTAREGAKFPIKWTAPEAINYGTFTIKSDVWSFGILLTEIVTHGRIPYPGMTNPEVIQNLERGYRMVRPDNCPEELYQLMRLCWKERPEDRPTFDYLRSVLEDFFTATEGQYQPQP

SEQ ID NO: 158 - protein, 222 aa, Artificial Sequence
  Synthetic sequence
MLGIWTLLPLVLTSVARLSSKSVNAQVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSNLGWLCLLLLPIPLIVWVLYCNHRNRRRVCKCPRPVVKSGDKPSLSARYV

SEQ ID NO: 159 - protein, 197 aa, Artificial Sequence
  Synthetic sequence
QVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSNLGWLCLLLLPIPLIVWVLYCNHRNRRRVCKCPRPVVKSGDKPSLSARYV

SEQ ID NO: 160 - protein, 628 aa, Artificial Sequence
  Synthetic sequence
MLGIWTLLPLVLTSVARLSSKSVNAQVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSNLGWLCLLLLPIPLIVWVGSGGGTGGGSGGSGGGTGGGSGMASSSGSSPRPAPDENEFPFGCPPTVCQDPKEPRALCCAGCLSENPRNGEDQICPKCRGEDLQSISPGSRLRTQEKAHPEVAEAGIGCPFAGVGCSFKGSPQSVQEHEVTSQTSHLNLLLGFMKQWKARLGCGLESGPMALEQNLSDLQLQAAVEVAGDLEVDCYRAPCSESQEELALQHFMKEKLLAELEGKLRVFENIVAVLNKEVEASHLALATSIHQSQLDRERILSLEQRVVELQQTLAQKDQALGKLEQSLRLMEEASFDGTFLWKITNVTRRCHESACGRTVSLFSPAFYTAKYGYKLCLRLYLNGDGTGKRTHLSLFIVIMRGEYDALLPWPFRNKVTFMLLDQNNREHAIDAFRPDLSSASFQRPQSETNVASGCPLFFPLSKLQSPKHAYVKDDTMFLKCIVETST

SEQ ID NO: 161 - protein, 603 aa, Artificial Sequence
  Synthetic sequence
QVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSNLGWLCLLLLPIPLIVWVGSGGGTGGGSGGSGGGTGGGSGMASSSGSSPRPAPDENEFPFGCPPTVCQDPKEPRALCCAGCLSENPRNGEDQICPKCRGEDLQSISPGSRLRTQEKAHPEVAEAGIGCPFAGVGCSFKGSPQSVQEHEVTSQTSHLNLLLGFMKQWKARLGCGLESGPMALEQNLSDLQLQAAVEVAGDLEVDCYRAPCSESQEELALQHFMKEKLLAELEGKLRVFENIVAVLNKEVEASHLALATSIHQSQLDRERILSLEQRVVELQQTLAQKDQALGKLEQSLRLMEEASFDGTFLWKITNVTRRCHESACGRTVSLFSPAFYTAKYGYKLCLRLYLNGDGTGKRTHLSLFIVIMRGEYDALLPWPFRNKVTFMLLDQNNREHAIDAFRPDLSSASFQRPQSETNVASGCPLFFPLSKLQSPKHAYVKDDTMFLKCIVETST

SEQ ID NO: 162 - protein, 238 aa, Artificial Sequence
  Synthetic sequence
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPCPSPLFPGPSKPFWVLVVVGGVLACYSLLVTVAFIIFWVRSKRSRGGHSDYMNMTPRRPGPTRKHYQPYAPPRDFAAYRS

SEQ ID NO: 163 - protein, 215 aa, Artificial Sequence
  Synthetic sequence
FLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPCPSPLFPGPSKPFWVLVVVGGVLACYSLLVTVAFIIFWVRSKRSRGGHSDYMNMTPRRPGPTRKHYQPYAPPRDFAAYRS

SEQ ID NO: 164 - protein, 204 aa, Artificial Sequence
  Synthetic sequence
MKWKALFTAAILQAQLPITEAQSFGLLDPKLCYLLDGILFIYGVILTALFLRSKRSRGGHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 165 - protein, 183 aa, Artificial Sequence
  Synthetic sequence
QSFGLLDPKLCYLLDGILFIYGVILTALFLRSKRSRGGHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 166 - protein, 204 aa, Artificial Sequence
  Synthetic sequence
MKWKALFTAAILQAQLPITEAQSFGLLDPKLCYLLDGILFIYGVILTALFLRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 167 - protein, 183 aa, Artificial Sequence
  Synthetic sequence
QSFGLLDPKLCYLLDGILFIYGVILTALFLRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 168 - protein, 21 aa, Artificial Sequence
  Synthetic sequence
MALPVTALLLPLALLLHAARP

SEQ ID NO: 169 - protein, 18 aa, Artificial Sequence
  Synthetic sequence
MLRLLLALNLFPSIQVTG

SEQ ID NO: 170 - protein, 25 aa, Artificial Sequence
  Synthetic sequence
MLGIWTLLPLVLTSVARLSSKSVNA

SEQ ID NO: 171 - protein, 23 aa, Artificial Sequence
  Synthetic sequence
MQIPQAPWPVVWAVLQLGWRPGW

SEQ ID NO: 172 - protein, 21 aa, Artificial Sequence
  Synthetic sequence
MKWKALFTAAILQAQLPITEA

SEQ ID NO: 173 - protein, 114 aa, Artificial Sequence; identical to SEQ ID NO: 67
  Synthetic sequence

SEQ ID NO: 174 - protein, 145 aa, Artificial Sequence
  Synthetic sequence
IPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVKGKHLCPSPLFPGPSKP

SEQ ID NO: 175 - protein, 450 aa, Artificial Sequence; identical to SEQ ID NO: 102
  Synthetic sequence

SEQ ID NO: 176 - protein, 438 aa, Artificial Sequence; identical to SEQ ID NO: 103
  Synthetic sequence Linker (G/S/T 22 amino acid linker)+ TRAF1 intracellullar sequence

SEQ ID NO: 177 - protein, 153 aa, Artificial Sequence
  Synthetic sequence CD28 intracellular sequence fused to CD3 gamma intracellular sequence
RSKRSRGGHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 178 - protein, 153 aa, Artificial Sequence
  Synthetic sequence 4-1BB intracellular sequence fused to CD3gamma intracellular sequence
RGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 179 - protein, 21 aa, Artificial sequence
  Synthetic sequence CD8beta signal peptide
MRPRLWLLLAAQLTVLHGNSV

SEQ ID NO: 180 - protein, 47 aa, Artificial sequence
  Synthetic sequence CD28 intracellular sequence with GG mutation and partial signalling mutation
HLCCRRRSKRSRGGHSDAMNMTARRAGPTRKHYQAYAAPRDFAAYRS

SEQ ID NO: 181 - protein, 416 aa, Artificial Sequence
  Synthetic sequence TRAF1 amino acid sequence
MASSSGSSPRPAPDENEFPFGCPPTVCQDPKEPRALCCAGCLSENPRNGEDQICPKCRGEDLQSISPGSRLRTQEKAHPEVAEAGIGCPFAGVGCSFKGSPQSVQEHEVTSQTSHLNLLLGFMKQWKARLGCGLESGPMALEQNLSDLQLQAAVEVAGDLEVDCYRAPCSESQEELALQHFMKEKLLAELEGKLRVFENIVAVLNKEVEASHLALATSIHQSQLDRERILSLEQRVVELQQTLAQKDQALGKLEQSLRLMEEASFDGTFLWKITNVTRRCHESACGRTVSLFSPAFYTAKYGYKLCLRLYLNGDGTGKRTHLSLFIVIMRGEYDALLPWPFRNKVTFMLLDQNNREHAIDAFRPDLSSASFQRPQSETNVASGCPLFFPLSKLQSPKHAYVKDDTMFLKCIVETST

SEQ ID NO: 182 - protein, 22 aa, Artificial Sequence
  Synthetic sequence linker
GSGGGTGGGSGGSGGGTGGGSG

SEQ ID NO: 183 - protein, 18 aa, Artificial Sequence
  Synthetic sequence Whitlow linker
GSTSGSGKPGSGEGSTKG

SEQ ID NO: 184 - protein, 15 aa, Artificial Sequence
  Synthetic sequence Linker
GGGGSGGGGSGGGGS

SEQ ID NO: 185 - protein, 31 aa, Artificial Sequence
  Synthetic sequence CD28 intracellular sequence with partial signaling mutation
DAMNMTARRAGPTRKHYQAYAAPRDFAAYRS

SEQ ID NO: 186 - protein, 31 aa, Artificial Sequence
  Synthetic sequence CD28 intracellular sequence with partial signaling mutation
DAMNMTARRAGPTRKHFQAFAAPRDFAAFRS

SEQ ID NO: 187 - protein, 7 aa, Artificial Sequence
  Synthetic sequence OX40 intacellular motif
GGSFRPI

SEQ ID NO: 188 - protein, 509 aa, Artificial Sequence
  Synthetic sequence Lck amino acid sequence
MGCGCSSHPEDDWMENIDVCENCHYPIVPLDGKGTLLIRNGSEVRDPLVTYEGSNPPASPLQDNLVIALHSYEPSHDGDLGFEKGEQLRILEQSGEWWKAQSLTTGQEGFIPFNFVAKANSLEPEPWFFKNLSRKDAERQLLAPGNTHGSFLIRESESTAGSFSLSVRDFDQNQGEVVKHYKIRNLDNGGFYISPRITFPGLHELVRHYTNASDGLCTRLSRPCQTQKPQKPWWEDEWEVPRETLKLVERLGAGQFGEVWMGYYNGHTKVAVKSLKQGSMSPDAFLAEANLMKQLQHQRLVRLYAVVTQEPIYIITEYMENGSLVDFLKTPSGIKLTINKLLDMAAQIAEGMAFIEERNYIHRDLRAANILVSDTLSCKIADFGLARLIEDNEYTAREGAKFPIKWTAPEAINYGTFTIKSDVWSFGILLTEIVTHGRIPYPGMTNPEVIQNLERGYRMVRPDNCPEELYQLMRLCWKERPEDRPTFDYLRSVLEDFFTATEGQYQPQP

SEQ ID NO: 189 - protein, 241 aa, Artificial Sequence
  Synthetic sequence GITR amino acid sequence
MAQHGAMGAFRALCGLALLCALSLGQRPTGGPGCGPGRLLLGTGTDARCCRVHTTRCCRDYPGEECCSEWDCMCVQPEFHCGDPCCTTCRHHPCPPGQGVQSQGKFSFGFQCIDCASGTFSGGHEGHCKPWTDCTQFGFLTVFPGNKTHNAVCVPGSPPAEPLGWLTVVLLAVAACVLLLTSAQLGLHIWQLRSQCMWPRETQLLLEVPPSTEDARSCQFPEEERGERSAEEKGRLGDLWV

SEQ ID NO: 190 - protein, 58 aa, Artificial Sequence
  Synthetic sequence GITR intracellular amino acid sequence
QLGLHIWQLRSQCMWPRETQLLLEVPPSTEDARSCQFPEEERGERSAEEKGRLGDLWV

SEQ ID NO: 191 - protein, 145 aa, Artificial Sequence
  Synthetic sequence Fas intracellular amino acid sequence
KRKEVQKTCRKHRKENQGSHESPTLNPETVAINLSDVDLSKYITTIAGVMTLSQVKGFVRKNGVNEAKIDEIKNDNVQDTAEQKVQLLRNWHQLHGKKEAYDTLIKDLKKANLCTLAEKIQTIILKDITSDSENSNFRNEIQSLV

SEQ ID NO: 192 - protein, 4 aa, Artificial Sequence
  Motif sequence
  VARIANT (1)...(4): Xaa = Any Amino Acid
PXXP

SEQ ID NO: 193 - protein, 4 aa, Artificial Sequence
  Motif sequence
  VARIANT (1)...(4): Xaa = Any Amino Acid
YXXM

SEQ ID NO: 194 - protein, 4 aa, Artificial Sequence
  Functional variant or portion of an ICOS costimulatory domain
YMFM

SEQ ID NO: 195 - protein, 9 aa, Artificial Sequence
  structure motif
LAGLIDADG

SEQ ID NO: 196 - protein, 6 aa, Artificial Sequence
  structure motif
GIYYIG

SEQ ID NO: 197 - protein, 5 aa, Artificial Sequence
  structure motif
  VARIANT 3: Xaa = D or E
  VARIANT 4: Xaa = Any Amino Acid
PDXXK